(19) United States
(12) Patent Application Publication
(10) Pub. No.: US 2026/0232823 A1
(43) Pub. Date: Aug. 13, 2026

(54) AZA-BENZAZEPINE IMMUNOCONJUGATES, AND USES THEREOF

(71) Applicant: BOLT BIOTHERAPEUTICS, INC., Redwood City, CA (US)

(72) Inventors: Romas KUDIRKA, Redwood City, CA (US); Matthew ZHOU, Redwood City, CA (US)

(73) Assignee: BOLT BIOTHERAPEUTICS, INC., Redwood City, CA (US)

(21) Appl. No.: 19/156,100

(22) PCT Filed: Feb. 13, 2024
(86) PCT No.: PCT/US2024/015579

§ 371 Date: Aug. 13, 2025

Related U.S. Application Data
(60) Provisional application No. 63/445,390, filed on Feb. 14, 2023.

(51) Int. Cl. A61K 47/68; A61P 35/00

(52) U.S. Cl. CPC A61K 47/6803; A61K 47/6849; A61K 47/6851; A61P 35/00

(57) ABSTRACT

The invention provides immunoconjugates of Formula I comprising an antibody linked by conjugation to one or more aza-benzazepine derivatives. The invention also provides aza-benzazepine derivative intermediate compositions comprising a reactive functional group. Such intermediate compositions are suitable substrates for formation of the immunoconjugates through a linker or linking moiety. The invention further provides methods of treating cancer with the immunoconjugates.

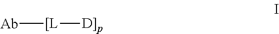

### CROSS REFERENCE TO RELATED APPLICATIONS

This non-provisional application claims the benefit of priority to U.S. Provisional Application No. 63/445,390, filed 14 Feb. 2023, which is incorporated by reference in its entirety.

### FIELD OF THE INVENTION

The invention relates generally to an immunoconjugate comprising an antibody conjugated to one or more aza-benzazepine molecules.

### BACKGROUND OF THE INVENTION

New compositions and methods for the delivery of antibodies and immune adjuvants are needed in order to reach inaccessible tumors and/or to expand treatment options for cancer patients and other subjects. The invention provides such compositions and methods.

### SUMMARY OF THE INVENTION

The invention is generally directed to an immunoconjugate comprising an antibody covalently attached by a linker to one or more aza-benzazepine TLR (toll-like receptor) agonist moieties having the formula:

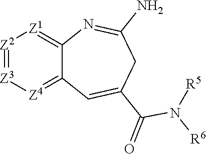

where one or two of Z¹, Z², Z³, and Z⁴ is N, and one of the substituents is attached to the linker. The various substituents are defined herein.

Another aspect of the invention is a method of preparing an immunoconjugate by conjugation of one or more aza-benzazepine-linker compounds with an antibody.

Another aspect of the invention is a pharmaceutical composition comprising a therapeutically effective amount of an immunoconjugate comprising an antibody covalently attached by a linker to one or more aza-benzazepine moieties, and one or more pharmaceutically acceptable diluent, vehicle, carrier or excipient.

Another aspect of the invention is an aza-benzazepine-linker compound.

Another aspect of the invention is a method for treating cancer comprising administering a therapeutically effective amount of an immunoconjugate comprising an antibody covalently attached to one or more aza-benzazepine moieties by a linker.

Another aspect of the invention is a use of an immunoconjugate comprising an antibody covalently attached to one or more aza-benzazepine moieties by a linker in the treatment of an illness, in particular cancer.

### DESCRIPTION OF THE DRAWINGS

FIG. 1 shows a plot of the hydrolysis of the amidine group of benzazepine comparator compound CBz-3 to form lactam comparator compound CBz-5 over time in PBS buffer at 40° C.

FIG. 2A shows a plot of the hydrolysis of the amidine group of benzazepine comparator compound CBz-1, and aza-benzazepine compounds azaBa-1 and azaBz-2 by percentage of starting compounds remaining over 2 days.

FIG. 2B shows a plot of the hydrolysis of the amidine group of benzazepine comparator compound CBz-1, and aza-benzazepine compounds azaBa-1 and azaBz-2 by the appearance of the corresponding lactam compounds over 2 days.

FIG. 3A shows a plot of the hydrolysis of the amidine group of benzazepine comparator compounds CBz-4 and CBz-6, and aza-benzazepine compounds azaBa-1 and azaBz-5 by percentage of starting compounds remaining over 2 days.

FIG. 3B shows a plot of the hydrolysis of the amidine group of benzazepine comparator compounds CBz-4 and CBz-6, and aza-benzazepine compounds azaBa-1 and azaBz-5 by the appearance of the corresponding lactam compounds over 2 days.

FIG. 4 shows a plot of the hydrolysis of the amidine group of aza-benzazepine compounds azaBa-3, azaBz-5, azaBz-6, azaBz-7, and azaBz-8 in PBS and Formulation buffer, by the appearance of the corresponding lactam compounds over 2 days. The amount of lactam is normalized for each sample at the start (to) for easier rate comparisons.

FIG. 5 shows a plot of the hydrolysis of the amidine group of benzazepine comparator compounds CBz-2 and CBz-7, and aza-benzazepine compounds azaBa-6 and azaBz-8 in PBS, by the appearance of the corresponding lactam compounds over 2 days. The amount of lactam is normalized for each sample at the start (to) for easier rate comparisons.

### DETAILED DESCRIPTION OF THE INVENTION

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents, which may be included within the scope of the invention as defined by the claims.

One skilled in the art will recognize many methods and materials similar or equivalent to those described herein, which could be used in the practice of the present invention. The invention is in no way limited to the methods and materials described.

### Definitions

The term “immunoconjugate” or “immune-stimulating antibody conjugate” refers to an antibody construct that is covalently bonded to an adjuvant moiety via a linker. the term “adjuvant” refers to a substance capable of eliciting an immune response in a subject exposed to the adjuvant.

“Adjuvant moiety” refers to an adjuvant that is covalently bonded to an antibody construct, e.g., through a linker, as described herein. The adjuvant moiety can elicit the immune response while bonded to the antibody construct or after cleavage (e.g., enzymatic cleavage) from the antibody construct following administration of an immunoconjugate to the subject.

“Adjuvant” refers to a substance capable of eliciting an immune response in a subject exposed to the adjuvant.

The terms “Toll-like receptor” and “TLR” refer to any member of a family of highly-conserved mammalian proteins which recognizes pathogen-associated molecular patterns and acts as key signaling elements in innate immunity. They are single-pass membrane-spanning receptors usually expressed on sentinel cells such as macrophages and dendritic cells, that recognize structurally conserved molecules derived from microbes. Once these microbes have reached physical barriers such as the skin or intestinal tract mucosa, they are recognized by TLRs, which activate immune cell responses. TLR polypeptides share a characteristic structure that includes an extracellular domain that has leucine-rich repeats, a transmembrane domain, and an intracellular domain that is involved in TLR signaling. The terms “Toll-like receptor 7” and “TLR7” refer to nucleic acids or polypeptides sharing at least about 70%, about 80%, about 90%, about 95%, about 96%, about 97%, about 98%, about 99%, or more sequence identity to a publicly-available TLR7 sequence, e.g., GenBank accession number AAZ99026 for human TLR7 polypeptide, or GenBank accession number AAK62676 for murine TLR7 polypeptide. The terms “Toll-like receptor 8” and “TLR8” refer to nucleic acids or polypeptides sharing at least about 70%, about 80%, about 90%, about 95%, about 96%, about 97%, about 98%, about 99%, or more sequence identity to a publicly-available TLR7 sequence, e.g., GenBank accession number AAZ95441 for human TLR8 polypeptide, or GenBank accession number AAK62677 for murine TLR8 polypeptide.

A “TLR agonist” is a compound that binds, directly or indirectly, to a TLR (e.g., TLR7 and/or TLR8) to induce TLR signaling. Any detectable difference in TLR signaling can indicate that an agonist stimulates or activates a TLR. Signaling differences can be manifested, for example, as changes in the expression of target genes, in the phosphorylation of signal transduction components, in the intracellular localization of downstream elements such as nuclear factor-κB (NF-κB), in the association of certain components (such as IL-1 receptor associated kinase (IRAK)) with other proteins or intracellular structures, or in the biochemical activity of components such as kinases (such as mitogen-activated protein kinase (MAPK)).

“Antibody” refers to a polypeptide comprising an antigen binding region (including the complementarity determining region (CDRs)) from an immunoglobulin gene or fragments thereof. The term “antibody” specifically encompasses monoclonal antibodies (including full length monoclonal antibodies), polyclonal antibodies, multispecific antibodies (e.g., bispecific antibodies), and antibody fragments that exhibit the desired biological activity. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa) connected by disulfide bonds. Each chain is composed of structural domains, which are referred to as immunoglobulin domains. These domains are classified into different categories by size and function, e.g., variable domains or regions on the light and heavy chains (V_{L} and V_{H}, respectively) and constant domains or regions on the light and heavy chains (C_{L} and C_{H}, respectively). The N-terminus of each chain defines a variable region of about 100 to 110 or more amino acids, referred to as the paratope, primarily responsible for antigen recognition, i.e., the antigen binding domain. Light chains are classified as either kappa or lambda. Heavy chains are classified as gamma, mu, alpha, delta, or epsilon, which in turn define the immunoglobulin classes, IgG, IgM, IgA, IgD and IgE, respectively. IgG antibodies are large molecules of about 150 kDa composed of four peptide chains. IgG antibodies contain two identical class γ heavy chains of about 50 kDa and two identical light chains of about 25 kDa, thus a tetrameric quaternary structure. The two heavy chains are linked to each other and to a light chain each by disulfide bonds. The resulting tetramer has two identical halves, which together form the Y-like shape. Each end of the fork contains an identical antigen binding domain. There are four IgG subclasses (IgG1, IgG2, IgG3, and IgG4) in humans, named in order of their abundance in serum (i.e., IgG1 is the most abundant).

Typically, the antigen binding domain of an antibody will be most critical in specificity and affinity of binding to cancer cells.

“Bispecific” antibodies (bsAbs) are antibodies that bind two distinct epitopes to cancer (Suurs F. V. et al (2019) Pharmacology & Therapeutics 201: 103-119). Bispecific antibodies may engage immune cells to destroy tumor cells, deliver payloads to tumors, and/or block tumor signaling pathways. An antibody that targets a particular antigen includes a bispecific or multispecific antibody with at least one antigen binding region that targets the particular antigen. In some embodiments, the targeted monoclonal antibody is a bispecific antibody with at least one antigen binding region that targets tumor cells. Such antigens include but are not limited to: mesothelin, prostate specific membrane antigen (PSMA), HER2, TROP2, CEA, EGFR, 5T4, Nectin4, CD19, CD20, CD22, CD30, CD70, B7H3, B7H4 (also known as 08E), protein tyrosine kinase 7 (PTK7), glypican-3, RG1, fucosyl-GMl, CTLA-4, and CD44 (WO 2017/196598).

In some embodiments, the antibody construct is an antigen-binding antibody “fragment,” which comprises at least an antigen-binding region of an antibody, alone or with other components that together constitute the antibody construct. Many different types of antibody “fragments” are known in the art, including, for instance, (i) a Fab fragment, which is a monovalent fragment consisting of the V_{L}, V_{H}, C_{L}, and CH₁ domains, (ii) a F(ab′)₂ fragment, which is a bivalent fragment comprising two Fab fragments linked by a disulfide bridge at the hinge region, (iii) a Fv fragment consisting of the V_{L} and V_{H} domains of a single arm of an antibody, (iv) a Fab′ fragment, which results from breaking the disulfide bridge of an F(ab′)₂ fragment using mild reducing conditions, (v) a disulfide-stabilized Fv fragment (dsFv), and (vi) a single chain Fv (scFv), which is a monovalent molecule consisting of the two domains of the Fv fragment (i.e., V_{L} and V_{H}) joined by a synthetic linker which enables the two domains to be synthesized as a single polypeptide chain. In some embodiments, the antibody construct is an antibody or a fusion protein comprising (i) an antigen binding domain and (ii) an Fc domain.

The antibody or antibody fragment can be part of a larger construct, for example, a conjugate or fusion construct of the antibody fragment to additional regions. For instance, in some embodiments, the antibody fragment can be fused to an Fc region as described herein. In other embodiments, the antibody fragment (e.g., a Fab or scFv) can be part of a chimeric antigen receptor or chimeric T-cell receptor, for instance, by fusing to a transmembrane domain (optionally with an intervening linker or “stalk” (e.g., hinge region)) and optional intercellular signaling domain. For instance, the antibody fragment can be fused to the gamma and/or delta chains of a t-cell receptor, so as to provide a T-cell receptor like construct that binds PD-L1. In yet another embodiment, the antibody fragment is part of a bispecific T-cell engager (BiTEs) comprising a CD1 or CD3 binding domain and linker.

In some embodiments, the antibody construct comprises an Fc domain. In certain embodiments, the antibody construct is an antibody. In certain embodiments, the antibody construct is a fusion protein. The antigen binding domain can be a single-chain variable region fragment (scFv). A single-chain variable region fragment (scFv), which is a truncated Fab fragment including the variable (V) domain of an antibody heavy chain linked to a V domain of a light antibody chain via a synthetic peptide, can be generated using routine recombinant DNA technology techniques. Similarly, disulfide-stabilized variable region fragments (dsFv) can be prepared by recombinant DNA technology. The antibody construct or antigen binding domain may comprise one or more variable regions (e.g., two variable regions) of an antigen binding domain of an anti-CEA antibody, each variable region comprising a CDR1, a CDR2, and a CDR3.

“Cysteine-mutant antibody” is an antibody in which one or more amino acid residues of an antibody are substituted with cysteine residues. A cysteine-mutant antibody may be prepared from the parent antibody by antibody engineering methods (Junutula, et al., (2008b) Nature Biotech., 26(8):925-932; Doman et al. (2009) Blood 114(13):2721-2729; U.S. Pat. Nos. 7,521,541; 7,723,485; US 2012/0121615; WO 2009/052249). Cysteine residues provide for site-specific conjugation of a adjuvant such as a TLR agonist to the antibody through the reactive cysteine thiol groups at the engineered cysteine sites but do not perturb immunoglobulin folding and assembly or alter antigen binding and effector functions. Cysteine-mutant antibodies can be conjugated to the TLR agonist-linker compound with uniform stoichiometry of the immunoconjugate (e.g., up to two TLR agonist moieties per antibody in an antibody that has a single engineered, mutant cysteine site). The TLR agonist-linker compound has a reactive electrophilic group to react specifically with the free cysteine thiol groups of the cysteine-mutant antibody.

“Epitope” means any antigenic determinant or epitopic determinant of an antigen to which an antigen binding domain binds (i.e., at the paratope of the antigen binding domain). Antigenic determinants usually consist of chemically active surface groupings of molecules, such as amino acids or sugar side chains, and usually have specific three dimensional structural characteristics, as well as specific charge characteristics.

The terms “Fc receptor” or “FcR” refer to a receptor that binds to the Fc region of an antibody. There are three main classes of Fc receptors: (1) FcγR which bind to IgG, (2) FcαR which binds to IgA, and (3) FcR which binds to IgE. The FcγR family includes several members, such as FcγI (CD64), FcγRIIA (CD32A), FcγRIIB (CD32B), FcγRIIIA (CD16A), and FcγRIIIB (CD16B). The Fcγ receptors differ in their affinity for IgG and also have different affinities for the IgG subclasses (e.g., IgG1, IgG2, IgG3, and IgG4).

Nucleic acid or amino acid sequence “identity,” as referenced herein, can be determined by comparing a nucleic acid or amino acid sequence of interest to a reference nucleic acid or amino acid sequence. The percent identity is the number of nucleotides or amino acid residues that are the same (i.e., that are identical) as between the optimally aligned sequence of interest and the reference sequence divided by the length of the longest sequence (i.e., the length of either the sequence of interest or the reference sequence, whichever is longer). Alignment of sequences and calculation of percent identity can be performed using available software programs. Examples of such programs include CLUSTAL-W, T-Coffee, and ALIGN (for alignment of nucleic acid and amino acid sequences), BLAST programs (e.g., BLAST 2.1, BL2SEQ, BLASTp, BLASTn, and the like) and FASTA programs (e.g., FASTA3x, FAS™, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probalistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)). Percent (%) identity of sequences can be also calculated, for example, as 100×[(identical positions)/min(TG_{A}, TG_{B})], where TG_{A} and TG_{B} are the sum of the number of residues and internal gap positions in peptide sequences A and B in the alignment that minimizes TG_{A} and TG_{B}. See, e.g., Russell et al., J. Mol Biol., 244: 332-350 (1994).

The “antibody construct” or “binding agent” comprises Ig heavy and light chain variable region polypeptides that together form the antigen binding site. Each of the heavy and light chain variable regions are polypeptides comprising three complementarity determining regions (CDR1, CDR2, and CDR3) connected by framework regions. The antibody construct can be any of a variety of types of binding agents known in the art that comprise Ig heavy and light chains. For instance, the binding agent can be an antibody, an antigen-binding antibody “fragment,” or a T-cell receptor.

“Biosimilar” refers to an approved antibody construct that has active properties similar to, for example, a PD-L1-targeting antibody construct previously approved such as atezolizumab (TECENTRIQ™, Genentech, Inc.), durvalumab (IMFINZI™, AstraZeneca), and avelumab (BAVENCIO™, EMD Serono, Pfizer); a HER2-targeting antibody construct previously approved such as trastuzumab (HERCEPTIN™, Genentech, Inc.), and pertuzumab (PERJETA™, Genentech, Inc.); or a CEA-targeting antibody such as labetuzumab (CEA-CIDE™, MN-14, hMN14, Immunomedics) CAS Reg. No. 219649-07-7).

“Biobetter” refers to an approved antibody construct that is an improvement of a previously approved antibody construct, such as atezolizumab, durvalumab, avelumab, trastuzumab, pertuzumab, and labetuzumab. The biobetter can have one or more modifications (e.g., an altered glycan profile, or a unique epitope) over the previously approved antibody construct.

“Amino acid” refers to any monomeric unit that can be incorporated into a peptide, polypeptide, or protein. Amino acids include naturally-occurring α-amino acids and their stereoisomers, as well as unnatural (non-naturally occurring) amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid). The amino acids can be glycosylated (e.g., N-linked glycans, O-linked glycans, phosphoglycans, C-linked glycans, or glypication) or deglycosylated. Amino acids may be referred to herein by either the commonly known three letter symbols or by the one-letter symbols recommended by the IUPAC-IUB Biochemical Nomenclature Commission.

Naturally-occurring amino acids are those encoded by the genetic code, as well as those amino acids that are later modified, e.g., hydroxyproline, γ-carboxyglutamate, and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Naturally-occurring amino acids include those formed in proteins by post-translational modification, such as citrulline (Cit).

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, an amino group) but have modified side-chain groups or modified peptide backbones, e.g., homoserine, norleucine, methionine sulfoxide, and methionine methyl sulfonium. “Amino acid mimetics” refer to chemical compounds that have a structure that is different from the general chemical structure of an amino acid, but that functions in a manner similar to a naturally-occurring amino acid.

“Linker” refers to a bifunctional or multifunctional moiety that covalently bonds two or more moieties such as an adjuvant moiety to an antibody in an immunoconjugate. Useful bonds for connecting linking moieties an adjuvant moiety to an antibody include, but are not limited to, amides, amines, esters, carbamates, disulfides, ureas, thioethers, thiocarbamates, thiocarbonates, and thioureas.

“Linking moiety” refers to a functional group that covalently bonds two or more moieties in a compound or material. For example, the linking moiety can serve to covalently bond an adjuvant moiety to an antibody in an immunoconjugate. Useful bonds for connecting linking moieties to proteins and other materials include, but are not limited to, amides, amines, esters, carbamates, ureas, thioethers, thiocarbamates, thiocarbonates, and thioureas.

“Divalent” refers to a chemical moiety that contains two points of attachment for linking two functional groups; polyvalent linking moieties can have additional points of attachment for linking further functional groups. Divalent radicals may be denoted with the suffix “diyl”. For example, divalent linking moieties include divalent polymer moieties such as divalent poly(ethylene glycol), divalent cycloalkyl, divalent heterocycloalkyl, divalent aryl, and divalent heteroaryl group. A “divalent cycloalkyl, heterocycloalkyl, aryl, or heteroaryl group” refers to a cycloalkyl, heterocycloalkyl, aryl, or heteroaryl group having two points of attachment for covalently linking two moieties in a molecule or material. Cycloalkyl, heterocycloalkyl, aryl, or heteroaryl groups can be substituted or unsubstituted. Cycloalkyl, heterocycloalkyl, aryl, or heteroaryl groups can be substituted with one or more groups selected from halo, hydroxy, amino, alkylamino, amido, acyl, nitro, cyano, alkoxy, and others.

A wavy line (“ [symbol] ”) represents a point of attachment of the specified chemical moiety. If the specified chemical moiety has two wavy lines (“ [symbol] ”) present, it will be understood that the chemical moiety can be used bilaterally, i.e., as read from left to right or from right to left. In some embodiments, a specified moiety having two wavy lines (“ [symbol] ”) present is considered to be used as read from left to right.

“Alkyl” refers to a straight (linear) or branched, saturated, aliphatic radical having the number of carbon atoms indicated. Alkyl can include any number of carbons, for example from one to twelve. Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, 1-heptyl, 1-octyl, and the like.

Alkyl groups can be substituted or unsubstituted. “Substituted alkyl” groups can be substituted with one or more groups selected from halo, hydroxy, amino, oxo (═O), alkylamino, amido, acyl, nitro, cyano, and alkoxy.

The term “alkyldiyl” refers to a divalent alkyl radical. Examples of alkyldiyl groups include, but are not limited to, methylene (—CH₂—), ethylene (—CH₂CH₂—), propylene (—CH₂CH₂CH₂—), and the like. An alkyldiyl group may also be referred to as an “alkylene” group.

“Alkenyl” refers to a straight (linear) or branched, unsaturated, aliphatic radical having the number of carbon atoms indicated and at least one carbon-carbon double bond, sp2. Alkenyl can include from two to about 12 or more carbons atoms. Alkenyl groups are radicals having “cis” and “trans” orientations, or alternatively, “E” and “Z” orientations. Examples include, but are not limited to, ethylenyl or vinyl (—CH═CH₂), allyl (—CH₂CH═CH₂). butenyl, pentenyl, and isomers thereof. Alkenyl groups can be substituted or unsubstituted. “Substituted alkenyl” groups can be substituted with one or more groups selected from halo, hydroxy, amino, oxo (═O), alkylamino, amido, acyl, nitro, cyano, and alkoxy.

The terms “alkenylene” or “alkenyldiyl” refer to a linear or branched-chain divalent hydrocarbon radical. Examples include, but are not limited to, ethylenylene or vinylene (—CH═CH—), allyl (—CH₂CH═CH—), and the like.

“Alkynyl” refers to a straight (linear) or branched, unsaturated, aliphatic radical having the number of carbon atoms indicated and at least one carbon-carbon triple bond, sp. Alkynyl can include from two to about 12 or more carbons atoms. For example, C₂-C₆ alkynyl includes, but is not limited to ethynyl (—C═CH), propynyl (propargyl, —CH₂C≡CH), butynyl, pentynyl, hexynyl, and isomers thereof. Alkynyl groups can be substituted or unsubstituted. “Substituted alkynyl” groups can be substituted with one or more groups selected from halo, hydroxy, amino, oxo (═O), alkylamino, amido, acyl, nitro, cyano, and alkoxy.

The term “alkynylene” or “alkynyldiyl” refer to a divalent alkynyl radical.

“Heteroalkyl” or “heteroalkylene” refer to a monovalent, straight or branched chain alkyl group, as defined above, comprising at least one heteroatom including but not limited to Si, N, O, P or S within the alkyl chain or at a terminus of the alkyl chain. In some embodiments, a heteroatom is within the alkyl chain. In other embodiments, a heteroatom is at a terminus of the alkylene and thus serves to join the alkyl to the remainder of the molecule. In some embodiments, a heteroalkyl group may have 1 to 12 carbon atoms (C₁-C₁₂ heteroalkyl). In some embodiments, a heteroalkyl group may have 1 to 24 carbon atoms (C₁-C₂₄ heteroalkyl). In some embodiments, a heteroalkyl group may have 1 to 40 carbon atoms (C₁-C₄₀ heteroalkyl). Unless stated otherwise specifically in the specification, a heteroalkyl group is optionally substituted. For example, heteroalkyl groups can be substituted with 1-6 fluoro (F) substituents, for example, on the carbon backbone (as —CHF— or —CF₂—) or on terminal carbons of straight chain or branched heteroalkyls (such as —CHF₂ or —CF₃). Examples of heteroalkyl groups include, but are not limited to, —CH₂CH₂OCH₃, —CH₂CH₂NHCH₃, —CH₂CH₂N(CH₃)₂, —C(═O)NHCH₂CH₂NHCH₃, —C(═O)N(CH₃)CH₂CH₂N(CH₃)₂, —C(═O)NHCH₂CH₂NHC(═O)CH₂CH₃, —C(═O)N(CH₃)CH₂CH₂N(CH₃)C(═O)CH₂CH₃, —OCH₂CH₂CH₂NH(CH₃), —OCH₂CH₂CH₂N(CH₃)₂, —OCH₂CH₂CH₂NHC(═O)CH₂CH₃, —OCH₂CH₂CH₂N(CH₃)C(═O)CH₂CH₃, —CH₂CH₂CH₂NH(CH₃), —OCH₂CH₂CH₂N(CH₃)₂, —CH₂CH₂CH₂NHC(═O)CH₂CH₃, —CH₂CH₂CH₂N(CH₃)C(═O)CH₂CH₃, —CH₂SCH₂CH₃, —CH₂CH₂S(O)CH₃, —NHCH₂CH₂NHC(═O)CH₂CH₃, —CH₂CH₂S(O)₂CH₃, —CH₂CH₂OCF₃, and —Si(CH₃)₃. Up to two heteroatoms may be consecutive, such as, for example, —CH₂NHOCH₃ and —CH₂OSi(CH₃)₃. A terminal polyethylene glycol (PEG) moiety is a type of heteroalkyl group. Exemplary heteroalkyl groups also include ethylene oxide (e.g., polyethylene oxide), propylene oxide, amino acid chains (i.e., short to medium length peptides such as containing 1-15 amino acids), and alkyl chains connected via a variety of functional groups such as amides, disulfides, ketones, phosphonates, phosphates, sulfates, sulfones, sulfonamides, esters, ethers, —S—, carbamates, ureas, thioureas, anhydrides, or the like (including combinations thereof). In some embodiments, a heteroalkyl group includes a polyamino acid having 1-10 amino acids. In some embodiments, a heteroalkyl group includes a polyamino acid having 1-5 amino acids.

Heteroalkyl groups include a solubilizing unit comprising one or more groups of polyglycine, polysarcosine, polyethyleneoxy (PEG), and a glycoside, or combinations thereof.

“Heteroalkenyl” refers to a heteroalkyl group, as defined above, that contains at least one carbon-carbon double bond. “Heteroalkynyl” refers to a heteroalkyl group, as defined above, that contains at least one carbon-carbon triple bond.

“Heteroalkyldiyl” refers to a divalent form of a heteroalkyl group as defined above. In some embodiments, a heteroalkyldiyl group may have 1 to 12 carbon atoms (C₁-C₁₂ heteroalkyldiyl). In some embodiments, a heteroalkyldiyl group may have 1 to 24 carbon atoms (C₁-C₂₄ heteroalkyldiyl). In some embodiments, a heteroalkyldiyl group may have 1 to 40 carbon atoms (C₁-C₄₀ heteroalkyldiyl). Examples of heteroalkyldiyl groups include, but are not limited to, —CH₂CH₂OCH₂—, —CH₂CH₂OCF₂—, —CH₂CH₂NHCH₂—, —CH₂OC(═O)NH—, —CH₂OP(═O)(OH)OCH₂—, —C(═O)NHCH₂CH₂NHCH₂—, —C(═O)N(CH₃)CH₂CH₂N(CH₃)CH₂—, —C(═O)NHCH₂CH₂NHC(═O)CH₂CH₂—, —C(═O)N(CH₃)CH₂CH₂N(CH₃)C(═O)CH₂CH₂—, —OCH₂CH₂OCH₂CH₂—, —OCH₂CH₂OCH₂C(═O)—, —OCH₂CH₂OCH₂CH₂C(═O)—, —OCH₂CH₂NHCH₂—, —OCH₂CH₂N(CH₃)CH₂—, —OCH₂CH₂CH₂NHCH₂—, —OCH₂CH₂CH₂N(CH₃)CH₂—, —OCH₂CH₂CH₂NHC(═O)CH₂CH₂—, —OCH₂CH₂CH₂N(CH₃)C(═O)CH₂CH₂—, —CH₂CH₂CH₂NHCH₂—, —CH₂CH₂CH₂N(CH₃)CH₂—, —CH₂CH₂CH₂NHC(═O)CH₂CH₂—, —CH₂CH₂CH₂N(CH₃)C(═O)CH₂CH₂—, —CH₂CH₂NHC(═O)—, —CH₂CH₂N(CH₃)CH₂—, —CH₂CH₂N⁺(CH₃)₂—, —NHCH₂CH₂(NH₂)CH₂—, and —NHCH₂CH₂(NHCH₃)CH₂—. A divalent polyethylene glycol (PEG) moiety with one to about 50 units of —OCH₂CH₂— is a type of heteroalkyldiyl group. “Heteroalkenyldiyl” refers to a divalent form of a heteroalkenyl group. “Heteroalkynyldiyl” refers to a divalent form of a heteroalkynyl group.

The terms “carbocycle”, “carbocyclyl”, “carbocyclic ring” and “cycloalkyl” refer to a saturated or partially unsaturated, monocyclic, fused bicyclic, or bridged polycyclic ring assembly containing from 3 to 12 ring atoms, or the number of atoms indicated. Saturated monocyclic carbocyclic rings include, for example, cyclopropyl, cyclobutyl, cyclopentyl, cyclohexyl, and cyclooctyl. Saturated bicyclic and polycyclic carbocyclic rings include, for example, norbornane, [2.2.2]bicyclooctane, decahydronaphthalene and adamantane. Carbocyclic groups can also be partially unsaturated, having one or more double or triple bonds in the ring. Representative carbocyclic groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene.

The term “cycloalkyldiyl” refers to a divalent cycloalkyl radical.

“Aryl” refers to a monovalent aromatic hydrocarbon radical of 6-20 carbon atoms (C₆-C₂₀) derived by the removal of one hydrogen atom from a single carbon atom of a parent aromatic ring system. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl.

The terms “arylene” or “aryldiyl” mean a divalent aromatic hydrocarbon radical of 6-20 carbon atoms (C₆-C₂₀) derived by the removal of two hydrogen atom from a two carbon atoms of a parent aromatic ring system. Some aryldiyl groups are represented in the exemplary structures as “Ar”. Aryldiyl includes bicyclic radicals comprising an aromatic ring fused to a saturated, partially unsaturated ring, or aromatic carbocyclic ring. Typical aryldiyl groups include, but are not limited to, radicals derived from benzene (phenyldiyl), substituted benzenes, naphthalene, anthracene, biphenylene, indenylene, indanylene, 1,2-dihydronaphthalene, 1,2,3,4-tetrahydronaphthyl, and the like. Aryldiyl groups are also referred to as “arylene”, and are optionally substituted with one or more substituents described herein.

The terms “heterocycle,” “heterocyclyl” and “heterocyclic ring” are used interchangeably herein and refer to a saturated or a partially unsaturated (i.e., having one or more double and/or triple bonds within the ring) carbocyclic radical of 3 to about 20 ring atoms in which at least one ring atom is a heteroatom selected from nitrogen, oxygen, phosphorus and sulfur, the remaining ring atoms being C, where one or more ring atoms is optionally substituted independently with one or more substituents described below. A heterocycle may be a monocycle having 3 to 7 ring members (2 to 6 carbon atoms and 1 to 4 heteroatoms selected from N, O, P, and S) or a bicycle having 7 to 10 ring members (4 to 9 carbon atoms and 1 to 6 heteroatoms selected from N, O, P, and S), for example: a bicyclo[4,5], [5,5], [5,6], or [6,6] system. Heterocycles are described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566. “Heterocyclyl” also includes radicals where heterocycle radicals are fused with a saturated, partially unsaturated ring, or aromatic carbocyclic or heterocyclic ring. Examples of heterocyclic rings include, but are not limited to, morpholin-4-yl, piperidin-1-yl, piperazinyl, piperazin-4-yl-2-one, piperazin-4-yl-3-one, pyrrolidin-1-yl, thiomorpholin-4-yl, S-dioxothiomorpholin-4-yl, azocan-1-yl, azetidin-1-yl, octahydropyrido[1,2-a]pyrazin-2-yl, [1,4]diazepan-1-yl, pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, homopiperazinyl, azetidinyl, oxetanyl, thietanyl, homopiperidinyl, oxepanyl, thiepanyl, oxazepinyl, diazepinyl, thiazepinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinylimidazolinyl, imidazolidinyl, 3-azabicyco[3.1.0]hexanyl, 3-azabicyclo[4.1.0]heptanyl, azabicyclo[2.2.2]hexanyl, 3H-indolyl quinolizinyl and N-pyridyl ureas. Spiro heterocyclyl moieties are also included within the scope of this definition. Examples of spiro heterocyclyl moieties include azaspiro[2.5]octanyl and azaspiro[2.4]heptanyl. Examples of a heterocyclic group wherein 2 ring atoms are substituted with oxo (═O) moieties are pyrimidinonyl and 1,1-dioxo-thiomorpholinyl. The heterocycle groups herein are optionally substituted independently with one or more substituents described herein.

The term “heterocyclyldiyl” refers to a divalent, saturated or a partially unsaturated (i.e., having one or more double and/or triple bonds within the ring) carbocyclic radical of 3 to about 20 ring atoms in which at least one ring atom is a heteroatom selected from nitrogen, oxygen, phosphorus and sulfur, the remaining ring atoms being C, where one or more ring atoms is optionally substituted independently with one or more substituents as described. Examples of 5-membered and 6-membered heterocyclyldiyls include morpholinyldiyl, piperidinyldiyl, piperazinyldiyl, pyrrolidinyldiyl, dioxanyldiyl, thiomorpholinyldiyl, and S-dioxothiomorpholinyldiyl.

The term “heteroaryl” refers to a monovalent aromatic radical of 5-, 6-, or 7-membered rings, and includes fused ring systems (at least one of which is aromatic) of 5-20 atoms, containing one or more heteroatoms independently selected from nitrogen, oxygen, and sulfur. Examples of heteroaryl groups are pyridinyl (including, for example, 2-hydroxypyridinyl), imidazolyl, imidazopyridinyl, pyrimidinyl (including, for example, 4-hydroxypyrimidinyl), pyrazolyl, triazolyl, pyrazinyl, tetrazolyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. Heteroaryl groups are optionally substituted independently with one or more substituents described herein.

The term “heteroaryldiyl” refers to a divalent aromatic radical of 5-, 6-, or 7-membered rings, and includes fused ring systems (at least one of which is aromatic) of 5-20 atoms, containing one or more heteroatoms independently selected from nitrogen, oxygen, and sulfur. Examples of 5-membered and 6-membered heteroaryldiyls include pyridyldiyl, imidazolyldiyl, pyrimidinyldiyl, pyrazolyldiyl, triazolyldiyl, pyrazinyldiyl, tetrazolyldiyl, furyldiyl, thienyldiyl, isoxazolyldiyldiyl, thiazolyldiyl, oxadiazolyldiyl, oxazolyldiyl, isothiazolyldiyl, and pyrrolyldiyl.

The heterocycle or heteroaryl groups may be carbon (carbon-linked), or nitrogen (nitrogen-linked) bonded where such is possible. By way of example and not limitation, carbon bonded heterocycles or heteroaryls are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline.

By way of example and not limitation, nitrogen bonded heterocycles or heteroaryls are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline.

The terms “halo” and “halogen,” by themselves or as part of another substituent, refer to a fluorine, chlorine, bromine, or iodine atom.

The term “carbonyl,” by itself or as part of another substituent, refers to C(═O) or —C(═O)—, i.e., a carbon atom double-bonded to oxygen and bound to two other groups in the moiety having the carbonyl.

As used herein, the phrase “quaternary ammonium salt” refers to a tertiary amine that has been quaternized with an alkyl substituent (e.g., a C₁-C₄ alkyl such as methyl, ethyl, propyl, or butyl).

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

The term “salt” refers to acid or base salts of the compounds of the disclosed herein. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. It is understood that the pharmaceutically acceptable salts are non-toxic. Pharmaceutically acceptable salts of the acidic compounds disclosed herein are salts formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts. Similarly acid addition salts, such as of mineral acids, organic carboxylic and organic sulfonic acids, e.g., hydrochloric acid, methanesulfonic acid, maleic acid, are also possible provided a basic group, such as pyridyl, constitutes part of the structure. The neutral forms of the compounds can be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

Any compound or Formula given herein, is intended to represent unlabeled forms as well as isotopically labeled forms of the compounds (i.e., “isotopic analogs”). Isotopically labeled compounds have structures depicted by the formulas given herein except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I and ¹²⁵I, respectively. Various isotopically labeled compounds of the present disclosure, for example those into which radioactive isotopes such as ³H, ¹³C and ¹⁴C are incorporated. Such isotopically labeled compounds may be useful for enhanced therapeutic activity, in metabolic studies, reaction kinetic studies, detection or imaging techniques, such as positron emission tomography (PET) or single-photon emission computed tomography (SPECT) including drug or substrate tissue distribution assays or in radioactive treatment of patients.

The disclosure also includes “deuterated analogs” of compounds described herein in which from 1 to n hydrogens attached to a carbon atom is/are replaced by deuterium (²H), in which n is the number of hydrogens in the molecule. Such compounds exhibit increased resistance to metabolism and are thus useful for increasing the half-life of any compound when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium. Deuterium labeled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F, ³H, or ¹¹C labeled compound may be useful for PET or SPECT or other imaging studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in a compound described herein. The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

The terms “treat,” “treatment,” and “treating” refer to any indicia of success in the treatment or amelioration of an injury, pathology, condition (e.g., cancer), or symptom (e.g., cognitive impairment), including any objective or subjective parameter such as abatement; remission; diminishing of symptoms or making the symptom, injury, pathology, or condition more tolerable to the patient; reduction in the rate of symptom progression; decreasing the frequency or duration of the symptom or condition; or, in some situations, preventing the onset of the symptom. The treatment or amelioration of symptoms can be based on any objective or subjective parameter, including, for example, the result of a physical examination.

The terms “cancer,” “neoplasm,” and “tumor” are used herein to refer to cells which exhibit autonomous, unregulated growth, such that the cells exhibit an aberrant growth phenotype characterized by a significant loss of control over cell proliferation. Cells of interest for detection, analysis, and/or treatment in the context of the invention include cancer cells (e.g., cancer cells from an individual with cancer), malignant cancer cells, pre-metastatic cancer cells, metastatic cancer cells, and non-metastatic cancer cells. Cancers of virtually every tissue are known. The phrase “cancer burden” refers to the quantum of cancer cells or cancer volume in a subject. Reducing cancer burden accordingly refers to reducing the number of cancer cells or the cancer cell volume in a subject. The term “cancer cell” as used herein refers to any cell that is a cancer cell (e.g., from any of the cancers for which an individual can be treated, e.g., isolated from an individual having cancer) or is derived from a cancer cell, e.g., clone of a cancer cell. For example, a cancer cell can be from an established cancer cell line, can be a primary cell isolated from an individual with cancer, can be a progeny cell from a primary cell isolated from an individual with cancer, and the like. In some embodiments, the term can also refer to a portion of a cancer cell, such as a sub-cellular portion, a cell membrane portion, or a cell lysate of a cancer cell. Many types of cancers are known to those of skill in the art, including solid tumors such as carcinomas, sarcomas, glioblastomas, melanomas, lymphomas, and myelomas, and circulating cancers such as leukemias.

As used herein, the term “cancer” includes any form of cancer, including but not limited to, solid tumor cancers (e.g., skin, lung, prostate, breast, gastric, bladder, colon, ovarian, pancreas, kidney, liver, glioblastoma, medulloblastoma, leiomyosarcoma, head & neck squamous cell carcinomas, melanomas, and neuroendocrine) and liquid cancers (e.g., hematological cancers); carcinomas; soft tissue tumors; sarcomas; teratomas; melanomas; leukemias; lymphomas; and brain cancers, including minimal residual disease, and including both primary and metastatic tumors.

“PD-L1 expression” refers to a cell that has a PD-L1 receptor on the cell's surface. As used herein “PD-L1 overexpression” refers to a cell that has more PD-L1 receptors as compared to corresponding non-cancer cell.

“HER2” refers to the protein human epidermal growth factor receptor 2.

“HER2 expression” refers to a cell that has a HER2 receptor on the cell's surface. For example, a cell may have from about 20,000 to about 50,000 HER2 receptors on the cell's surface. As used herein “HER2 overexpression” refers to a cell that has more than about 50,000 HER2 receptors. For example, a cell 2, 5, 10, 100, 1,000, 10,000, 100,000, or 1,000,000 times the number of HER2 receptors as compared to corresponding non-cancer cell (e.g., about 1 or 2 million HER2 receptors). It is estimated that HER2 is overexpressed in about 25% to about 30% of breast cancers.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, and invasion of surrounding or distant tissues or organs, such as lymph nodes.

As used herein, the phrases “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs, therefore, tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part that is not directly connected to the organ of the original cancerous tumor. Metastasis can also be defined as several steps of a process, such as the departure of cancer cells from an original tumor site, and migration and/or invasion of cancer cells to other parts of the body.

The phrases “effective amount” and “therapeutically effective amount” refer to a dose or amount of a substance such as an immunoconjugate that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); Goodman & Gilman's The Pharmacological Basis of Therapeutics, 11^{th} Edition (McGraw-Hill, 2006); and Remington: The Science and Practice of Pharmacy, 22^{nd} Edition, (Pharmaceutical Press, London, 2012)). In the case of cancer, the therapeutically effective amount of the immunoconjugate may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the immunoconjugate may prevent growth and/or kill existing cancer cells, it may be cytostatic and/or cytotoxic. For cancer therapy, efficacy can, for example, be measured by assessing the time to disease progression (TTP) and/or determining the response rate (RR)

“Recipient,” “individual,” “subject,” “host,” and “patient” are used interchangeably and refer to any mammalian subject for whom diagnosis, treatment, or therapy is desired (e.g., humans). “Mammal” for purposes of treatment refers to any animal classified as a mammal, including humans, domestic and farm animals, and zoo, sports, or pet animals, such as dogs, horses, cats, cows, sheep, goats, pigs, camels, etc. In certain embodiments, the mammal is human.

The phrase “synergistic adjuvant” or “synergistic combination” in the context of this invention includes the combination of two immune modulators such as a receptor agonist, cytokine, and adjuvant polypeptide, that in combination elicit a synergistic effect on immunity relative to either administered alone. Particularly, the immunoconjugates disclosed herein comprise synergistic combinations of the claimed adjuvant and antibody construct. These synergistic combinations upon administration elicit a greater effect on immunity, e.g., relative to when the antibody construct or adjuvant is administered in the absence of the other moiety. Further, a decreased amount of the immunoconjugate may be administered (as measured by the total number of antibody constructs or the total number of adjuvants administered as part of the immunoconjugate) compared to when either the antibody construct or adjuvant is administered alone.

As used herein, the term “administering” refers to parenteral, intravenous, intraperitoneal, intramuscular, intratumoral, intralesional, intranasal, or subcutaneous administration, oral administration, administration as a suppository, topical contact, intrathecal administration, or the implantation of a slow-release device, e.g., a mini-osmotic pump, to the subject.

The terms “about” and “around,” as used herein to modify a numerical value, indicate a close range surrounding the numerical value. Thus, if “X” is the value, “about X” or “around X” indicates a value of from 0.9X to 1.1X, e.g., from 0.95X to 1.05X or from 0.99X to 1.01X. A reference to “about X” or “around X” specifically indicates at least the values X, 0.95X, 0.96X, 0.97X, 0.98X, 0.99X, 1.01X, 1.02X, 1.03X, 1.04X, and 1.05X. Accordingly, “about X” and “around X” are intended to teach and provide written description support for a claim limitation of, e.g., “0.98X.”

### Antibodies

The immunoconjugate of the invention comprises an antibody. Included in the scope of the embodiments of the invention are functional variants of the antibody constructs or antigen binding domain described herein. The term “functional variant” as used herein refers to an antibody construct having an antigen binding domain with substantial or significant sequence identity or similarity to a parent antibody construct or antigen binding domain, which functional variant retains the biological activity of the antibody construct or antigen binding domain of which it is a variant. Functional variants encompass, for example, those variants of the antibody constructs or antigen binding domain described herein (the parent antibody construct or antigen binding domain) that retain the ability to recognize target cells to a similar extent, the same extent, or to a higher extent, as the parent antibody construct or antigen binding domain.

In reference to the antibody construct or antigen binding domain, the functional variant can, for instance, be at least about 30%, about 50%, about 75%, about 80%, about 85%, about 90%, about 91%, about 92%, about 93%, about 94%, about 95%, about 96%, about 97%, about 98%, about 99% or more identical in amino acid sequence to the antibody construct or antigen binding domain.

A functional variant can, for example, comprise the amino acid sequence of the parent antibody construct or antigen binding domain with at least one conservative amino acid substitution. Alternatively, or additionally, the functional variants can comprise the amino acid sequence of the parent antibody construct or antigen binding domain with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent antibody construct or antigen binding domain.

The antibodies comprising the immunoconjugates of the invention include Fc engineered variants. In some embodiments, the mutations in the Fc region that result in modulated binding to one or more Fc receptors can include one or more of the following mutations: SD (S239D), SDIE (S239D/I332E), SE (S267E), SELF (S267E/L328F), SDIE (S239D/I332E), SDIEAL (S239D/I332E/A330L), GA (G236A), ALIE (A330L/I332E), GASDALIE (G236A/S239D/A330L/I332E), V9 (G237D/P238D/P271G/A330R), and V11 (G237D/P238D/H268D/P271G/A330R), and/or one or more mutations at the following amino acids: E345R, E233, G237, P238, H268, P271, L328 and A330. Additional Fc region modifications for modulating Fc receptor binding are described in, for example, US 2016/0145350, U.S. Pat. Nos. 7,416,726 and 5,624,821, which are hereby incorporated by reference in their entireties herein.

The antibodies comprising the immunoconjugates of the invention include glycan variants, such as afucosylation. In some embodiments, the Fc region of the binding agents are modified to have an altered glycosylation pattern of the Fc region compared to the native non-modified Fc region.

Amino acid substitutions of the inventive antibody constructs or antigen binding domains are preferably conservative amino acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g., Lys, His, Arg, etc.), an uncharged amino acid with a polar side chain substituted for another uncharged amino acid with a polar side chain (e.g., Asn, Gln, Ser, Thr, Tyr, etc.), an amino acid with a beta-branched side-chain substituted for another amino acid with a beta-branched side-chain (e.g., Ile, Thr, and Val), an amino acid with an aromatic side-chain substituted for another amino acid with an aromatic side chain (e.g., His, Phe, Trp, and Tyr), etc.

The antibody construct or antigen binding domain can consist essentially of the specified amino acid sequence or sequences described herein, such that other components, e.g., other amino acids, do not materially change the biological activity of the antibody construct or antigen binding domain functional variant.

In some embodiments, the antibodies in the immunoconjugates contain a modified Fc region, wherein the modification modulates the binding of the Fc region to one or more Fc receptors.

In some embodiments, the antibodies in the immunoconjugates (e.g., antibodies conjugated to at least two adjuvant moieties) contain one or more modifications (e.g., amino acid insertion, deletion, and/or substitution) in the Fc region that results in modulated binding (e.g., increased binding or decreased binding) to one or more Fc receptors (e.g., FcγRI (CD64), FcγRIIA (CD32A), FcγRIIB (CD32B), FcγRIIIA (CD16a), and/or FcγRIIIB (CD16b)) as compared to the native antibody lacking the mutation in the Fc region. In some embodiments, the antibodies in the immunoconjugates contain one or more modifications (e.g., amino acid insertion, deletion, and/or substitution) in the Fc region that reduce the binding of the Fc region of the antibody to FcγRIIB. In some embodiments, the antibodies in the immunoconjugates contain one or more modifications (e.g., amino acid insertion, deletion, and/or substitution) in the Fc region of the antibody that reduce the binding of the antibody to FcγRIIB while maintaining the same binding or having increased binding to FcγRI (CD64), FcγRIIA (CD32A), and/or FcRγIIIA (CD16a) as compared to the native antibody lacking the mutation in the Fc region. In some embodiments, the antibodies in the immunoconjugates contain one of more modifications in the Fc region that increase the binding of the Fc region of the antibody to FcγRIIB.

In some embodiments, the modulated binding is provided by mutations in the Fc region of the antibody relative to the native Fc region of the antibody. The mutations can be in a CH2 domain, a CH3 domain, or a combination thereof. A “native Fc region” is synonymous with a “wild-type Fc region” and comprises an amino acid sequence that is identical to the amino acid sequence of an Fc region found in nature or identical to the amino acid sequence of the Fc region found in the native antibody (e.g., cetuximab). Native sequence human Fc regions include a native sequence human IgG1 Fc region, native sequence human IgG2 Fc region, native sequence human IgG3 Fc region, and native sequence human IgG4 Fc region, as well as naturally occurring variants thereof. Native sequence Fc includes the various allotypes of Fcs (Jefferis et al., (2009) mAbs, 1(4):332-338).

In some embodiments, the Fc region of the antibodies of the immunoconjugates are modified to have an altered glycosylation pattern of the Fc region compared to the native non-modified Fc region.

Human immunoglobulin is glycosylated at the Asn297 residue in the Cγ2 domain of each heavy chain. This N-linked oligosaccharide is composed of a core heptasaccharide, N-acetylglucosamine4Mannose3 (GlcNAc4Man3). Removal of the heptasaccharide with endoglycosidase or PNGase F is known to lead to conformational changes in the antibody Fc region, which can significantly reduce antibody-binding affinity to activating FcγR and lead to decreased effector function. The core heptasaccharide is often decorated with galactose, bisecting GlcNAc, fucose, or sialic acid, which differentially impacts Fc binding to activating and inhibitory FcγR. Additionally, it has been demonstrated that α2,6-sialyation enhances anti-inflammatory activity in vivo, while afucosylation leads to improved FcγRIIIa binding and a 10-fold increase in antibody-dependent cellular cytotoxicity and antibody-dependent phagocytosis. Specific glycosylation patterns, therefore, can be used to control inflammatory effector functions.

In some embodiments, the modification to alter the glycosylation pattern is a mutation. For example, a substitution at Asn297. In some embodiments, Asn297 is mutated to glutamine (N297Q). Methods for controlling immune response with antibodies that modulate FcγR-regulated signaling are described, for example, in U.S. Pat. No. 7,416,726, US 2007/0014795 and US 2008/0286819, which are hereby incorporated by reference in their entireties.

In some embodiments, the antibodies of the immunoconjugates are modified to contain an engineered Fab region with a non-naturally occurring glycosylation pattern. For example, hybridomas can be genetically engineered to secrete afucosylated mAb, desialylated mAb or deglycosylated Fc with specific mutations that enable increased FcRγIIIa binding and effector function. In some embodiments, the antibodies of the immunoconjugates are engineered to be afucosylated.

In some embodiments, the entire Fc region of an antibody in the immunoconjugates is exchanged with a different Fc region, so that the Fab region of the antibody is conjugated to a non-native Fc region. For example, the Fab region of cetuximab, which normally comprises an IgG1 Fc region, can be conjugated to IgG2, IgG3, IgG4, or IgA, or the Fab region of nivolumab, which normally comprises an IgG4 Fc region, can be conjugated to IgG1, IgG2, IgG3, IgA1, or IgG2. In some embodiments, the Fc modified antibody with a non-native Fc domain also comprises one or more amino acid modification, such as the S228P mutation within the IgG4 Fc, that modulate the stability of the Fc domain described. In some embodiments, the Fc modified antibody with a non-native Fc domain also comprises one or more amino acid modifications described herein that modulate Fc binding to FcR.

In some embodiments, the modifications that modulate the binding of the Fc region to FcR do not alter the binding of the Fab region of the antibody to its antigen when compared to the native non-modified antibody. In other embodiments, the modifications that modulate the binding of the Fc region to FcR also increase the binding of the Fab region of the antibody to its antigen when compared to the native non-modified antibody.

In some embodiments, the antibodies in the immunoconjugates contain a modified Fc region, wherein the modification modulates the binding of the Fc region to one or more Fc receptors.

In some embodiments, the Fc region is modified by inclusion of a transforming growth factor beta 1 (TGFβ1) receptor, or a fragment thereof, that is capable of binding TGFβ1. For example, the receptor can be TGFβ receptor II (TGFβRII). In some embodiments, the TGFβ receptor is a human TGFβ receptor. In some embodiments, the IgG has a C-terminal fusion to a TGFβRII extracellular domain (ECD) as described in U.S. Pat. No. 9,676,863, incorporated herein. An “Fc linker” may be used to attach the IgG to the TGFβRII extracellular domain. The Fc linker may be a short, flexible peptide that allows for the proper three-dimensional folding of the molecule while maintaining the binding-specificity to the targets. In some embodiments, the N-terminus of the TGFβ receptor is fused to the Fc of the antibody construct (with or without an Fc linker). In some embodiments, the C-terminus of the antibody construct heavy chain is fused to the TGFβ receptor (with or without an Fc linker). In some embodiments, the C-terminal lysine residue of the antibody construct heavy chain is mutated to alanine.

In some embodiments, the antibodies in the immunoconjugates are glycosylated.

In some embodiments, the antibody in the immunoconjugates is a cysteine-engineered antibody which provides for site-specific conjugation of an adjuvant, label, or drug moiety to the antibody through cysteine substitutions at sites where the engineered cysteines are available for conjugation but do not perturb immunoglobulin folding and assembly or alter antigen binding and effector functions (Junutula, et al., 2008b Nature Biotech., 26(8):925-932; Dornan et al. (2009) Blood 114(13):2721-2729; U.S. Pat. Nos. 7,521,541; 7,723,485; US 2012/0121615; WO 2009/052249). Cysteine-engineered antibodies can be conjugated to the aza-benzazepine adjuvant moiety via an aza-benzazepine-linker compound with uniform stoichiometry (e.g., up to two aza-benzazepine moieties per antibody in an antibody that has a single engineered cysteine site).

In some embodiments, cysteine-engineered antibodies are used to prepare immunoconjugates. Immunoconjugates may have a reactive cysteine thiol residue introduced at a site on the light chain, such as the 149-lysine site (LC K149C), or on the heavy chain such as the 122-serine site (HC S122C), as numbered by Kabat numbering. In other embodiments, the cysteine-engineered antibodies have a cysteine residue introduced at the 118-alanine site (EU numbering) of the heavy chain (HC A118C). This site is alternatively numbered 121 by Sequential numbering or 114 by Kabat numbering. In other embodiments, the cysteine-engineered antibodies have a cysteine residue introduced at sites described in Bhakta, S. et al, (2013) “Engineering THIOMABs for Site-Specific Conjugation of Thiol-Reactive Linkers”, Laurent Ducry (ed.), Antibody-Drug Conjugates, Methods in Molecular Biology, vol. 1045, pages 189-203; WO 2011/156328; U.S. Pat. No. 9,000,130.

In an exemplary embodiment, the immunoconjugates of the invention comprise an antibody construct that comprises an antigen binding domain that specifically recognizes and binds PD-L1.

Programmed Death-Ligand 1 (PD-L1, cluster of differentiation 274, CD274, B7-homolog 1, or B7-H1) belongs to the B7 protein superfamily, and is a ligand of programmed cell death protein 1 (PD-1, PDCD1, cluster of differentiation 279, or CD279). PD-L1 can also interact with B7.1 (CD80) and such interaction is believed to inhibit T cell priming. The PD-L1/PD-1 axis plays a large role in suppressing the adaptive immune response. More specifically, it is believed that engagement of PD-L1 with its receptor, PD-1, delivers a signal that inhibits activation and proliferation of T-cells. Agents that bind to PD-L1 and prevent the ligand from binding to the PD-1 receptor prevent this immunosuppression, and can, therefore, enhance an immune response when desired, such as for the treatment of cancers, or infections.

PD-L1/PD-1 pathway also contributes to preventing autoimmunity and therefore agonistic agents against PD-L1 or agents that deliver immune inhibitory payloads may help treatment of autoimmune disorders.

Several antibodies targeting PD-L1 have been developed for the treatment of cancer, including atezolizumab (TECENTRIQ™), durvalumab (IMFINZI™), and avelumab (BAVENCIO™). Nevertheless, there continues to be a need for new PD-L1 antibody constructs, including agents that bind PD-L1 with high affinity and effectively prevent PD-L1/PD-1 signaling and agents that can deliver therapeutic payloads to PD-L1 expressing cells. In addition, there is a need for new PD-L1-binding agents to treat autoimmune disorders and infections.

A method is provided of delivering a TLR agonist payload to a cell expressing PD-L1 comprising administering to the cell, or mammal comprising the cell, an immunoconjugate comprising an anti-PD-L1 antibody covalently attached to a linker which is covalently attached to one or more TLR agonist moieties.

Also provided is a method for enhancing or reducing or inhibiting an immune response in a mammal, and a method for treating a disease, disorder, or condition in a mammal that is responsive to PD-L1 inhibition, which methods comprise administering a PD-L1 immunoconjugate thereof, to the mammal.

The invention provides a PD-L1 antibody comprising an immunoglobulin heavy chain variable region polypeptide and an immunoglobulin light chain variable region polypeptide. The PD-L1 antibody specifically binds PD-L1. The binding specificity of the antibody allows for targeting PD-L1 expressing cells, for instance, to deliver therapeutic payloads to such cells. In some embodiments, the PD-L1 antibody binds to human PD-L1. However, antibodies that bind to any PD-L1 fragment, homolog or paralog also are encompassed.

In some embodiments, the PD-L1 antibody binds PD-L1 without substantially inhibiting or preventing PD-L1 from binding to its receptor, PD-1. However, in other embodiments, the PD-L1 antibody can completely or partially block (inhibit or prevent) binding of PD-L1 to its receptor, PD-1, such that the antibody can be used to inhibit PD-L1/PD-1 signaling (e.g., for therapeutic purposes). The antibody or antigen-binding antibody fragment can be monospecific for PD-L1, or can be bispecific or multi-specific. For instance, in bivalent or multivalent antibodies or antibody fragments, the binding domains can be different targeting different epitopes of the same antigen or targeting different antigens. Methods of constructing multivalent binding constructs are known in the art. Bispecific and multispecific antibodies are known in the art. Furthermore, a diabody, triabody, or tetrabody can be provided, which is a dimer, trimer, or tetramer of polypeptide chains each comprising a V_{H} connected to a V_{L} by a peptide linker that is too short to allow pairing between the V_{H} and V_{L} on the same polypeptide chain, thereby driving the pairing between the complementary domains on different V_{H}-V_{L} polypeptide chains to generate a multimeric molecule having two, three, or four functional antigen binding sites. Also, bis-scFv fragments, which are small scFv fragments with two different variable domains can be generated to produce bispecific bis-scFv fragments capable of binding two different epitopes. Fab dimers (Fab2) and Fab trimers (Fab3) can be produced using genetic engineering methods to create multispecific constructs based on Fab fragments.

The PD-L1 antibody can be, or can be obtained from, a human antibody, a non-human antibody, a humanized antibody, or a chimeric antibody, or corresponding antibody fragments. A “chimeric” antibody is an antibody or fragment thereof typically comprising human constant regions and non-human variable regions. A “humanized” antibody is a monoclonal antibody typically comprising a human antibody scaffold but with non-human origin amino acids or sequences in at least one CDR (e.g., 1, 2, 3, 4, 5, or all six CDRs).

The PD-L1 antibody can be internalizing, as described in WO 2021/150701 and incorporated by reference herein, or the PD-L1 antibody can be non-internalizing, as described in WO 2021/150702 and incorporated by reference herein.

In an exemplary embodiment, the immunoconjugates of the invention comprise an antibody construct that comprises an antigen binding domain that specifically recognizes and binds HER2.

A number of anti-HER2 monoclonal antibodies are approved and under clinical development (Costa, R L B et al (2020) Breast Cancer 6(10):1-11.

In certain embodiments, immunoconjugates of the invention comprise an anti-HER2 antibody such as those prepared by the methods of Example 201. In one embodiment of the invention, an anti-HER2 antibody of an immunoconjugate of the invention comprises a humanized anti-HER2 antibody, e.g., huMAb4D5-1, huMAb4D5-2, huMAb4D5-3, huMAb4D5-4, huMAb4D5-5, huMAb4D5-6, huMAb4D5-7 and huMAb4D5-8, as described in Table 3 of U.S. Pat. No. 5,821,337, which is specifically incorporated by reference herein. Those antibodies contain human framework regions with the complementarity-determining regions of a murine antibody (4D5) that binds to HER2. The humanized antibody huMAb4D5-8 is also referred to as trastuzumab, commercially available under the tradename HERCEPTIN™ (Genentech, Inc.).

Trastuzumab (CAS 180288-69-1, huMAb4D5-8, rhuMAb HER2, HERCEPTIN®, Genentech, Inc.) is a recombinant DNA-derived, IgG1 kappa, monoclonal antibody that is a humanized version of a murine anti-HER2 antibody (4D5) that selectively binds with high affinity in a cell-based assay (Kd=5 nM) to the extracellular domain of HER2 (U.S. Pat. Nos. 5,677,171; 5,821,337; 6,054,297; 6,165,464; 6,339,142; 6,407,213; 6,639,055; 6,719,971; 6,800,738; 7,074,404; Coussens et al (1985) Science 230:1132-9; Slamon et al (1989) Science 244:707-12; Slamon et al (2001) New Engl. J Med. 344:783-792).

In an embodiment of the invention, the antibody construct or antigen binding domain comprises the CDR regions of trastuzumab. In an embodiment of the invention, the anti-HER2 antibody further comprises the framework regions of the trastuzumab. In an embodiment of the invention, the anti-HER2 antibody further comprises one or both variable regions of trastuzumab.

In another embodiment of the invention, an anti-HER2 antibody of an immunoconjugate of the invention comprises a humanized anti-HER2 antibody, e.g., humanized 2C4, as described in U.S. Pat. No. 7,862,817. An exemplary humanized 2C4 antibody is pertuzumab (CAS Reg. No. 380610-27-5), PERJETA™ (Genentech, Inc.). Pertuzumab is a HER dimerization inhibitor (HDI) and functions to inhibit the ability of HER2 to form active heterodimers or homodimers with other HER receptors (such as EGFR/HER1, HER2, HER3 and HER4). See, for example, Harari and Yarden, Oncogene 19:6102-14 (2000); Yarden and Sliwkowski. Nat Rev Mol Cell Biol 2:127-37 (2001); Sliwkowski Nat Struct Biol 10:158-9 (2003); Cho et al. Nature 421:756-60 (2003); and Malik et al. Pro Am Soc Cancer Res 44:176-7 (2003). PERJETA™ is approved for the treatment of breast cancer.

In an embodiment of the invention, the antibody construct or antigen binding domain comprises the CDR regions of pertuzumab. In an embodiment of the invention, the anti-HER2 antibody further comprises the framework regions of the pertuzumab. In an embodiment of the invention, the anti-HER2 antibody further comprises one or both variable regions of pertuzumab.

Margetuximab (MGAH22, MARGENZA™, MacroGenics, Inc.), CAS Reg. No. 1350624-75-7, is an FDA-approved anti-HER2 monoclonal antibody. The Fc region of margetuximab is optimized for increased binding to the activating Fc gamma Rs but decreased binding to the inhibitory Fc.gamma.Rs on immune effector cells (Nordstrom, J L, et al (2011) Breast Cancer Res. 13(6):R123; Rugo, H S, et al (2021) JAMA Oncol.;7(4):573-584; Markham, A. (2021) Drugs 81: 599-604). Margetuximab is approved by the FDA for treatment of patients with relapsed or refractory advanced breast cancer whose tumors express HER2 at the 2+ level by immunohistochemistry and lack evidence of HER2 gene amplification by FISH.

HT-19 is another anti-HER2 monoclonal antibody that binds to an epitope in human HER2 distinct from the epitope of trastuzumab or pertuzumab. HT-19 was shown to inhibit HER2 signaling comparable to trastuzumab and enhance HER2 degradation in combination with trastuzumab and pertuzumab. XMT-1522 is an antibody-drug conjugate comprising the HT-19 antibody (Bergstrom D. A. et al., (2015) Cancer Res.; 75:LB-231).

In an exemplary embodiment, the immunoconjugates of the invention comprise an antibody construct that comprises an antigen binding domain that specifically recognizes and binds CEA. Carcinoembryonic antigen-related cell adhesion molecule 5 (CEACAM5) also known as CD66e (Cluster of Differentiation 66e), is a member of the carcinoembryonic antigen (CEA) gene family.

Elevated expression of carcinoembryonic antigen (CEA, CD66e, CEACAM5) has been implicated in various biological aspects of neoplasia, especially tumor cell adhesion, metastasis, the blocking of cellular immune mechanisms, and having antiapoptosis functions. CEA is also used as a blood marker for many carcinomas. Labetuzumab (CEA-CIDE™, Immunomedics, CAS Reg. No. 219649-07-7), also known as MN-14 and hMN14, is a humanized IG1 monoclonal antibody and has been studied for the treatment of colorectal cancer (Blumenthal, R. et al (2005) Cancer Immunology Immunotherapy 54(4):315-327). Labetuzumab conjugated to a camptothecin analog (labetuzumab govitecan, IMMU-130) targets carcinoembryonic antigen-related cell adhesion mol. 5 (CEACAM5) and is being studied in patients with relapsed or refractory metastatic colorectal cancer (Sharkey, R. et al, (2018), Molecular Cancer Therapeutics 17(1):196-203; Cardillo, T. et al (2018) Molecular Cancer Therapeutics 17(1):150-160). In an embodiment of the invention, the CEA-targeting antibody construct or antigen binding domain comprises the Variable light chain (VL kappa) of hMN-14/labetuzumab as disclosed in U.S. Pat. No. 6,676,924, which is incorporated by reference herein for this purpose.

In an embodiment of the invention, the heavy chain (HC) of a CEA-targeting antibody is selected from SEQ ID NO: 1-5.

| | | | SEQ |
|---|---|---|---|
| | | Table 3 | ID |
| Heavy chain (HC) | Length | reference | NO: |
| EVQLVESGGGVVQPGRSLRLSCSSSGFDFTTYWMSWVRQAPGKGLEWVA | 449 | CEA.3-G1f, | 1 |
| EIHPDSSTINYAPSLKDRFTISRDNSKNTLFLQMDSLRPEDTGVYFCAS | | hMN-14/ | |
| LYFGFPWFAYWGQGTPVTVSSASTKGPSVFPLAPSSKSTSGGTAALGCL | | labetuzumab | |
| VKDYFPEPVTVSWNSGALTSGVHTFPAVLQSSGLYSLSSVVTVPSSSLG | | as human | |
| TQTYICNVNHKPSNTKVDKRVEPKSCDKTHTCPPCPAPELLGGPSVFLF | | IgG1f | |
| PPKPKDTLMISRTPEVTCVVVDVSHEDPEVKFNWYVDGVEVHNAKTKPR | | also: CEA.3- | |
| EEQYNSTYRVVSVLTVLHQDWLNGKEYKCKVSNKALPAPIEKTISKAKG | | G1fnf, | |
| QPREPQVYTLPPSREEMTKNQVSLTCLVKGFYPSDIAVEWESNGQPENN | | nonfucosylated | |
| YKTTPPVLDSDGSFFLYSKLTVDKSRWQQGNVFSCSVMHEALHNHYTQK | | hMN-14/ | |
| SLSLSPGK | | labetuzumab | |
| | | with IgG1f | |
| EVQLQESGPGLVKPGGSLSLSCAASGFVFSSYDMSWVRQTPERGLEWVA | 450 | CEA.6-G1f, | 2 |
| YISSGGGITYAPSTVKGRFTVSRDNAKNTLYLQMNSLTSEDTAVYYCAA | | tusamitamab/ | |
| HYFGSSGPFAYWGQGTLVTVSSASTKGPSVFPLAPSSKSTSGGTAALGC | | huMAb2-3 | |
| LVKDYFPEPVTVSWNSGALTSGVHTFPAVLQSSGLYSLSSVVTVPSSSL | | | |
| GTQTYICNVNHKPSNTKVDKRVEPKSCDKTHTCPPCPAPELLGGPSVFL | | | |
| FPPKPKDTLMISRTPEVTCVVVDVSHEDPEVKFNWYVDGVEVHNAKTKP | | | |
| REEQYNSTYRVVSVLTVLHQDWLNGKEYKCKVSNKALPAPIEKTISKAK | | | |
| GQPREPQVYTLPPSREEMTKNQVSLTCLVKGFYPSDIAVEWESNGQPEN | | | |
| NYKTTPPVLDSDGSFFLYSKLTVDKSRWQQGNVFSCSVMHEALHNHYTQ | | | |
| KSLSLSPGK | | | |
| EVQLVESGGGLVQPGRSLRLSCAASGFTVSSYWMHWVRQAPGKGLEWVG | 451 | CEA.9- | 3 |
| FIRNKANSGTTEYAASVKGRFTISRDDSKNTLYLQMNSLRAEDTAVYYC | | G1fhL2, | |
| ARDRGLRFYFDYWGQGTTVTVSSASTKGPSVFPLAPSSKSTSGGTAALG | | CEA.5/huA5B7 | |
| CLVKDYFPEPVTVSWNSGALTSGVHTFPAVLQSSGLYSLSSVVTVPSSS | | with VH-G54S | |
| LGTQTYICNVNHKPSNTKVDKRVEPKSCDKTHTCPPCPAPELLGGPSVF | | | |
| LFPPKPKDTLMISRTPEVTCVVVDVSHEDPEVKFNWYVDGVEVHNAKTK | | | |
| PREEQYNSTYRVVSVLTVLHQDWLNGKEYKCKVSNKALPAPIEKTISKA | | | |
| KGQPREPQVYTLPPSREEMTKNQVSLTCLVKGFYPSDIAVEWESNGQPE | | | |
| NNYKTTPPVLDSDGSFFLYSKLTVDKSRWQQGNVFSCSVMHEALHNHYT | | | |
| QKSLSLSPGK | | | |
| EVQLVESGGGVVQPGRSLRLSCSSSGFDFTTYWMSWVRQAPGKGLEWVA | 449 | CEA.3-G1f- | 4 |
| EIHPDSSTINYAPSLKDRFTISRDNSKNTLFLQMDSLRPEDTGVYFCAS | | G236A, hMN-14/ | |
| LYFGFPWFAYWGQGTPVTVSSASTKGPSVFPLAPSSKSTSGGTAALGCL | | labetuzumab | |
| VKDYFPEPVTVSWNSGALTSGVHTFPAVLQSSGLYSLSSVVTVPSSSLG | | with human | |
| TQTYICNVNHKPSNTKVDKRVEPKSCDKTHTCPPCPAPELLAGPSVFLF | | IgG1f-G236A | |
| PPKPKDTLMISRTPEVTCVVVDVSHEDPEVKFNWYVDGVEVHNAKTKPR | | | |
| EEQYNSTYRVVSVLTVLHQDWLNGKEYKCKVSNKALPAPIEKTISKAKG | | | |
| QPREPQVYTLPPSREEMTKNQVSLTCLVKGFYPSDIAVEWESNGQPENN | | | |
| YKTTPPVLDSDGSFFLYSKLTVDKSRWQQGNVFSCSVMHEALHNHYTQK | | | |
| SLSLSPGK | | | |
| EVQLVESGGGVVQPGRSLRLSCSSSGFDFTTYWMSWVRQAPGKGLEWVA | 449 | CEA.3-G1f- | 5 |
| EIHPDSSTINYAPSLKDRFTISRDNSKNTLFLQMDSLRPEDTGVYFCAS | | SDIE, hMN-14/ | |
| LYFGFPWFAYWGQGTPVTVSSASTKGPSVFPLAPSSKSTSGGTAALGCL | | labetuzumab | |
| VKDYFPEPVTVSWNSGALTSGVHTFPAVLQSSGLYSLSSVVTVPSSSLG | | with human | |
| TQTYICNVNHKPSNTKVDKRVEPKSCDKTHTCPPCPAPELLGGPDVFLF | | IgG1f-SDIE | |
| PPKPKDTLMISRTPEVTCVVVDVSHEDPEVKFNWYVDGVEVHNAKTKPR | | | |
| EEQYNSTYRVVSVLTVLHQDWLNGKEYKCKVSNKALPAPEEKTISKAKG | | | |
| QPREPQVYTLPPSREEMTKNQVSLTCLVKGFYPSDIAVEWESNGQPENN | | | |
| YKTTPPVLDSDGSFFLYSKLTVDKSRWQQGNVFSCSVMHEALHNHYTQK | | | |
| SLSLSPGK | | | |

In an embodiment of the invention, the light chain (LC) of a CEA-targeting antibody is selected from SEQ TD NO: 6-9.

| | | | SEQ |
|---|---|---|---|
| | | Table 3 | ID |
| Light chain (LC) | Length | reference | NO: |
| DIQLTQSPSSLSASVGDRVTITCKASQDVGTSVAWYQQKPGKAPKLL | 213 | CEA.3-Glf, | 6 |
| IYWTSTRHTGVPSRFSGSGSGTDFTFTISSLQPEDIATYYCQQYSLY | | hMN-14/ | |
| RSFGQGTKVEIKRTVAAPSVFIFPPSDEQLKSGTASVVCLLNNFYPR | | labetuzumab | |
| EAKVQWKVDNALQSGNSQESVTEQDSKDSTYSLSSTLTLSKADYEKH | | as human | |
| KVYACEVTHQGLSSPVTKSFNRGEC | | IgG1f | |
| | | also: CEA.3- | |
| | | G1f-G236A, | |
| | | hMN-14/ | |
| | | labetuzumab | |
| | | with human | |
| | | IgG1f-G236A | |
| | | also: CEA.3- | |
| | | G1f-SDIE, | |
| | | hMN-14/ | |
| | | labetuzumab | |
| | | with human | |
| | | IgG1f-SDIE | |
| DIQMTQSPASLSASVGDRVTITCRASENIFSYLAWYQQKPGKSPKLL | 214 | CEA.6-G1f, | 7 |
| VYNTRTLAEGVPSRFSGSGSGTDFSLTISSLQPEDFATYYCQHHYGT | | tusamitamab/ | |
| PFTFGSGTKLEIKRTVAAPSVFIFPPSDEQLKSGTASVVCLLNNFYP | | huMAb2-3 | |
| REAKVQWKVDNALQSGNSQESVTEQDSKDSTYSLSSTLTLSKADYEK | | | |
| HKVYACEVTHQGLSSPVTKSFNRGEC | | | |
| QAVLTQPASLSASPGASASLTCTLRRGINVGAYSIYWYQQKPGSPPQ | 222 | CEA.9- | 8 |
| YLLRYKSDSDKQQGSGVSSRFSASKDASANAGILLISGLQSEDEADY | | G1fhL2, | |
| YCMIWHSGASAVFGGGTKLTVLGQPKAAPSVTLEPPSSEELQANKAT | | CEA.5/huA5B7 | |
| LVCLISDFYPGAVTVAWKADSSPVKAGVETTTPSKQSNNKYAASSYL | | with VH-G54S | |
| SLTPEQWKSHRSYSCQVTHEGSTVEKTVAPTECS | | | |
| DIQLTQSPSSLSASVGDRVTITCKASQDVGTSVAWYQQKPGKAPKLL | 213 | CEA.3-G1f- | 9 |
| IYWTSTRHTGVPSRFSGSGSGTDFTFTISSLQPEDIATYYCQQYSLY | | SDIE, hMN-14/ | |
| RSFGQGTKVEIKRTVAAPSVFIFPPSDEQLKSGTASVVCLLNNFYPR | | labetuzumab | |
| EAKVQWKVDNALQSGNSQESVTEQDSKDSTYSLSSTLTLSKADYEKH | | with human | |
| KVYACEVTHQGLSSPVTKSFNRGEC | | IgG1f-SDIE | |

In an exemplary embodiment, the immunoconjugates of the invention comprise an antibody construct that comprises an antigen binding domain that specifically recognizes and binds TROP2. Tumor-associated calcium signal transducer 2 (TROP-2) is a. Transurban glycoprotein encoded by the TACSTD2 gene (Linnenbach A J, et al (1993) Mol Cell Biol. 13(3): 1507-15, Calabrese G, et al (2001) Cytogenet Cell Genet. 92(1-2). 164-5). TROP2 is an intracellular calcium signal transducer that is differentially expressed in many cancers and signals cells for self-renewal, proliferation, invasion, and survival, TROP2 is considered a stern cell marker and is expressed in many normal tissues, though in contrast, it is overexpressed in many cancers (Ohmachi T, et al., (2006) Clin. Cancer Res., 12(10), 3057-3063; Muhlmann G, et al., (2009) J. Clin. Pathol., 62(2), 152-158; Fong D, et al., (2008) Br. J. Cancer, 99(8), 1290-1295; Fong D, et al., (2008) Mod. Pathol., 21(2), 186-191; Ning S, et al., (2013) Neurol. Sci., 34(10), 1745-1750). Overexpression of TROP2 is of prognostic significance, Several ligands have been proposed that interact with TROP2. TROP2 signals the cells via different pathways and it is transcriptionally regulated by a complex network of several transcription factors.

Human TROP2 (TACSTD2: tumor-associated calcium signal transducer 2, GA733-1, EGP-1, M1S1; hereinafter, referred to as hTROP2) is a single-pass transmembrane type 1 cell membrane protein consisting of 323 amino acid residues. While the presence of a cell membrane protein involved in immune resistance, which is common to human trophoblasts and cancer cells (Faulk W P, et al., Proc. Natl. Acad. Sci. 75(4):1947-1951 (1978)), has previously been suggested, an antigen molecule recognized by a monoclonal antibody against a cell membrane protein in a human choriocarcinoma cell line was identified and designated as TROP2 as one of the molecules expressed in human trophoblasts (Lipinski M, et al., Proc. Natl. Acad. Sci. 78(8), 5147-5150 (1981)). This molecule was also designated as tumor antigen GA733-1 recognized by a mouse monoclonal antibody GA733 (Linnenbach A J, et al., Proc. Natl. Acad. Sci. 86(1), 27-31 (1989)) obtained by immunization with a gastric cancer cell line or an epithelial glycoprotein (EGP-1; Basu A, et al., Int. J. Cancer, 62 (4), 472-479 (1995)) recognized by a mouse monoclonal antibody RS7-3G11 obtained by immunization with non-small cell lung cancer cells. In 1995, however, the TROP2 gene was cloned, and all of these molecules were confirmed to be identical molecules (Fornaro M, et al., Int. J. Cancer, 62(5), 610-618 (1995)). The DNA sequence and amino acid sequence of hTROP2 are available on a public database and can be referred to, for example, under Accession Nos. NM_002353 and NP_002344 (NCBI).

In response to such information suggesting the association with cancer, a plurality of anti-hTROP2 antibodies have been established so far and studied for their antitumor effects. Among these antibodies, there is disclosed, for example, an unconjugated antibody that exhibits in itself antitumor activity in nude mouse xenograft models (WO 2008/144891; WO 2011/145744; WO 2011/155579; WO 2013/077458) as well as an antibody that exhibits antitumor activity as ADC with a cytotoxic drug (WO 2003/074566; WO 2011/068845; WO 2013/068946; U.S. Pat. No. 7,999,083). However, the strength or coverage of their activity is still insufficient, and there are unsatisfied medical needs for hTROP2 as a therapeutic target.

TROP2 expression in cancer cells has been correlated with drug resistance. Several strategies target TROP2 on cancer cells that include antibodies, antibody fusion proteins, chemical inhibitors, nanoparticles, etc. The in vitro studies and pre-clinical studies, using these various therapeutic treatments, have resulted in significant inhibition of tumor cell growth both in vitro and in vivo in mice. Clinical studies have explored the potential application of TROP2 as both a prognostic biomarker and as a therapeutic target to reverse resistance.

Sacituzumab govitecan (TRODELVY®, Immunomedics, IMMU-132), an antibody-drug conjugate comprising a TROP2-directed antibody linked to a topoisomerase inhibitor drug, is indicated for the treatment of metastatic triple-negative breast cancer (mTNBC) in adult patients that have received at least two prior therapies. The TROP2 antibody in sacituzumab govitecan is conjugated to SN-38, the active metabolite of irinotecan (US 2016/0297890; WO 2015/098099).

In an embodiment of the invention, the TROP2-targeting antibody construct or antigen binding domain comprises the light chain CDR (complementarity determining region) of hRS7 (humanized RS7), (U.S. Pat. No. 7,238,785, incorporated by reference herein).

In an exemplary embodiment, the immunoconjugates of the invention comprise an antibody construct that comprises an antigen binding domain that specifically recognizes and binds Caprin-1 (Ellis J A, Luzio J P (1995) J Biol Chem. 270(35):20717-23; Wang B, et al (2005) J Immunol. 175 (7):4274-82; Solomon S, et al (2007) Mol Cell Biol. 27(6):2324-42). Caprin-1 is also known as GPIAP1, GPIP137, GRIP137, M11S1, RNG105, p137GPI, and cell cycle associated protein 1.

Cytoplasmic activation/proliferation-associated protein-1 (caprin-1) is an RNA-binding protein that participates in the regulation of cell cycle control-associated genes. Caprin-1 selectively binds to c-Myc and cyclin D2 mRNAs, which accelerates cell progression through the G₁ phase into the S phase, enhances cell viability and promotes cell growth, indicating that it may serve an important role in tumorigenesis (Wang B, et al (2005) J Immunol. 175:4274-4282). Caprin-1 acts alone or in combination with other RNA-binding proteins, such as RasGAP SH3-domain-binding protein 1 and fragile X mental retardation protein. In the tumorigenesis process, caprin-1 primarily functions by activating cell proliferation and upregulating the expression of immune checkpoint proteins. Through the formation of stress granules, caprin-1 is also involved in the process by which tumor cells adapt to adverse conditions, which contributes to radiation and chemotherapy resistance. Given its role in various clinical malignancies, caprin-1 holds the potential to be used as a biomarker and a target for the development of novel therapeutics (Yang, Z-S, et al (2019) Oncology Letters 18:15-21).

Antibodies that target caprin-1 for treatment and detection have been described (WO 2011/096519; WO 2013/125654; WO 2013/125636; WO 2013/125640; WO 2013/125630; WO 2013/018889; WO 2013/018891; WO 2013/018883; WO 2013/018892; WO 2014/014082; WO 2014/014086; WO 2015/020212; WO 2018/079740).

In an exemplary embodiment, the immunoconjugates of the invention comprise an antibody construct that comprises an antigen binding domain that specifically recognizes and binds Claudin-1.

Claudin-1 is a member of the transmembrane protein family claudins located in cell-cell tight junctions and it acts as a co-receptor for HCV entry into hepatic cells (Kniesel U, et al (2000). Cell. Mol. Neurobiol. 20(1):57-76; Furuse M, et al (1998). J. Cell Biol. 141(7):1539-50; Swissbelm K, et al (2005) Adv. Drug Deliv. Rev. 57(6):919-28). Claudin 1 is also known as Senescence-associated epithelial membrane protein, senescence-associated epithelial membrane protein 1, CLDN1, CLD1, ILVASC, SEMP1.

Claudins are abundant in luminal epithelial sheets where they maintain epithelial cell polarity. Claudin-1 is expressed in most tissues such as bladder, fallopian tube, liver, pancreas, prostate, and skin.

In an exemplary embodiment, the immunoconjugates of the invention comprise an antibody construct that comprises an antigen binding domain that specifically recognizes and binds Nectin-4.

The nectins are a protein family of cell adhesion molecules involved in calcium-dependent cell adhesion (Takai Y. et al (2003) Cancer Science 94(8):655-67; Fuchs, A. et al (2006) Seminars in Cancer Biology 16(5):359-366; Miyoshi J. et al (2007) American journal of nephrology 27(6):590-604). Nectins play an important role in the bonding between cells in many different tissues, including the intermediate junction of epithelial cells or the chemical synapse of nerve cells.

### Antibody Targets

In some embodiments, the antibody of an immunoconjugate is capable of binding one or more targets selected from (e.g., specifically binds to a target selected from) 5T4, ABL, ABCF1, ACVR1, ACVR1B, ACVR2, ACVR2B, ACVRL1, ADORA2A, Aggrecan, AGR2, AICDA, AIF1, AIGI, AKAP1, AKAP2, AMH, AVIHR2, ANGPT1, ANGPT2, ANGPTL3, ANGPTL4, ANPEP, APC, APOC1, AR, aromatase, ATX, AX1, AZGP1 (zinc-a-glycoprotein), B7.1, B7.2, B7-H1, BAD, BAFF, BAG1, BAI1, BCR, BCL2, BCL6, BDNF, BLNK, BLR1 (MDR15), BIyS, BMP1, BMP2, BMP3B (GDFIO), BMP4, BMP6, BMP8, BMPRTA, BMPR1B, BMPR2, BPAG1 (plectin), BRCA1, C19orflO (IL27w), C3, C4A, C5, C5R1, CANT1, CAPRIN-1, CASP1, CASP4, CAV1, CCBP2 (D6/JAB61), CCLI (1-309), CCLI1 (eotaxin), CCL13 (MCP-4), CCL15 (MIP-Id), CCL16 (HCC-4), CCL17 (TARC), CCL18 (PARC), CCL19 (MIP-3b), CCL2 (MCP-1), MCAF, CCL20 (MIP-3a), CCL21 (MEP-2), SLC, exodus-2, CCL22(MDC/STC-1), CCL23 (MPIF-I), CCL24 (MPIF-2/eotaxin-2), CCL25 (TECK), CCL26 (eotaxin-3), CCL27 (CTACK/TLC), CCL28, CCL3 (MIP-Ia), CCL4 (MIPIb), CCL5 (RANTES), CCL7 (MCP-3), CCL8 (mcp-2), CCNA1, CCNA2, CCND1, CCNE1, CCNE2, CCR1 (CKR1/HM145), CCR2 (mcp-IRB/RA), CCR3 (CKR3/CMKBR3), CCR4, CCR5 (CMKBR5/ChemR13), CCR6 (CMKBR6/CKR-L3/STRL22/DRY6), CCR7 (CKR7/EBI1), CCR8 (CMKBR8/TERI/CKR-L1), CCR9 (GPR-9-6), CCRL1 (VSHK1), CCRL2 (L-CCR), CD164, CD19, CDIC, CD2, CD20, CD21, CD200, CD-22, CD24, CD27, CD28, CD3, CD33, CD35, CD37, CD38, CD3E, CD3G, CD3Z, CD4, CD38, CD40, CD40L, CD44, CD45RB, CD47, CD52, CD69, CD72, CD74, CD79A, CD79B, CD8, CD80, CD81, CD83, CD86, CD137, CD152, CD274, CDH1 (Ecadherin), CDH1O, CDH12, CDH13, CDH18, CDH19, CDH2O, CDH5, CDH7, CDH8, CDH9, CDK2, CDK3, CDK4, CDK5, CDK6, CDK7, CDK9, CDKN1A (p21Wap1/Cip1), CDKN1B (p27Kip1), CDKN1C, CDKN2A (p16INK4a), CDKN2B, CDKN2C, CDKN3, CEBPB, CERI, CHGA, CHGB, Chitinase, CHST1O, CKLFSF2, CKLFSF3, CKLFSF4, CKLFSF5, CKLFSF6, CKLFSF7, CKLFSF8, CLDN3, CLDN7 (claudin-7), CLDN18.2 (claudin 18.2), CLN3, CLU (clusterin), CMKLR1, CMKOR1 (RDC1), CNR1, COL18A1, COLIA1, COL4A3, COL6A1, CR2, Cripto, CRP, CSF1 (M-CSF), CSF2 (GM-CSF), CSF3 (GCSF), CTL8, CTNNB1 (b-catenin), CTSB (cathepsin B), CX3CL1 (SCYD1), CX3CR1 (V28), CXCL1 (GRO1), CXCL1O (IP-IO), CXCLI1 (1-TAC/IP-9), CXCL12 (SDF1), CXCL13, CXCL14, CXCL16, CXCL2 (GRO2), CXCL3 (GRO3), CXCL5 (ENA-78/LIX), CXCL6 (GCP-2), CXCL9 (MIG), CXCR3 (GPR9/CKR-L2), CXCR4, CXCR6 (TYMSTR/STRL33/Bonzo), CYB5, CYC1, CYSLTR1, DAB2IP, DES, DKFZp451J0118, DNCL1, DPP4, E2F1, Engel, Edge, Fennel, EFNA3, EFNB2, EGF, EGFR, ELAC2, ENG, Enola, ENO2, ENO3, EPHA1, EPHA2, EPHA3, EPHA4, EPHA5, EPHA6, EPHA7, EPHA8, EPHA9, EPRA10, EPHB1, EPHB2, EPHB3, EPHB4, EPHB5, EPHB6, EPHRIN-A1, EPHRIN-A2, EPHRINA3, EPHRIN-A4, EPHRIN-A5, EPHRIN-A6, EPHRIN-B1, EPHRIN-B2, EPHRIN-B3, EPHB4, EPG, ERBB2 (Her-2), EREG, ERK8, Estrogen receptor, Earl, ESR2, F3 (TF), FADD, famesyltransferase, FasL, FASNf, FCER1A, FCER2, FCGR3A, FGF, FGF1 (aFGF), FGF10, FGF11, FGF12, FGF12B, FGF13, FGF14, FGF16, FGF17, FGF18, FGF19, FGF2 (bFGF). FGF20, FGF21, FGF22, FGF23, FGF3 (int-2), FGF4 (HST), FGF5, FGF6 (HST-2), FGF7 (KGF), FGF8, FGF9, FGFR3, FIGF (VEGFD), FILI (EPSILON), FBL1 (ZETA), FLJ12584, FLJ25530, FLRT1 (fibronectin), FLT1, FLT-3, FOS, FOSL1 (FRA-1), FY (DARC), GABRP (GABAa), GAGEB1, GAGEC1, GALNAC4S-6ST, GATA3, GD2, GDF5, GFI1, GGT1, GM-CSF, GNAS1, GNRH1, GPR2 (CCR10), GPR31, GPR44, GPR81 (FKSG80), GRCC1O (C1O), GRP, GSN (Gelsolin), GSTP1, HAVCR2, HDAC, HDAC4, HDAC5, HDAC7A, HDAC9, Hedgehog, HGF, HIF1A, HIP1, histamine and histamine receptors, HLA-A, HLA-DRA, HLA-E, HM74, HMOXI, HSP90, HUMCYT2A, ICEBERG, ICOSL, ID2, IFN-a, IFNA1, IFNA2, IFNA4, IFNA5, EFNA6, BFNA7, IFNB1, IFNgamma, IFNW1, IGBP1, IGF1, IGFIR, IGF2, IGFBP2, IGFBP3, IGFBP6, DL-1, ILIO, ILIORA, ILIORB, IL-1, IL1R1 (CD121a), IL1R2 (CD121b), IL-IRA, IL-2, IL2RA (CD25), IL2RB (CD122), IL2RG (CD132), IL-4, IL-4R (CD123), IL-5, IL5RA (CD125), IL3RB (CD131), IL-6, IL6RA, (CD126), IR6RB (CD130), IL-7, IL7RA (CD127), IL-8, CXCR1 (IL8RA), CXCR2, (IL8RB/CD128), IL-9, IL9R (CD129), IL-10, IL1ORA (CD210), IL1ORB (CDW210B), IL-11, IL IRA, IL-12, IL-12A, IL-12B, IL-12RB1, IL-12RB2, IL-13, IL13RA1, IL13RA2, IL14, IL15, IL15RA, IL16, IL17, IL17A, IL17B, IL17C, IL17R, IL18, IL18BP, IL18R1, IL18RAP, IL19, ILIA, ILIB, ILIF10, ILIF5, IL1F6, ILIF7, IL1F8, DL1F9, ILIHYI, ILIR1, ILIR2, ILIRAP, ILIRAPL1, ILIRAPL2, ILIRL1, IL1RL2, ILIRN, IL2, IL20, IL20RA, IL21R, IL22, IL22R, IL22RA2, IL23, DL24, IL25, IL26, IL27, IL28A, IL28B, IL29, IL2RA, IL2RB, IL2RG, IL3, IL30, IL3RA, IL4, IL4, IL6ST (glycoprotein 130), ILK, INHA, INHBA, INSL3, INSL4, IRAK1, IRAK2, ITGA1, ITGA2, ITGA3, ITGA6 (.alpha.6 integrin), ITGAV, ITGB3, ITGB4 (.beta.4 integrin), JAG1, JAK1, JAK3, JTB, JUN, K6HF, KAI1, KDR, KITLG, KLF5 (GC Box BP), KLF6, KLK10, KLK12, KLK13, KLK14, KLK15, KLK3, KLK4, KLK5, KLK6, KLK9, KRT1, KRT19 (Keratin 19), KRT2A, KRTHB6 (hair-specific type II keratin), LAMA5, LEP (leptin), Lingo-p75, Lingo-Troy, LPS, LTA (TNF-b)), LTB, LTB4R (GPR16), LTB4R2, LTBR, MACMARCKS, MAG or OMgp, MAP2K7 (c-Jun), MCP-1, MDK, MIB1, midkine, MIF, MISRII, MJP-2, MK, MKI67 (Ki-67), MMP2, MMP9, MS4A1, MSMB, MT3 (metallothionectin-UI), mTOR, MTSS1, MUC1 (mucin), MYC, MYD88, NCK2, neurocan, Nectin-4, NFKBI, NFKB2, NGFB (NGF), NGFR, NgR-Lingo, NgRNogo66, (Nogo), NgR-p75, NgR-Troy, NMEI (NM23A), NOTCH, NOTCH1, NOX5, NPPB, NROB1, NROB2, NRID1, NR1D2, NR1H2, NR1H3, NR1H4, NR112, NR113, NR2C1, NR2C2, NR2E1, NR2E3, NR2F1, NR2F2, NR2F6, NR3C1, NR3C2, NR4A1, NR4A2, NR4A3, NR5A1, NR5A2, NR6A1, NRP1, NRP2, NT5E, NTN4, ODZI, OPRDI, P2RX7, PAP, PART1, PATE, PAWR, PCA3, PCDGF, PCNA, PDGFA, PDGFB, PDGFRA, PDGFRB, PECAMI, peg-asparaginase, PF4 (CXCL4), PGF, PGR, phosphacan, PIAS2, PI3 Kinase, PIK3CG, PLAU (uPA), PLG, PLXDCI, PKC, PKC-beta, PPBP (CXCL7), PPID, PR1, PRKCQ, PRKD1, PRL, PROC, PROK2, PSAP, PSCA, PTAFR, PTEN, PTGS2 (COX-2), PIN, RAC2 (P21Rac2), RANK, RANK ligand, RARB, RGS1, RGS13, RGS3, RNFI1O (ZNF144), Ron, ROBO2, RXR, S100A2, SCGB 1D2 (lipophilin B), SCGB2A1 (mammaglobin 2), SCGB2A2 (mammaglobin 1), SCYE1 (endothelial Monocyte-activating cytokine), SDF2, SERPENA1, SERPINA3, SERPINB5 (maspin), SERPINEI (PAI-I), SERPINFI, SHIP-1, SHIP-2, SHB1, SHB2, SHBG, SfcAZ, SLC2A2, SLC33A1, SLC43A1, SLIT2, SPP1, SPRR1B (Spr1), ST6GAL1, STAB1, STATE, STEAP, STEAP2, TB4R2, TBX21, TCP1O, TDGF1, TEK, TGFA, TGFB1, TGFB1I1, TGFB2, TGFB3, TGFBI, TGEBR1, TGFBR2, TGFBR3, THIL, THBS1 (thrombospondin-1), THBS2, THBS4, THPO, TIE (Tie-1), TIMP3, tissue factor, TLR1, TLR2, TLR3, TLR4, TLR5, TLR6, TLR7, TLR8, TLR9, TLR10, TLR11, TNF, TNF-α, TNFAIP2 (B94), TNFAIP3, TNFRSF11A, TNFRSF1A, TNFRSF1B, TNFRSF21, TNFRSF5, TNFRSF6 (Fas), TNFRSF7, TNFRSF8, TNFRSF9, TNFSF1O (TRAIL), TNFSF 11 (TRANCE), TNFSF12 (APO3L), TNFSF13 (April), TNFSF13B, TNSF14 (HVEM-L), TNFRSF14 (HVEM), TNFSF15 (VEGI), TNFSF18, TNFSF4 (OX40 ligand), TNFSF5 (CD40 ligand). TNFSF6 (FasL), TNFSF7 (CD27 ligand), TNFSF8 (CD30 ligand), TNFSF9 (4-1BB ligand), TOLLIP, Toll-like receptors, TOP2A (topoisomerase 1ia), TP53, TPM1, TPM2, TRADD, TRAF1, TRAF2, TRAF3, TRAF4, TRAF5, TRAF6, TRKA, TREM1, TREM2, TROP2, TRPC6, TSLP, TWEAK, Tyrosinase, uPAR, VEGF, VEGFB, VEGFC, versican, VHL C5, VLA-4, Wnt-1, XCL1 (tymphotactin), XCL2 (SCM-Ib), XCRI (GPR5/CCXCR1), YYI, ZFPM2, CLEC4C (BDCA-2, DLEC, CD303, CLECSF7), CLEC4D (MCL, CLECSF8), CLEC4E (Mincle), CLEC6A (Dectin-2). CLEC5A (MDL-1, CLECSF5), CLEC1B (CLEC-2), CLEC9A (DNGR-1), CLEC7A (Dectin-1), PDGFRa, SLAMF7, GP6 (GPVI), LILRA1 (CD85I), LILRA2 (CD85H, ILT1), LILRA4 (CD85G, ILT7), LILRA5 (CD85F, ILT11), LILRA6 (CD85b, ILT8), NCR1 (CD335, LY94, NKp46), NCR3 (CD335, LY94, NKp46), NCR3 (CD337, NKp30), OSCAR, TARM1, CD300C, CD300E, CD300LB (CD300B), CD300LD (CD300D), KIR2DL4 (CD158D), KIR2DS, KLRC2 (CD159C, NKG2C), KLRK1 (CD314, NKG2D), NCR2 (CD336, NKp44), PILRB, SIGLEC1 (CD169, SN), SIGLEC14, SIGLEC15 (CD33L3), SIGLEC16, SIRPalpha, SIRPB1 (CD172B), TREM1 (CD354), TREM2, and KLRF1 (NKp80).

In some embodiments, the antibody binds to an FcR.gamma-coupled receptor. In some embodiments, the FcR.gamma-coupled receptor is selected from the group consisting of GP6 (GPVI), LILRA1 (CD85I), LILRA2 (CD85H, ILT1), LILRA4 (CD85G, ILT7), LILRA5 (CD85F, ILT11), LILRA6 (CD85b, ILT8), NCR1 (CD335, LY94, NKp46), NCR3 (CD335, LY94, NKp46), NCR3 (CD337, NKp30), OSCAR, and TARM1.

In some embodiments, the antibody binds to a DAP12-coupled receptor. In some embodiments, the DAP12-coupled receptor is selected from the group consisting of CD300C, CD300E, CD300LB (CD300B), CD300LD (CD300D), KIR2DL4 (CD158D), KIR2DS, KLRC2 (CD159C, NKG2C), KLRK1 (CD314, NKG2D), NCR2 (CD336, NKp44). PILRB, SIGLEC1 (CD169, SN), SIGLEC14, SIGLEC15 (CD33L3), SIGLEC16, SIRPB1 (CD172B), TREM1 (CD354), and TREM2.

In some embodiments, the antibody binds to a hemITAM-bearing receptor. In some embodiments, the hemITAM-bearing receptor is KLRF1 (NKp80).

In some embodiments, the antibody is capable of binding one or more targets selected from CLEC4C (BDCA-2, DLEC, CD303, CLECSF7), CLEC4D (MCL, CLECSF8), CLEC4E (Mincle), CLEC6A (Dectin-2), CLEC5A (MDL-1, CLECSF5), CLEC1B (CLEC-2), CLEC9A (DNGR-1), and CLEC7A (Dectin-1). In some embodiments, the antibody is capable of binding CLEC6A (Dectin-2) or CLEC5A. In some embodiments, the antibody is capable of binding CLEC6A (Dectin-2).

In some embodiments, the antibody is capable of binding one or more targets selected from (e.g., specifically binds to a target selected from): ATP5I (Q06185), OAT (P29758), AIFM1 (Q9ZOX1), AOFA (Q64133), MTDC (P18155), CMC1 (Q8BH59), PREP (Q8K411), YMEL1 (O88967), LPPRC (Q6PB66), LONM (Q8CGK3), ACON (Q99KI0), ODO1 (Q60597), IDHP (P54071), ALDH2 (P47738), ATPB (P56480), AATM (P05202), TMM93 (Q9CQW0), ERGI3 (Q9CQE7), RTN4 (Q99P72), CL041 (Q8BQR4), ERLN2 (Q8BFZ9), TERA (Q01853), DAD1 (P61804), CALX (P35564), CALU (O35887), VAPA (Q9WV55), MOGS (Q80UM7), GANAB (Q8BHN3), ERO1A (Q8R180), UGGG1 (Q6P5E4), P4HA1 (Q60715), HYEP (Q9D379), CALR (P14211), AT2A2 (O55143), PDIA4 (P08003), PDIA1 (P09103), PDIA3 (P27773), PDIA6 (Q922R8), CLH (Q68FD5), PPIB (P24369), TCPG (P80318), MOT4 (P57787), NICA (P57716), BASI (P18572), VAPA (Q9WV55), ENV2 (P11370), VAT1 (Q62465), 4F2 (P10852), ENOA (P17182), ILK (O55222), GPNMB (Q99P91), ENV1 (P10404), ERO1A (Q8R180), CLH, (Q68FD5), DSG1A (Q61495), AT1A1 (Q8VDN2), HYOU1 (Q9JKR6), TRAP1 (Q9CQN1), GRP75 (P38647), ENPL (P08113), CH60 (P63038), and CH10 (Q64433). In the preceding list, accession numbers are shown in parentheses.

In some embodiments, the antibody binds to an antigen selected from CDH1, CD19, CD20, CD29, CD30, CD38, CD40, CD47, EpCAM, MUC1, MUC16, EGFR, Her2, SLAMF7, and gp75. In some embodiments, the antigen is selected from CD19, CD20, CD47, EpCAM, MUC1, MUC16, EGFR, and HER2. In some embodiments, the antibody binds to an antigen selected from the Tn antigen and the Thomsen-Friedenreich antigen.

In some embodiments, the antibody or Fc fusion protein is selected from: abagovomab, abatacept (also known as ORENCIA®), abciximab (also known as REOPRO®), c7E3 Fab), adalimumab (also known as HUMIRA®), adecatumumab, alemtuzumab (also known as CAMPATH®), MabCampath or Campath-1H), altumomab, afelimomab, anatumomab mafenatox, anetumumab, anrukizumab, apolizumab, arcitumomab, aselizumab, atlizumab, atorolimumab, bapineuzumab, basiliximab (also known as SIMULECT®), bavituximab, bectumomab (also known as LYMPHOSCAN®), belimumab (also known as LYMPHO-STAT-B®), bertilimumab, besilesomab, bevacizumab (also known as AVASTIN®), biciromab brallobarbital, bivatuzumab mertansine, campath, canakinumab (also known as ACZ885), cantuzumab mertansine, capromab (also known as PROSTASCIN®), catumaxomab (also known as REMOVAB®), cedelizumab (also known as CIMZIA®), certolizumab pegol, cetuximab (also known as ERBITUX®), clenoliximab, dacetuzumab, dacliximab, daclizumab (also known as ZENAPAX®), denosumab (also known as AMG 162), detumomab, dorlimomab aritox, dorlixizumab, duntumumab, durimulumab, durmulumab, ecromeximab, eculizumab (also known as SOLIRIS®), edobacomab, edrecolomab (also known as Mab17-1A, PANOREX®), efalizumab (also known as RAPTIVA®), efungumab (also known as MYCOGRAB®), elsilimomab, enlimomab pegol, epitumomab cituxetan, efalizumab, epitumomab, epratuzumab, erlizumab, ertumaxomab (also known as REXOMUN®), etanercept (also known as ENBREL®), etaracizumab (also known as etaratuzumab, VITAXIN®, ABEGRIN®), exbivirumab, fanolesomab (also known as NEUTROSPEC®), faralimomab, felvizumab, fontolizumab (also known as HUZAF®), galiximab, gantenerumab, gavilimomab (also known as ABXCBL®), gemtuzumab ozogamicin (also known as MYLOTARG®), golimumab (also known as CNTO 148), gomiliximab, ibalizumab (also known as TNX-355), ibritumomab tiuxetan (also known as ZEVALIN®), igovomab, imciromab, infliximab (also known as REMICADE®), inolimomab, inotuzumab ozogamicin, ipilimumab (also known as MDX-010, MDX-101), iratumumab, keliximab, labetuzumab, lemalesomab, lebrilizumab, lerdelimumab, lexatumumab (also known as, HGS-ETR2, ETR2-ST01), lexitumumab, libivirumab, lintuzumab, lucatumumab, lumiliximab, mapatumumab (also known as HGSETR1, TRM-1), maslimomab, matuzumab (also known as EMD72000), mepolizumab (also known as BOSATRIA®), metelimumab, milatuzumab, minretumomab, mitumomab, morolimumab, motavizwnab (also known as NUMAX®), muromonab (also known as OKT3), nacolomab tafenatox, naptumomab estafenatox, natalizumab (also known as TYSABRI®, ANTEGREN®), nebacumab, nerelimomab, nimotuzumab (also known as THERACIM hR3®, THERA-CIM-hR3®, THERALOC®), nofetumomab merpentan (also known as VERLUMA®), ocrelizumab, odulimomab, ofatumumab, omalizumab (also known as XOLAIR®), oregovomab (also known as OVAREX®), otelixizumab, pagibaximab, palivizumab (also known as SYNAGIS®), panitumumab (also known as ABX-EGF, VECTIBIX®), pascolizumab, pemtumomab (also known as THERAGYN®), pertuzumab (also known as 2C4, OMNITARG®), pexelizumab, pintumomab, priliximab, pritumumab, ranibizumab (also known as LUCENTIS®), raxibacumab, regavirumab, reslizumab, rituximab (also known as RITUXAN®, MabTHERA®), rovelizumab, ruplizumab, satumomab, sevirumab, sibrotuzumab, siplizumab (also known as MEDI-507), sontuzumab, stamulumab (also known as MYO-029), sulesomab (also known as LEUKOSCAN®), tacatuzumab tetraxetan, tadocizumab, talizumab, taplitumomab paptox, tefibazumab (also known as AUREXIS®), telimomab aritox, teneliximab, teplizumab, ticilimumab, tocilizumab (also known as ACTEMRA®), toralizumab, tositumomab, trastuzumab (also known as HERCEPTIN®), tremelimumab (also known as CP-675,206), tucotuzumab celmoleukin, tuvirumab, urtoxazumab, ustekinumab (also known as CNTO 1275), vapaliximab, veltuzumab, vepalimomab, visilizumab (also known as NUVION®), volociximab (also known as M200), votumumab (also known as HUMASPECT®), zalutumumab, zanolimumab (also known as HuMAX-CD4), ziralimumab, zolimomab aritox, daratumumab, elotuxumab, obintunzumab, olaratumab, brentuximab vedotin, afibercept, abatacept, belatacept, afibercept, etanercept, romiplostim, SBT-040 (sequences listed in US 2017/0158772. In some embodiments, the antibody is rituximab.

### Immune Checkpoint Inhibitors

In some embodiments, the antibody of an immunoconjugate is an immune checkpoint inhibitor. In some embodiments, the immune checkpoint inhibitor reduces the expression or activity of one or more immune checkpoint proteins. In another embodiment, the immune checkpoint inhibitor reduces the interaction between one or more immune checkpoint proteins and their ligands. Inhibitory nucleic acids that decrease the expression and/or activity of immune checkpoint molecules can also be used in the methods disclosed herein.

Immune checkpoint inhibitors nivolumab and atezolizumab can be modified to include an IgG1 Fc, and subsequently converted into an immunoconjugate of the invention.

Most checkpoint antibodies are designed not to have effector function to kill cells, but rather to block the signaling. Immunoconjugates of the present invention can add back the “effector functionality” needed to elicit myeloid cell activation and pro-inflammatory responses.

In some embodiments, the immune checkpoint inhibitor is cytotoxic T-lymphocyte antigen 4 (CTLA4, also known as CD152), T cell immunoreceptor with Ig and ITIM domains (TIGIT), glucocorticoid-induced TNFR-related protein (GITR, also known as TNFRSF18), inducible T cell costimulatory (ICOS, also known as CD278), CD96, poliovirus receptor-related 2 (PVRL2, also known as CD112R, programmed cell death protein 1 (PD-1, also known as CD279), programmed cell death 1 ligand 1 (PD-L1, also known as B7-H3 and CD274), programmed cell death ligand 2 (PD-L2, also known as B7-DC and CD273), lymphocyte activation gene-3 (LAG-3, also known as CD223), B7-H4, killer immunoglobulin receptor (KIR), Tumor Necrosis Factor Receptor superfamily member 4 (TNFRST4, also known as OX40 and CD134) and its ligand OX40L (CD252), indoleamine 2,3-dioxygenase 1 (IDO-1), indoleamine 2,3-dioxygenase 2 (IDO-2), carcinoembryonic antigen-related cell adhesion molecule 1 (CEACAM1), B and T lymphocyte attenuator (BTLA, also known as CD272), T-cell membrane protein 3 (TIM3), the adenosine A2A receptor (A2Ar), and V-domain Ig suppressor of T cell activation (VISTA protein). In some embodiments, the immune checkpoint inhibitor is an inhibitor of CTLA4, PD-1, or PD-L1.

In some embodiments, the antibody is selected from: ipilimumab (also known as YERVOY®) pembrolizumab (also known as KEYTRUDA®), nivolumab (also known as OPDIVO®), atezolizumab (also known as TECENTRIQ®), avelumab (also known as BAVENCIO®), and durvalumab (also known as IMFINZI®).

In some embodiments, the immune checkpoint inhibitor is an inhibitor of CTLA4. In some embodiments, the immune checkpoint inhibitor is an antibody against CTLA4. In some embodiments, the immune checkpoint inhibitor is a monoclonal antibody against CTLA4. In some embodiments, the immune checkpoint inhibitor is a human or humanized antibody against CTLA4. In some embodiments, the immune checkpoint inhibitor reduces the expression or activity of one or more immune checkpoint proteins, such as CTLA4.

In some embodiments, the immune checkpoint inhibitor is an inhibitor of PD-1. In some embodiments, the immune checkpoint inhibitor is an antibody against PD-1. In some embodiments, the immune checkpoint inhibitor is a monoclonal antibody against PD-1. In some embodiments, the immune checkpoint inhibitor is a human or humanized antibody against PD-1. In some embodiments, the immune checkpoint inhibitor reduces the expression or activity of one or more immune checkpoint proteins, such as PD-1.

In some embodiments, the immune checkpoint inhibitor is an inhibitor of PD-L1. In some embodiments, the immune checkpoint inhibitor is an antibody against PD-L1. In some embodiments, the immune checkpoint inhibitor is a monoclonal antibody against PD-L1. In some embodiments, the immune checkpoint inhibitor is a human or humanized antibody against PD-L1. In some embodiments, the immune checkpoint inhibitor reduces the expression or activity of one or more immune checkpoint proteins, such as PD-L1. In some embodiments, the immune checkpoint inhibitor reduces the interaction between PD-1 and PD-L1.

In some embodiments, the immune checkpoint inhibitor is an inhibitor of PD-L2. In some embodiments, the immune checkpoint inhibitor is an antibody against PD-L2. In some embodiments, the immune checkpoint inhibitor is a monoclonal antibody against PD-L2. In some embodiments, the immune checkpoint inhibitor is a human or humanized antibody against PD-L2. In some embodiments, the immune checkpoint inhibitor reduces the expression or activity of one or more immune checkpoint proteins, such as PD-L2. In some embodiments, the immune checkpoint inhibitor reduces the interaction between PD-1 and PD-L2.

In some embodiments, the immune checkpoint inhibitor is an inhibitor of LAG-3. In some embodiments, the immune checkpoint inhibitor is an antibody against LAG-3. In some embodiments, the immune checkpoint inhibitor is a monoclonal antibody against LAG-3. In some embodiments, the immune checkpoint inhibitor is a human or humanized antibody against LAG-3. In some embodiments, the immune checkpoint inhibitor reduces the expression or activity of one or more immune checkpoint proteins, such as LAG-3.

In some embodiments, the immune checkpoint inhibitor is an inhibitor of B7-H4. In some embodiments, the immune checkpoint inhibitor is an antibody against B7-H4. In some embodiments, the immune checkpoint inhibitor is a monoclonal antibody against B7-H4. In some embodiments, the immune checkpoint inhibitor is a human or humanized antibody against B7-H4. In some embodiments, the immune checkpoint inhibitor reduces the expression or activity of one or more immune checkpoint proteins, such as B7-H4.

In some embodiments, the immune checkpoint inhibitor is an inhibitor of KIR. In some embodiments, the immune checkpoint inhibitor is an antibody against KIR. In some embodiments, the immune checkpoint inhibitor is a monoclonal antibody against KIR. In some embodiments, the immune checkpoint inhibitor is a human or humanized antibody against KIR. In some embodiments, the immune checkpoint inhibitor reduces the expression or activity of one or more immune checkpoint proteins, such as KIR.

In some embodiments, the immune checkpoint inhibitor is an inhibitor of TNFRSF4. In some embodiments, the immune checkpoint inhibitor is an antibody against TNFRSF4. In some embodiments, the immune checkpoint inhibitor is a monoclonal antibody against TNFRSF4. In some embodiments, the immune checkpoint inhibitor is a human or humanized antibody against TNFRSF4. In some embodiments, the immune checkpoint inhibitor reduces the expression or activity of one or more immune checkpoint proteins, such as TNFRSF4.

In some embodiments, the immune checkpoint inhibitor is an inhibitor of OX40L. In some embodiments, the immune checkpoint inhibitor is an antibody against OX40L. In some embodiments, the immune checkpoint inhibitor is a monoclonal antibody against OX40L. In some embodiments, the immune checkpoint inhibitor is a human or humanized antibody against OX40L. In some embodiments, the immune checkpoint inhibitor reduces the expression or activity of one or more immune checkpoint proteins, such as OX40L. In some embodiments, the immune checkpoint inhibitor reduces the interaction between TNFRSF4 and OX40L.In some embodiments, the immune checkpoint inhibitor is an inhibitor of IDO-1. In some embodiments, the immune checkpoint inhibitor is an antibody against IDO-1. In some embodiments, the immune checkpoint inhibitor is a monoclonal antibody against IDO-1, in some embodiments, the immune checkpoint inhibitor is a human or humanized antibody against IDO-1. In some embodiments, the immune checkpoint inhibitor reduces the expression or activity of one or more immune checkpoint proteins, such as IDO-1.

In some embodiments, the immune checkpoint inhibitor is an inhibitor of IDO-2. In some embodiments, the immune checkpoint inhibitor is an antibody against IDO-2. In some embodiments, the immune checkpoint inhibitor is a monoclonal antibody against IDO-2. In some embodiments, the immune checkpoint inhibitor is a human or humanized antibody against IDO-2. In some embodiments, the immune checkpoint inhibitor reduces the expression or activity of one or more immune checkpoint proteins, such as IDO-2.

In some embodiments, the immune checkpoint inhibitor is an inhibitor of CEACAM1. In some embodiments, the immune checkpoint inhibitor is an antibody against CEACAM1. In some embodiments, the immune checkpoint inhibitor is a monoclonal antibody against CEACAM1. In some embodiments, the immune checkpoint inhibitor is a human or humanized antibody against CEACAM1. In some embodiments, the immune checkpoint inhibitor reduces the expression or activity of one or more immune checkpoint proteins, such as CEACAM1.

In some embodiments, the immune checkpoint inhibitor is an inhibitor of BTLA. In some embodiments, the immune checkpoint inhibitor is an antibody against BTLA. In some embodiments, the immune checkpoint inhibitor is a monoclonal antibody against BTLA. In some embodiments, the immune checkpoint inhibitor is a human or humanized antibody against BMA. In some embodiments, the immune checkpoint inhibitor reduces the expression or activity of one or more immune checkpoint proteins, such as BTLA.

In some embodiments, the immune checkpoint inhibitor is an inhibitor of TIM3. In some embodiments, the immune checkpoint inhibitor is an antibody against TIM3. In some embodiments, the immune checkpoint inhibitor is a monoclonal antibody against TIM3. In some embodiments, the immune checkpoint inhibitor is a human or humanized antibody against TIM3. In some embodiments, the immune checkpoint inhibitor reduces the expression or activity of one or more immune checkpoint proteins, such as TIM3.

In some embodiments, the immune checkpoint inhibitor is an inhibitor of A2Ar. In some embodiments, the immune checkpoint inhibitor is an antibody against A2Ar. In some embodiments, the immune checkpoint inhibitor is a monoclonal antibody against A2Ar. In some embodiments, the immune checkpoint inhibitor is a human or humanized antibody against A2Ar. In some embodiments, the immune checkpoint inhibitor reduces the expression or activity of one or more immune checkpoint proteins, such as A2Ar.

In some embodiments, the immune checkpoint inhibitor is an inhibitor of VISTA protein. In some embodiments, the immune checkpoint inhibitor is an antibody against VISTA protein. In some embodiments, the immune checkpoint inhibitor is a monoclonal antibody against VISTA protein. In some embodiments, the immune checkpoint inhibitor is a human or humanized antibody against VISTA protein. In some embodiments, the immune checkpoint inhibitor reduces the expression or activity of one or more immune checkpoint proteins, such as VISTA protein.

### Aza-Benzazepine Adjuvant Compounds

The immunoconjugate of the invention comprises an aza-benzazepine adjuvant moiety. The adjuvant moiety described herein elicits an immune response (i.e., an immunostimulatory agent). Generally, the adjuvant moiety described herein is a TLR agonist. TLRs are type-I transmembrane proteins that are responsible for the initiation of innate immune responses in vertebrates. TLRs recognize a variety of pathogen-associated molecular patterns from bacteria, viruses, and fungi and act as a first line of defense against invading pathogens. TLRs elicit overlapping yet distinct biological responses due to differences in cellular expression and in the signaling pathways that they initiate. Once engaged (e.g., by a natural stimulus or a synthetic TLR agonist), TLRs initiate a signal transduction cascade leading to activation of nuclear factor-KB (NF-κB) via the adapter protein myeloid differentiation primary response gene 88 (MyD88) and recruitment of the IL-1 receptor associated kinase (IRAK). Phosphorylation of IRAK then leads to recruitment of TNF-receptor associated factor 6 (TRAF6), which results in the phosphorylation of the NF-κB inhibitor I-KB. As a result, NF-κB enters the cell nucleus and initiates transcription of genes whose promoters contain NF-κB binding sites, such as cytokines. Additional modes of regulation for TLR signaling include TIR-domain containing adapter-inducing interferon-β (TRIF)-dependent induction of TNF-receptor associated factor 6 (TRAF6) and activation of MyD88 independent pathways via TRIF and TRAF3, leading to the phosphorylation of interferon response factor three (IRF3). Similarly, the MyD88 dependent pathway also activates several IRF family members, including IRF5 and IRF7 whereas the TRIF dependent pathway also activates the NF-κB pathway.

Typically, the adjuvant moiety described herein is a TLR7 and/or TLR8 agonist. TLR7 and TLR8 are both expressed in monocytes and dendritic cells. In humans, TLR7 is also expressed in plasmacytoid dendritic cells (pDCs) and B cells. TLR8 is expressed mostly in cells of myeloid origin, i.e., monocytes, granulocytes, and myeloid dendritic cells. TLR7 and TLR8 are capable of detecting the presence of “foreign” single-stranded RNA within a cell, as a means to respond to viral invasion. Treatment of TLR8-expressing cells, with TLR8 agonists can result in production of high levels of IL-12, IFN-γ, IL-1, TNF-α, IL-6, and other inflammatory cytokines. Similarly, stimulation of TLR7-expressing cells, such as pDCs, with TLR7 agonists can result in production of high levels of IFN-α and other inflammatory cytokines. TLR7/TLR8 engagement and resulting cytokine production can activate dendritic cells and other antigen-presenting cells, driving diverse innate and acquired immune response mechanisms leading to tumor destruction.

It was discovered that the amidine functional group of benzazepine adjuvant compounds, moieties conjugated to antibodies as immunoconjugates (WO 2020/252294; WO 2021/067242; WO 2022/125884; WO 2022/125891; WO 2022/125904; WO 2022/125908; WO 2022/125915), undergoes hydrolysis to the lactam functional group. This degradative hydrolysis renders the lactam benzazepine compounds inactive as TLR 7/8 agonists. For example, comparator lactam compounds CBz-8 and CBz-9 (Table 1b) were inactive in the HEK assay (Example 202).

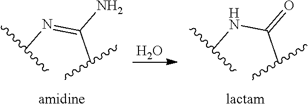 [SMILES: */N=C(\*)N ; O ; *NC(*)=O]

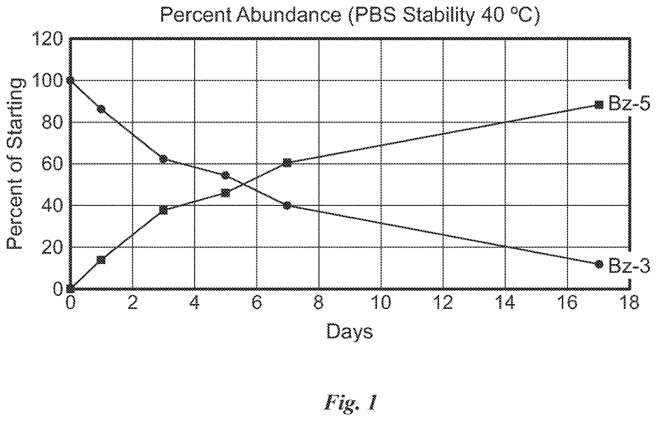

Amidine benzazepine comparator compound CBz-3 (Table 1b) degrades in PBS buffer (pH 7.4) at 40° C. to produce lactam benzazepine comparator compound CBz-5 (Table 1b) at 90% at 17 days. FIG. 1 shows a plot of the hydrolysis of the amidine group of CBz-3 to form CBz-5 over time in PBS buffer at 40° C. In human plasma at room temperature after 24 hours, 15% of CBz-3 degrades to CBz-5.

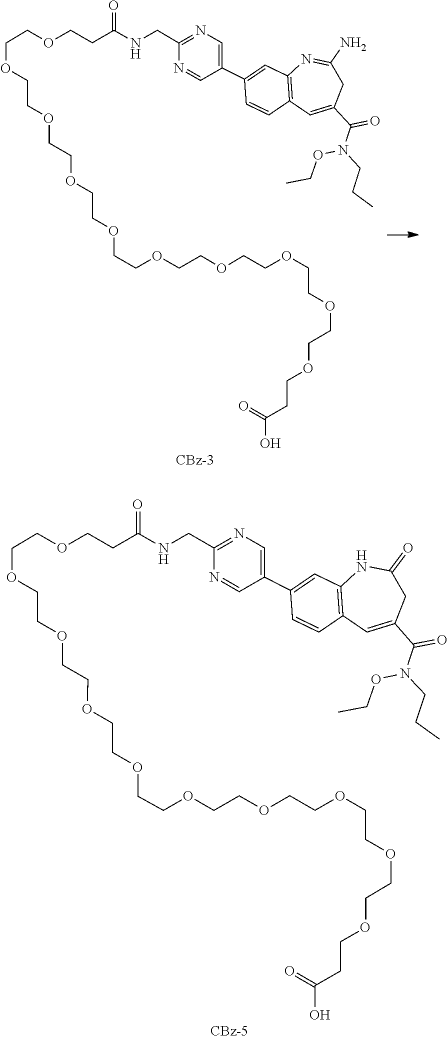 [SMILES: CCCN(OCC)C(=O)C1=Cc2ccc(-c3cnc(CNC(=O)CCOCCOCCOCCOCCOCCOCCOCCOCCOCCOCCC(=O)O)nc3)cc2N=C(N)C1 ; CCCN(OCC)C(=O)C1=Cc2ccc(-c3cnc(CNC(=O)CCOCCOCCOCCOCCOCCOCCOCCOCCOCCOCCC(=O)O)nc3)cc2NC(=O)C1]

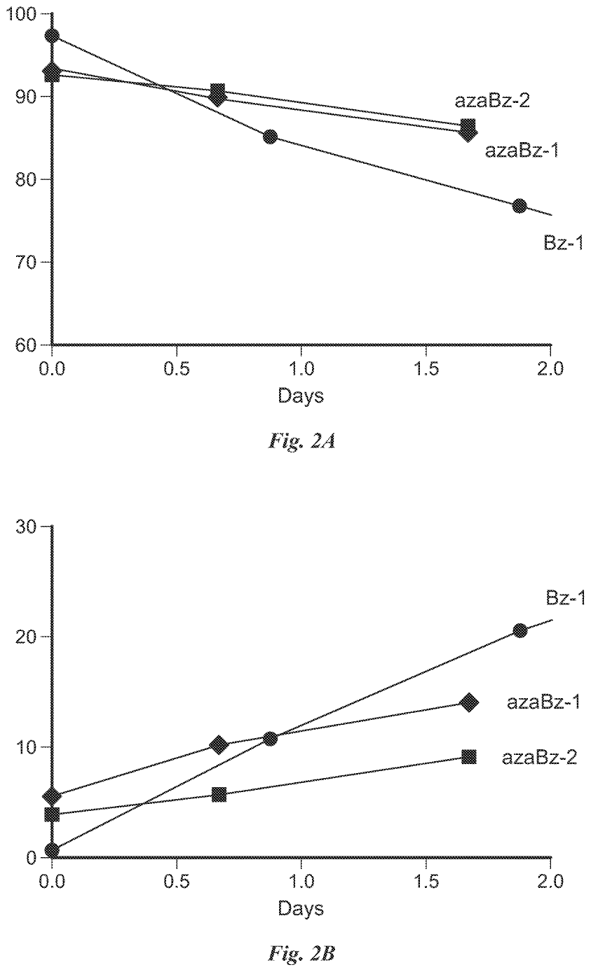

The rate of degradation can be modulated by nitrogen substitution of carbon in the membered ring of the benzazepine. Aza-benzazepine compounds azaBz-1 and azaBz-2 introduce a single nitrogen each compared to benzazepine compound CBz-1. Amidine hydrolysis of the three compounds in PBS at 40° C. were measured by disappearance of starting amidine and the appearance of lactam product. FIG. 2A shows a plot of the hydrolysis of the amidine group of benzazepine compound CBz-1, and aza-benzazepine compounds azaBa-1 and azaBz-2 by percentage of starting compounds remaining over 2 days. FIG. 2B shows a plot of the hydrolysis of the amidine group of CBz-1, and aza-benzazepine compounds azaBa-1 and azaBz-2 by the appearance of the corresponding lactam compounds over 2 days. No other degradation products were detected.

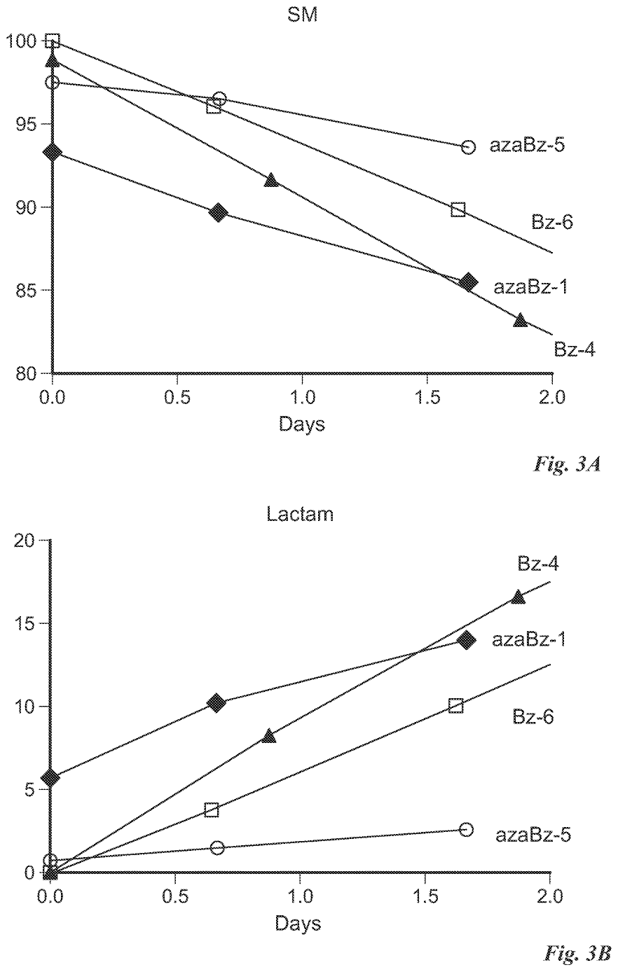

Adding a sulfonate group at the 8 position of both a benzazepine and an aza-benzazepine compound conferred stability and slowed hydrolysis. FIG. 3A shows a plot of the hydrolysis of the amidine group of benzazepine compounds CBz-4 and 8-sulfonate CBz-6, and aza-benzazepine compounds azaBa-1 and 8-sulfonate azaBz-5 by percentage of starting compounds remaining over 2 days. FIG. 3B shows a plot of the hydrolysis of the amidine group of benzazepine comparator compounds CBz-4 and 8-sulfonate CBz-6, and aza-benzazepine compounds azaBa-1 and 8-sulfonate azaBz-5 by the appearance of the corresponding lactam compounds over 2 days.

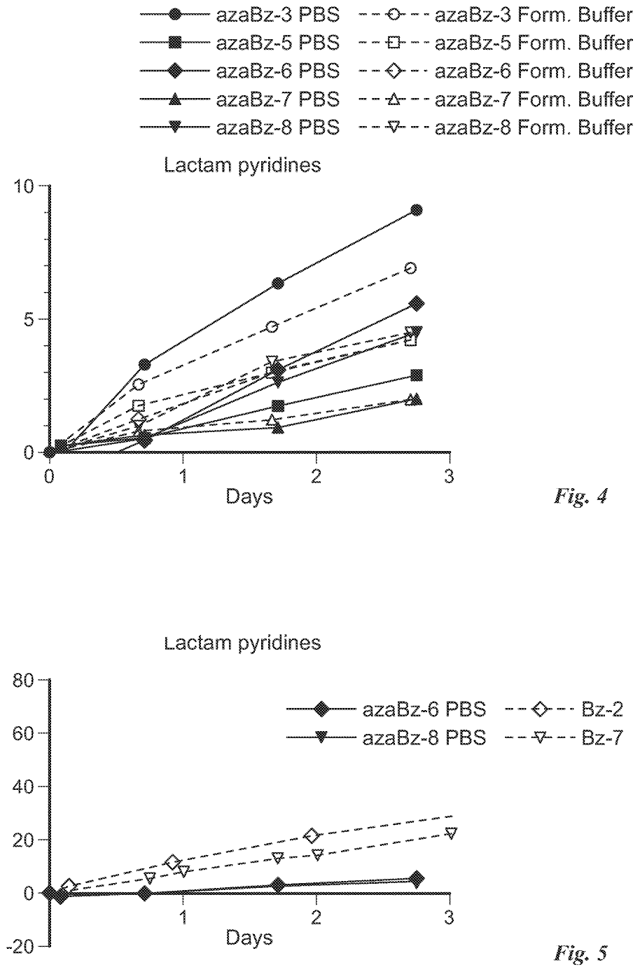

A nitrogen at the 7-position is stabilizing and slows hydrolysis in aza-benzazepine compounds with a variety of substituents at the 8-position. FIG. 4 shows a plot of the hydrolysis of the amidine group of aza-benzazepine compounds azaBa-3, azaBz-5, azaBz-6, azaBz-7, and azaBz-8 in PBS and Formulation buffer, by the appearance of the corresponding lactam compounds over 2 days. The amount of lactam is normalized for each sample at the start (to) for easier rate comparisons. The half-life of each compound was measured in PBS (pH 7.4) at 37° C. and in formulation buffer (pH 6) at 22° C. as follows:

| | | t ½ in |
|---|---|---|
| | t ½ in PBS | formulation buffer |
| | (pH 7.4) at | (pH 6) at |
| azaBz No. | 37° C. (days) | 22° C. (days) |
| azaBz-3 | 20 | 30 |
| azaBz-5 | 70 | 50 |
| azaBz-6 | 30 | 30 |
| azaBz-7 | 100 | 100 |
| azaBz-8 | 40 | 40 |

The hydrolytic degradation rates of benzazepine and 7-azabenzazepine compounds were directly compared in PBS (pH 7.4) at 37° C. to mimic in vivo effects and in formulation buffer to simulate storage and lifetime effects. The half-lives (t½) of benzazepine compounds CBz-2 and CBz-7 were 6 days and 8 days, respectively. The half-lives (t½) of aza-benzazepine compounds azaBa-6 and azaBz-8 were 30 days and 40 days, respectively. FIG. 5 shows a plot of the hydrolysis of the amidine group of benzazepine compounds CBz-2 and CBz-7, and aza-benzazepine compounds azaBa-6 and azaBz-8 in PBS, by the appearance of the corresponding lactam compounds over 2 days. The amount of lactam is normalized for each sample at the start (to) for easier rate comparisons. The 7-aza modification is stabilizing in PBS and formulation buffer by about 5-fold relative to the corresponding benzazepine compounds.

Exemplary aza-benzazepine compounds (azaBza) of Table 1a and comparator compounds (CBz) of Table 1b were synthesized, purified, and characterized by mass spectrometry and shown to have the expected mass. Additional experimental procedures are found in the Examples. Activity against HEK293 NFKB reporter cells expressing human TLR7 or human TLR8 was measured according to Example 202. Certain aza-benzazepine compounds demonstrate the surprising and unexpected property of TLR8 agonist selectivity which may predict useful therapeutic activity to treat cancer and other disorders. For example, azaBz-24 demonstrated TLR7/8 selectivity with an EC50 of 842 nM against TLR7 and 196 nM against TLR8. Also, azaBz-2 showed no response against TLR7 and an EC50 of 5.5 micromolar (uM) against TLR8.

TABLE 1a
Aza-benzazepine compounds (azaBz)

| azaBz | | |
|---|---|---|
| No. | Structure | MW |
| azaBz-1 | 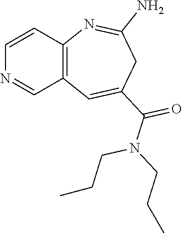 [SMILES: CCCN(CCC)C(=O)C1=Cc2cnccc2N=C(N)C1] | 286.4 |
| azaBz-2 | 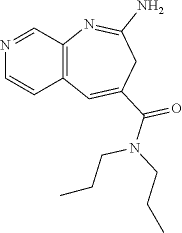 [SMILES: CCCN(CCC)C(=O)C1=Cc2ccncc2N=C(N)C1] | 286.4 |
| azaBz-3 | 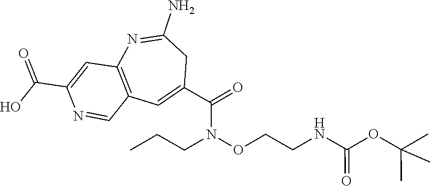 [SMILES: CCCN(OCCNC(=O)OC(C)(C)C)C(=O)C1=Cc2cnc(C(=O)O)cc2N=C(N)C1] | 447.5 |
| azaBz-4 | 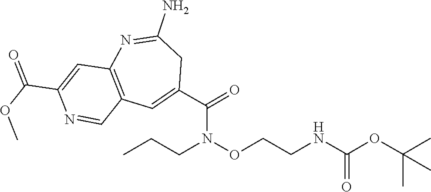 [SMILES: CCCN(OCCNC(=O)OC(C)(C)C)C(=O)C1=Cc2cnc(C(=O)OC)cc2N=C(N)C1] | 461.5 |
| azaBz-5 | 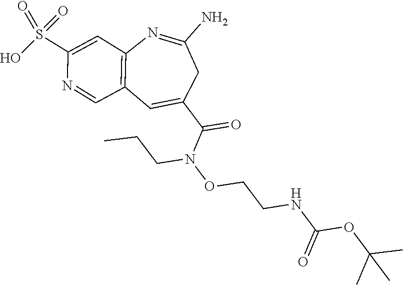 [SMILES: CCCN(OCCNC(=O)OC(C)(C)C)C(=O)C1=Cc2cnc(S(=O)(=O)O)cc2N=C(N)C1] | 483.5 |
| azaBz-6 | 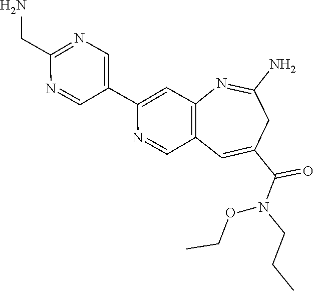 [SMILES: CCCN(OCC)C(=O)C1=Cc2cnc(-c3cnc(CN)nc3)cc2N=C(N)C1] | 395.5 |
| azaBz-7 | 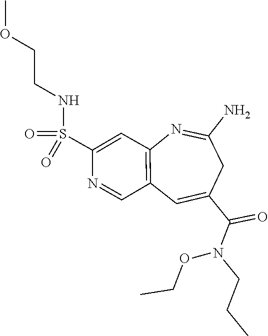 [SMILES: CCCN(OCC)C(=O)C1=Cc2cnc(S(=O)(=O)NCCOC)cc2N=C(N)C1] | 425.5 |
| azaBz-8 | 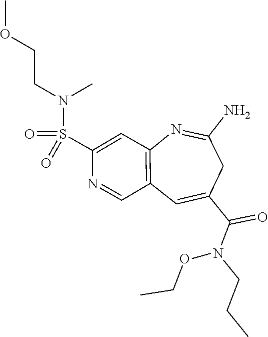 [SMILES: CCCN(OCC)C(=O)C1=Cc2cnc(S(=O)(=O)N(C)CCOC)cc2N=C(N)C1] | 439.5 |
| azaBz-9 | 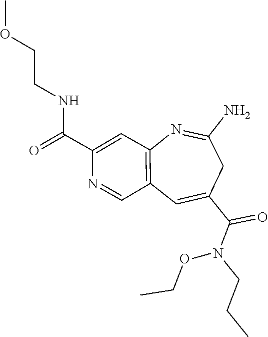 [SMILES: CCCN(OCC)C(=O)C1=Cc2cnc(C(=O)NCCOC)cc2N=C(N)C1] | 389.5 |
| azaBz-10 | 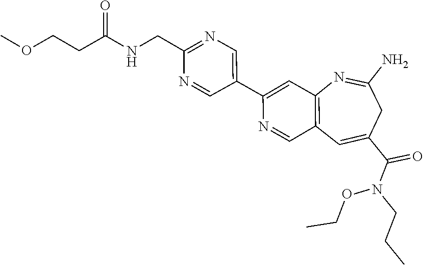 [SMILES: CCCN(OCC)C(=O)C1=Cc2cnc(-c3cnc(CNC(=O)CCOC)nc3)cc2N=C(N)C1] | 481.6 |
| azaBz-11 | 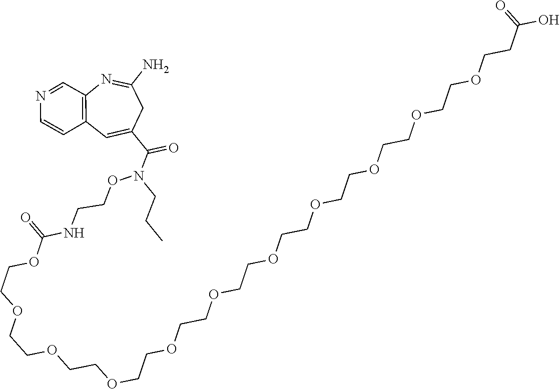 [SMILES: CCCN(OCCNC(=O)OCCOCCOCCOCCOCCOCCOCCOCCOCCOCCOCCC(=O)O)C(=O)C1=Cc2ccncc2N=C(N)C1] | 860.0 |
| azaBz-12 | 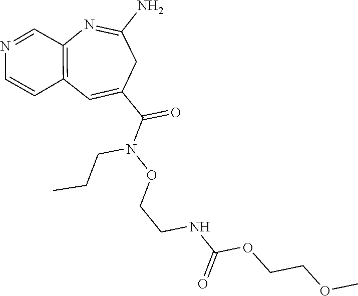 [SMILES: CCCN(OCCNC(=O)OCCOC)C(=O)C1=Cc2ccncc2N=C(N)C1] | 405.5 |
| azaBz-13 | 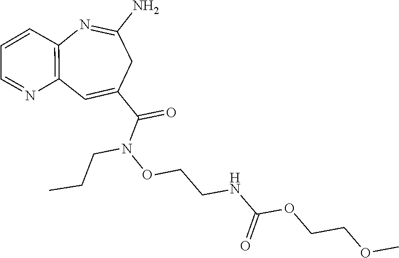 [SMILES: CCCN(OCCNC(=O)OCCOC)C(=O)C1=Cc2ncccc2N=C(N)C1] | 405.5 |
| azaBz-14 | 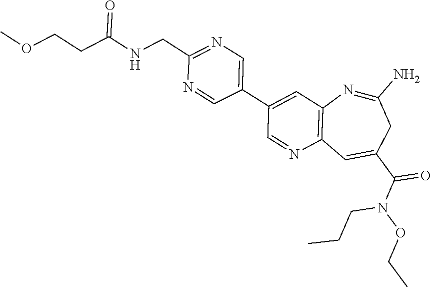 [SMILES: CCCN(OCC)C(=O)C1=Cc2ncc(-c3cnc(CNC(=O)CCOC)nc3)cc2N=C(N)C1] | 481.6 |
| azaBz-15 | 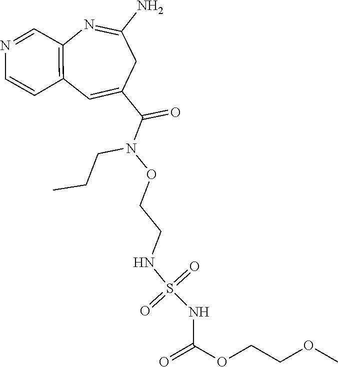 [SMILES: CCCN(OCCNS(=O)(=O)NC(=O)OCCOC)C(=O)C1=Cc2ccncc2N=C(N)C1] | 484.5 |
| azaBz-16 | 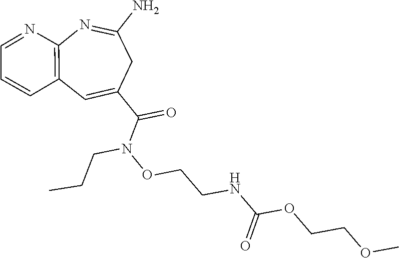 [SMILES: CCCN(OCCNC(=O)OCCOC)C(=O)C1=Cc2cccnc2N=C(N)C1] | 405.5 |
| azaBz-17 | 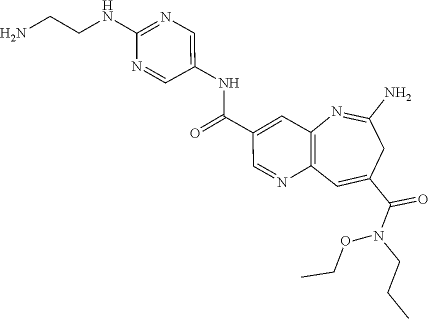 [SMILES: CCCN(OCC)C(=O)C1=Cc2ncc(C(=O)Nc3cnc(NCCN)nc3)cc2N=C(N)C1] | 467.5 |
| azaBz-18 | 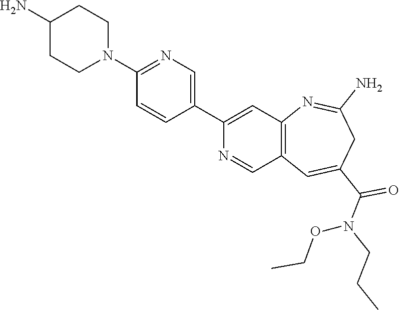 [SMILES: CCCN(OCC)C(=O)C1=Cc2cnc(-c3ccc(N4CCC(N)CC4)nc3)cc2N=C(N)C1] | 506.6 |
| azaBz-19 | 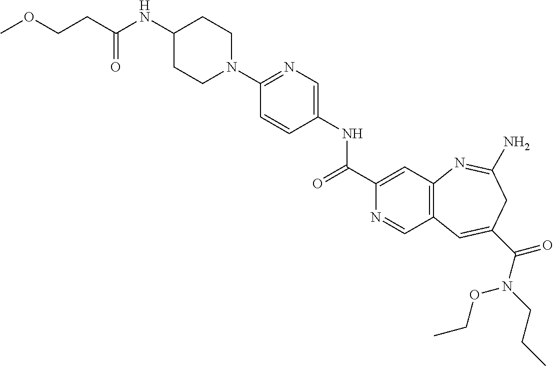 [SMILES: CCCN(OCC)C(=O)C1=Cc2cnc(C(=O)Nc3ccc(N4CCC(NC(=O)CCOC)CC4)nc3)cc2N=C(N)C1] | 592.7 |
| azaBz-20 | 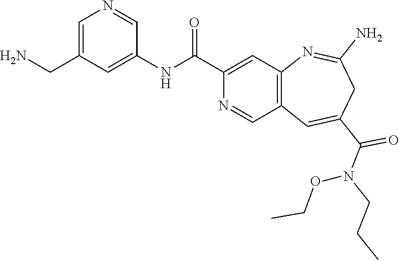 [SMILES: CCCN(OCC)C(=O)C1=Cc2cnc(C(=O)Nc3cncc(CN)c3)cc2N=C(N)C1] | 437.5 |
| azaBz-21 | 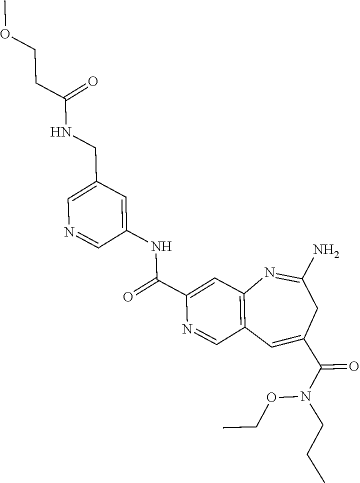 [SMILES: CCCN(OCC)C(=O)C1=Cc2cnc(C(=O)Nc3cncc(CNC(=O)CCOC)c3)cc2N=C(N)C1] | 523.6 |
| azaBz-22 | 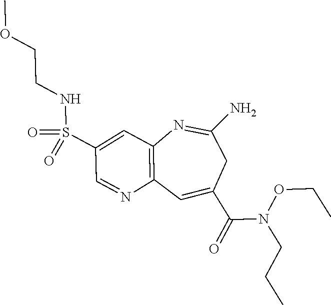 [SMILES: CCCN(OCC)C(=O)C1=Cc2ncc(S(=O)(=O)NCCOC)cc2N=C(N)C1] | 425.5 |
| azaBz-23 | 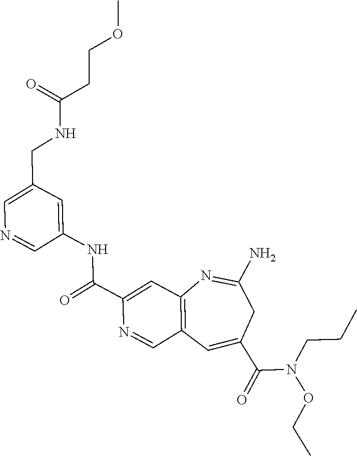 [SMILES: CCCN(OCC)C(=O)C1=Cc2cnc(C(=O)Nc3cncc(CNC(=O)CCOC)c3)cc2N=C(N)C1] | 523.6 |
| azaBz-24 | 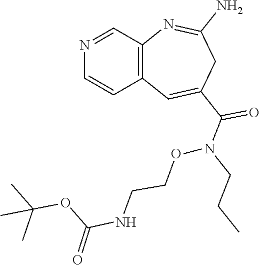 [SMILES: CCCN(OCCNC(=O)OC(C)(C)C)C(=O)C1=Cc2ccncc2N=C(N)C1] | 403.5 |

TABLE 1b
Comparator benzazepine amidine and lactam compounds (CBz)

| CBz | | |
|---|---|---|
| No. | Structure | MW |
| CBz-1 | 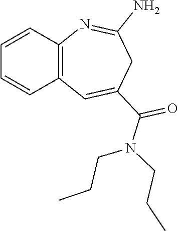 [SMILES: CCCN(CCC)C(=O)C1=Cc2ccccc2N=C(N)C1] | 285.4 |
| CBz-2 | 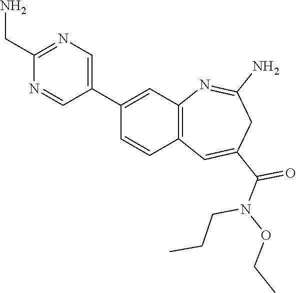 [SMILES: CCCN(OCC)C(=O)C1=Cc2ccc(-c3cnc(CN)nc3)cc2N=C(N)C1] | 394.5 |
| CBz-3 | 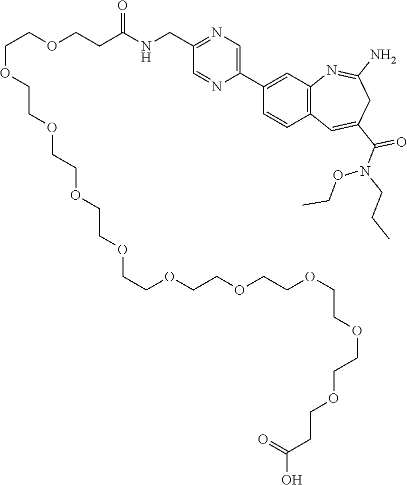 [SMILES: CCCN(OCC)C(=O)C1=Cc2ccc(-c3cnc(CNC(=O)CCOCCOCCOCCOCCOCCOCCOCCOCCOCCOCCC(=O)O)cn3)cc2N=C(N)C1] | 935.1 |
| CBz-4 | 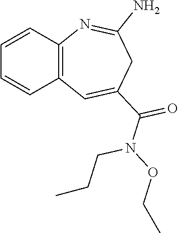 [SMILES: CCCN(OCC)C(=O)C1=Cc2ccccc2N=C(N)C1] | 287.4 |
| CBz-5 | 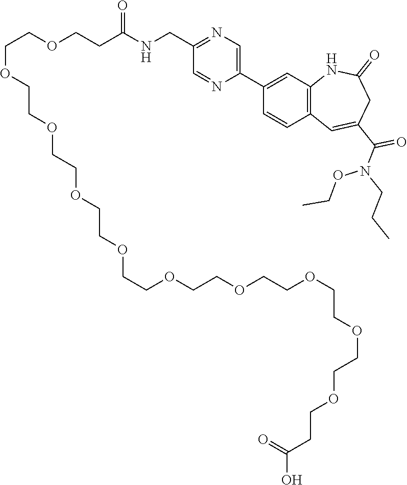 [SMILES: CCCN(OCC)C(=O)C1=Cc2ccc(-c3cnc(CNC(=O)CCOCCOCCOCCOCCOCCOCCOCCOCCOCCOCCC(=O)O)cn3)cc2NC(=O)C1] | 936.1 |
| CBz-6 | 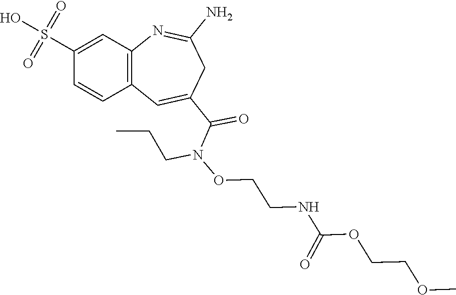 [SMILES: CCCN(OCCNC(=O)OCCOC)C(=O)C1=Cc2ccc(S(=O)(=O)O)cc2N=C(N)C1] | 484.5 |
| CBz-7 | 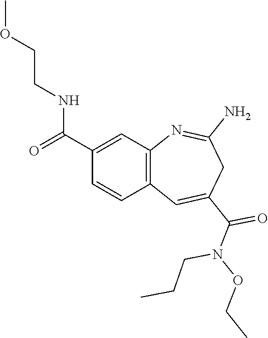 [SMILES: CCCN(OCC)C(=O)C1=Cc2ccc(C(=O)NCCOC)cc2N=C(N)C1] | 388.5 |
| CBz-8 | 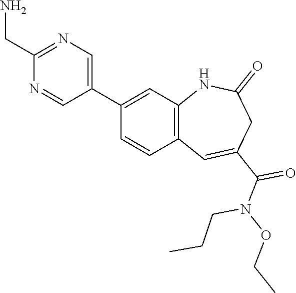 [SMILES: CCCN(OCC)C(=O)C1=Cc2ccc(-c3cnc(CN)nc3)cc2NC(=O)C1] | 395.5 |
| CBz-9 | 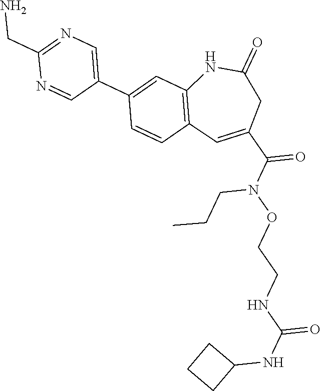 [SMILES: CCCN(OCCNC(=O)NC1CCC1)C(=O)C1=Cc2ccc(-c3cnc(CN)nc3)cc2NC(=O)C1] | 507.6 |

aza-Benzazepine-Linker Compounds

The immunoconjugates of the invention are prepared by conjugation of an antibody with an aza-benzazepine linker compound, azaBzL. The aza-benzazepine linker compounds comprise an aza-benzazepine (azaBz) moiety covalently attached to a linker unit. The linker units comprise functional groups and subunits which affect stability, permeability, solubility, and other pharmacokinetic, safety, and efficacy properties of the immunoconjugates. The linker unit includes a reactive functional group which reacts, i.e. conjugates, with a reactive functional group of the antibody. For example, a nucleophilic group such as a lysine side chain amino of the antibody reacts with an electrophilic reactive functional group of the azaBz-L compound to form the immunoconjugate. Also, for example, a cysteine thiol of the antibody reacts with a maleimide, bromoacetamide, or disulfide group of the azaBza-L linker compound to form the immunoconjugate.

Reactive electrophilic functional groups (Q in Formula II) suitable for the azaBza-L linker compounds include, but are not limited to, N-hydroxysuccinimidyl (NHS) esters and N-hydroxysulfosuccinimidyl (sulfo-NHS) esters (amine reactive); carbodiimides (amine and carboxyl reactive); hydroxymethyl phosphines (amine reactive); maleimides (thiol reactive); halogenated acetamides such as N-iodoacetamides (thiol reactive); aryl azides (primary amine reactive); fluorinated aryl azides (reactive via carbon-hydrogen (C—H) insertion); pentafluorophenyl (PFP) esters (amine reactive); tetrafluorophenyl (TFP) esters (amine reactive); imidoesters (amine reactive); isocyanates (hydroxyl reactive); vinyl sulfones (thiol, amine, and hydroxyl reactive); pyridyl disulfides (thiol reactive); and benzophenone derivatives (reactive via C—H bond insertion). Further reagents include, but are not limited, to those described in Hermanson, Bioconjugate Techniques 2^{nd} Edition, Academic Press, 2008.

A linker may comprise one or more linker units or components. Exemplary linker components include 6-maleimidocaproyl (“MC”), maleimidopropanoyl (“MP”), valine-citrulline (“val-cit” or “ve”), alanine-phenylalanine (“ala-phe”), phenylalanine-lysine (phe-lys), p-aminobenzyloxycarbonyl (a “PAB”), N-succinimidyl 4-(2-pyridylthio) pentanoate (“SPP”), and 4-(N-maleimidomethyl) cyclohexane-1 carboxylate (“MCC”). Various linker components are known in the art, some of which are described herein.

A linker may be a “cleavable linker,” facilitating release of a drug. Nonlimiting exemplary cleavable linkers include acid-labile linkers (e.g., comprising hydrazone), protease-sensitive, peptidase-substrate linkers (U.S. Pat. No. 7,498,298), photolabile linkers, or disulfide-containing linkers (Chari et al., Cancer Research 52:127-131 (1992); U.S. Pat. No. 5,208,020).

Generally, the linker (L) may be cleavable or non-cleavable. Cleavable linkers may include a peptide sequence which is a substrate for certain proteases such as Cathepsins which recognize and cleave the peptide linker unit, separating the phenyl glutarimide moiety from the antibody (Caculitan N G, et al (2017) Cancer Res. 77(24):7027-7037).

Cleavable linker may include labile functionality such as an acid-sensitive disulfide group (Kellogg, B A et al (2011) Bioconjugate Chem. 22, 717-727; Ricart, A. D. et al (2011) Clin. Cancer Res. 17, 6417-6427; Pillow, T., et al (2017) Chem. Sci. 8:366-370; Zhang D, et al (2016) ACS Med Chem Let. 7(11):988-993).

In some embodiments, the linker is non-cleavable under physiological conditions. As used herein, the term “physiological conditions” refers to a temperature range of 20-40 degrees Celsius, atmospheric pressure (i.e., 1 atm), a pH of about 6 to about 8, and the one or more physiological enzymes, proteases, acids, and bases. One advantage of a non-cleavable linker between the antibody and PG moiety in an antibody conjugate is minimizing premature phenyl glutarimide moiety release and corresponding toxicity.

In some embodiments, the linker comprises a trivalent, branch point as part of an amino acid unit (e.g., lysine) wherein additional linker units are attached via the side chain amine of lysine or linked to other sites of an amino acid unit (U.S. Pat. No. 11,173,214). A similar motif could be utilized with a glutamic acid of an amino acid unit. An exemplary additional linker unit is a monovalent solubilizing unit such as one or more units of polyglycine, polysarcosine, polyethyleneoxy (PEG), and a glycoside, or combinations thereof. The solubilizing unit may bear a group at the terminus such as an amino acid, amino, hydroxyl, hydrogen, carboxylic acid, glycerol, or a sugar such as pentaerythritol, maltitol, sorbitol, xylitol, erythritol, isomalt, or combinations thereof.

In some embodiments, an amino acid unit or peptide unit comprises one or more amino acids selected from the group consisting of glycine, alanine, serine, threonine, cysteine, valine, leucine, isoleucine, methionine, proline, phenylalanine, tyrosine, tryptophan, aspartic acid, glutamic acid, asparagine, glutamine, histidine, lysine, arginine, sarcosine, and beta-alanine.

In one embodiment, the invention includes an amino acid unit or a peptide linking unit, i.e. L or linker, between the antibody and the azabenzazepine (azaBz) moiety, comprising a peptide comprising a linear sequence of specific amino acid residues which can be selectively cleaved by a protease such as a cathepsin, caspase, a tumor-associated elastase enzyme or an enzyme with protease-like or elastase-like activity. The peptide radical may be two to about twelve amino acids. Enzymatic cleavage of a bond within the peptide linker releases an active form of the azabenzazepine (azaBz) moiety. This leads to an increase in the tissue specificity of the antibody conjugates and thus to an additional decrease of toxicity of the conjugates according to the invention in other tissue types. Release of an active azaBz moiety from an antibody conjugate can occur due to the action of lysosomal proteases such as cathepsin and plasmin which may be present at elevated levels in certain tumor tissues. The lysosomal enzyme can be, for example, cathepsin B, β-glucuronidase, or β-galactosidase.

A cleavable peptide of a peptide linker unit can be selected from tetrapeptides such as Gly-Phe-Leu-Gly, Ala-Leu-Ala-Leu, tripeptides such as Glu-Val-Cit, or dipeptides such as Val-Cit, Val-Ala, Ala-Ala, and Phe-Lys.

The linker provides sufficient stability of the immunoconjugate in biological media, such as culture medium or serum, as well as the desired intracellular action within tumor tissue as a result of its specific enzymatic or hydrolytic cleavability with release of the azaBz moiety.

The enzymatic activity of a protease, cathepsin, or elastase can catalyze cleavage of a covalent bond of the antibody conjugate under physiological conditions. The enzymatic activity being the expression product of cells associated with tumor tissue. The enzymatic activity on the cleavage site of the targeting peptide converts the antibody conjugate to an active azaBz adjuvant free of targeting antibody and linking group. The cleavage site may be specifically recognized by the enzyme. Cathepsin or elastase may catalyze the cleavage of a specific peptidic bond between the C-terminal amino acid residue of the specific peptide and the azaBz moiety of the immunoconjugate.

In one embodiment, the invention includes a linking unit, i.e. L or linker, between the antibody and the azaBz moiety, comprising a substrate for glucuronidase (Jeffrey S C, et al (2006) Bioconjug Chem. 17(3):831-40; U.S. Pat. Nos. 11,413,353; 11,173,214), or sulfatase (Bargh J D, et al (2020) Chem Sci. 11(9):2375-2380) cleavage. In particular, L includes a Gluc unit and comprises a formula selected from:

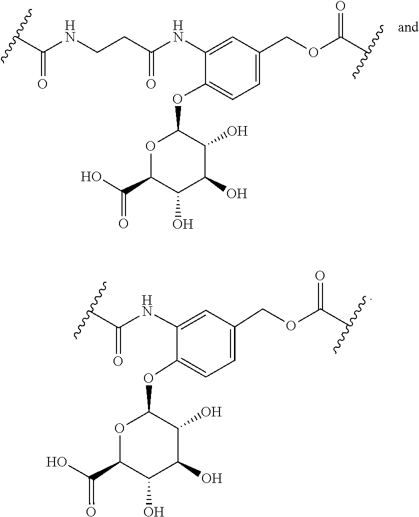 [SMILES: *C(=O)NCCC(=O)Nc1cc(COC(*)=O)ccc1O[C@@H]1O[C@H](C(=O)O)[C@@H](O)[C@H](O)[C@H]1O ; *C(=O)Nc1cc(COC(*)=O)ccc1O[C@@H]1O[C@H](C(=O)O)[C@@H](O)[C@H](O)[C@H]1O]

Specific cleavage of the immunoconjugate takes advantage of the presence of tumor infiltrating cells of the immune system and leukocyte-secreted enzymes, to promote the activation of an anticancer drug at the tumor site.

Reactive electrophilic reactive functional groups (Q in Formula II) suitable for the azabenzazepine linker compound (azaBz-L) include, but are not limited to, N-hydroxysuccinimidyl (NHS) esters and N-hydroxysulfosuccinimidyl (sulfo-NHS) esters (amine reactive); carbodiimides (amine and carboxyl reactive); hydroxymethyl phosphines (amine reactive); maleimides (thiol reactive); halogenated acetamides such as N-iodoacetamides (thiol reactive); aryl azides (primary amine reactive); fluorinated aryl azides (reactive via carbon-hydrogen (C—H) insertion); pentafluorophenyl (PFP) esters (amine reactive); tetrafluorophenyl (TFP) and sulfotetrafluorophenyl (STP) esters (amine reactive); imidoesters (amine reactive); isocyanates (hydroxyl reactive); vinyl sulfones (thiol, amine, and hydroxyl reactive); pyridyl disulfides (thiol reactive); and benzophenone derivatives (reactive via C—H bond insertion). Further reagents include, but are not limited, to those described in Hermanson, Bioconjugate Techniques 2^{nd} Edition, Academic Press, 2008.

Some linkers such as those comprising peptide units and substrates for protease may be labile in the blood stream, thereby releasing unacceptable amounts of the drug prior to internalization in a target cell (Khot, A. et al (2015) Bioanalysis 7(13):1633-1648). Other linkers may provide stability in the bloodstream, but intracellular release effectiveness may be negatively impacted. Linkers that provide for desired intracellular release may have poor stability in the bloodstream. In addition, in standard conjugation processes, the amount of adjuvant/drug moiety loaded on the antibody, i.e. drug loading, the amount of aggregate that is formed in the conjugation reaction, and the yield of final purified conjugate that can be obtained are interrelated. Aggregate formation may be correlated to the number of equivalents of drug moieties conjugated to the antibody. Under high drug loading, formed aggregates must be removed for therapeutic applications. As a result, drug loading-mediated aggregate formation decreases antibody conjugate yield and can render process scale-up difficult.

Although cleavable linkers, for example with protease-substrate peptide units or immolative units such as para-aminobenzyloxycarbonyl, can provide certain advantages, linkers need not be cleavable. For non-cleavable linkers, azaBz adjuvant moiety release may not depend on the differential properties between the plasma and some cytoplasmic compartments. The release of a adjuvant moiety or its metabolite can occur after internalization of the immunoconjugate of via antigen-mediated endocytosis and delivery to lysosomal compartment, where the targeting moiety (or binding fragment thereof) can be degraded to the level of amino acids through intracellular proteolytic degradation. This process can release an adjuvant moiety or its metabolite. The released adjuvant moiety or metabolite thereof may be more hydrophilic and less membrane permeable, which can lead to less bystander effects and less non-specific toxicities compared to conjugates with a cleavable linker. Immunoconjugates with non-cleavable linkers can have greater stability in circulation than immunoconjugates with cleavable linkers. Non-cleavable linkers can include alkylene chains, or can be polymeric, such as, for example, based upon polyalkylene glycol polymers (PEG), amide polymers, or can include segments of alkylene chains, polyalkylene glycols and/or amide polymers. The linker can contain a PEG having from 2 to 50 ethylene glycol (PEG) units, or from 2 to 10 ethylene glycol (PEG) units.

Conjugation of the adjuvant azaBz moiety to a glycan group of an antibody may improve linkage stability, homogeneity, aggregation, and various pharmacokinetic properties of the immunoconjugate relative to conjugation to a native or engineered cysteine residue (Zhou, Q., et al (2014) Bioconjugate Chem. 25(3), 510-520; Okeley, N. M., et al (2013) Bioconjugate Chem. 24(10):1650-1655; U.S. Pat. No. 10,072,096; WO2015057063; WO2021248048). Some glycan remodeling methods use recombinant microbial transglutaminase to enable efficient, site-specific conjugation of drug-linker intermediates to position HC-Q295 of native, fully glycosylated IgG-type antibodies (Dickgeisser, S., et al (2020) Bioconjugate Chemistry 31(4), 1070-1076). The native glycan and modified glycan groups and the methods of conjugation may be those taught in Qasba, P. K. (2015) Bioconjugate Chem. 26:2170-2175; Jaramillo, M. L. et al, (2023) MABS, VOL. 15, NO. 1:1-15; Zhang, X., et al (2021) ACS Chem. Biol. 16:2502-2514, each of which are incorporated by reference herein.

The invention provides solutions to the limitations and challenges to the design, preparation and use of immunoconjugates. Some linkers may be labile in the blood stream, thereby releasing unacceptable amounts of the adjuvant/drug prior to internalization in a target cell (Khot, A. et al (2015) Bioanalysis 7(13):1633-1648). Other linkers may provide stability in the bloodstream, but intracellular release effectiveness may be negatively impacted. Linkers that provide for desired intracellular release typically have poor stability in the bloodstream. Alternatively stated, bloodstream stability and intracellular release are typically inversely related. In addition, in standard conjugation processes, the amount of adjuvant/drug moiety loaded on the antibody, i.e. drug loading, the amount of aggregate that is formed in the conjugation reaction, and the yield of final purified conjugate that can be obtained are interrelated. For example, aggregate formation is generally positively correlated to the number of equivalents of adjuvant/drug moiety and derivatives thereof conjugated to the antibody. Under high drug loading, formed aggregates must be removed for therapeutic applications. As a result, drug loading-mediated aggregate formation decreases immunoconjugate yield and can render process scale-up difficult.

Exemplary embodiments include an aza-benzazepine linker compound of Formula II:

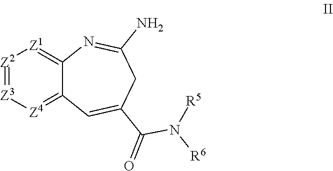

wherein
Z¹ is selected from CR¹ and N;
Z² is selected from CR² and N;
Z³ is selected from CR³ and N;
Z⁴ is selected from CR⁴ and N;
where one or two of Z¹, Z², Z³, and Z⁴ are N;
R¹, R², R³, R⁴, R⁵, and R⁶ are independently selected from the group consisting of H, C(═O), C(═O)N(R⁵), O, N(R⁵), S, S(O)₂, S(O)₂N(R), C₁-C₁₂ alkyl, C₂-C₆ alkenyl, C₂-C₆ alkynyl, C₃-C₁₂ carbocyclyl, C₆-C₂₀ aryl, C₂-C₉ heterocyclyl, and C₁-C₂₀ heteroaryl, each of which are independently and optionally substituted with one or more groups selected from:
—(C₁-C₁₂ alkyldiyl)-N(R⁷)—*;
—(C₁-C₁₂ alkyldiyl)-N(R⁷)₂;
—(C₁-C₁₂ alkyldiyl)-OR⁷;
—(C₃-C₁₂ carbocyclyl);
—(C₃-C₁₂ carbocyclyl)-*;
—(C₃-C₁₂ carbocyclyl)-(C₁-C₁₂ alkyldiyl)-NR⁷—*;
—(C₃-C₁₂ carbocyclyl)-(C₁-C₁₂ alkyldiyl)-N(R⁷)₂;
—(C₃-C₁₂ carbocyclyl)-NR⁷—C(═NR⁷)NR⁷—*;
—(C₆-C₂₀ aryl);
—(C₆-C₂₀ aryldiyl)-*;
—(C₆-C₂₀ aryldiyl)-N(R⁷)—*;
—(C₆-C₂₀ aryldiyl)-(C₁-C₁₂ alkyldiyl)-N(R⁷)—*;
—(C₆-C₂₀ aryldiyl)-(C₁-C₁₂ alkyldiyl)-(C₂-C₂₀ heterocyclyldiyl)-*;
—(C₆-C₂₀ aryldiyl)-(C₁-C₁₂ alkyldiyl)-N(R⁷)₂;
—(C₆-C₂₀ aryldiyl)-(C₁-C₁₂ alkyldiyl)-NR⁷—C(═NR^{7a})N(R⁷)—*;
—(C₂-C₂₀ heterocyclyl);
—(C₂-C₂₀ heterocyclyl)-*;
—(C₂-C₉ heterocyclyl)-(C₁-C₁₂ alkyldiyl)-NR⁷—*;
—(C₂-C₉ heterocyclyl)-(C₁-C₁₂ alkyldiyl)-N(R⁷)₂;
—(C₂-C₉ heterocyclyl)-C(═O)—(C₁-C₁₂ alkyldiyl)-N(R⁷)—*;
—(C₂-C₉ heterocyclyl)-NR⁷—C(═NR^{7a})NR⁷—*;
—(C₂-C₉ heterocyclyl)-NR⁷—(C₆-C₂₀ aryldiyl)-(C₁-C₁₂ alkyldiyl)-N(R⁷)—*;
—(C₂-C₉ heterocyclyl)-(C₆-C₂₀ aryldiyl)-*;
—(C₁-C₂₀ heteroaryl);
—(C₁-C₂₀ heteroaryl)-*;
—(C₁-C₂₀ heteroaryl)-(C₁-C₁₂ alkyldiyl)-N(R⁷)—*;
—(C₁-C₂₀ heteroaryl)-(C₁-C₁₂ alkyldiyl)-N(R⁷)₂;
—(C₁-C₂₀ heteroaryl)-NR⁷—C(═NR^{7a})N(R⁷)—*;
—(C₁-C₂₀ heteroaryl)-N(R⁷)C(═O)—(C₁-C₁₂ alkyldiyl)-N(R⁷)—*;
—C(═O)—*;
—C(═O)—(C₁-C₁₂ alkyldiyl)-N(R⁷)—*;
—C(═O)—(C₂-C₂₀ heterocyclyldiyl)-*;
—C(═O)N(R⁷)₂;
—C(═O)N(R⁷)—*;
—C(═O)N(R⁷)—(C₁-C₁₂ alkyldiyl)-N(R⁷)C(═O)R⁷;
—C(═O)N(R⁷)—(C₁-C₁₂ alkyldiyl)-N(R⁷)C(═O)N(R⁷)₂;
—C(═O)NR⁷—(C₁-C₁₂ alkyldiyl)-N(R⁷)CO₂R⁷;
—C(═O)NR⁵—(C₁-C₁₂ alkyldiyl)-N(R⁵⁷)C(═NR^{57a})N(R⁵⁷)₂;
—C(═O)NR⁵—(C₁-C₁₂ alkyldiyl)-NR⁵⁷C(═NR^{7a})R⁷;
—C(═O)NR⁵—(C₁-C₈ alkyldiyl)-NR⁷(C₂-C₅ heteroaryl);
—C(═O)NR⁷—(C₁-C₂₀ heteroaryldiyl)-N(R⁷)—*;
—C(═O)NR⁷—(C₁-C₂₀ heteroaryldiyl)-*;
—C(═O)NR⁷—(C₁-C₂₀ heteroaryldiyl)-(C₁-C₁₂ alkyldiyl)-N(R⁷)₂;
—C(═O)NR⁷—(C₁-C₂₀ heteroaryldiyl)-(C₂-C₂₀ heterocyclyldiyl)-C(═O)NR⁷—(C₁-C₁₂ alkyldiyl)-NR⁷—*;
—N(R⁷)₂;
—N(R⁷)—*;
—N(R⁷)C(═O)R⁷;
—N(R⁷)C(═O)—*;
—N(R⁷)C(═O)N(R⁷)₂;
—N(R⁷)C(═O)N(R⁷)—*;
—N(R⁷)CO₂R⁷;
—NR⁷C(═NR^{7a})N(R⁷)₂;
—NR⁷C(═NR^{7a})N(R⁷)—*;
—NR⁷C(═NR^{7a})R⁷;
—N(R⁷)C(═O)—(C₁-C₁₂ alkyldiyl)-N(R⁷)—*;
—N(R⁷)—(C₂-C₅ heteroaryl);
—N(R⁷)—S(═O)₂—(C₁-C₁₂ alkyl);
—O—(C₁-C₁₂ alkyl);
—O—(C₁-C₁₂ alkyldiyl)-N(R⁷)₂;
—O—(C₁-C₁₂ alkyldiyl)-N(R⁷)—*;
—O—C(═O)N(R⁷)₂;
—O—C(═O)N(R⁷)—*;
—O—(R⁷)—*;
—OR⁷;
—S(═O)₂—(C₂-C₂₀ heterocyclyldiyl)-*;
—S(═O)₂—(C₂-C₂₀ heterocyclyldiyl)-(C₁-C₁₂ alkyldiyl)-N(R⁷)₂;
—S(═O)₂—(C₂-C₂₀ heterocyclyldiyl)-(C₁-C₁₂ alkyldiyl)-NR⁷—*; and
—S(═O)₂—(C₂-C₂₀ heterocyclyldiyl)-(C₁-C₁₂ alkyldiyl)-OH;
or R⁵ and R⁶ together form a 5- or 6-membered heterocyclyl ring;
R⁷ is independently selected from the group consisting of H, C₆-C₂₀ aryl, C₃-C₁₂ carbocyclyl, C₆-C₂₀ aryldiyl, C₁-C₁₂ alkyl, and C₁-C₁₂ alkyldiyl, or two R⁵ groups together form a 5- or 6-membered heterocyclyl ring;
R^{7a} is selected from the group consisting of C₆-C₂₀ aryl and C₁-C₂₀ heteroaryl;
where the asterisk * indicates the attachment site of L, and where one of R¹, R², R³, R⁴, R⁵ and R⁶ is attached to L; and
alkyl, alkyldiyl, alkenyl, alkenyldiyl, alkynyl, alkynyldiyl, aryl, aryldiyl, carbocyclyl, carbocyclyldiyl, heterocyclyl, heterocyclyldiyl, heteroaryl, and heteroaryldiyl are independently and optionally substituted with one or more groups independently selected from F, Cl, Br, I, —CN, —CH₃, —CH₂CH₃, —CH═CH₂, —C═CH, —C═CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —O(CH₂CH₂O)ᵣ—(CH₂)ₘCO₂H, —O(CH₂CH₂O)~H, —OCH₂F, —OCHF₂, —OCF₃, —OP(O)(OH)₂, —S(O)₂N(CH₃)₂, —SCH₃, —S(O)₂CH₃, and —S(O)₃H.

An exemplary embodiment of L of Formula II is selected from the group consisting of:
Q-C(═O)-PEG-;
Q-C(═O)-PEG-C(═O)N(R⁸)—(C₁-C₁₂ alkyldiyl)-C(═O)-Gluc-;
Q-C(═O)-PEG-O—;
Q-C(═O)-PEG-O—C(═O)—;
Q-C(═O)-PEG-C(═O)—;
Q-C(═O)-PEG-C(═O)-PEP-;
Q-C(═O)-PEG-N(R⁸)—;
Q-C(═O)-PEG-N(R⁸)—C(═O)—;
Q-C(═O)-PEG-N(R⁸)-PEG-C(═O)-PEP-;
Q-C(═O)-PEG-N*(R⁸)₂-PEG-C(═O)-PEP-;
Q-C(═O)-PEG-C(═O)-PEP-N(R⁸)—(C₁-C₁₂ alkyldiyl)-;
Q-C(═O)-PEG-C(═O)-PEP-N(R⁸)—(C₁-C₁₂ alkyldiyl)N(R⁸)C(═O)—(C₂-C₅ monoheterocyclyldiyl)-;
Q-C(═O)-PEG-SS-(C₁-C₁₂ alkyldiyl)-OC(═O)—;
Q-C(═O)-PEG-SS-(C₁-C₁₂ alkyldiyl)-C(═O)—;
Q-C(═O)—(C₁-C₁₂ alkyldiyl)-C(═O)-PEP-;
Q-C(═O)—(C₁-C₁₂ alkyldiyl)-C(═O)-PEP-N(R⁸)—(C₁-C₁₂ alkyldiyl)-;
Q-C(═O)—(C₁-C₁₂ alkyldiyl)-C(═O)-PEP-N(R⁸)—(C₁-C₁₂ alkyldiyl)-N(R⁸)—C(═O);
Q-C(═O)—(C₁-C₁₂ alkyldiyl)-C(═O)-PEP-N(R⁸)—(C₁-C₁₂ alkyldiyl)-N(R⁸)C(═O)—(C₂-C₅ monoheterocyclyldiyl)-;
Q-(CH₂)ₘ—C(═O)N(R⁸)-PEG-;
Q-(CH₂)ₘ—C(═O)N(R⁸)-PEG-C(═O)N(R⁸)—(C₁-C₁₂ alkyldiyl)-C(═O)-Gluc-;
Q-(CH₂)ₘ—C(═O)N(R⁸)-PEG-O—;
Q-(CH₂)ₘ—C(═O)N(R⁸)-PEG-O—C(═O)—;
Q-(CH₂)ₘ—C(═O)N(R⁸)-PEG-C(═O)—;
Q-(CH₂)ₘ—C(═O)N(R⁸)-PEG-N(R⁸)—;
Q-(CH₂)ₘ—C(═O)N(R⁸)-PEG-N(R⁸)—C(═O)—;
Q-(CH₂)ₘ—C(═O)N(R⁸)-PEG-C(═O)-PEP-;
Q-(CH₂)ₘC(═O)N(R⁸)-PEG-SS-(C₁-C₁₂ alkyldiyl)-OC(═O)—;
Q-(CH₂)ₘC(═O)-PEP-N(R⁸)—(C₁-C₁₂ alkyldiyl)-;
Q-(CH₂)ₘC(═O)-PEP-N(R⁸)—(C₁-C₁₂ alkyldiyl)N(R⁸)C(═O)—; and
Q-(CH₂)ₘC(═O)-PEP-N(R⁸)—(C₁-C₁₂ alkyldiyl)N(R⁸)C(═O)—(C₂-C₅ monoheterocyclyldiyl)-;
R⁸ is independently H or C₁-C₆ alkyl;
PEG has the formula: —(CH₂CH₂O)ₙ—(CH₂)ₘ—; m is an integer from 1 to 5, and n is an integer from 1 to 50;
Gluc has the formula:

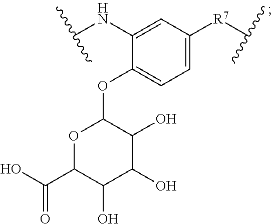

PEP has the formula:

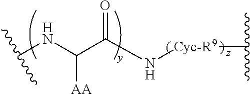

where AA is independently selected from a natural or unnatural amino acid side chain, or one or more of AA, and an adjacent nitrogen atom form a 5-membered ring proline amino acid, and the wavy line indicates a point of attachment;
Cyc is selected from C₆-C₂₀ aryldiyl and C₁-C₂₀ heteroaryldiyl, optionally substituted with one or more groups selected from F, Cl, NO₂, —OH, —OCH₃, and a glucuronic acid having the structure:

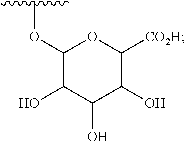 [SMILES: *OC1OC(C(=O)O)C(O)C(O)C1O]

R⁹ is selected from the group consisting of —CH(R¹⁰)O—, —CH₂—, —CH₂N(R¹⁰)—, and —CH(R¹⁰)O—C(═O)—, where R¹⁰ is selected from H, C₁-C₆ alkyl, C(═O)—C₁-C₆ alkyl, and —C(═O)N(R¹)₂, where R¹¹ is independently selected from the group consisting of H, C₁-C₁₂ alkyl, and —(CH₂CH₂O)ₙ—(CH₂)ₘ—OH, where m is an integer from 1 to 5, and n is an integer from 2 to 50, or two R¹¹ groups together form a 5- or 6-membered heterocyclyl ring;
y is an integer from 2 to 12; and
z is 0 or 1.

An exemplary embodiment of Q is selected from the group consisting of N-hydroxysuccinimidyl, N-hydroxysulfosuccinimidyl, maleimide, and phenoxy substituted with one or more groups independently selected from F, Cl, NO₂, and SO₃⁻.

An exemplary embodiment of Q is selected from:

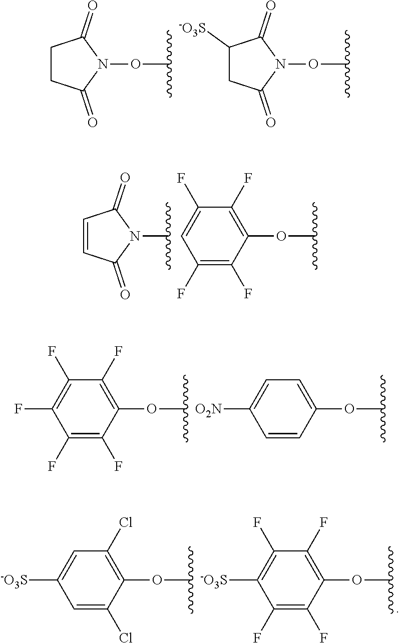

An exemplary embodiment of Q is phenoxy substituted with one or more F.

An exemplary embodiment of Q is 2,3,5,6-tetrafluorophenoxy.

An exemplary embodiment of the aza-benzazepine linker compound of Formula II is selected from Tables 2a and 2b. Each compound was synthesized, purified, and characterized by mass spectrometry and shown to have the mass indicated. Additional experimental procedures are found in the Examples. The aza-benzazepine linker compounds of Tables 2a and 2b (azaBzL) demonstrate the surprising and unexpected property of TLR8 agonist selectivity which may predict useful therapeutic activity to treat cancer and other disorders. The aza-benzazepine linker intermediate, Formula II compounds of Tables 2a and 2b are used in conjugation with antibodies by the methods of Example 201 to form the Immunoconjugates of Tables 3a and 3b.

TABLE 2a
Aza-Benzazepine Linker (azaBzL) compounds

| azaBzL | | |
|---|---|---|
| No. | Structure | MW |
| azaBzL-1 | 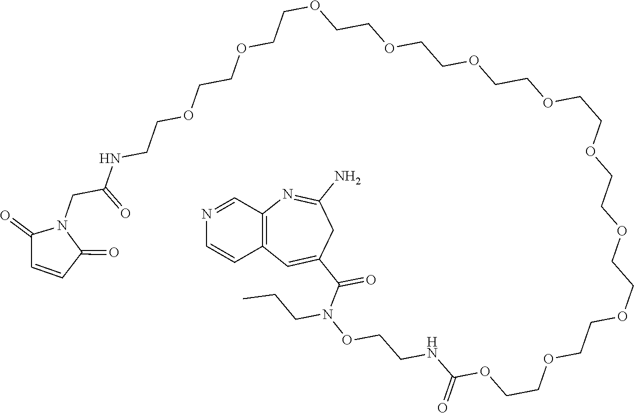 [SMILES: CCCN(OCCNC(=O)OCCOCCOCCOCCOCCOCCOCCOCCOCCOCCOCCNC(=O)CN1C(=O)C=CC1=O)C(=O)C1=Cc2ccncc2N=C(N)C1] | 968.1 |
| azaBzL-2 | 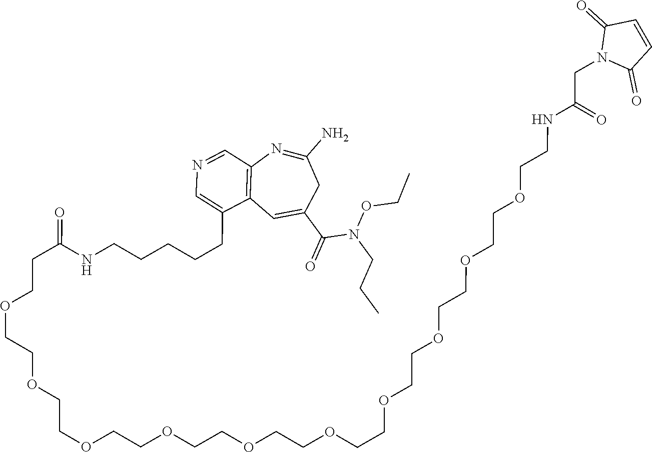 [SMILES: CCCN(OCC)C(=O)C1=Cc2c(CCCCCNC(=O)CCOCCOCCOCCOCCOCCOCCOCCOCCOCCOCCNC(=O)CN3C(=O)C=CC3=O)cncc2N=C(N)C1] | 1022.2 |
| azaBzL-3 | 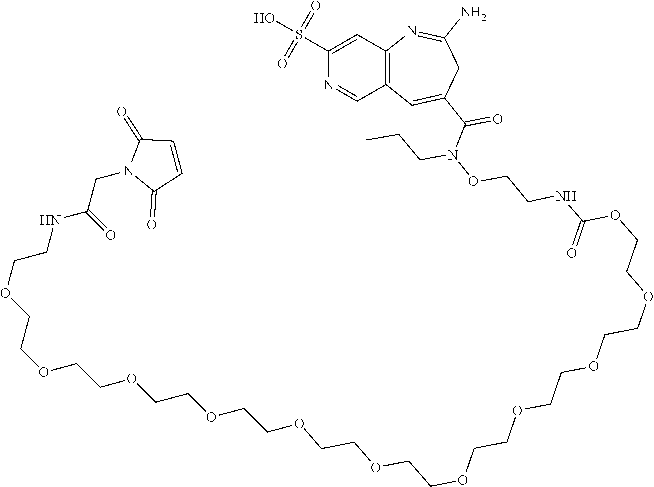 [SMILES: CCCN(OCCNC(=O)OCCOCCOCCOCCOCCOCCOCCOCCOCCOCCOCCNC(=O)CN1C(=O)C=CC1=O)C(=O)C1=Cc2cnc(S(=O)(=O)O)cc2N=C(N)C1] | 1048.1 |
| azaBzL-4 | 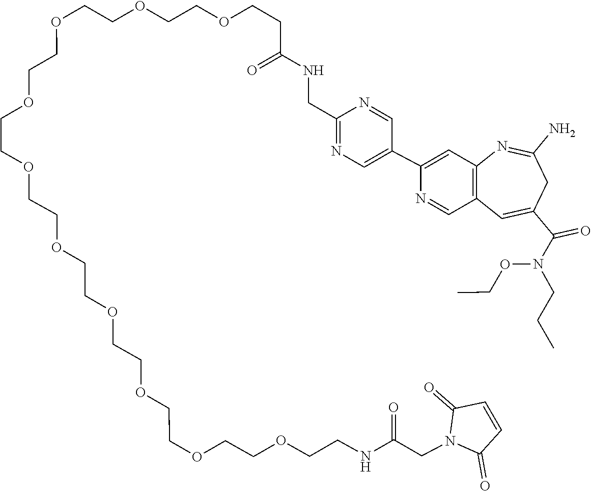 [SMILES: CCCN(OCC)C(=O)C1=Cc2cnc(-c3cnc(CNC(=O)CCOCCOCCOCCOCCOCCOCCOCCOCCOCCOCCNC(=O)CN4C(=O)C=CC4=O)nc3)cc2N=C(N)C1] | 1044.2 |
| azaBzL-5 | 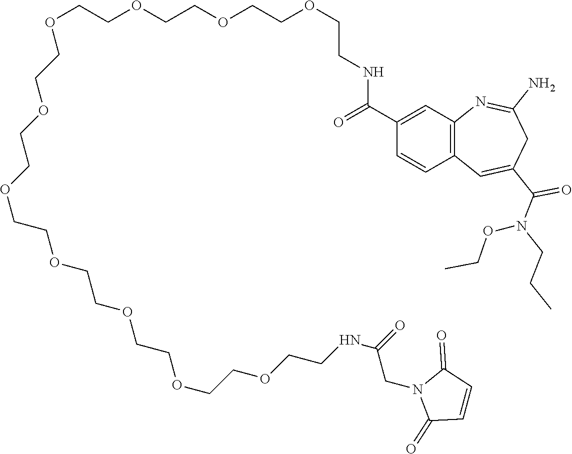 [SMILES: CCCN(OCC)C(=O)C1=Cc2ccc(C(=O)NCCOCCOCCOCCOCCOCCOCCOCCOCCOCCOCCNC(=O)CN3C(=O)C=CC3=O)cc2N=C(N)C1] | 952.1 |
| azaBzL-6 | 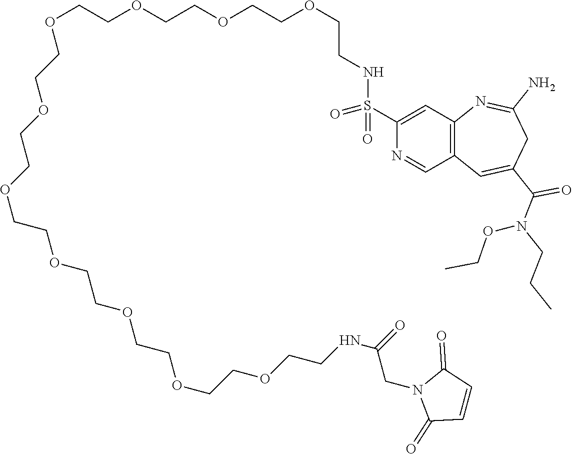 [SMILES: CCCN(OCC)C(=O)C1=Cc2cnc(S(=O)(=O)NCCOCCOCCOCCOCCOCCOCCOCCOCCOCCOCCNC(=O)CN3C(=O)C=CC3=O)cc2N=C(N)C1] | 988.1 |
| azaBzL-7 | 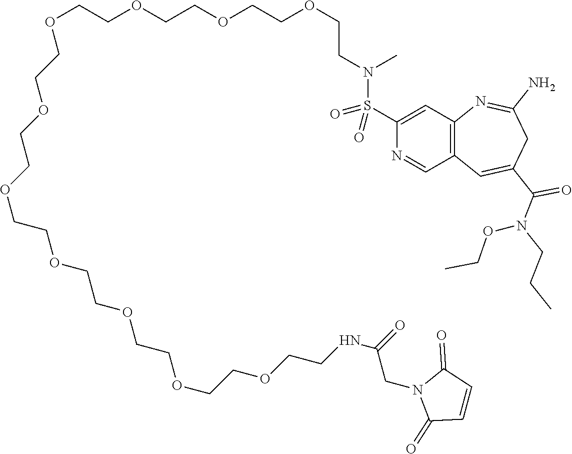 [SMILES: CCCN(OCC)C(=O)C1=Cc2cnc(S(=O)(=O)N(C)CCOCCOCCOCCOCCOCCOCCOCCOCCOCCOCCNC(=O)CN3C(=O)C=CC3=O)cc2N=C(N)C1] | 1002.1 |
| azaBzL-8 | 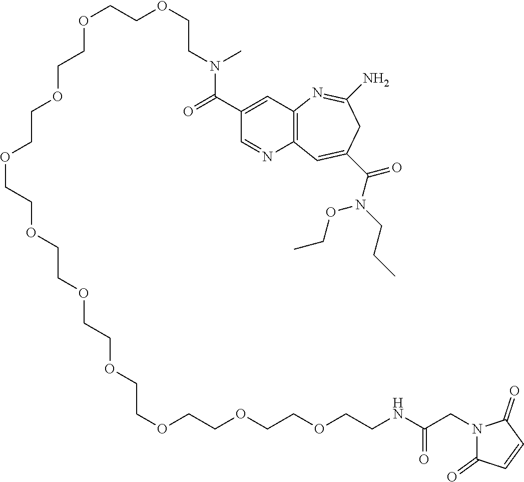 | 966.1 |
| azaBzL-9 | 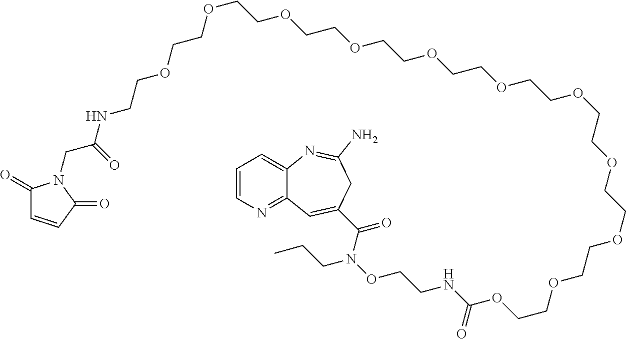 [SMILES: CCCN(OCCNC(=O)OCCOCCOCCOCCOCCOCCOCCOCCOCCOCCOCCNC(=O)CN1C(=O)C=CC1=O)C(=O)C1=Cc2ncccc2N=C(N)C1] | 968.1 |
| azaBzL-10 | 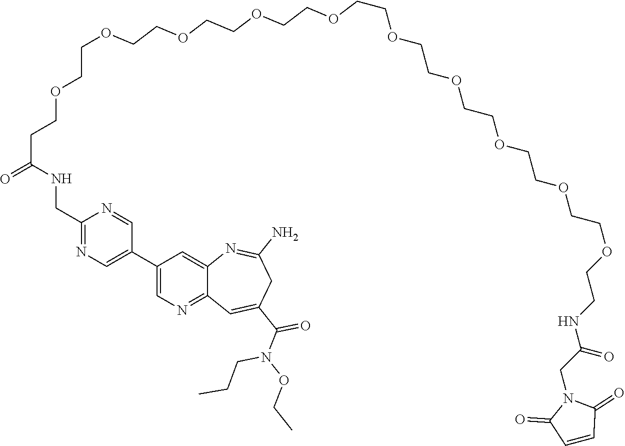 [SMILES: CCCN(OCC)C(=O)C1=Cc2ncc(-c3cnc(CNC(=O)CCOCCOCCOCCOCCOCCOCCOCCOCCOCCOCCNC(=O)CN4C(=O)C=CC4=O)nc3)cc2N=C(N)C1] | 1044.2 |
| azaBzL-11 | 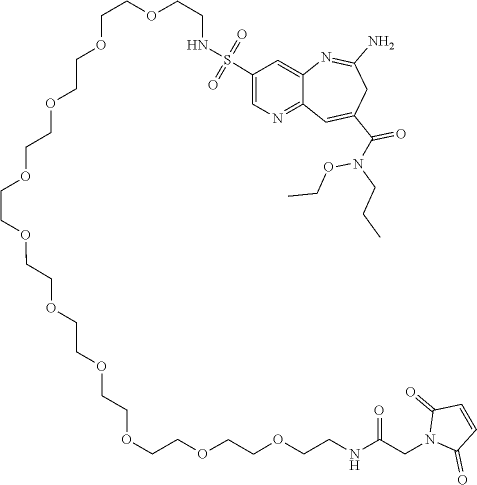 | 988.1 |
| azaBzL-12 | 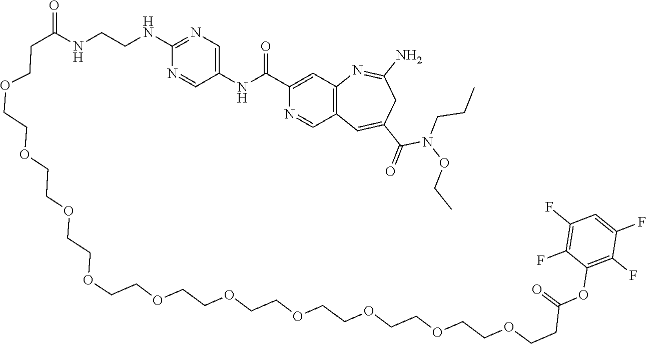 [SMILES: CCCN(OCC)C(=O)C1=Cc2cnc(C(=O)Nc3cnc(NCCNC(=O)CCOCCOCCOCCOCCOCCOCCOCCOCCOCCOCCC(=O)Oc4c(F)c(F)cc(F)c4F)nc3)cc2N=C(N)C1] | 1156.2 |
| azaBzL-13 | 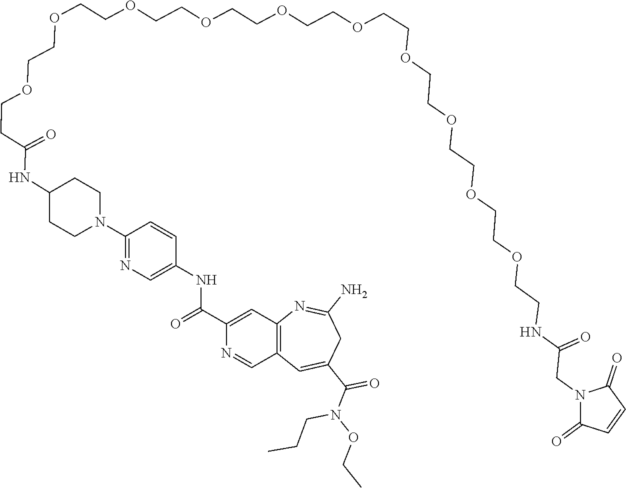 [SMILES: CCCN(OCC)C(=O)C1=Cc2cnc(C(=O)Nc3ccc(N4CCC(NC(=O)CCOCCOCCOCCOCCOCCOCCOCCOCCOCCOCCNC(=O)CN5C(=O)C=CC5=O)CC4)nc3)cc2N=C(N)C1] | 1155.3 |
| azaBzL-14 | 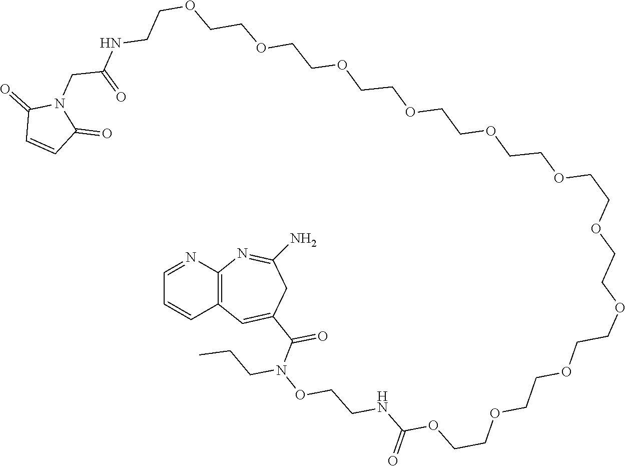 [SMILES: CCCN(OCCNC(=O)OCCOCCOCCOCCOCCOCCOCCOCCOCCOCCOCCNC(=O)CN1C(=O)C=CC1=O)C(=O)C1=Cc2cccnc2N=C(N)C1] | 968.1 |
| azaBzL-15 | 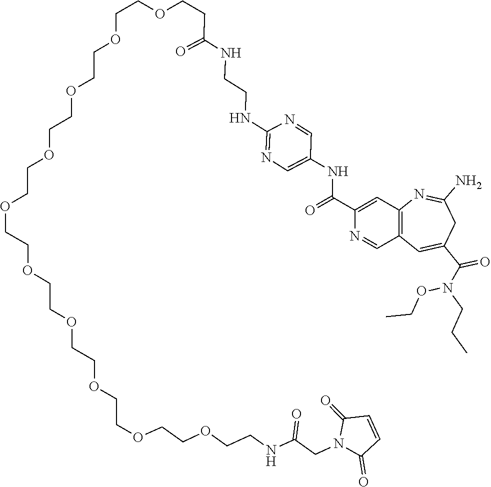 | 1116.2 |
| azaBzL-16 | 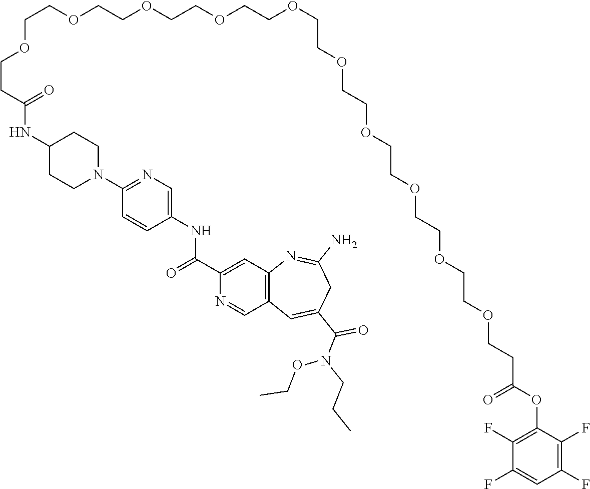 [SMILES: CCCN(OCC)C(=O)C1=Cc2cnc(C(=O)Nc3ccc(N4CCC(NC(=O)CCOCCOCCOCCOCCOCCOCCOCCOCCOCCOCCC(=O)Oc5c(F)c(F)cc(F)c5F)CC4)nc3)cc2N=C(N)C1] | 1195.3 |
| azaBzL-17 | 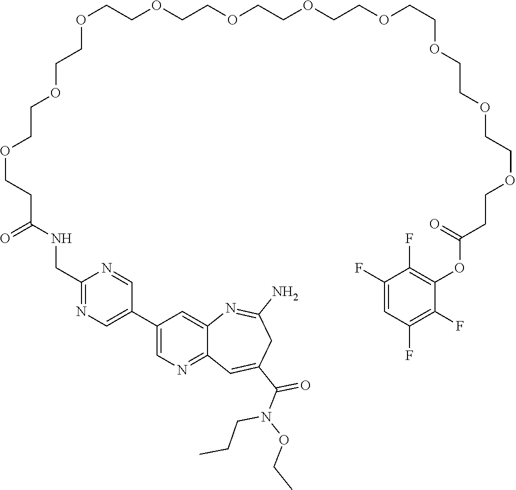 [SMILES: CCCN(OCC)C(=O)C1=Cc2ncc(-c3cnc(CNC(=O)CCOCCOCCOCCOCCOCCOCCOCCOCCOCCOCCC(=O)Oc4c(F)c(F)cc(F)c4F)nc3)cc2N=C(N)C1] | 1084.1 |
| azaBzL-18 | 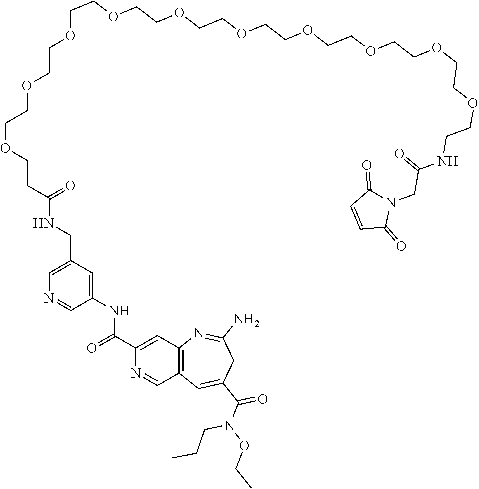 [SMILES: CCCN(OCC)C(=O)C1=Cc2cnc(C(=O)Nc3cncc(CNC(=O)CCOCCOCCOCCOCCOCCOCCOCCOCCOCCOCCNC(=O)CN4C(=O)C=CC4=O)c3)cc2N=C(N)C1] | 1086.2 |
| azaBzL-19 | 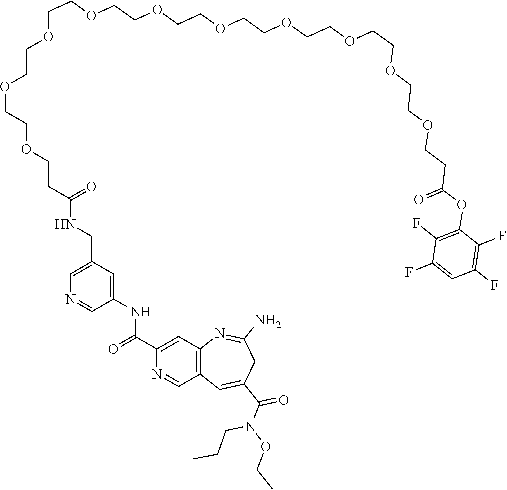 [SMILES: CCCN(OCC)C(=O)C1=Cc2cnc(C(=O)Nc3cncc(CNC(=O)CCOCCOCCOCCOCCOCCOCCOCCOCCOCCOCCC(=O)Oc4c(F)c(F)cc(F)c4F)c3)cc2N=C(N)C1] | 1126.2 |

TABLE 2b
Aza-Benzazepine Linker (azaBzL) compounds

| | | |
|---|---|---|
| azaBzL-20 | 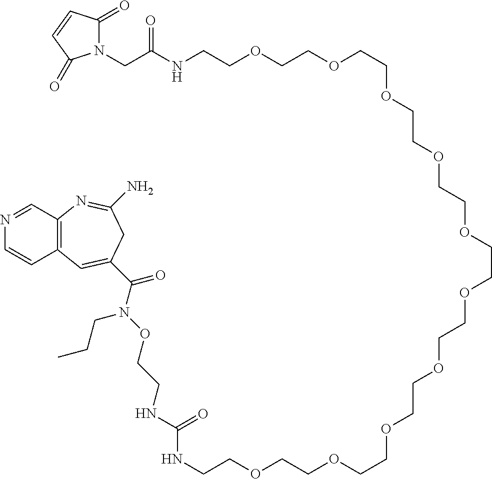 [SMILES: CCCN(OCCNC(=O)NCCOCCOCCOCCOCCOCCOCCOCCOCCOCCOCCNC(=O)CN1C(=O)C=CC1=O)C(=O)C1=Cc2ccncc2N=C(N)C1] | 967.1 |
| azaBzL-21 | 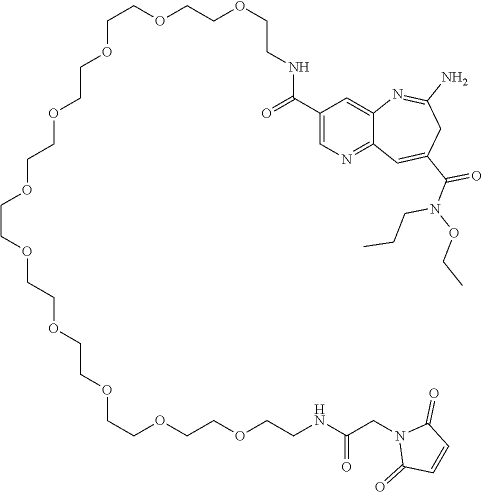 [SMILES: CCCN(OCC)C(=O)C1=Cc2ncc(C(=O)NCCOCCOCCOCCOCCOCCOCCOCCOCCOCCOCCNC(=O)CN3C(=O)C=CC3=O)cc2N=C(N)C1] | 952.1 |
| azaBzL-22 | 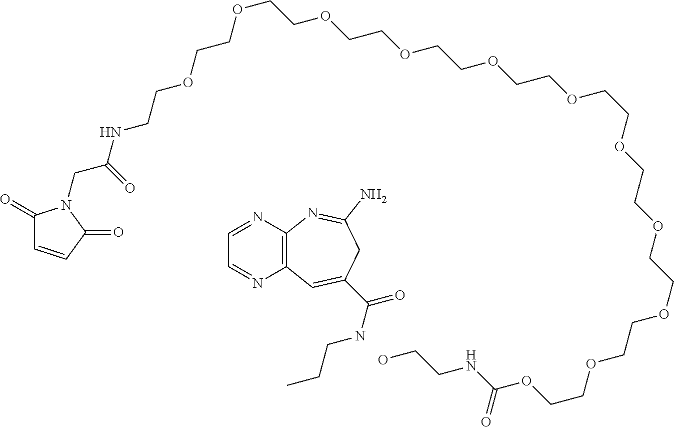 | 969.1 |
| azaBzL-23 | 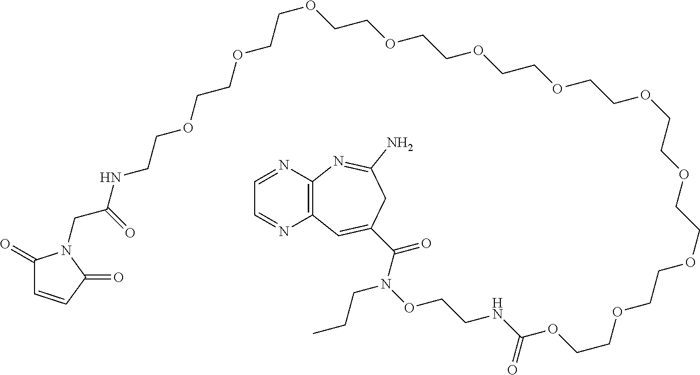 [SMILES: CCCN(OCCNC(=O)OCCOCCOCCOCCOCCOCCOCCOCCOCCOCCOCCNC(=O)CN1C(=O)C=CC1=O)C(=O)C1=Cc2nccnc2N=C(N)C1] | 969.1 |
| azaBzL-24 | 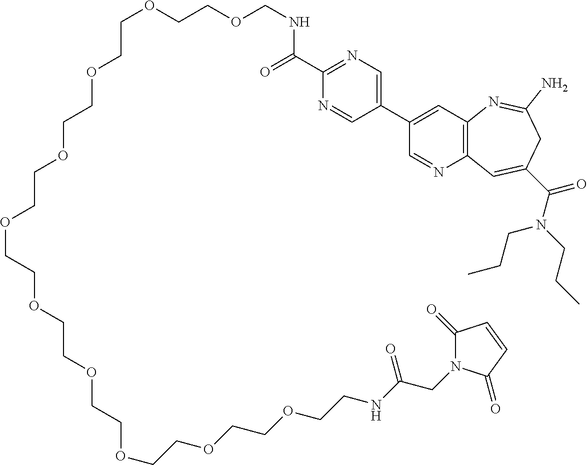 [SMILES: CCCN(CCC)C(=O)C1=Cc2ncc(-c3cnc(C(=O)NCOCCOCCOCCOCCOCCOCCOCCOCCOCCOCCNC(=O)CN4C(=O)C=CC4=O)nc3)cc2N=C(N)C1] | 1042.2 |
| azaBzL-25 | 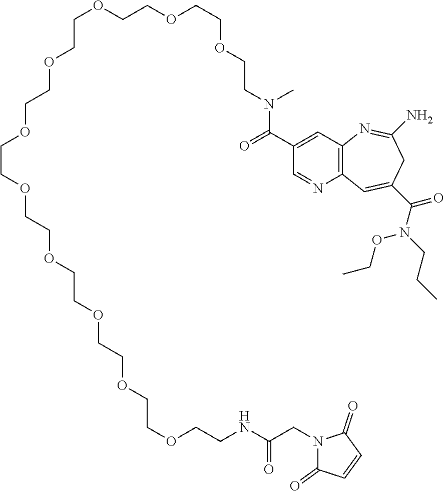 | 966.1 |
| azaBzL-26 | 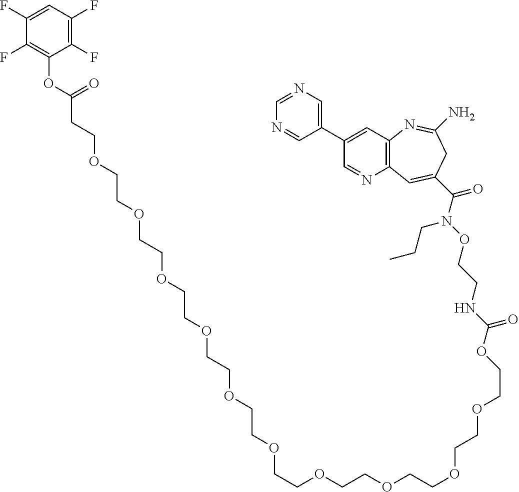 [SMILES: CCCN(OCCNC(=O)OCCOCCOCCOCCOCCOCCOCCOCCOCCOCCOCCC(=O)Oc1c(F)c(F)cc(F)c1F)C(=O)C1=Cc2ncc(-c3cncnc3)cc2N=C(N)C1] | 1086.1 |
| azaBzL-27 | 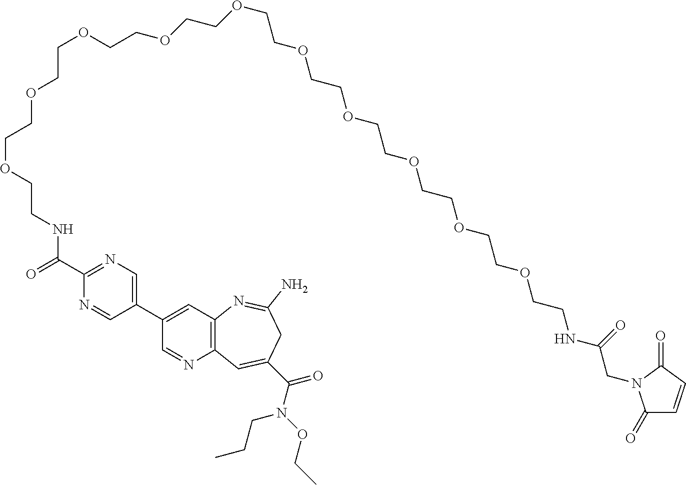 [SMILES: CCCN(OCC)C(=O)C1=Cc2ncc(-c3cnc(C(=O)NCCOCCOCCOCCOCCOCCOCCOCCOCCOCCOCCNC(=O)CN4C(=O)C=CC4=O)nc3)cc2N=C(N)C1] | 1030.1 |
| azaBzL-28 | 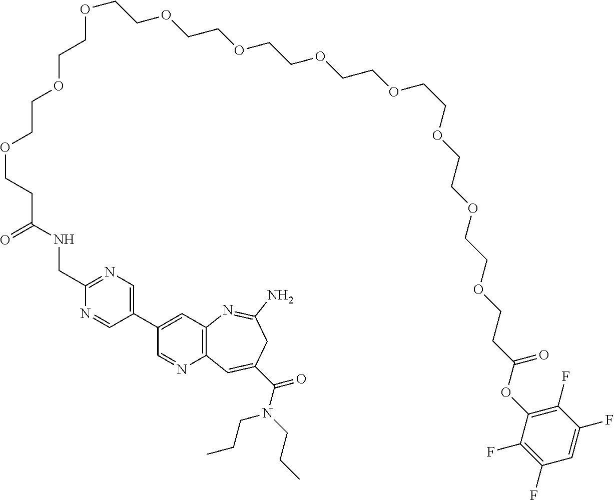 [SMILES: CCCN(CCC)C(=O)C1=Cc2ncc(-c3cnc(CNC(=O)CCOCCOCCOCCOCCOCCOCCOCCOCCOCCOCCC(=O)Oc4c(F)c(F)cc(F)c4F)nc3)cc2N=C(N)C1] | 1082.2 |
| azaBzL-29 | 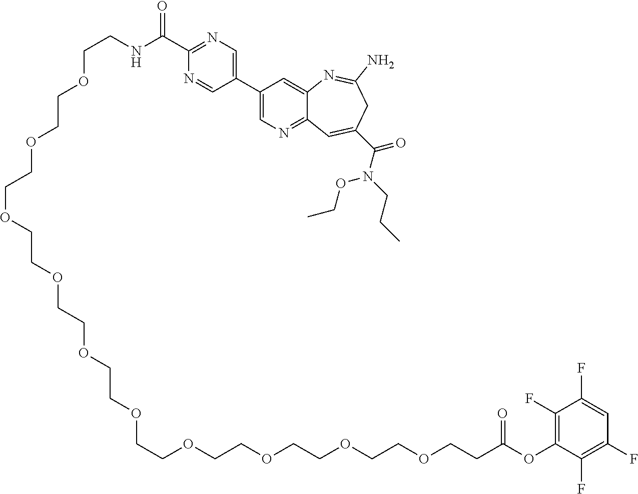 [SMILES: CCCN(OCC)C(=O)C1=Cc2ncc(-c3cnc(C(=O)NCCOCCOCCOCCOCCOCCOCCOCCOCCOCCOCCC(=O)Oc4c(F)c(F)cc(F)c4F)nc3)cc2N=C(N)C1] | 1070.1 |
| azaBzL-30 | 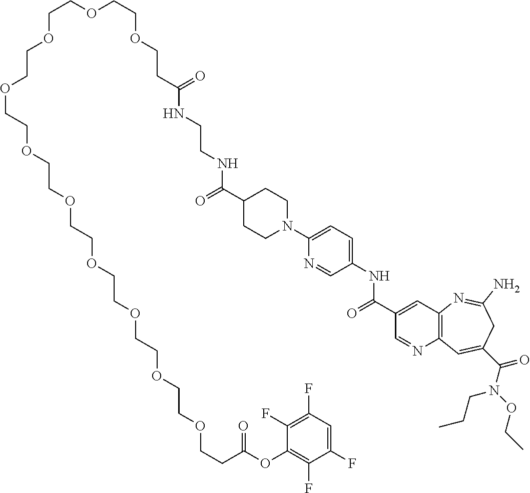 [SMILES: CCCN(OCC)C(=O)C1=Cc2ncc(C(=O)Nc3ccc(N4CCC(C(=O)NCCNC(=O)CCOCCOCCOCCOCCOCCOCCOCCOCCOCCOCCC(=O)Oc5c(F)c(F)cc(F)c5F)CC4)nc3)cc2N=C(N)C1] | 1266.4 |
| azaBzL-31 | 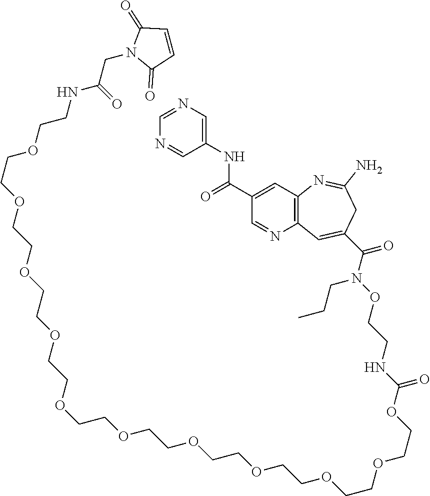 [SMILES: CCCN(OCCNC(=O)OCCOCCOCCOCCOCCOCCOCCOCCOCCOCCOCCNC(=O)CN1C(=O)C=CC1=O)C(=O)C1=Cc2ncc(C(=O)Nc3cncnc3)cc2N=C(N)C1] | 1089.2 |
| azaBzL-32 | 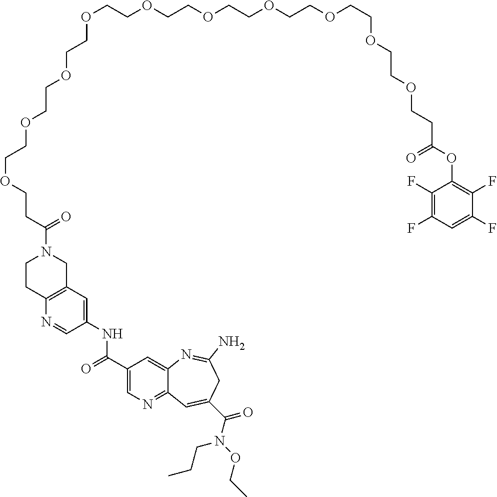 [SMILES: CCCN(OCC)C(=O)C1=Cc2ncc(C(=O)Nc3cnc4c(c3)CN(C(=O)CCOCCOCCOCCOCCOCCOCCOCCOCCOCCOCCC(=O)Oc3c(F)c(F)cc(F)c3F)CC4)cc2N=C(N)C1] | 1152.2 |
| azaBzL-33 | 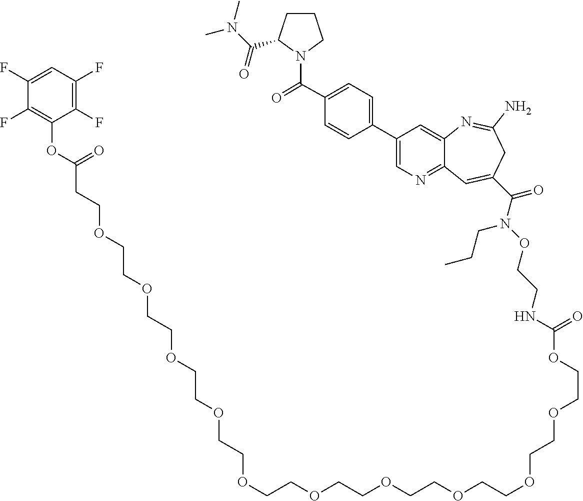 [SMILES: CCCN(OCCNC(=O)OCCOCCOCCOCCOCCOCCOCCOCCOCCOCCOCCC(=O)Oc1c(F)c(F)cc(F)c1F)C(=O)C1=Cc2ncc(-c3ccc(C(=O)N4CCC[C@H]4C(=O)N(C)C)cc3)cc2N=C(N)C1] | 1251.6 |
| azaBzL-34 | 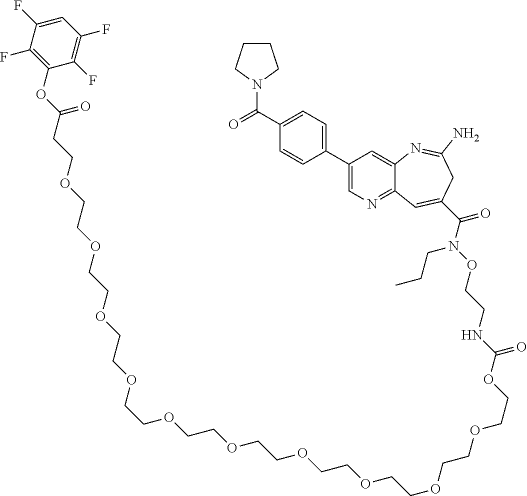 [SMILES: CCCN(OCCNC(=O)OCCOCCOCCOCCOCCOCCOCCOCCOCCOCCOCCC(=O)Oc1c(F)c(F)cc(F)c1F)C(=O)C1=Cc2ncc(-c3ccc(C(=O)N4CCCC4)cc3)cc2N=C(N)C1] | 1181.2 |
| azaBzL-35 | 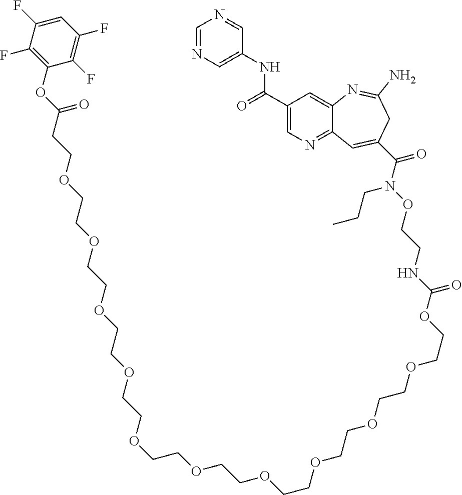 [SMILES: CCCN(OCCNC(=O)OCCOCCOCCOCCOCCOCCOCCOCCOCCOCCOCCC(=O)Oc1c(F)c(F)cc(F)c1F)C(=O)C1=Cc2ncc(C(=O)Nc3cncnc3)cc2N=C(N)C1] | 1129.1 |
| azaBzL-36 | 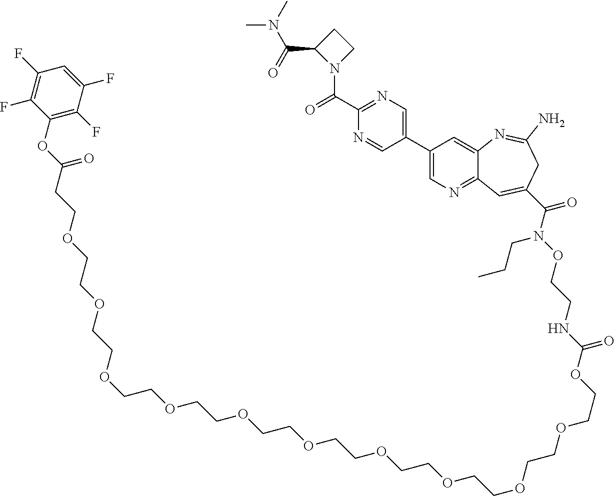 [SMILES: CCCN(OCCNC(=O)OCCOCCOCCOCCOCCOCCOCCOCCOCCOCCOCCC(=O)Oc1c(F)c(F)cc(F)c1F)C(=O)C1=Cc2ncc(-c3cnc(C(=O)N4CC[C@@H]4C(=O)N(C)C)nc3)cc2N=C(N)C1] | 1240.3 |
| azaBzL-37 | 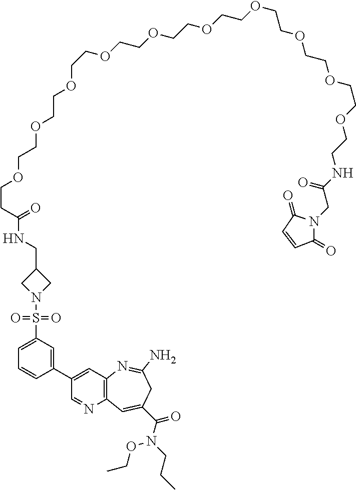 [SMILES: CCCN(OCC)C(=O)C1=Cc2ncc(-c3cccc(S(=O)(=O)N4CC(CNC(=O)CCOCCOCCOCCOCCOCCOCCOCCOCCOCCOCCNC(=O)CN5C(=O)C=CC5=O)C4)c3)cc2N=C(N)C1] | 1161.3 |
| azaBzL-38 | 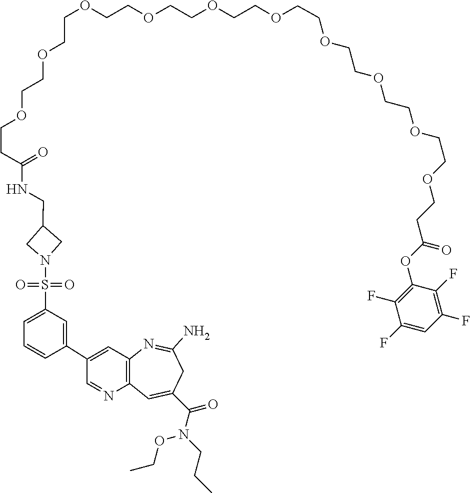 [SMILES: CCCN(OCC)C(=O)C1=Cc2ncc(-c3cccc(S(=O)(=O)N4CC(CNC(=O)CCOCCOCCOCCOCCOCCOCCOCCOCCOCCOCCC(=O)Oc5c(F)c(F)cc(F)c5F)C4)c3)cc2N=C(N)C1] | 1201.3 |
| azaBzL-39 | 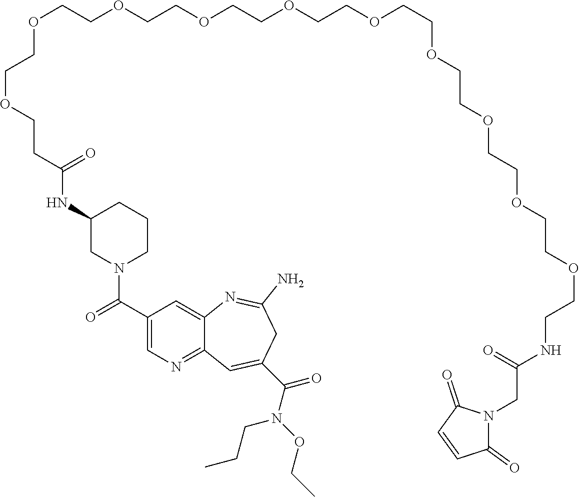 [SMILES: CCCN(OCC)C(=O)C1=Cc2ncc(C(=O)N3CCC[C@H](NC(=O)CCOCCOCCOCCOCCOCCOCCOCCOCCOCCOCCNC(=O)CN4C(=O)C=CC4=O)C3)cc2N=C(N)C1] | 1063.2 |
| azaBzL-40 | 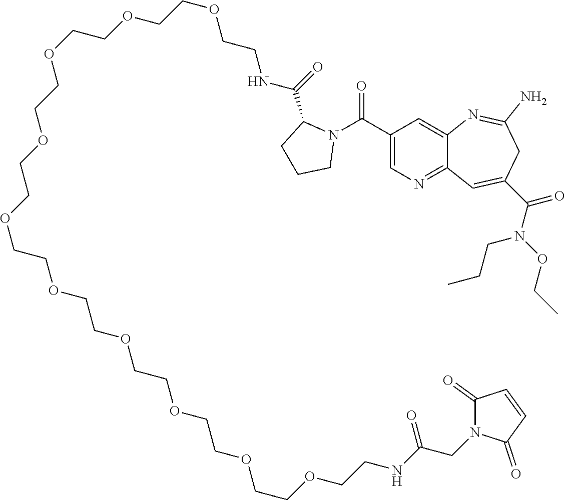 [SMILES: CCCN(OCC)C(=O)C1=Cc2ncc(C(=O)N3CCC[C@@H]3C(=O)NCCOCCOCCOCCOCCOCCOCCOCCOCCOCCOCCNC(=O)CN3C(=O)C=CC3=O)cc2N=C(N)C1] | 1049.2 |
| azaBzL-41 | 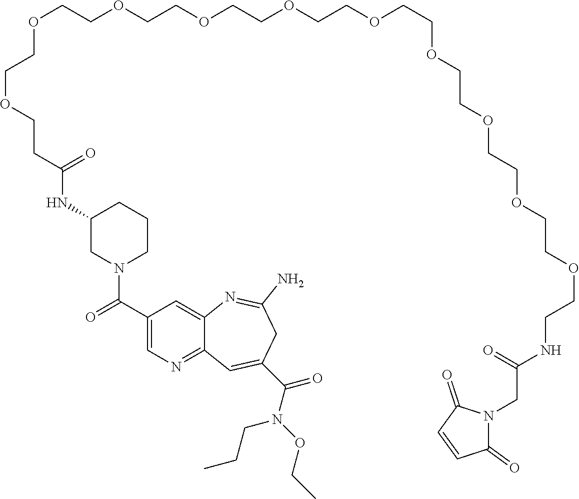 [SMILES: CCCN(OCC)C(=O)C1=Cc2ncc(C(=O)N3CCC[C@@H](NC(=O)CCOCCOCCOCCOCCOCCOCCOCCOCCOCCOCCNC(=O)CN4C(=O)C=CC4=O)C3)cc2N=C(N)C1] | 1063.2 |
| azaBzL-42 | 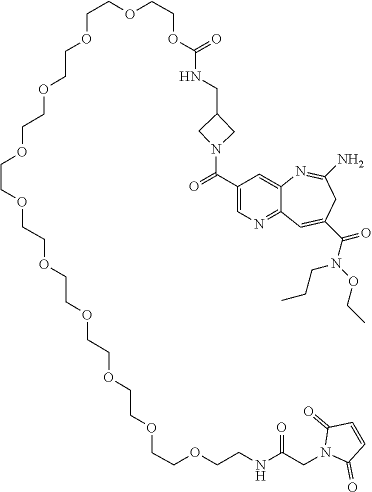 | 1065.2 |
| azaBzL-43 | 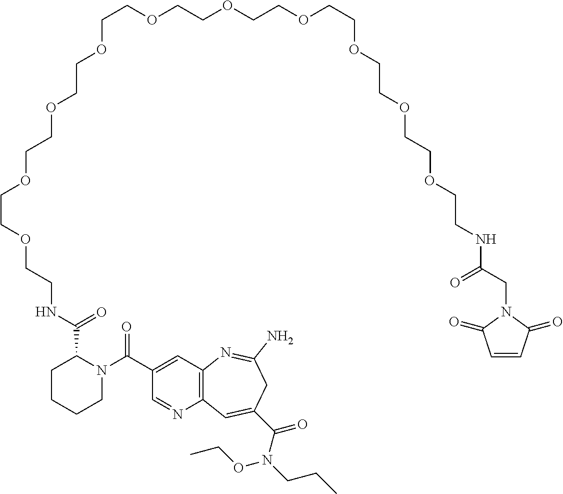 [SMILES: CCCN(OCC)C(=O)C1=Cc2ncc(C(=O)N3CCCC[C@@H]3C(=O)NCCOCCOCCOCCOCCOCCOCCOCCOCCOCCOCCNC(=O)CN3C(=O)C=CC3=O)cc2N=C(N)C1] | 1063.2 |
| azaBzL-44 | 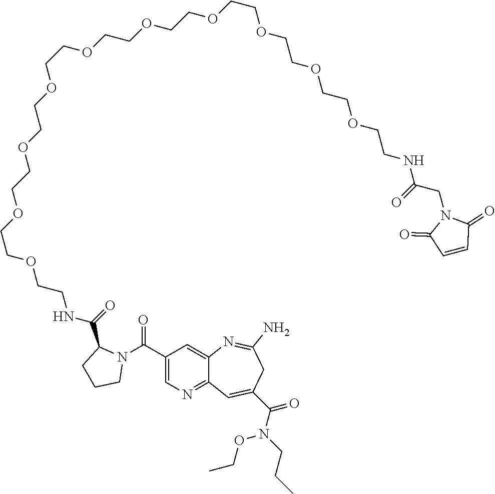 [SMILES: CCCN(OCC)C(=O)C1=Cc2ncc(C(=O)N3CCC[C@H]3C(=O)NCCOCCOCCOCCOCCOCCOCCOCCOCCOCCOCCNC(=O)CN3C(=O)C=CC3=O)cc2N=C(N)C1] | 1049.2 |
| azaBzL-45 | 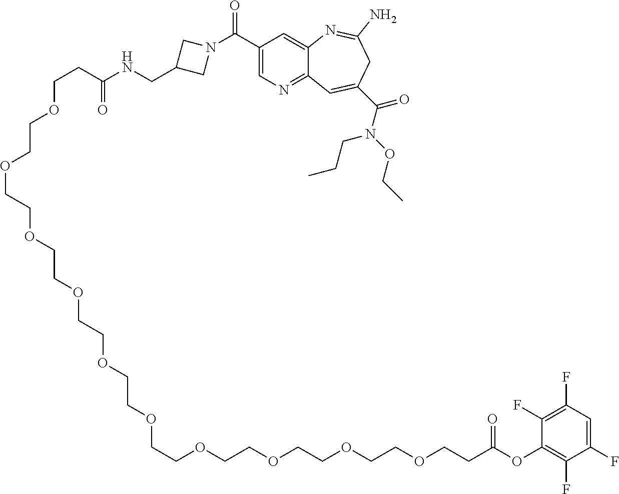 [SMILES: CCCN(OCC)C(=O)C1=Cc2ncc(C(=O)N3CC(CNC(=O)CCOCCOCCOCCOCCOCCOCCOCCOCCOCCOCCC(=O)Oc4c(F)c(F)cc(F)c4F)C3)cc2N=C(N)C1] | 1089.1 |
| azaBzL-46 | 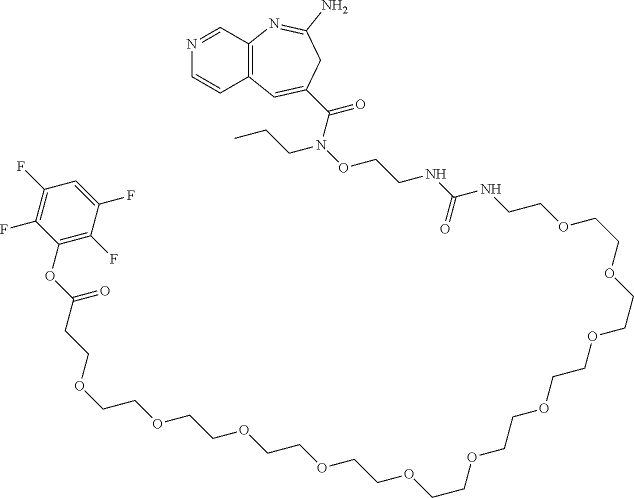 [SMILES: CCCN(OCCNC(=O)NCCOCCOCCOCCOCCOCCOCCOCCOCCOCCOCCC(=O)Oc1c(F)c(F)cc(F)c1F)C(=O)C1=Cc2ccncc2N=C(N)C1] | 1007.0 |
| azaBzL-47 | 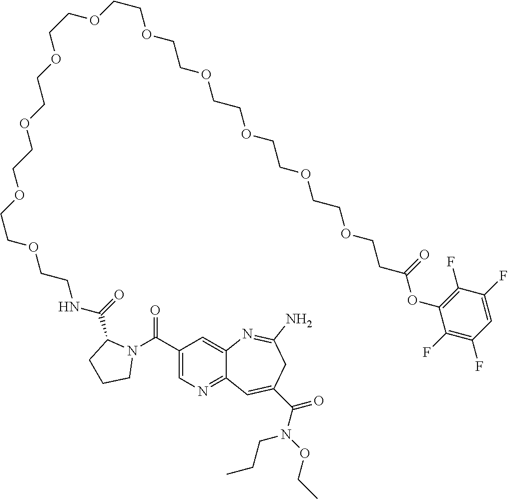 [SMILES: CCCN(OCC)C(=O)C1=Cc2ncc(C(=O)N3CCC[C@@H]3C(=O)NCCOCCOCCOCCOCCOCCOCCOCCOCCOCCOCCC(=O)Oc3c(F)c(F)cc(F)c3F)cc2N=C(N)C1] | 1089.1 |
| azaBzL-48 | 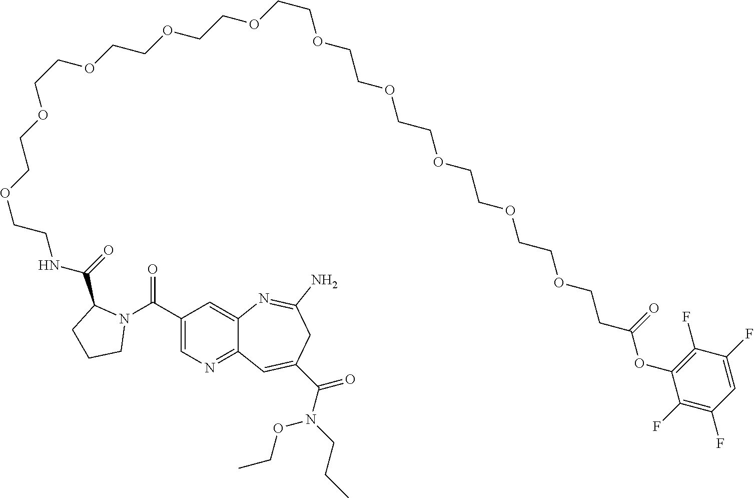 [SMILES: CCCN(OCC)C(=O)C1=Cc2ncc(C(=O)N3CCC[C@H]3C(=O)NCCOCCOCCOCCOCCOCCOCCOCCOCCOCCOCCC(=O)Oc3c(F)c(F)cc(F)c3F)cc2N=C(N)C1] | 1089.1 |
| azaBzL-49 | 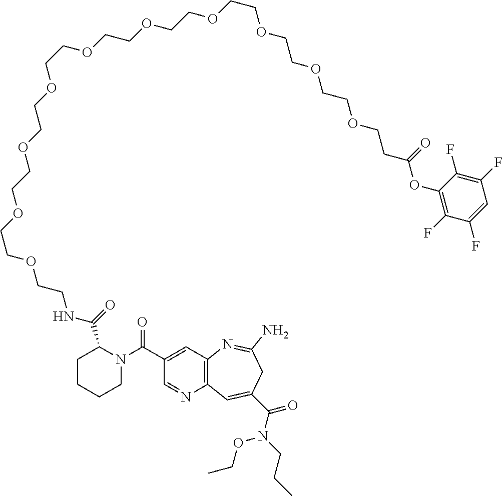 [SMILES: CCCN(OCC)C(=O)C1=Cc2ncc(C(=O)N3CCCC[C@@H]3C(=O)NCCOCCOCCOCCOCCOCCOCCOCCOCCOCCOCCC(=O)Oc3c(F)c(F)cc(F)c3F)cc2N=C(N)C1] | 1103.2 |
| azaBzL-50 | 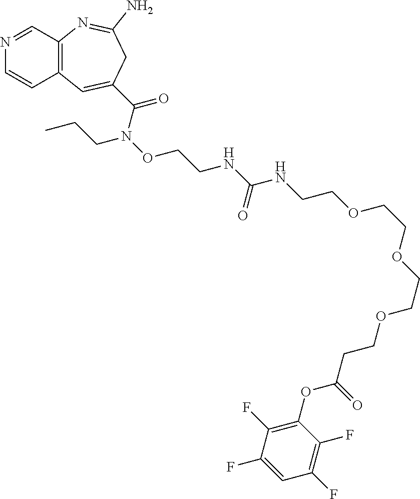 [SMILES: CCCN(OCCNC(=O)NCCOCCOCCOCCC(=O)Oc1c(F)c(F)cc(F)c1F)C(=O)C1=Cc2ccncc2N=C(N)C1] | 698.7 |
| azaBzL-51 | 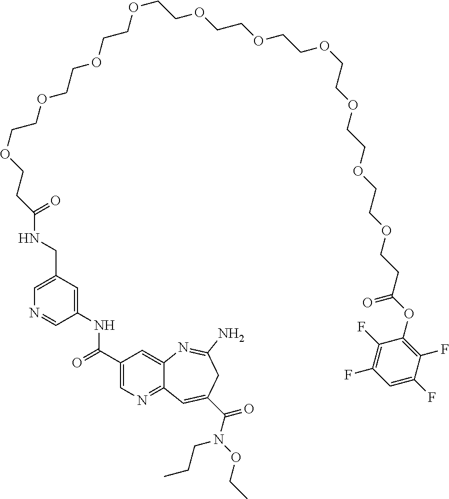 [SMILES: CCCN(OCC)C(=O)C1=Cc2ncc(C(=O)Nc3cncc(CNC(=O)CCOCCOCCOCCOCCOCCOCCOCCOCCOCCOCCC(=O)Oc4c(F)c(F)cc(F)c4F)c3)cc2N=C(N)C1] | 1126.2 |
| azaBzL-52 | 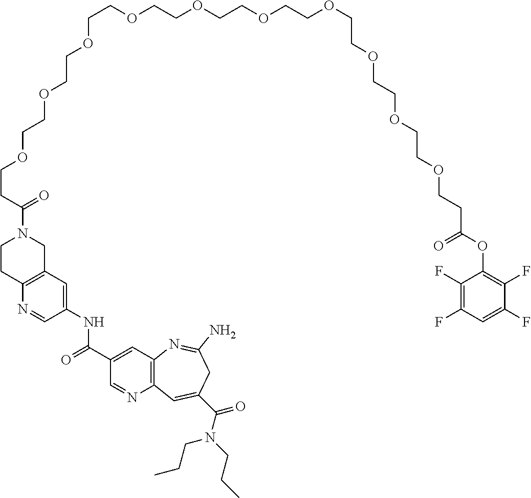 [SMILES: CCCN(CCC)C(=O)C1=Cc2ncc(C(=O)Nc3cnc4c(c3)CN(C(=O)CCOCCOCCOCCOCCOCCOCCOCCOCCOCCOCCC(=O)Oc3c(F)c(F)cc(F)c3F)CC4)cc2N=C(N)C1] | 1150.2 |
| azaBzL-53 | 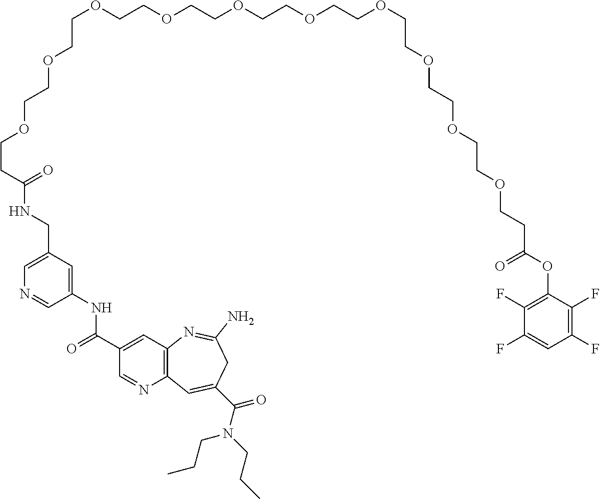 [SMILES: CCCN(CCC)C(=O)C1=Cc2ncc(C(=O)Nc3cncc(CNC(=O)CCOCCOCCOCCOCCOCCOCCOCCOCCOCCOCCC(=O)Oc4c(F)c(F)cc(F)c4F)c3)cc2N=C(N)C1] | 1124.2 |
| azaBzL-54 | 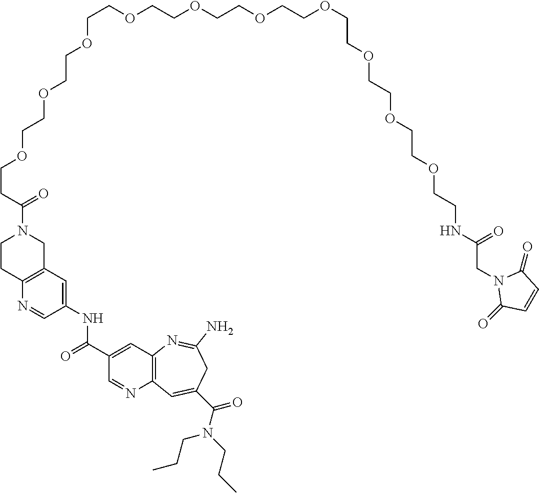 [SMILES: CCCN(CCC)C(=O)C1=Cc2ncc(C(=O)Nc3cnc4c(c3)CN(C(=O)CCOCCOCCOCCOCCOCCOCCOCCOCCOCCOCCNC(=O)CN3C(=O)C=CC3=O)CC4)cc2N=C(N)C1] | 1109.6 |
| azaBzL-55 | 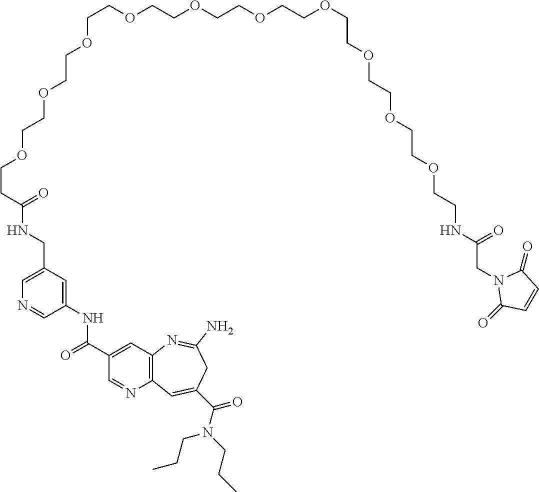 [SMILES: CCCN(CCC)C(=O)C1=Cc2ncc(C(=O)Nc3cncc(CNC(=O)CCOCCOCCOCCOCCOCCOCCOCCOCCOCCOCCNC(=O)CN4C(=O)C=CC4=O)c3)cc2N=C(N)C1] | 1084.2 |
| azaBzL-56 | 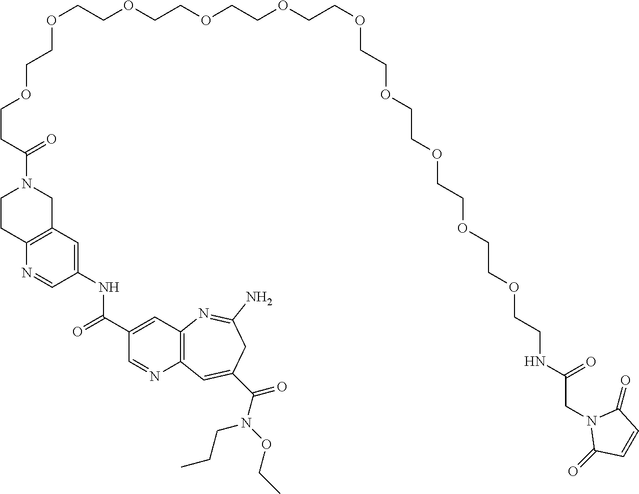 [SMILES: CCCN(OCC)C(=O)C1=Cc2ncc(C(=O)Nc3cnc4c(c3)CN(C(=O)CCOCCOCCOCCOCCOCCOCCOCCOCCOCCOCCNC(=O)CN3C(=O)C=CC3=O)CC4)cc2N=C(N)C1] | 1112.2 |
| azaBzL-57 | 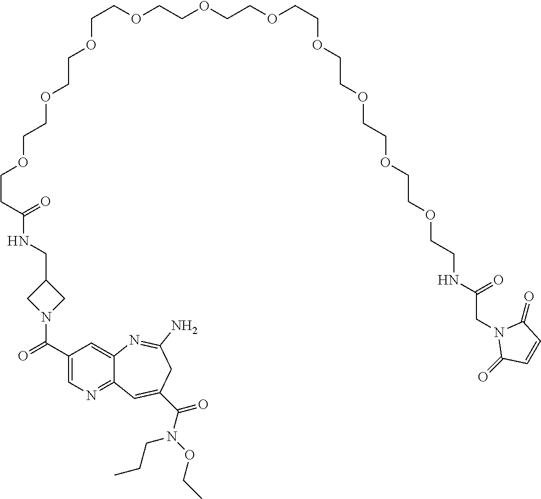 [SMILES: CCCN(OCC)C(=O)C1=Cc2ncc(C(=O)N3CC(CNC(=O)CCOCCOCCOCCOCCOCCOCCOCCOCCOCCOCCNC(=O)CN4C(=O)C=CC4=O)C3)cc2N=C(N)C1] | 1049.2 |
| azaBzL-58 | 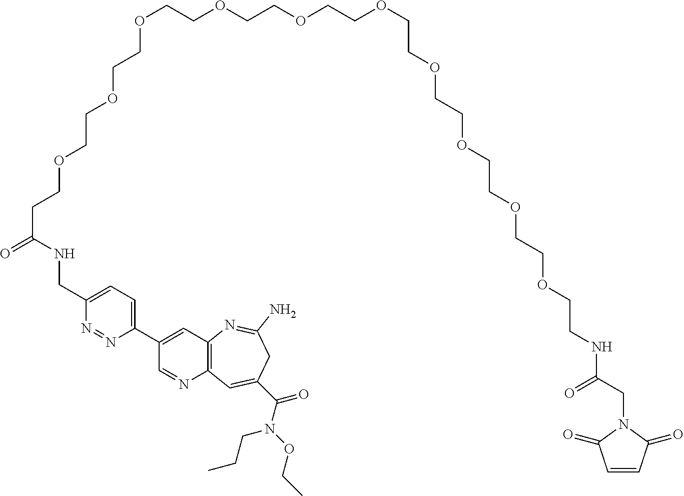 [SMILES: CCCN(OCC)C(=O)C1=Cc2ncc(-c3ccc(CNC(=O)CCOCCOCCOCCOCCOCCOCCOCCOCCOCCOCCNC(=O)CN4C(=O)C=CC4=O)nn3)cc2N=C(N)C1] | 1044.2 |
| azaBzL-59 | 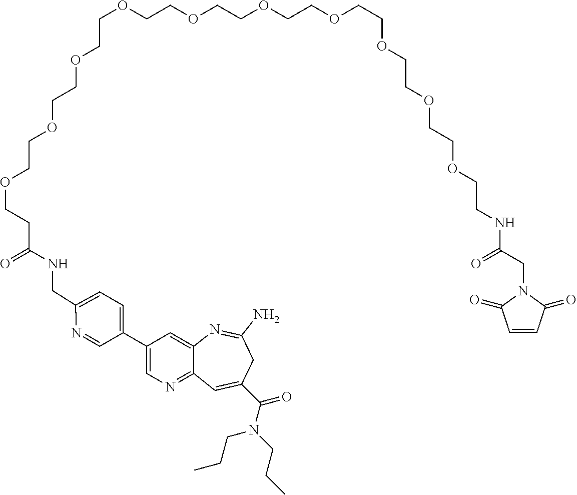 [SMILES: CCCN(CCC)C(=O)C1=Cc2ncc(-c3ccc(CNC(=O)CCOCCOCCOCCOCCOCCOCCOCCOCCOCCOCCNC(=O)CN4C(=O)C=CC4=O)nc3)cc2N=C(N)C1] | 1041.2 |
| azaBzL-60 | 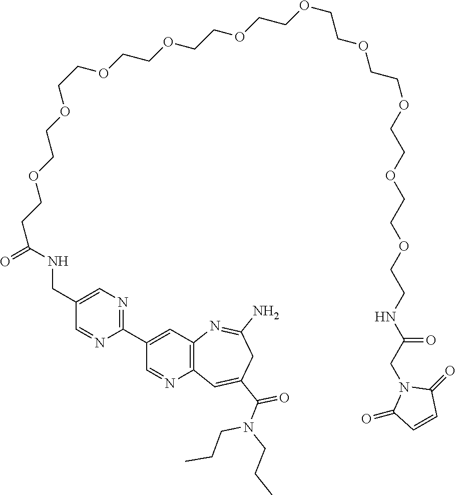 [SMILES: CCCN(CCC)C(=O)C1=Cc2ncc(-c3ncc(CNC(=O)CCOCCOCCOCCOCCOCCOCCOCCOCCOCCOCCNC(=O)CN4C(=O)C=CC4=O)cn3)cc2N=C(N)C1] | 1042.2 |
| azaBzL-61 | 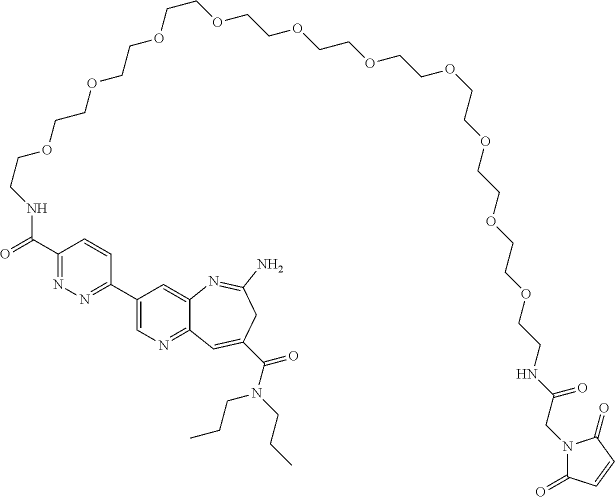 [SMILES: CCCN(CCC)C(=O)C1=Cc2ncc(-c3ccc(C(=O)NCCOCCOCCOCCOCCOCCOCCOCCOCCOCCOCCNC(=O)CN4C(=O)C=CC4=O)nn3)cc2N=C(N)C1] | 1028.2 |
| azaBzL-62 | 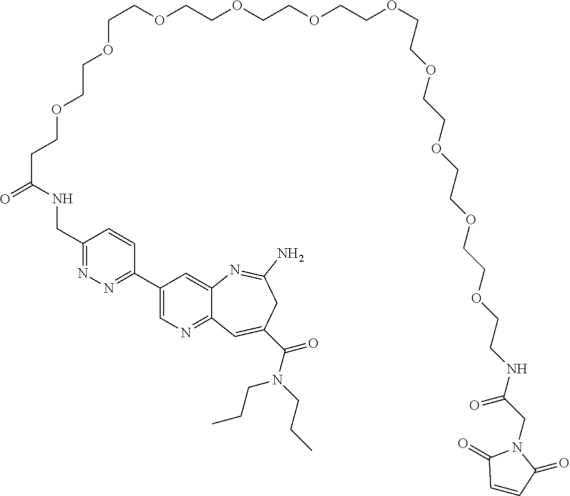 [SMILES: CCCN(CCC)C(=O)C1=Cc2ncc(-c3ccc(CNC(=O)CCOCCOCCOCCOCCOCCOCCOCCOCCOCCOCCNC(=O)CN4C(=O)C=CC4=O)nn3)cc2N=C(N)C1] | 1042.2 |
| azaBzL-63 | 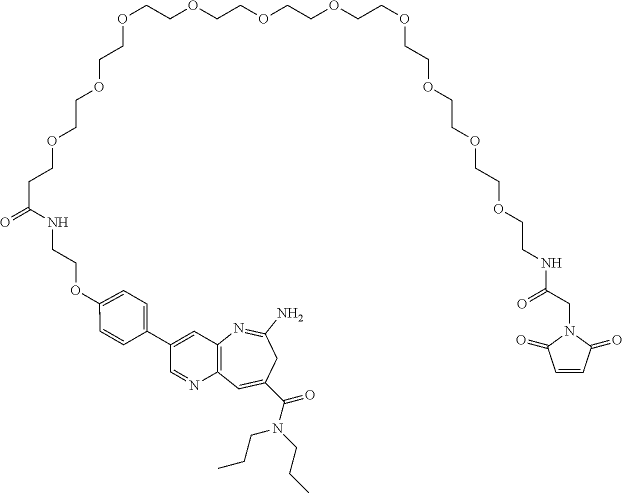 [SMILES: CCCN(CCC)C(=O)C1=Cc2ncc(-c3ccc(OCCNC(=O)CCOCCOCCOCCOCCOCCOCCOCCOCCOCCOCCNC(=O)CN4C(=O)C=CC4=O)cc3)cc2N=C(N)C1] | 1070.2 |
| azaBzL-64 | 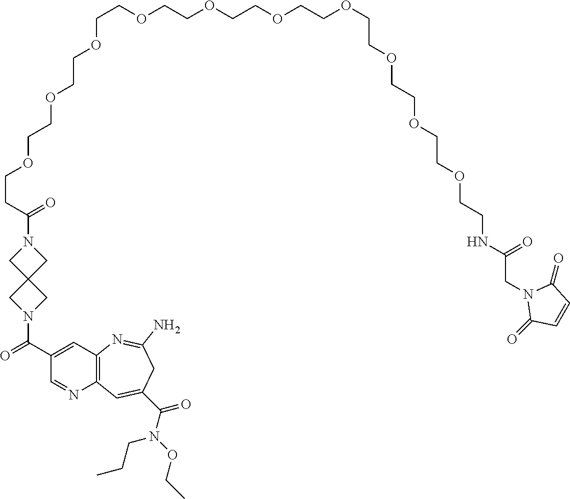 [SMILES: CCCN(OCC)C(=O)C1=Cc2ncc(C(=O)N3CC4(CN(C(=O)CCOCCOCCOCCOCCOCCOCCOCCOCCOCCOCCNC(=O)CN5C(=O)C=CC5=O)C4)C3)cc2N=C(N)C1] | 1061.2 |
| azaBzL-65 | 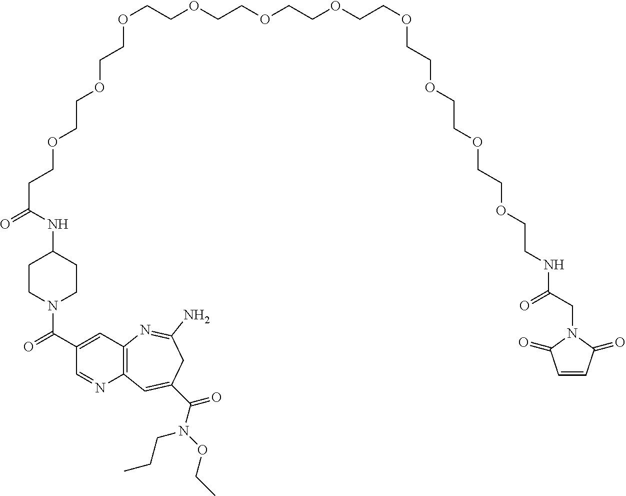 [SMILES: CCCN(OCC)C(=O)C1=Cc2ncc(C(=O)N3CCC(NC(=O)CCOCCOCCOCCOCCOCCOCCOCCOCCOCCOCCNC(=O)CN4C(=O)C=CC4=O)CC3)cc2N=C(N)C1] | 1063.2 |
| azaBzL-66 | 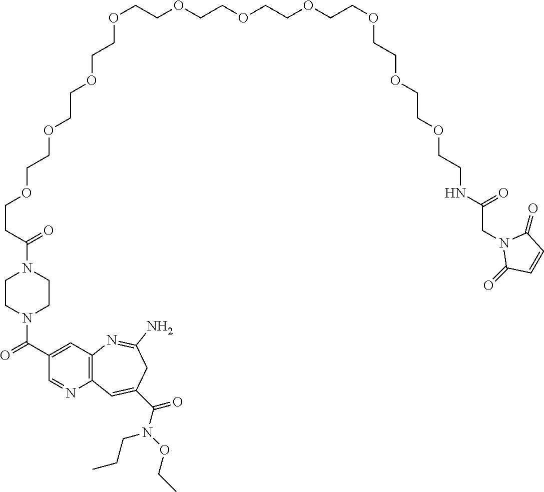 [SMILES: CCCN(OCC)C(=O)C1=Cc2ncc(C(=O)N3CCN(C(=O)CCOCCOCCOCCOCCOCCOCCOCCOCCOCCOCCNC(=O)CN4C(=O)C=CC4=O)CC3)cc2N=C(N)C1] | 1049.2 |
| azaBzL-67 | 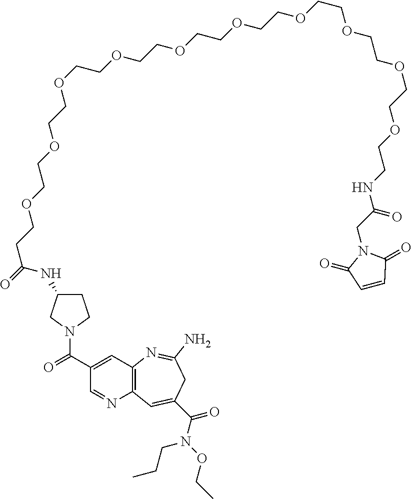 [SMILES: CCCN(OCC)C(=O)C1=Cc2ncc(C(=O)N3CC[C@@H](NC(=O)CCOCCOCCOCCOCCOCCOCCOCCOCCOCCOCCNC(=O)CN4C(=O)C=CC4=O)C3)cc2N=C(N)C1] | 1049.2 |
| azaBzL-68 | 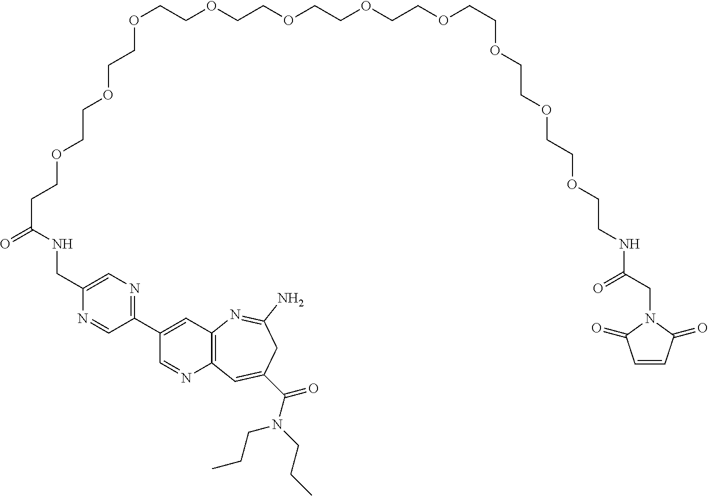 [SMILES: CCCN(CCC)C(=O)C1=Cc2ncc(-c3cnc(CNC(=O)CCOCCOCCOCCOCCOCCOCCOCCOCCOCCOCCNC(=O)CN4C(=O)C=CC4=O)cn3)cc2N=C(N)C1] | 1042.2 |
| azaBzL-69 | 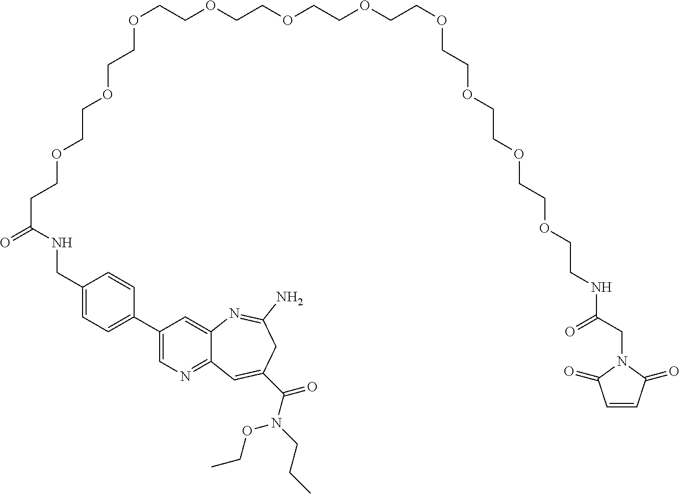 [SMILES: CCCN(OCC)C(=O)C1=Cc2ncc(-c3ccc(CNC(=O)CCOCCOCCOCCOCCOCCOCCOCCOCCOCCOCCNC(=O)CN4C(=O)C=CC4=O)cc3)cc2N=C(N)C1] | 1042.2 |
| azaBzL-70 | 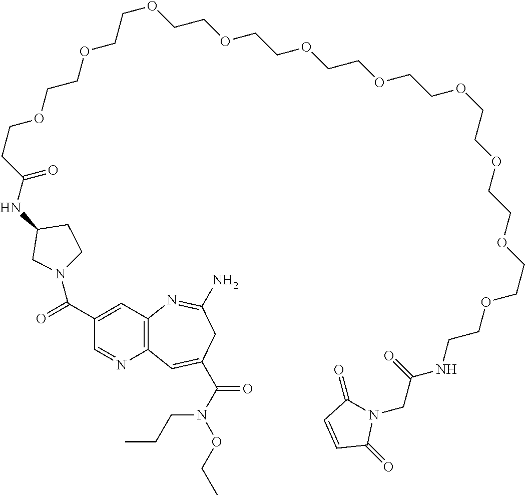 [SMILES: CCCN(OCC)C(=O)C1=Cc2ncc(C(=O)N3CC[C@H](NC(=O)CCOCCOCCOCCOCCOCCOCCOCCOCCOCCOCCNC(=O)CN4C(=O)C=CC4=O)C3)cc2N=C(N)C1] | 1049.2 |
| azaBzL-71 | 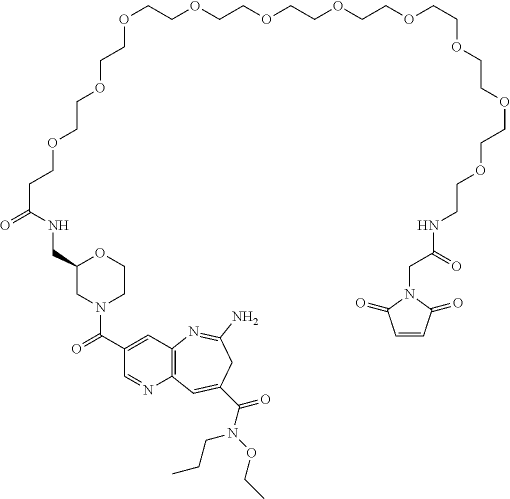 [SMILES: CCCN(OCC)C(=O)C1=Cc2ncc(C(=O)N3CCO[C@H](CNC(=O)CCOCCOCCOCCOCCOCCOCCOCCOCCOCCOCCNC(=O)CN4C(=O)C=CC4=O)C3)cc2N=C(N)C1] | 1079.2 |
| azaBzL-72 | 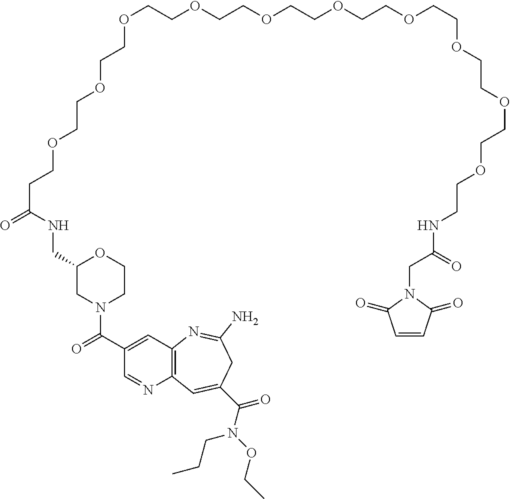 [SMILES: CCCN(OCC)C(=O)C1=Cc2ncc(C(=O)N3CCO[C@@H](CNC(=O)CCOCCOCCOCCOCCOCCOCCOCCOCCOCCOCCNC(=O)CN4C(=O)C=CC4=O)C3)cc2N=C(N)C1] | 1079.2 |
| azaBzL-73 | 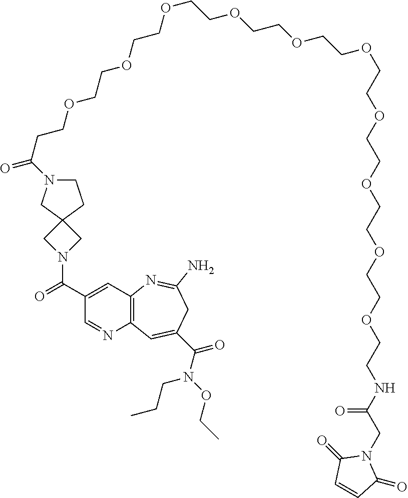 [SMILES: CCCN(OCC)C(=O)C1=Cc2ncc(C(=O)N3CC4(CCN(C(=O)CCOCCOCCOCCOCCOCCOCCOCCOCCOCCOCCNC(=O)CN5C(=O)C=CC5=O)C4)C3)cc2N=C(N)C1] | 1075.2 |
| azaBzL-74 | 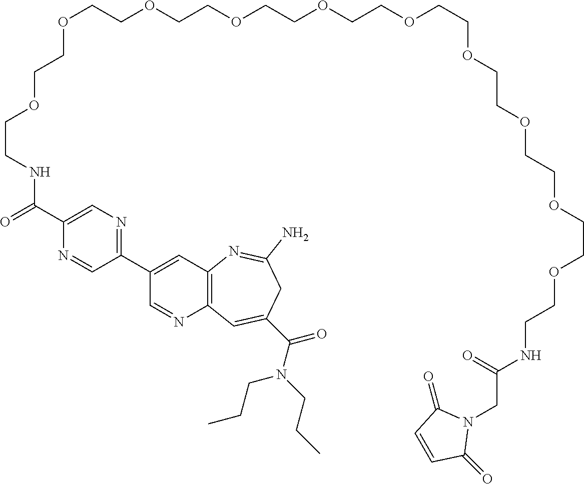 [SMILES: CCCN(CCC)C(=O)C1=Cc2ncc(-c3cnc(C(=O)NCCOCCOCCOCCOCCOCCOCCOCCOCCOCCOCCNC(=O)CN4C(=O)C=CC4=O)cn3)cc2N=C(N)C1] | 1028.2 |
| azaBzL-75 | 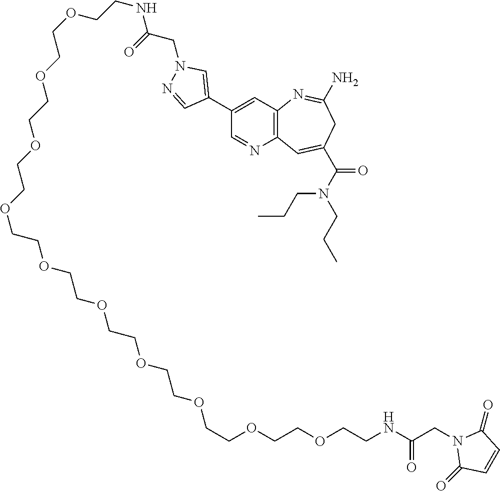 [SMILES: CCCN(CCC)C(=O)C1=Cc2ncc(-c3cnn(CC(=O)NCCOCCOCCOCCOCCOCCOCCOCCOCCOCCOCCNC(=O)CN4C(=O)C=CC4=O)c3)cc2N=C(N)C1] | 1030.2 |
| azaBzL-76 | 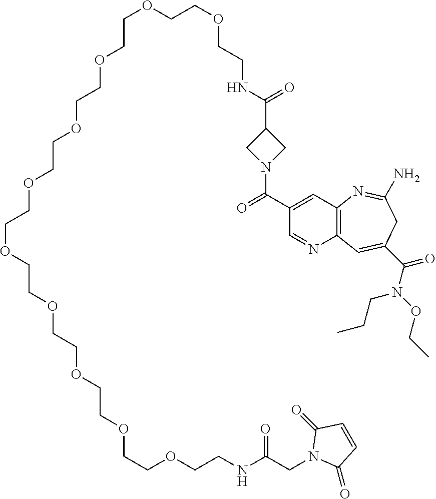 [SMILES: CCCN(OCC)C(=O)C1=Cc2ncc(C(=O)N3CC(C(=O)NCCOCCOCCOCCOCCOCCOCCOCCOCCOCCOCCNC(=O)CN4C(=O)C=CC4=O)C3)cc2N=C(N)C1] | 1035.2 |
| azaBzL-77 | 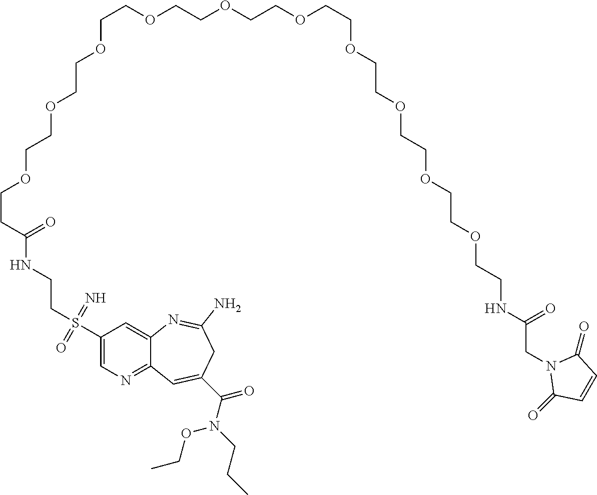 [SMILES: CCCN(OCC)C(=O)C1=Cc2ncc(S(=N)(=O)CCNC(=O)CCOCCOCCOCCOCCOCCOCCOCCOCCOCCOCCNC(=O)CN3C(=O)C=CC3=O)cc2N=C(N)C1] | 1043.2 |
| azaBzL-78 | 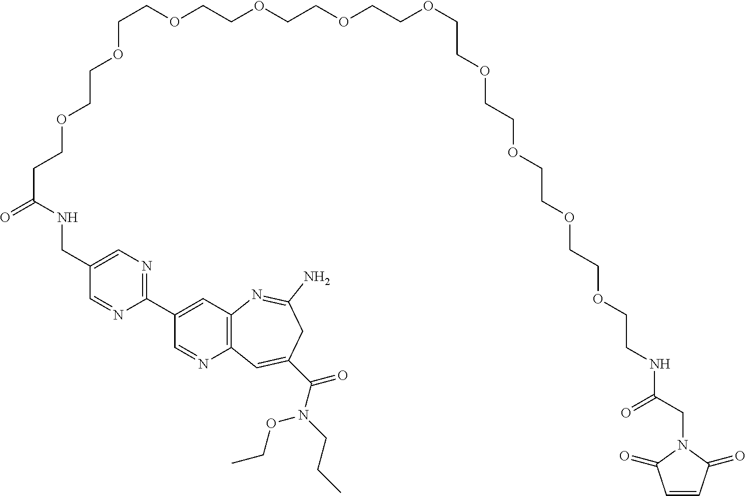 [SMILES: CCCN(OCC)C(=O)C1=Cc2ncc(-c3ncc(CNC(=O)CCOCCOCCOCCOCCOCCOCCOCCOCCOCCOCCNC(=O)CN4C(=O)C=CC4=O)cn3)cc2N=C(N)C1] | 1044.2 |
| azaBzL-79 | 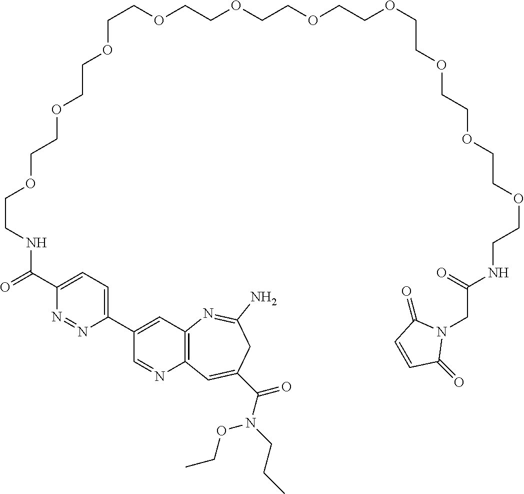 [SMILES: CCCN(OCC)C(=O)C1=Cc2ncc(-c3ccc(C(=O)NCCOCCOCCOCCOCCOCCOCCOCCOCCOCCOCCNC(=O)CN4C(=O)C=CC4=O)nn3)cc2N=C(N)C1] | 1030.1 |
| azaBzL-80 | 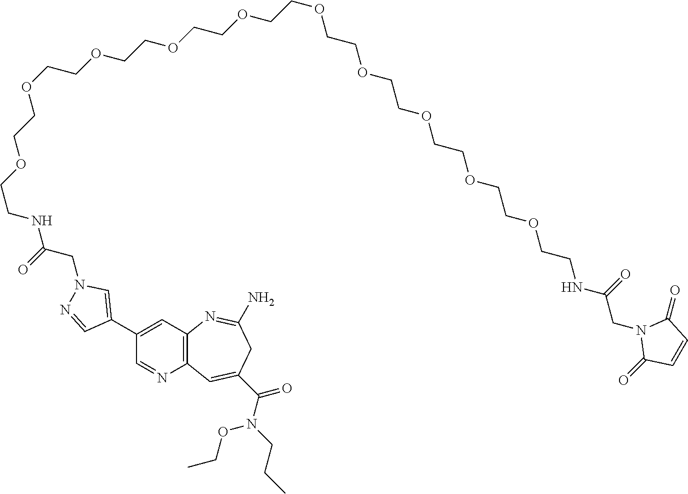 [SMILES: CCCN(OCC)C(=O)C1=Cc2ncc(-c3cnn(CC(=O)NCCOCCOCCOCCOCCOCCOCCOCCOCCOCCOCCNC(=O)CN4C(=O)C=CC4=O)c3)cc2N=C(N)C1] | 1032.2 |
| azaBzL-81 | 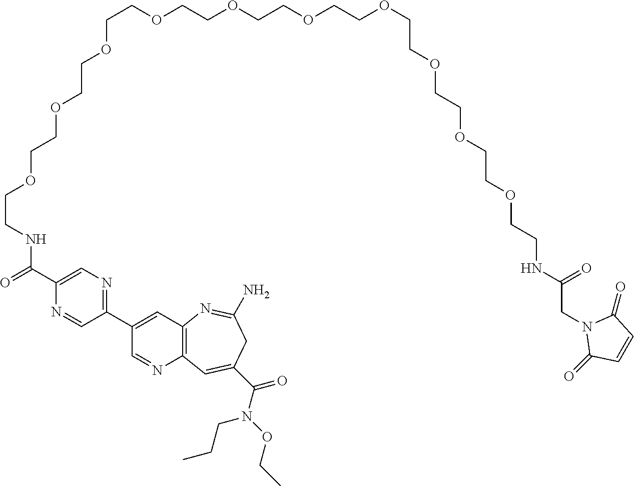 [SMILES: CCCN(OCC)C(=O)C1=Cc2ncc(-c3cnc(C(=O)NCCOCCOCCOCCOCCOCCOCCOCCOCCOCCOCCNC(=O)CN4C(=O)C=CC4=O)cn3)cc2N=C(N)C1] | 1030.1 |
| azaBzL-82 | 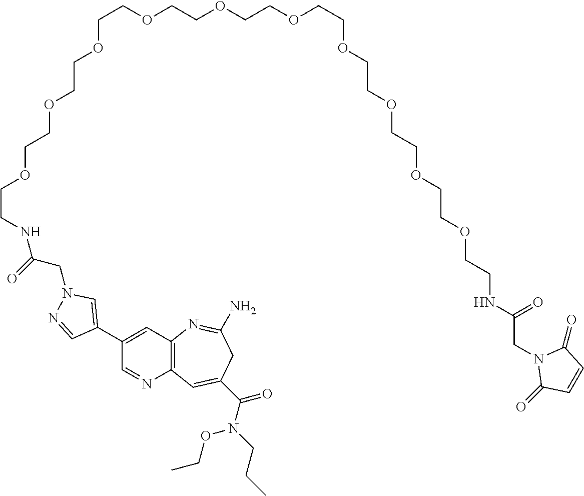 [SMILES: CCCN(OCC)C(=O)C1=Cc2ncc(-c3cnn(CC(=O)NCCOCCOCCOCCOCCOCCOCCOCCOCCOCCOCCNC(=O)CN4C(=O)C=CC4=O)c3)cc2N=C(N)C1] | 1032.2 |
| azaBzL-83 | 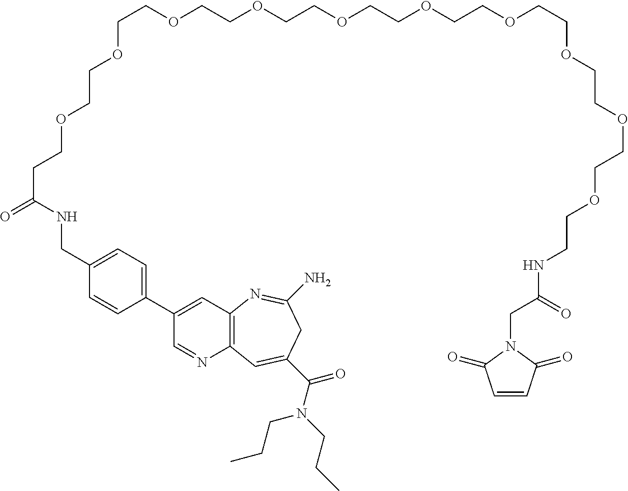 [SMILES: CCCN(CCC)C(=O)C1=Cc2ncc(-c3ccc(CNC(=O)CCOCCOCCOCCOCCOCCOCCOCCOCCOCCOCCNC(=O)CN4C(=O)C=CC4=O)cc3)cc2N=C(N)C1] | 1040.2 |
| azaBzL-84 | 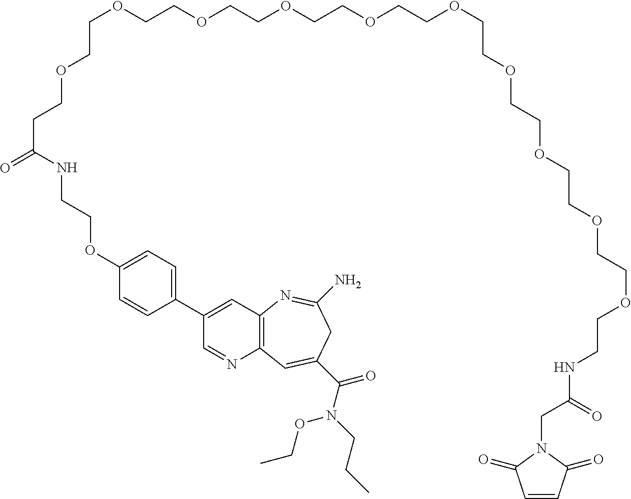 [SMILES: CCCN(OCC)C(=O)C1=Cc2ncc(-c3ccc(OCCNC(=O)CCOCCOCCOCCOCCOCCOCCOCCOCCOCCOCCNC(=O)CN4C(=O)C=CC4=O)cc3)cc2N=C(N)C1] | 1072.2 |
| azaBzL-85 | 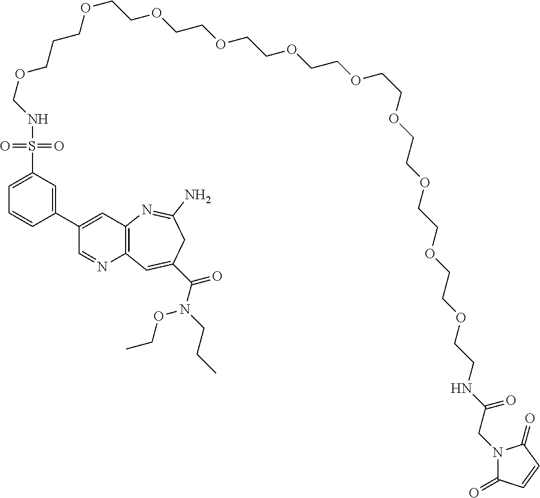 [SMILES: CCCN(OCC)C(=O)C1=Cc2ncc(-c3cccc(S(=O)(=O)NCOCCCOCCOCCOCCOCCOCCOCCOCCOCCOCCNC(=O)CN4C(=O)C=CC4=O)c3)cc2N=C(N)C1] | 1064.2 |
| azaBzL-86 | 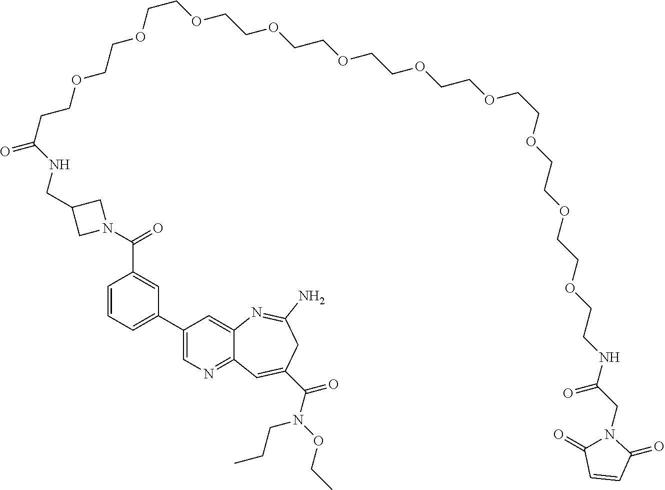 [SMILES: CCCN(OCC)C(=O)C1=Cc2ncc(-c3cccc(C(=O)N4CC(CNC(=O)CCOCCOCCOCCOCCOCCOCCOCCOCCOCCOCCNC(=O)CN5C(=O)C=CC5=O)C4)c3)cc2N=C(N)C1] | 1125.3 |
| azaBzL-87 | 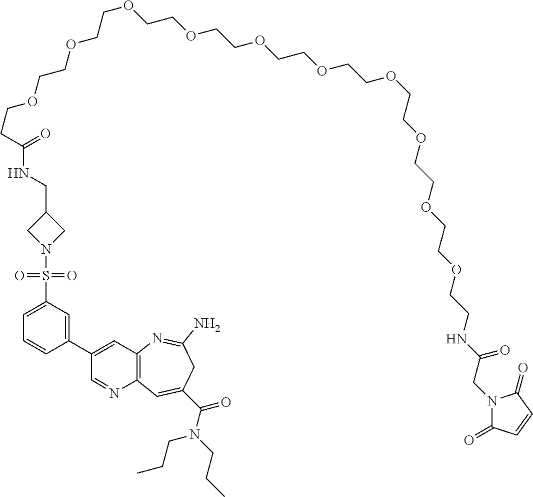 [SMILES: CCCN(CCC)C(=O)C1=Cc2ncc(-c3cccc(S(=O)(=O)N4CC(CNC(=O)CCOCCOCCOCCOCCOCCOCCOCCOCCOCCOCCNC(=O)CN5C(=O)C=CC5=O)C4)c3)cc2N=C(N)C1] | 1159.4 |
| azaBzL-88 | 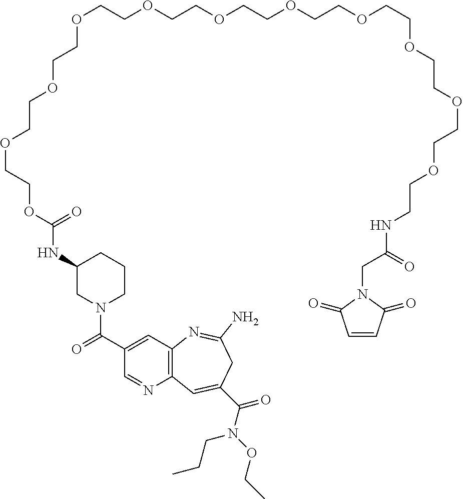 [SMILES: CCCN(OCC)C(=O)C1=Cc2ncc(C(=O)N3CCC[C@H](NC(=O)OCCOCCOCCOCCOCCOCCOCCOCCOCCOCCOCCNC(=O)CN4C(=O)C=CC4=O)C3)cc2N=C(N)C1] | 1079.2 |
| azaBzL-89 | 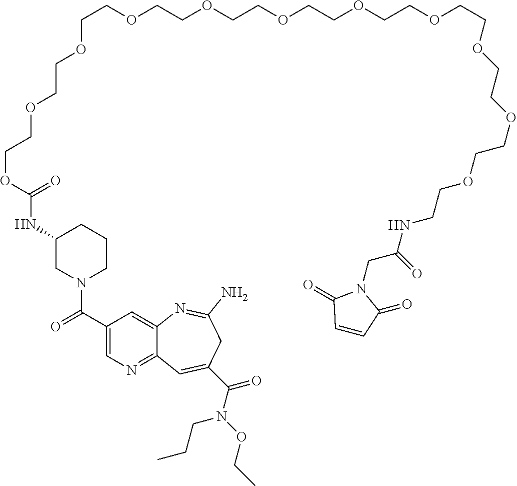 [SMILES: CCCN(OCC)C(=O)C1=Cc2ncc(C(=O)N3CCC[C@@H](NC(=O)OCCOCCOCCOCCOCCOCCOCCOCCOCCOCCOCCNC(=O)CN4C(=O)C=CC4=O)C3)cc2N=C(N)C1] | 1079.2 |
| azaBzL-90 | 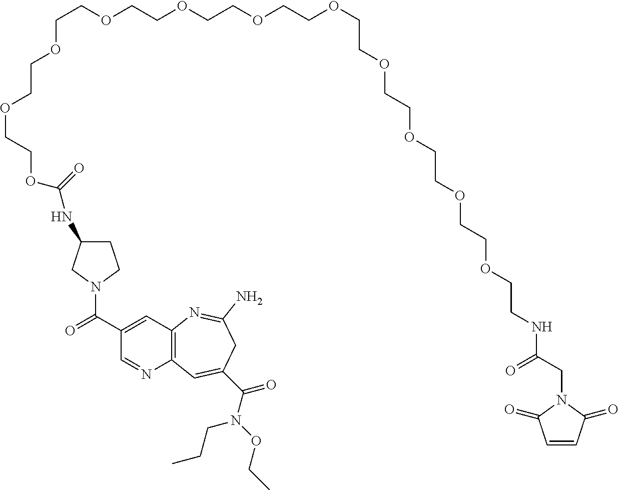 [SMILES: CCCN(OCC)C(=O)C1=Cc2ncc(C(=O)N3CC[C@H](NC(=O)OCCOCCOCCOCCOCCOCCOCCOCCOCCOCCOCCNC(=O)CN4C(=O)C=CC4=O)C3)cc2N=C(N)C1] | 1065.2 |
| azaBzL-91 | 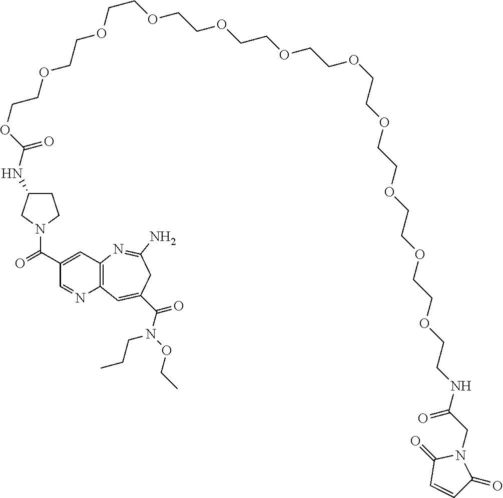 [SMILES: CCCN(OCC)C(=O)C1=Cc2ncc(C(=O)N3CC[C@@H](NC(=O)OCCOCCOCCOCCOCCOCCOCCOCCOCCOCCOCCNC(=O)CN4C(=O)C=CC4=O)C3)cc2N=C(N)C1] | 1065.2 |
| azaBzL-92 | 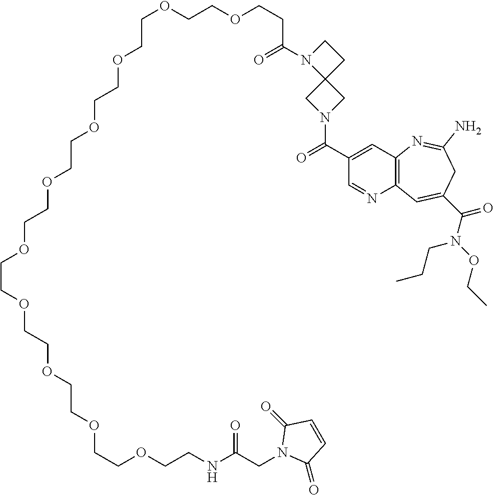 [SMILES: CCCN(OCC)C(=O)C1=Cc2ncc(C(=O)N3CC4(CCN4C(=O)CCOCCOCCOCCOCCOCCOCCOCCOCCOCCOCCNC(=O)CN4C(=O)C=CC4=O)C3)cc2N=C(N)C1] | 1061.2 |
| azaBzL-93 | 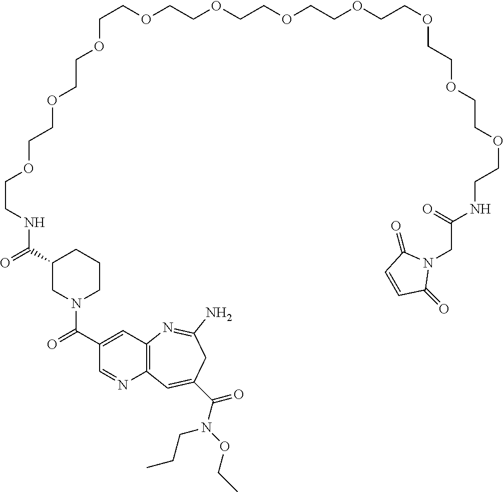 [SMILES: CCCN(OCC)C(=O)C1=Cc2ncc(C(=O)N3CCC[C@@H](C(=O)NCCOCCOCCOCCOCCOCCOCCOCCOCCOCCOCCNC(=O)CN4C(=O)C=CC4=O)C3)cc2N=C(N)C1] | 1063.2 |
| azaBzL-94 | 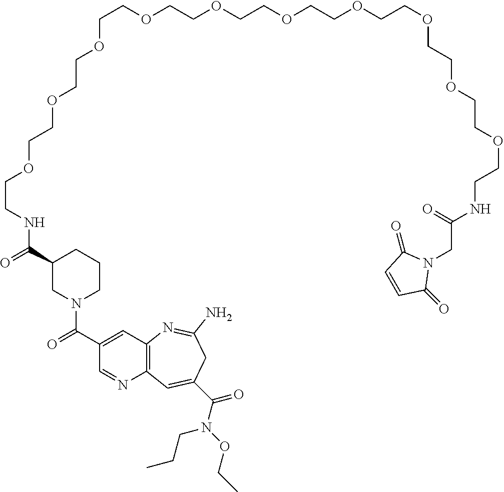 [SMILES: CCCN(OCC)C(=O)C1=Cc2ncc(C(=O)N3CCC[C@H](C(=O)NCCOCCOCCOCCOCCOCCOCCOCCOCCOCCOCCNC(=O)CN4C(=O)C=CC4=O)C3)cc2N=C(N)C1] | 1063.2 |
| azaBzL-95 | 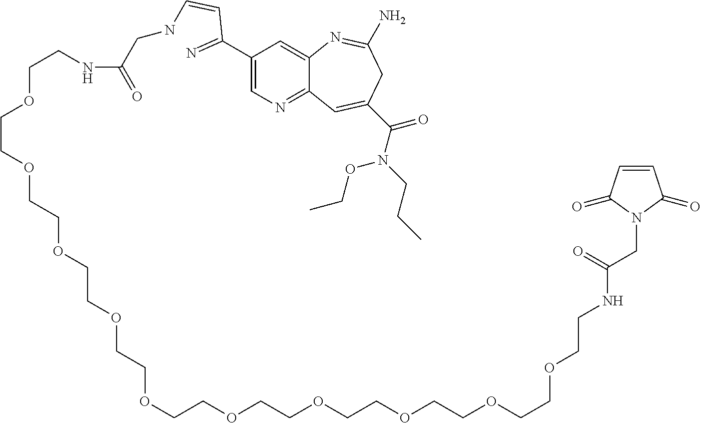 [SMILES: CCCN(OCC)C(=O)C1=Cc2ncc(C(=N)/C=C\NCC(=O)NCCOCCOCCOCCOCCOCCOCCOCCOCCOCCOCCNC(=O)CN3C(=O)C=CC3=O)cc2N=C(N)C1] | 1075.2 |
| azaBzL-96 | 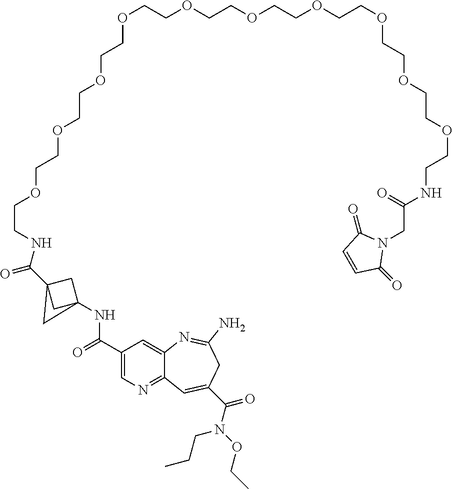 [SMILES: CCCN(OCC)C(=O)C1=Cc2ncc(C(=O)NC34CC(C(=O)NCCOCCOCCOCCOCCOCCOCCOCCOCCOCCOCCNC(=O)CN5C(=O)C=CC5=O)(C3)C4)cc2N=C(N)C1] | 1061.2 |
| azaBzL-97 | 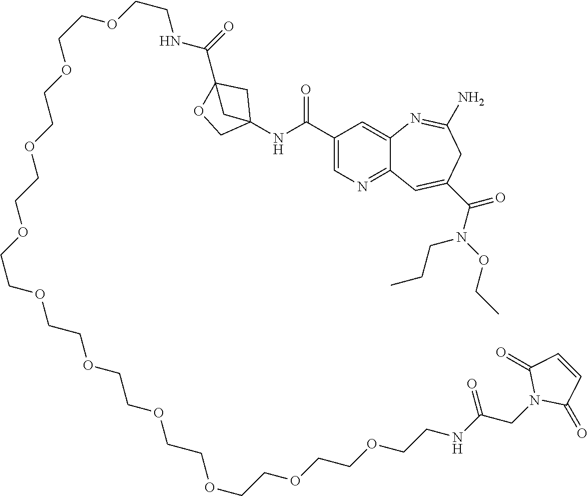 [SMILES: CCCN(OCC)C(=O)C1=Cc2ncc(C(=O)NC34COC(C(=O)NCCOCCOCCOCCOCCOCCOCCOCCOCCOCCOCCNC(=O)CN5C(=O)C=CC5=O)(C3)C4)cc2N=C(N)C1] | 1077.2 |
| azaBzL-98 | 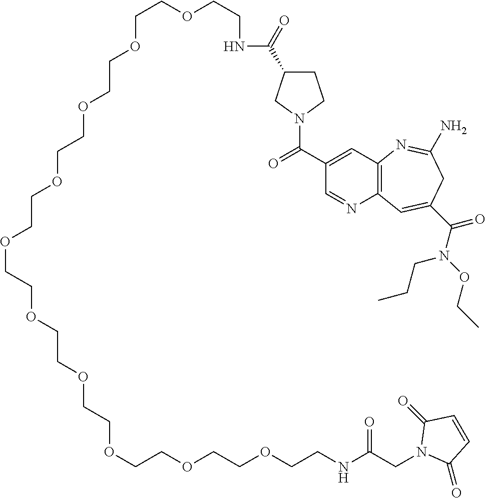 [SMILES: CCCN(OCC)C(=O)C1=Cc2ncc(C(=O)N3CC[C@@H](C(=O)NCCOCCOCCOCCOCCOCCOCCOCCOCCOCCOCCNC(=O)CN4C(=O)C=CC4=O)C3)cc2N=C(N)C1] | 1049.2 |
| azaBzL-99 | 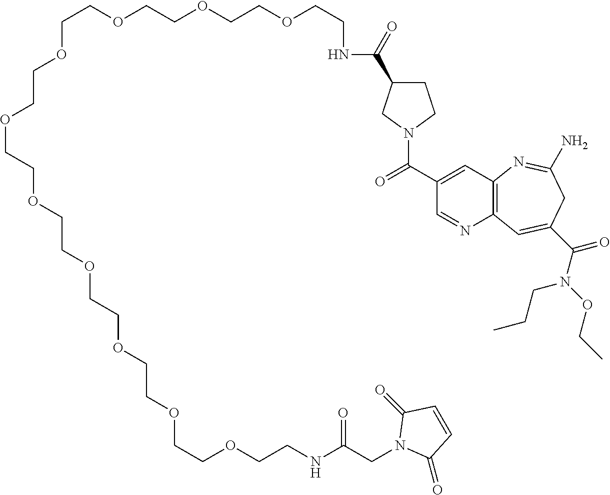 [SMILES: CCCN(OCC)C(=O)C1=Cc2ncc(C(=O)N3CC[C@H](C(=O)NCCOCCOCCOCCOCCOCCOCCOCCOCCOCCOCCNC(=O)CN4C(=O)C=CC4=O)C3)cc2N=C(N)C1] | 1049.2 |
| azaBzL-100 | 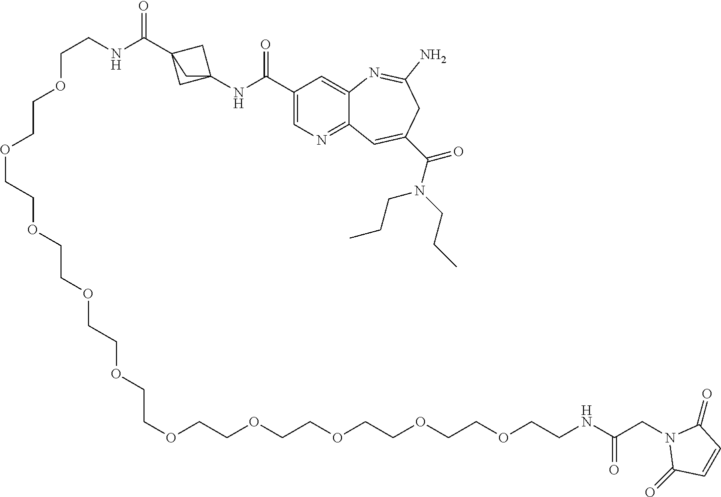 [SMILES: CCCN(CCC)C(=O)C1=Cc2ncc(C(=O)NC34CC(C(=O)NCCOCCOCCOCCOCCOCCOCCOCCOCCOCCOCCNC(=O)CN5C(=O)C=CC5=O)(C3)C4)cc2N=C(N)C1] | 1059.2 |
| azaBzL-101 | 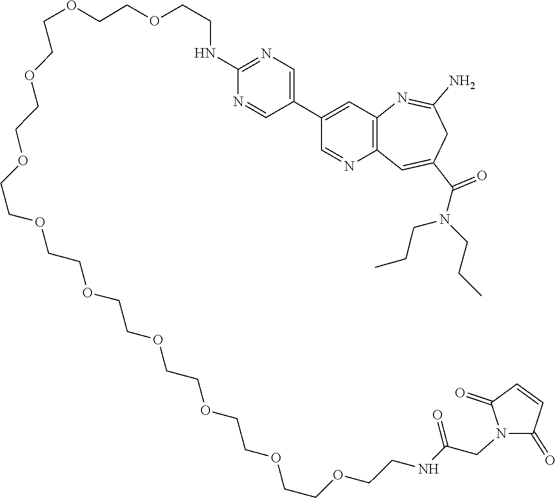 [SMILES: CCCN(CCC)C(=O)C1=Cc2ncc(-c3cnc(NCCOCCOCCOCCOCCOCCOCCOCCOCCOCCOCCNC(=O)CN4C(=O)C=CC4=O)nc3)cc2N=C(N)C1] | 1000.2 |
| azaBzL-102 | 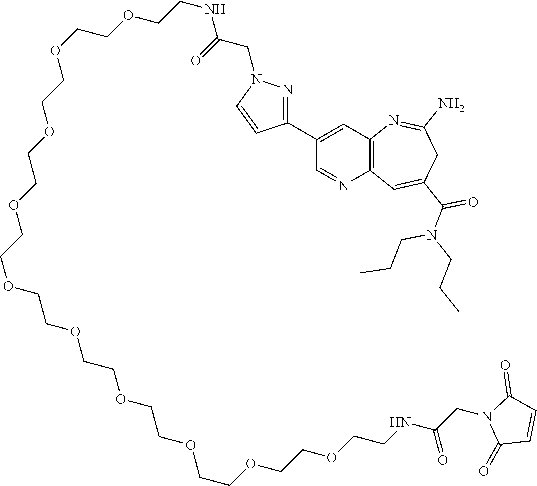 [SMILES: CCCN(CCC)C(=O)C1=Cc2ncc(-c3ccn(CC(=O)NCCOCCOCCOCCOCCOCCOCCOCCOCCOCCOCCNC(=O)CN4C(=O)C=CC4=O)n3)cc2N=C(N)C1] | 1030.2 |
| azaBzL-103 | 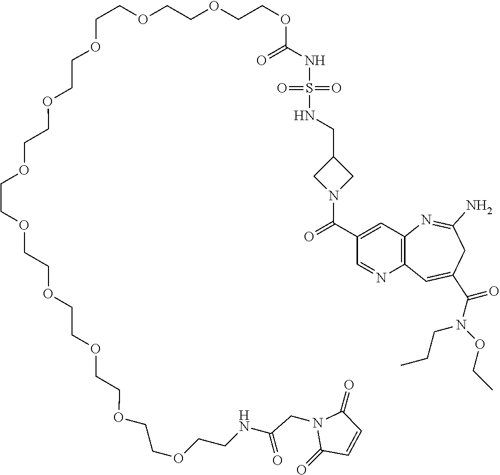 [SMILES: CCCN(OCC)C(=O)C1=Cc2ncc(C(=O)N3CC(CNS(=O)(=O)NC(=O)OCCOCCOCCOCCOCCOCCOCCOCCOCCOCCOCCNC(=O)CN4C(=O)C=CC4=O)C3)cc2N=C(N)C1] | 1144.3 |
| azaBzL-104 | 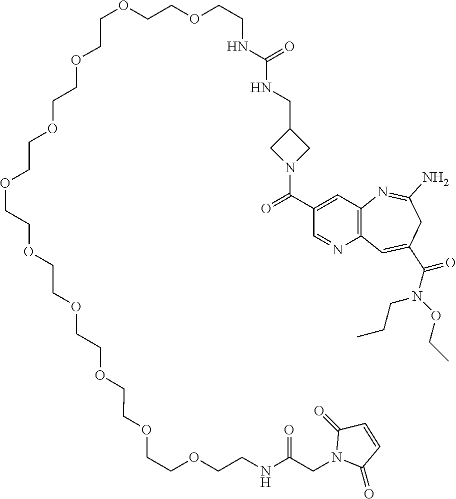 [SMILES: CCCN(OCC)C(=O)C1=Cc2ncc(C(=O)N3CC(CNC(=O)NCCOCCOCCOCCOCCOCCOCCOCCOCCOCCOCCNC(=O)CN4C(=O)C=CC4=O)C3)cc2N=C(N)C1] | 1064.2 |
| azaBzL-105 | 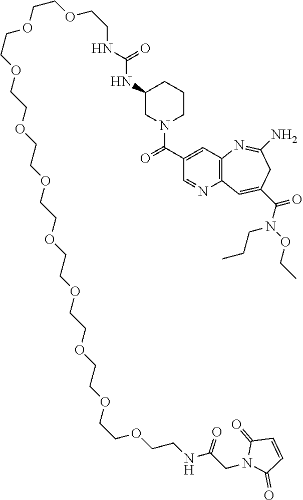 [SMILES: CCCN(OCC)C(=O)C1=Cc2ncc(C(=O)N3CCC[C@H](NC(=O)NCCOCCOCCOCCOCCOCCOCCOCCOCCOCCOCCNC(=O)CN4C(=O)C=CC4=O)C3)cc2N=C(N)C1] | 1078.2 |
| azaBzL-106 | 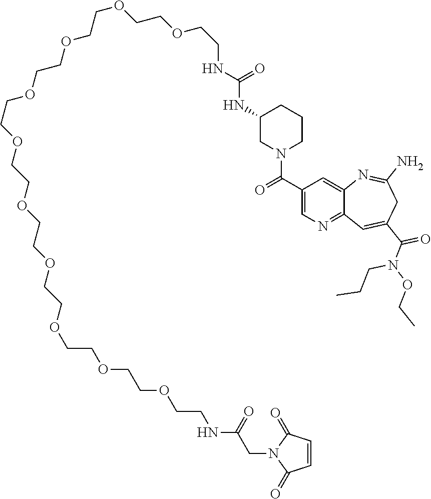 [SMILES: CCCN(OCC)C(=O)C1=Cc2ncc(C(=O)N3CCC[C@@H](NC(=O)NCCOCCOCCOCCOCCOCCOCCOCCOCCOCCOCCNC(=O)CN4C(=O)C=CC4=O)C3)cc2N=C(N)C1] | 1078.2 |
| azaBzL-107 | 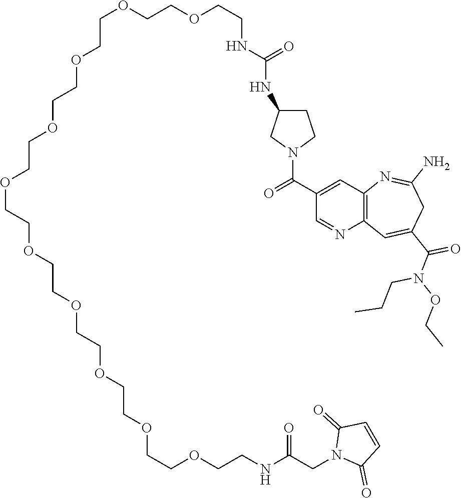 [SMILES: CCCN(OCC)C(=O)C1=Cc2ncc(C(=O)N3CC[C@H](NC(=O)NCCOCCOCCOCCOCCOCCOCCOCCOCCOCCOCCNC(=O)CN4C(=O)C=CC4=O)C3)cc2N=C(N)C1] | 1064.2 |
| azaBzL-108 | 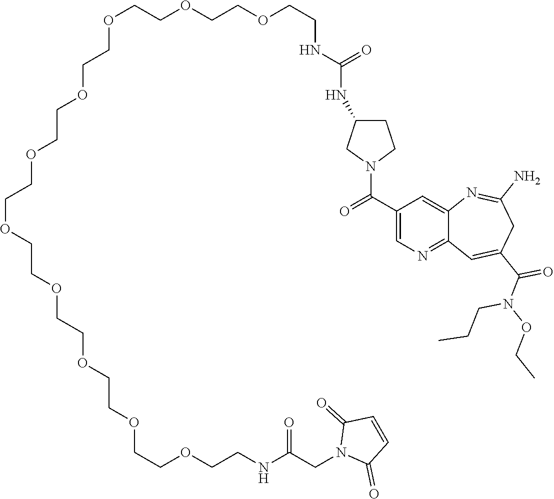 [SMILES: CCCN(OCC)C(=O)C1=Cc2ncc(C(=O)N3CC[C@@H](NC(=O)NCCOCCOCCOCCOCCOCCOCCOCCOCCOCCOCCNC(=O)CN4C(=O)C=CC4=O)C3)cc2N=C(N)C1] | 1064.2 |
| azaBzL-109 | 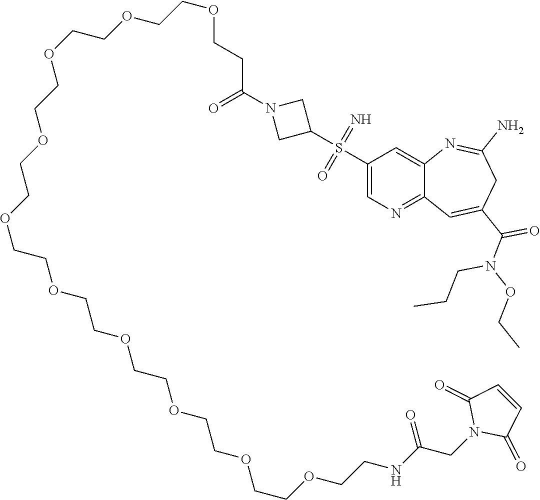 [SMILES: CCCN(OCC)C(=O)C1=Cc2ncc(S(=N)(=O)C3CN(C(=O)CCOCCOCCOCCOCCOCCOCCOCCOCCOCCOCCNC(=O)CN4C(=O)C=CC4=O)C3)cc2N=C(N)C1] | 1055.2 |
| azaBzL-110 | 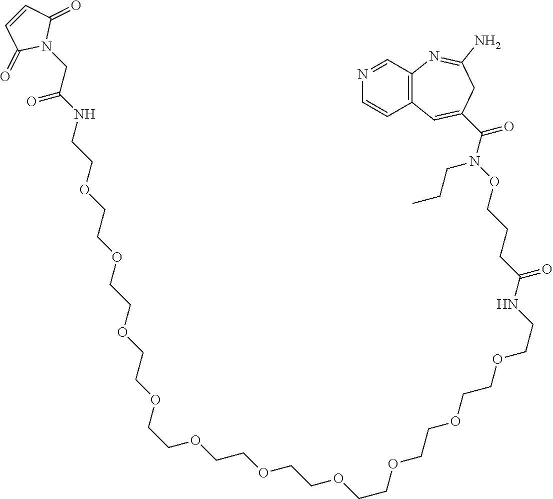 [SMILES: CCCN(OCCCC(=O)NCCOCCOCCOCCOCCOCCOCCOCCOCCOCCOCCNC(=O)CN1C(=O)C=CC1=O)C(=O)C1=Cc2ccncc2N=C(N)C1] | 966.1 |
| azaBzL-111 | 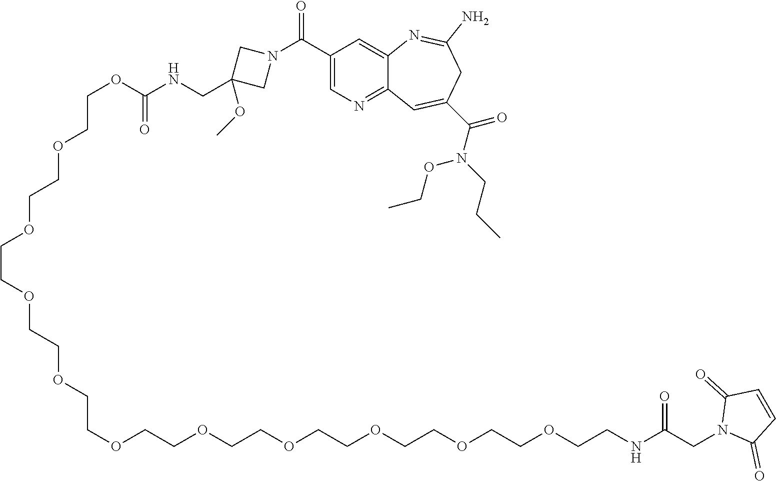 [SMILES: CCCN(OCC)C(=O)C1=Cc2ncc(C(=O)N3CC(CNC(=O)OCCOCCOCCOCCOCCOCCOCCOCCOCCOCCOCCNC(=O)CN4C(=O)C=CC4=O)(OC)C3)cc2N=C(N)C1] | 1095.2 |
| azaBzL-112 | 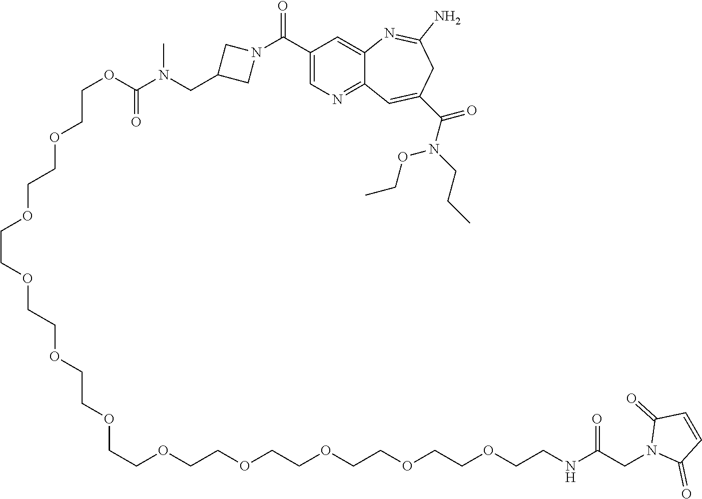 [SMILES: CCCN(OCC)C(=O)C1=Cc2ncc(C(=O)N3CC(CN(C)C(=O)OCCOCCOCCOCCOCCOCCOCCOCCOCCOCCOCCNC(=O)CN4C(=O)C=CC4=O)C3)cc2N=C(N)C1] | 1079.2 |
| azaBzL-113 | 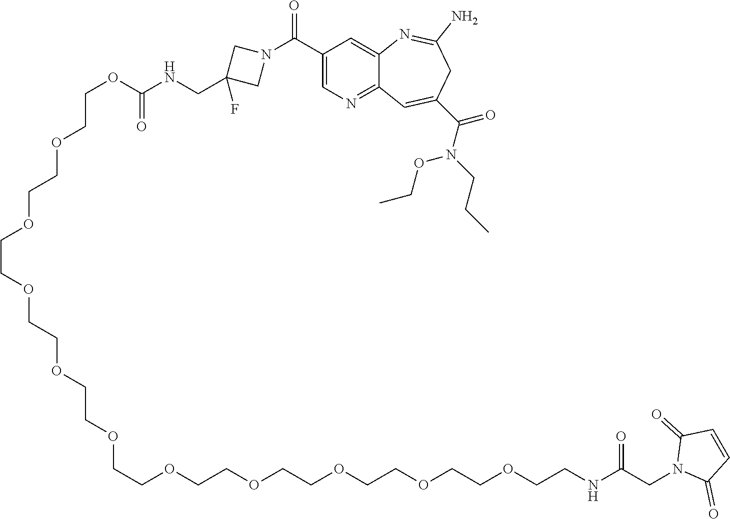 [SMILES: CCCN(OCC)C(=O)C1=Cc2ncc(C(=O)N3CC(F)(CNC(=O)OCCOCCOCCOCCOCCOCCOCCOCCOCCOCCOCCNC(=O)CN4C(=O)C=CC4=O)C3)cc2N=C(N)C1] | 1083.2 |
| azaBzL-114 | 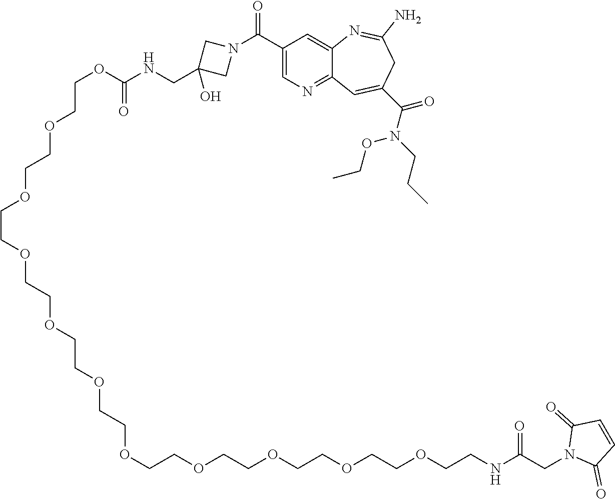 [SMILES: CCCN(OCC)C(=O)C1=Cc2ncc(C(=O)N3CC(O)(CNC(=O)OCCOCCOCCOCCOCCOCCOCCOCCOCCOCCOCCNC(=O)CN4C(=O)C=CC4=O)C3)cc2N=C(N)C1] | 1081.2 |
| azaBzL-115 | 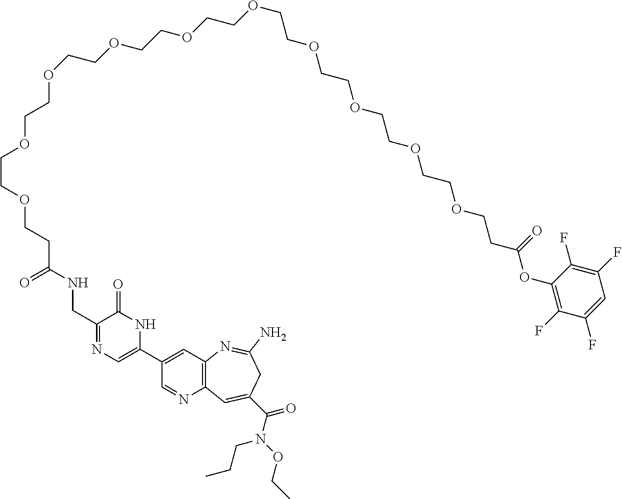 [SMILES: CCCN(OCC)C(=O)C1=Cc2ncc(-c3cnc(CNC(=O)CCOCCOCCOCCOCCOCCOCCOCCOCCOCCOCCC(=O)Oc4c(F)c(F)cc(F)c4F)c(=O)[nH]3)cc2N=C(N)C1] | 1100.2 |
| azaBzL-116 | 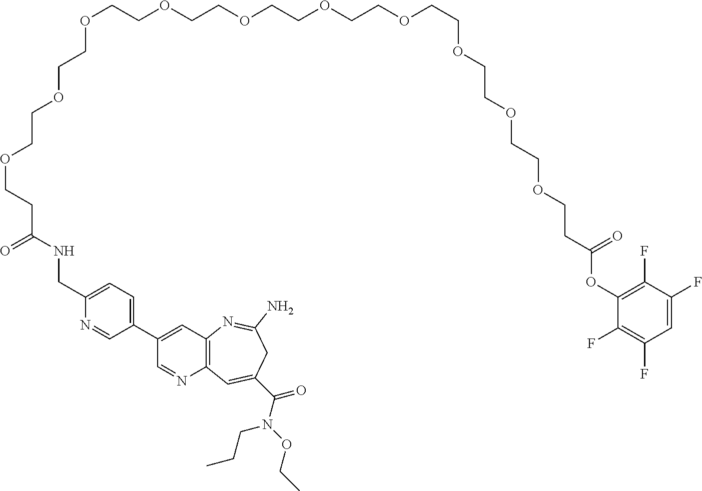 [SMILES: CCCN(OCC)C(=O)C1=Cc2ncc(-c3ccc(CNC(=O)CCOCCOCCOCCOCCOCCOCCOCCOCCOCCOCCC(=O)Oc4c(F)c(F)cc(F)c4F)nc3)cc2N=C(N)C1] | 1083.1 |
| azaBzL-117 | 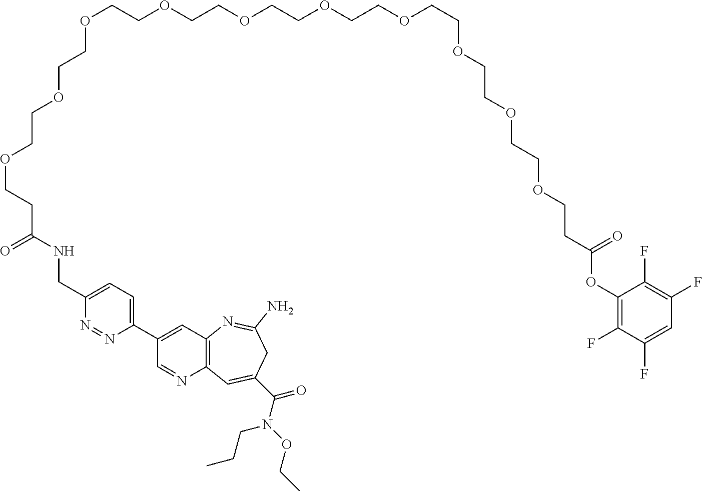 [SMILES: CCCN(OCC)C(=O)C1=Cc2ncc(-c3ccc(CNC(=O)CCOCCOCCOCCOCCOCCOCCOCCOCCOCCOCCC(=O)Oc4c(F)c(F)cc(F)c4F)nn3)cc2N=C(N)C1] | 1084.1 |
| azaBzL-118 | 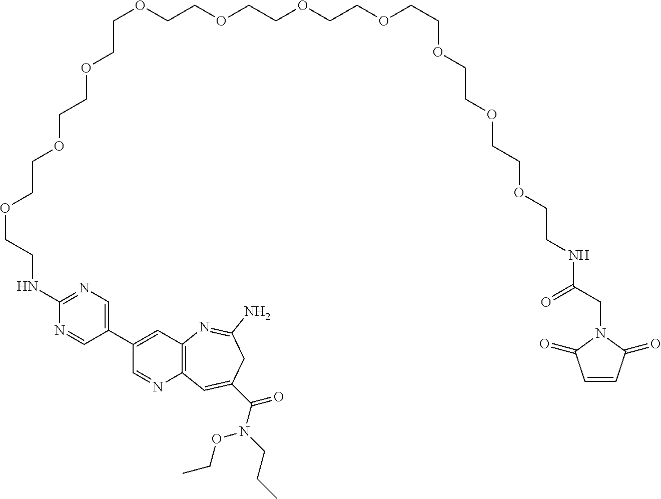 [SMILES: CCCN(OCC)C(=O)C1=Cc2ncc(-c3cnc(NCCOCCOCCOCCOCCOCCOCCOCCOCCOCCOCCNC(=O)CN4C(=O)C=CC4=O)nc3)cc2N=C(N)C1] | 1002.1 |
| azaBzL-119 | 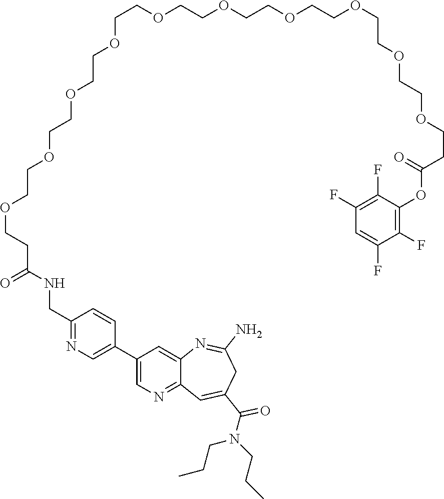 [SMILES: CCCN(CCC)C(=O)C1=Cc2ncc(-c3ccc(CNC(=O)CCOCCOCCOCCOCCOCCOCCOCCOCCOCCOCCC(=O)Oc4c(F)c(F)cc(F)c4F)nc3)cc2N=C(N)C1] | 1081.2 |
| azaBzL-120 | 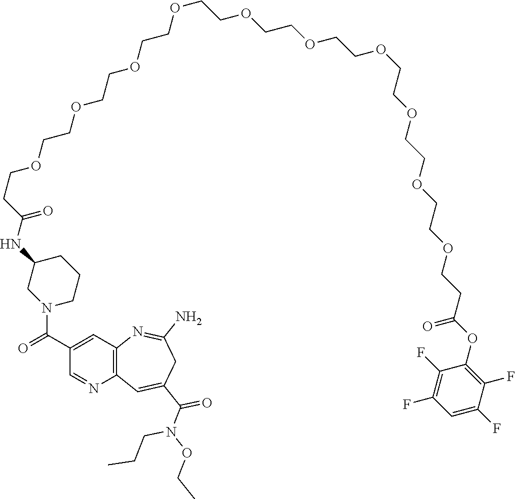 [SMILES: CCCN(OCC)C(=O)C1=Cc2ncc(C(=O)N3CCC[C@H](NC(=O)CCOCCOCCOCCOCCOCCOCCOCCOCCOCCOCCC(=O)Oc4c(F)c(F)cc(F)c4F)C3)cc2N=C(N)C1] | 1103.2 |
| azaBzL-121 | 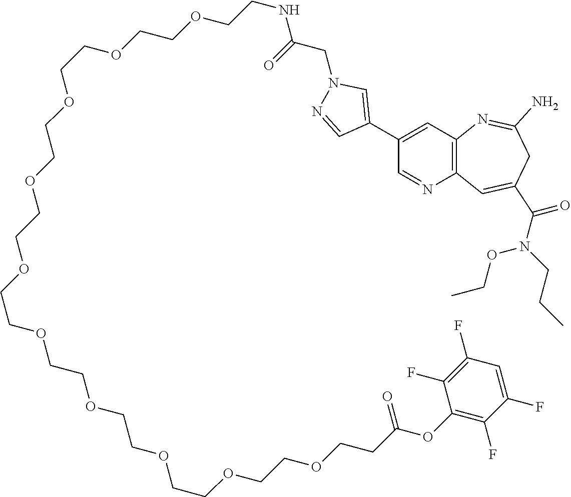 [SMILES: CCCN(OCC)C(=O)C1=Cc2ncc(-c3cnn(CC(=O)NCCOCCOCCOCCOCCOCCOCCOCCOCCOCCOCCC(=O)Oc4c(F)c(F)cc(F)c4F)c3)cc2N=C(N)C1] | 1072.1 |
| azaBzL-122 | 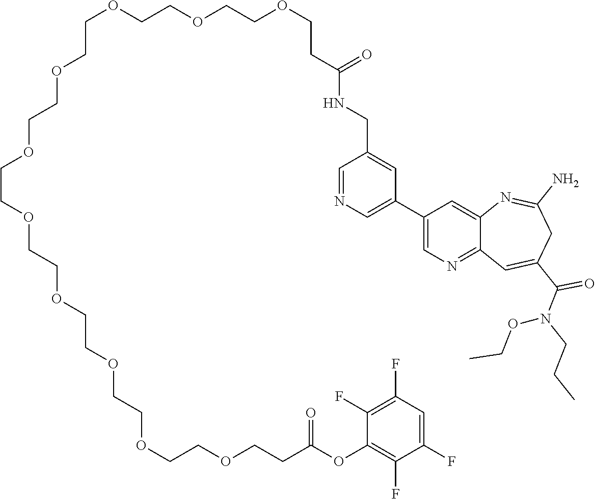 [SMILES: CCCN(OCC)C(=O)C1=Cc2ncc(-c3cncc(CNC(=O)CCOCCOCCOCCOCCOCCOCCOCCOCCOCCOCCC(=O)Oc4c(F)c(F)cc(F)c4F)c3)cc2N=C(N)C1] | 1083.1 |
| azaBzL-123 | 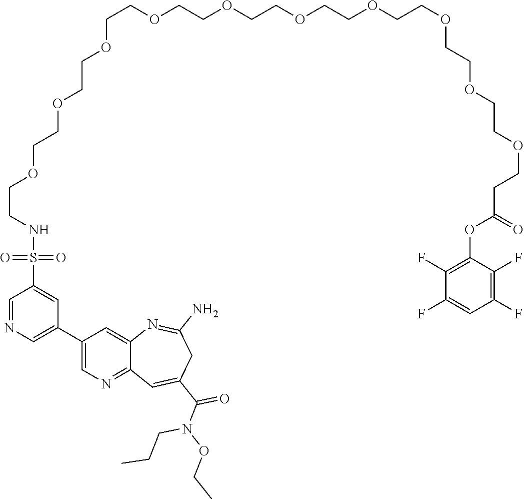 [SMILES: CCCN(OCC)C(=O)C1=Cc2ncc(-c3cncc(S(=O)(=O)NCCOCCOCCOCCOCCOCCOCCOCCOCCOCCOCCC(=O)Oc4c(F)c(F)cc(F)c4F)c3)cc2N=C(N)C1] | 1105.2 |
| azaBzL-124 | 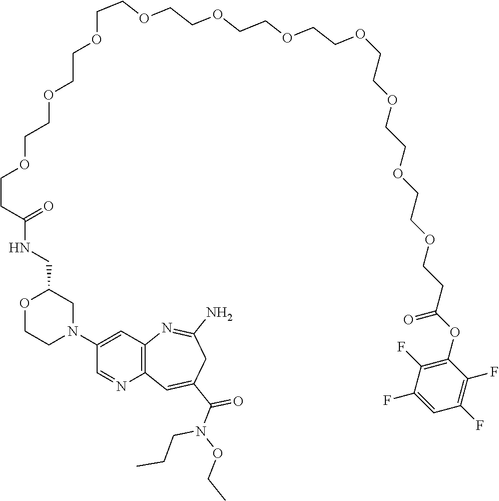 [SMILES: CCCN(OCC)C(=O)C1=Cc2ncc(N3CCO[C@H](CNC(=O)CCOCCOCCOCCOCCOCCOCCOCCOCCOCCOCCC(=O)Oc4c(F)c(F)cc(F)c4F)C3)cc2N=C(N)C1] | 1091.2 |
| azaBzL-125 | 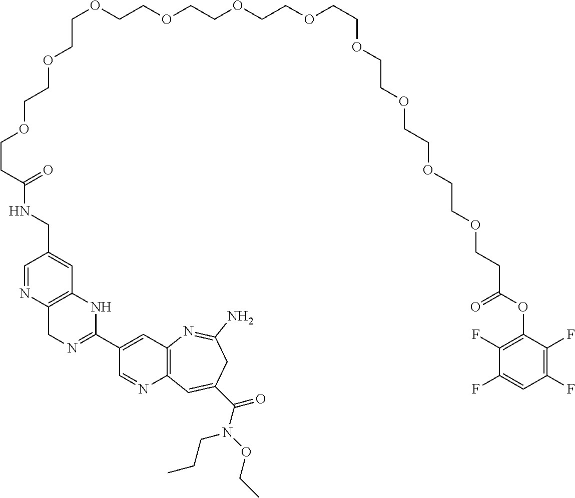 [SMILES: CCCN(OCC)C(=O)C1=Cc2ncc(C3=NCc4ncc(CNC(=O)CCOCCOCCOCCOCCOCCOCCOCCOCCOCCOCCC(=O)Oc5c(F)c(F)cc(F)c5F)cc4N3)cc2N=C(N)C1] | 1137.2 |
| azaBzL-126 | 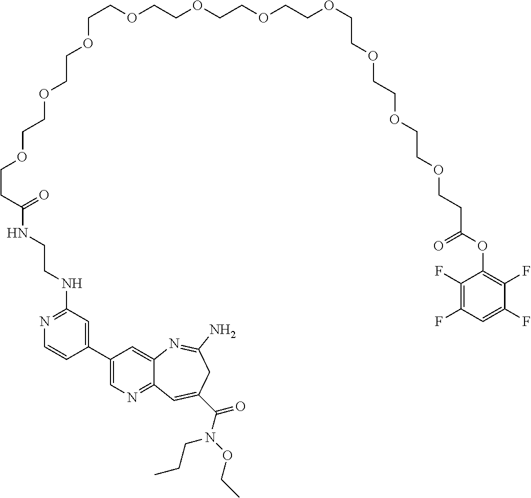 [SMILES: CCCN(OCC)C(=O)C1=Cc2ncc(-c3ccnc(NCCNC(=O)CCOCCOCCOCCOCCOCCOCCOCCOCCOCCOCCC(=O)Oc4c(F)c(F)cc(F)c4F)c3)cc2N=C(N)C1] | 1112.2 |
| azaBzL-127 | 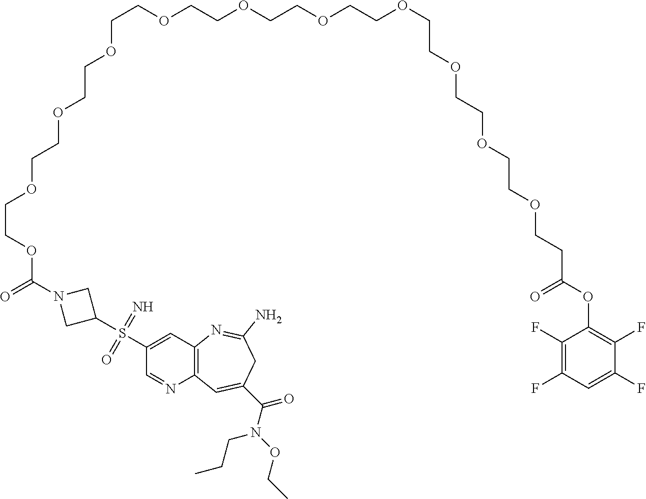 [SMILES: CCCN(OCC)C(=O)C1=Cc2ncc(S(=N)(=O)C3CN(C(=O)OCCOCCOCCOCCOCCOCCOCCOCCOCCOCCOCCC(=O)Oc4c(F)c(F)cc(F)c4F)C3)cc2N=C(N)C1] v | 1111.2 |
| azaBzL-128 | 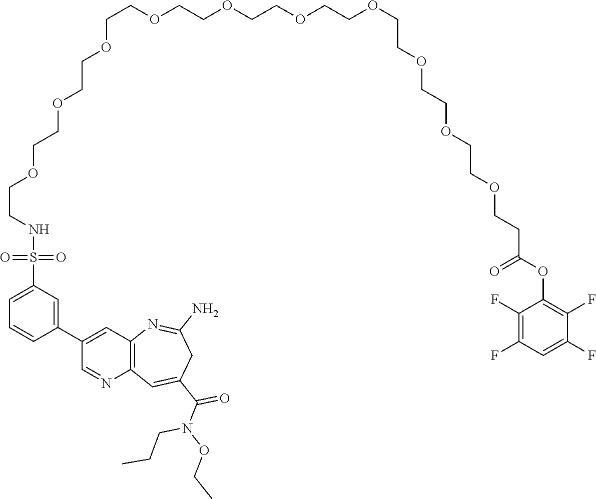 [SMILES: CCCN(OCC)C(=O)C1=Cc2ncc(-c3cccc(S(=O)(=O)NCCOCCOCCOCCOCCOCCOCCOCCOCCOCCOCCC(=O)Oc4c(F)c(F)cc(F)c4F)c3)cc2N=C(N)C1] | 1104.2 |
| azaBzL-129 | 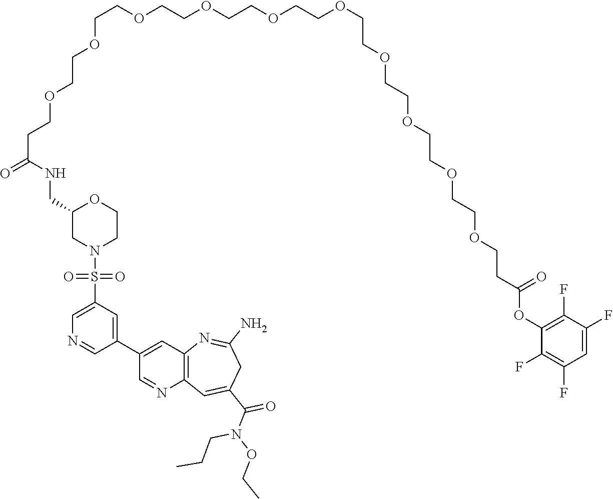 [SMILES: CCCN(OCC)C(=O)C1=Cc2ncc(-c3cncc(S(=O)(=O)N4CCO[C@@H](CNC(=O)CCOCCOCCOCCOCCOCCOCCOCCOCCOCCOCCC(=O)Oc5c(F)c(F)cc(F)c5F)C4)c3)cc2N=C(N)C1] | 1232.3 |
| azaBzL-130 | 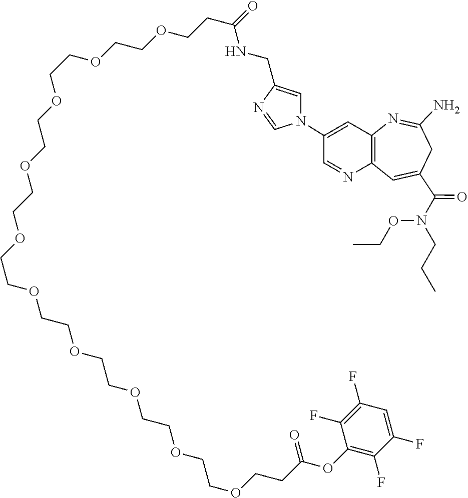 [SMILES: CCCN(OCC)C(=O)C1=Cc2ncc(-n3cnc(CNC(=O)CCOCCOCCOCCOCCOCCOCCOCCOCCOCCOCCC(=O)Oc4c(F)c(F)cc(F)c4F)c3)cc2N=C(N)C1] | 1072.1 |
| azaBzL-131 | 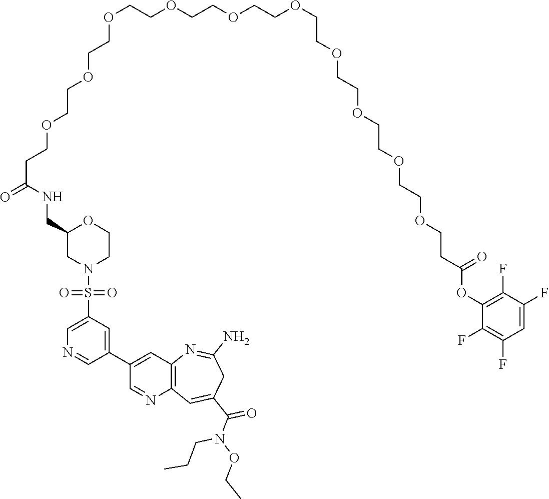 [SMILES: CCCN(OCC)C(=O)C1=Cc2ncc(-c3cncc(S(=O)(=O)N4CCO[C@H](CNC(=O)CCOCCOCCOCCOCCOCCOCCOCCOCCOCCOCCC(=O)Oc5c(F)c(F)cc(F)c5F)C4)c3)cc2N=C(N)C1] | 1232.3 |
| azaBzL-132 | 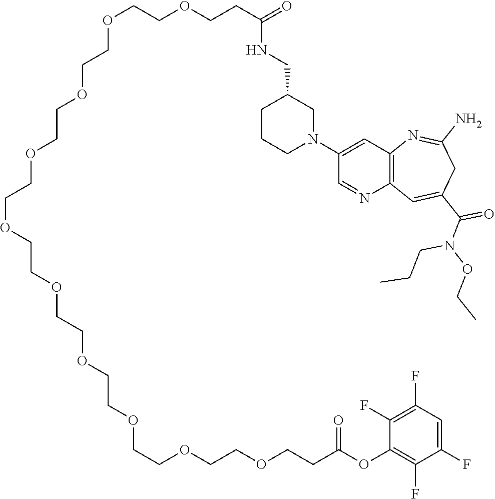 [SMILES: CCCN(OCC)C(=O)C1=Cc2ncc(N3CCC[C@H](CNC(=O)CCOCCOCCOCCOCCOCCOCCOCCOCCOCCOCCC(=O)Oc4c(F)c(F)cc(F)c4F)C3)cc2N=C(N)C1] | 1089.2 |
| azaBzL-133 | 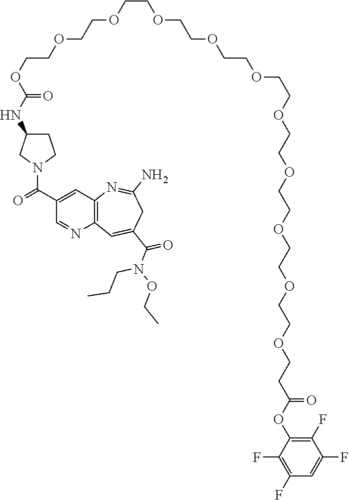 [SMILES: CCCN(OCC)C(=O)C1=Cc2ncc(C(=O)N3CC[C@H](NC(=O)OCCOCCOCCOCCOCCOCCOCCOCCOCCOCCOCCC(=O)Oc4c(F)c(F)cc(F)c4F)C3)cc2N=C(N)C1] | 1105.1 |
| azaBzL-134 | 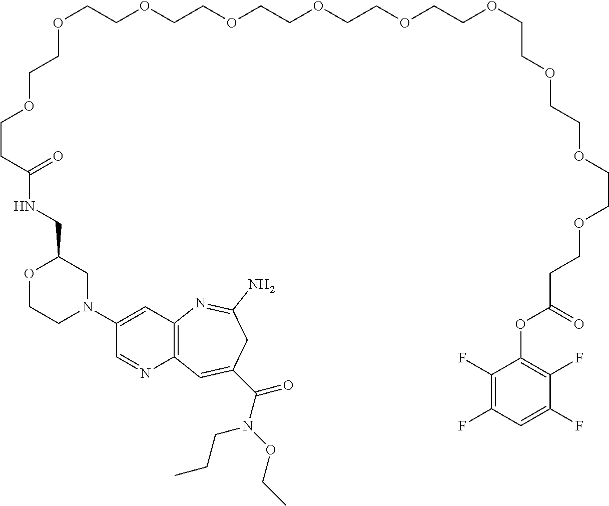 [SMILES: CCCN(OCC)C(=O)C1=Cc2ncc(N3CCO[C@@H](CNC(=O)CCOCCOCCOCCOCCOCCOCCOCCOCCOCCOCCC(=O)Oc4c(F)c(F)cc(F)c4F)C3)cc2N=C(N)C1] | 1091.2 |
| azaBzL-135 | 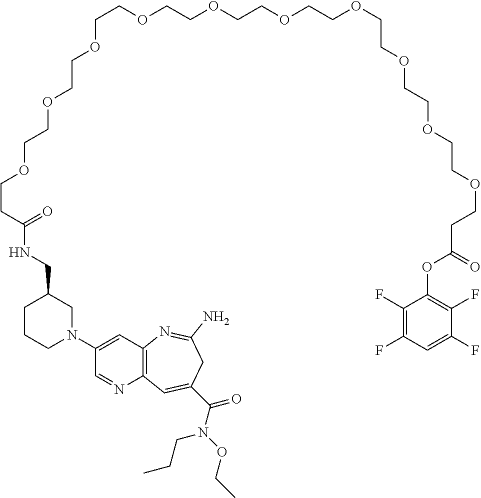 [SMILES: CCCN(OCC)C(=O)C1=Cc2ncc(N3CCC[C@@H](CNC(=O)CCOCCOCCOCCOCCOCCOCCOCCOCCOCCOCCC(=O)Oc4c(F)c(F)cc(F)c4F)C3)cc2N=C(N)C1] | 1089.2 |
| azaBzL-136 | 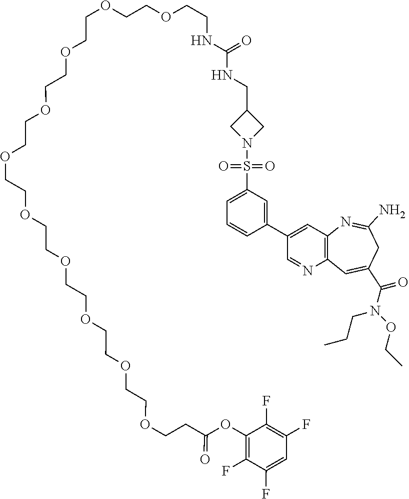 [SMILES: CCCN(OCC)C(=O)C1=Cc2ncc(-c3cccc(S(=O)(=O)N4CC(CNC(=O)NCCOCCOCCOCCOCCOCCOCCOCCOCCOCCOCCC(=O)Oc5c(F)c(F)cc(F)c5F)C4)c3)cc2N=C(N)C1] | 1217.3 |
| azaBzL-137 | 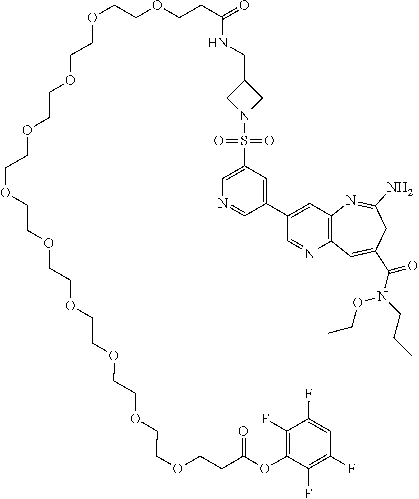 [SMILES: CCCN(OCC)C(=O)C1=Cc2ncc(-c3cncc(S(=O)(=O)N4CC(CNC(=O)CCOCCOCCOCCOCCOCCOCCOCCOCCOCCOCCC(=O)Oc5c(F)c(F)cc(F)c5F)C4)c3)cc2N=C(N)C1] | 1202.3 |
| azaBzL-138 | 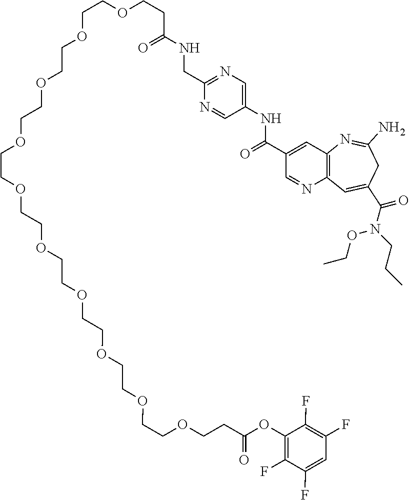 [SMILES: CCCN(OCC)C(=O)C1=Cc2ncc(C(=O)Nc3cnc(CNC(=O)CCOCCOCCOCCOCCOCCOCCOCCOCCOCCOCCC(=O)Oc4c(F)c(F)cc(F)c4F)nc3)cc2N=C(N)C1] | 1127.2 |
| azaBzL-139 | 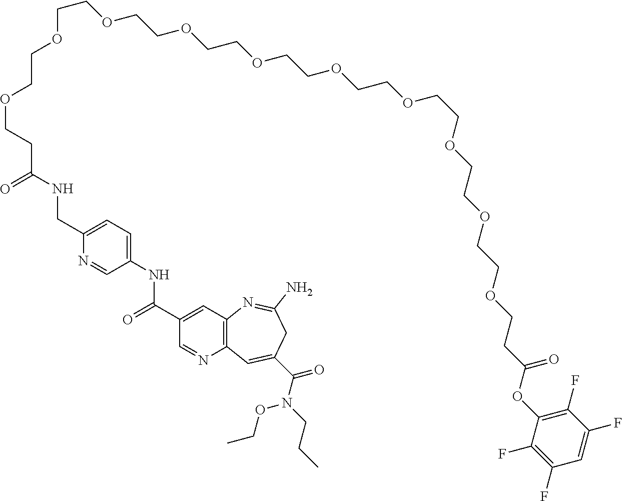 [SMILES: CCCN(OCC)C(=O)C1=Cc2ncc(C(=O)Nc3ccc(CNC(=O)CCOCCOCCOCCOCCOCCOCCOCCOCCOCCOCCC(=O)Oc4c(F)c(F)cc(F)c4F)nc3)cc2N=C(N)C1] | 1126.2 |
| azaBzL-140 | 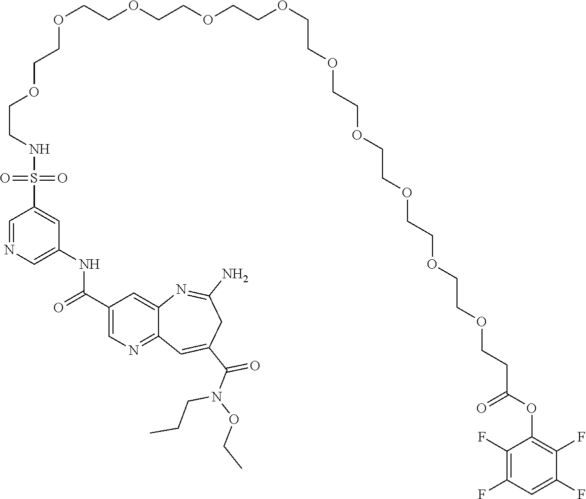 [SMILES: CCCN(OCC)C(=O)C1=Cc2ncc(C(=O)Nc3cncc(S(=O)(=O)NCCOCCOCCOCCOCCOCCOCCOCCOCCOCCOCCC(=O)Oc4c(F)c(F)cc(F)c4F)c3)cc2N=C(N)C1] | 1148.2 |
| azaBzL-141 | 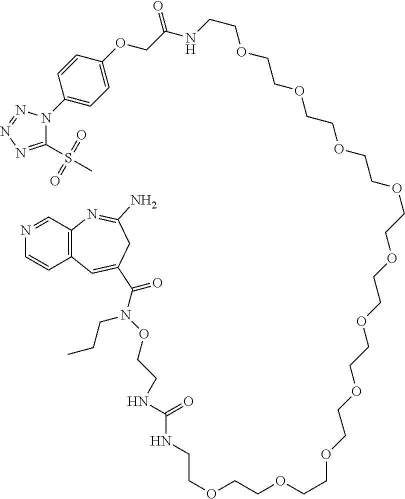 [SMILES: CCCN(OCCNC(=O)NCCOCCOCCOCCOCCOCCOCCOCCOCCOCCOCCNC(=O)COc1ccc(-n2nnnc2S(C)(=O)=O)cc1)C(=O)C1=Cc2ccncc2N=C(N)C1] | 1110.2 |
| azaBzL-142 | 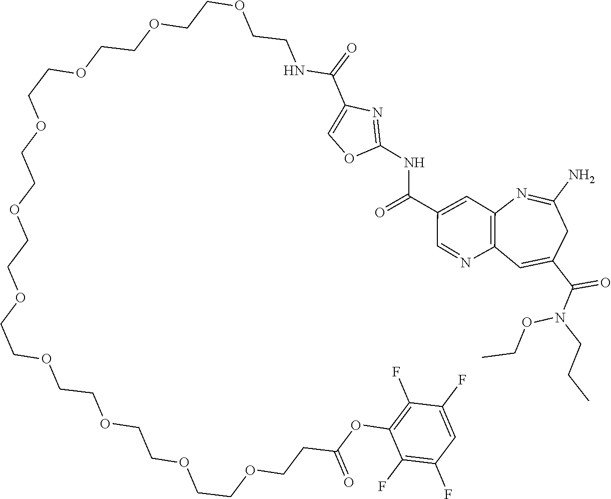 [SMILES: CCCN(OCC)C(=O)C1=Cc2ncc(C(=O)Nc3nc(C(=O)NCCOCCOCCOCCOCCOCCOCCOCCOCCOCCOCCC(=O)Oc4c(F)c(F)cc(F)c4F)co3)cc2N=C(N)C1] | 1102.1 |
| azaBzL-143 | 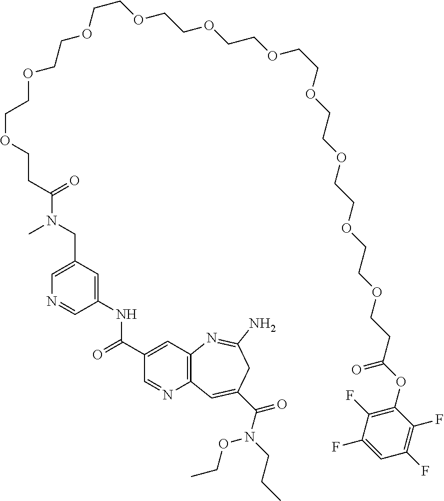 [SMILES: CCCN(OCC)C(=O)C1=Cc2ncc(C(=O)Nc3cncc(CN(C)C(=O)CCOCCOCCOCCOCCOCCOCCOCCOCCOCCOCCC(=O)Oc4c(F)c(F)cc(F)c4F)c3)cc2N=C(N)C1] | 1140.2 |
| azaBzL-144 | 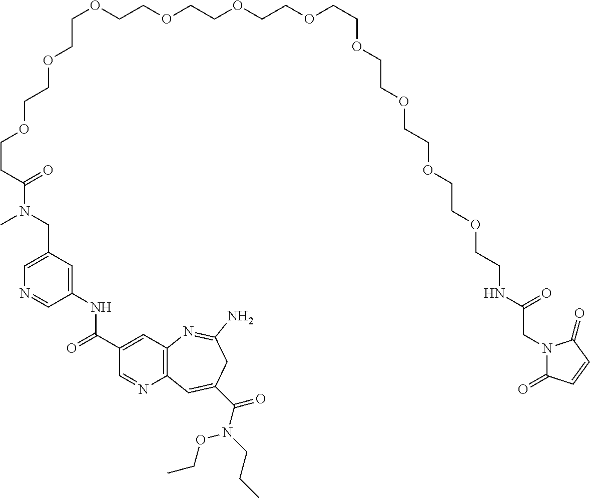 [SMILES: CCCN(OCC)C(=O)C1=Cc2ncc(C(=O)Nc3cncc(CN(C)C(=O)CCOCCOCCOCCOCCOCCOCCOCCOCCOCCOCCNC(=O)CN4C(=O)C=CC4=O)c3)cc2N=C(N)C1] | 1100.2 |
| azaBzL-145 | 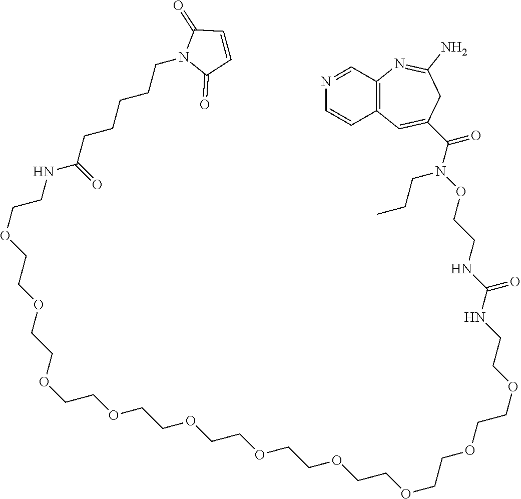 [SMILES: CCCN(OCCNC(=O)NCCOCCOCCOCCOCCOCCOCCOCCOCCOCCOCCNC(=O)CCCCCN1C(=O)C=CC1=O)C(=O)C1=Cc2ccncc2N=C(N)C1] | 1023.2 |
| azaBzL-146 | 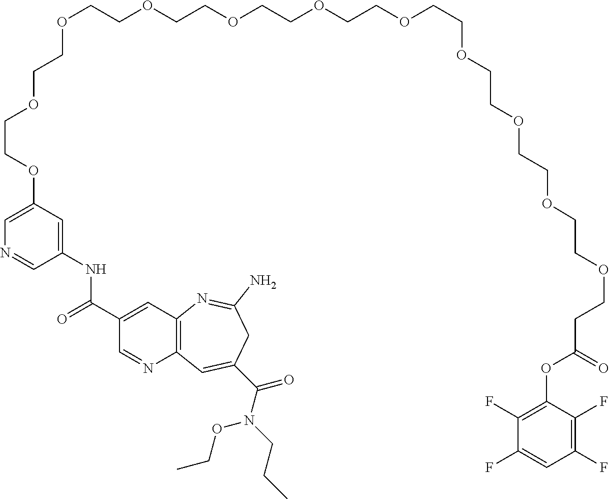 [SMILES: CCCN(OCC)C(=O)C1=Cc2ncc(C(=O)Nc3cncc(OCCOCCOCCOCCOCCOCCOCCOCCOCCOCCOCCC(=O)Oc4c(F)c(F)cc(F)c4F)c3)cc2N=C(N)C1] | 1085.1 |
| azaBzL-147 | 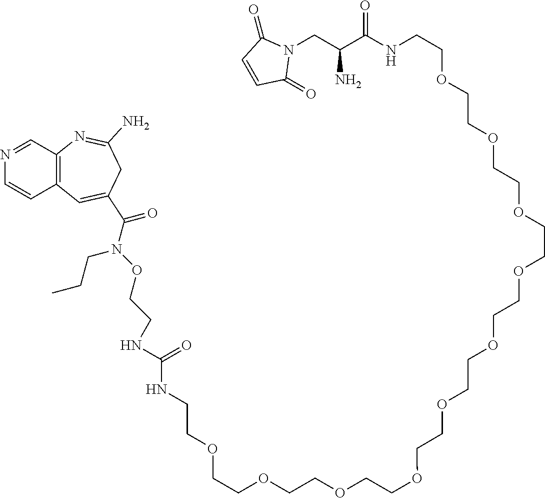 [SMILES: CCCN(OCCNC(=O)NCCOCCOCCOCCOCCOCCOCCOCCOCCOCCOCCNC(=O)[C@@H](N)CN1C(=O)C=CC1=O)C(=O)C1=Cc2ccncc2N=C(N)C1] | 996.1 |
| azaBzL-148 | 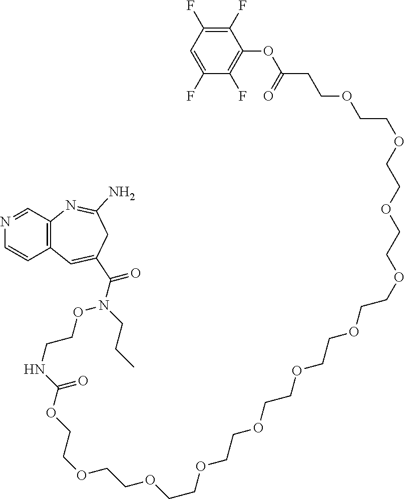 [SMILES: CCCN(OCCNC(=O)OCCOCCOCCOCCOCCOCCOCCOCCOCCOCCOCCC(=O)Oc1c(F)c(F)cc(F)c1F)C(=O)C1=Cc2ccncc2N=C(N)C1] | 1008.0 |
| azaBzL-149 | 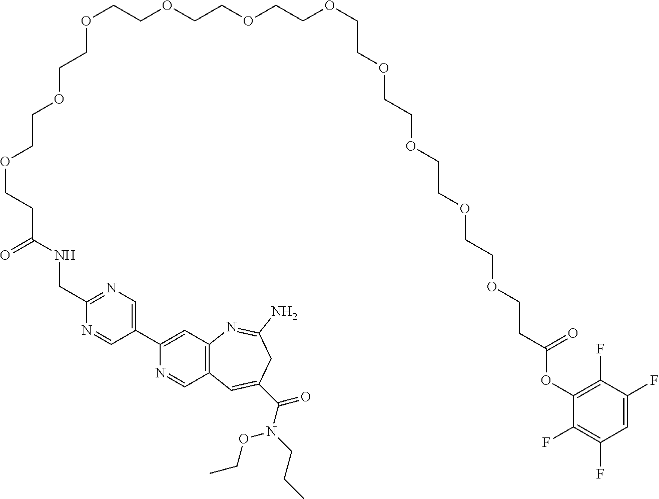 [SMILES: CCCN(OCC)C(=O)C1=Cc2cnc(-c3cnc(CNC(=O)CCOCCOCCOCCOCCOCCOCCOCCOCCOCCOCCC(=O)Oc4c(F)c(F)cc(F)c4F)nc3)cc2N=C(N)C1] | 1084.1 |
| azaBzL-150 | 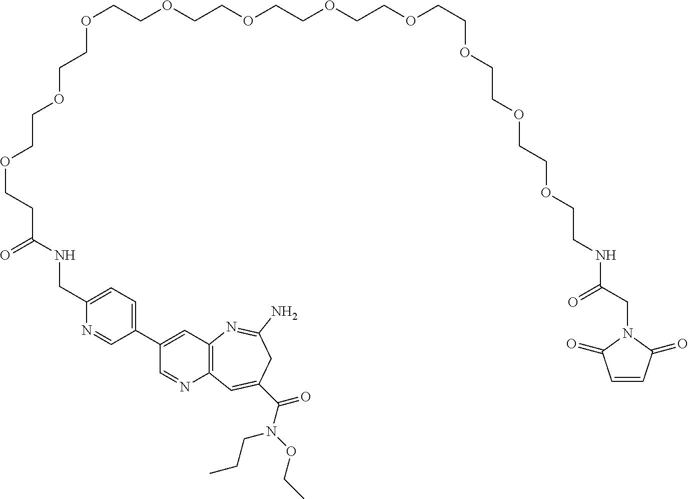 [SMILES: CCCN(OCC)C(=O)C1=Cc2ncc(-c3ccc(CNC(=O)CCOCCOCCOCCOCCOCCOCCOCCOCCOCCOCCNC(=O)CN4C(=O)C=CC4=O)nc3)cc2N=C(N)C1] | 1043.2 |

Comparator linker compounds (CL) from Table 2c have: (i) an activated ester, tetrafluorophenyl or sulfotetrafluorophenyl group which reacts with a lysine residue, or (ii) a maleimide group which reacts with a cysteine residue of an antibody to form an immunoconjugate with an antibody and a TLR-agonist-linker moiety according to Example 201. Comparator linker compounds CL-4,5,6,7,8 have an aza-benzazepine, lactam structure.

TABLE 2c
TLR agonist-linker Comparator Compounds (CL)

| CL No. | Structure | MW |
|---|---|---|
| CL-1 | 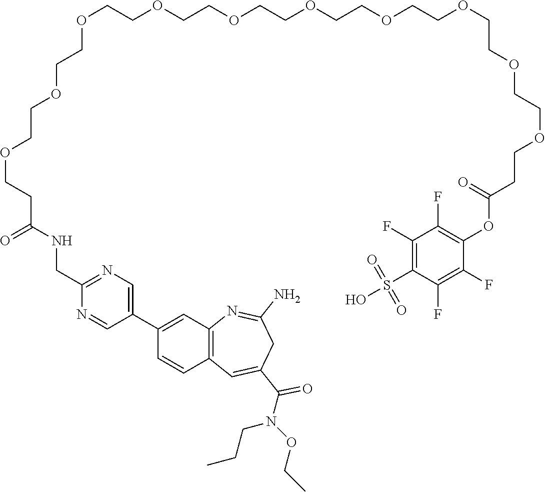 [SMILES: CCCN(OCC)C(=O)C1=Cc2ccc(-c3cnc(CNC(=O)CCOCCOCCOCCOCCOCCOCCOCCOCCOCCOCCC(=O)Oc4c(F)c(F)c(S(=O)(=O)O)c(F)c4F)nc3)cc2N=C(N)C1] | 1163.2 |
| CL-2 | 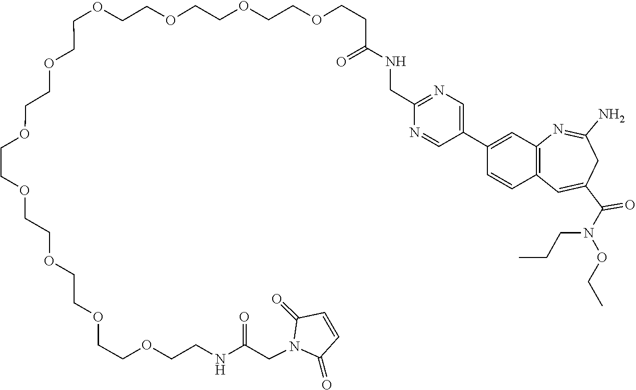 [SMILES: CCCN(OCC)C(=O)C1=Cc2ccc(-c3cnc(CNC(=O)CCOCCOCCOCCOCCOCCOCCOCCOCCOCCOCCNC(=O)CN4C(=O)C=CC4=O)nc3)cc2N=C(N)C1] | 1043.2 |
| CL-3 | 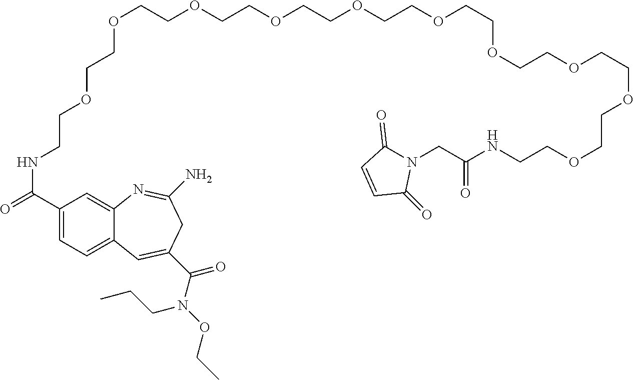 [SMILES: CCCN(OCC)C(=O)C1=Cc2ccc(C(=O)NCCOCCOCCOCCOCCOCCOCCOCCOCCOCCOCCNC(=O)CN3C(=O)C=CC3=O)cc2N=C(N)C1] | 951.1 |
| CL-4 | 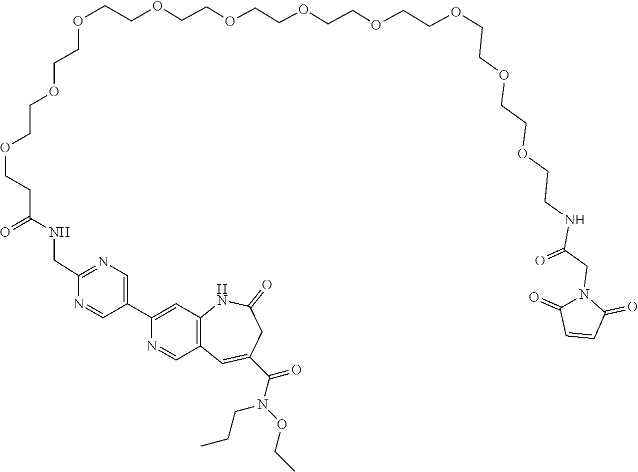 [SMILES: CCCN(OCC)C(=O)C1=Cc2cnc(-c3cnc(CNC(=O)CCOCCOCCOCCOCCOCCOCCOCCOCCOCCOCCNC(=O)CN4C(=O)C=CC4=O)nc3)cc2NC(=O)C1] | 1045.2 |
| CL-5 | 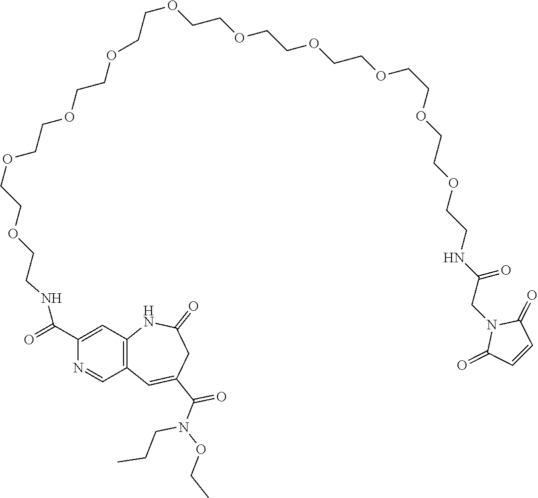 [SMILES: CCCN(OCC)C(=O)C1=Cc2cnc(C(=O)NCCOCCOCCOCCOCCOCCOCCOCCOCCOCCOCCNC(=O)CN3C(=O)C=CC3=O)cc2NC(=O)C1] | 953.1 |
| CL-6 | 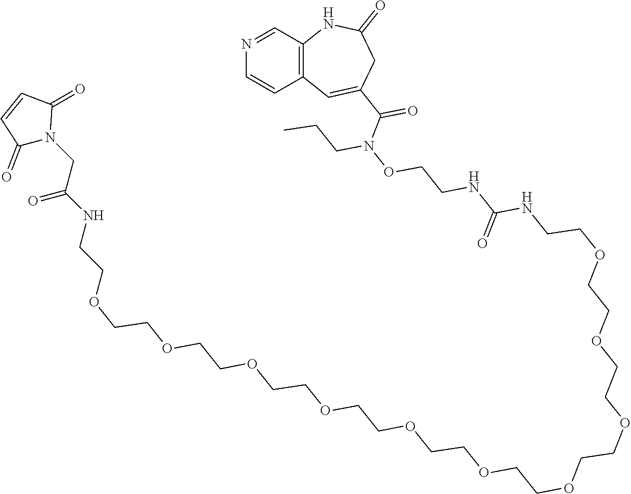 [SMILES: CCCN(OCCNC(=O)NCCOCCOCCOCCOCCOCCOCCOCCOCCOCCOCCNC(=O)CN1C(=O)C=CC1=O)C(=O)C1=Cc2ccncc2NC(=O)C1] | 968.1 |
| CL-7 | 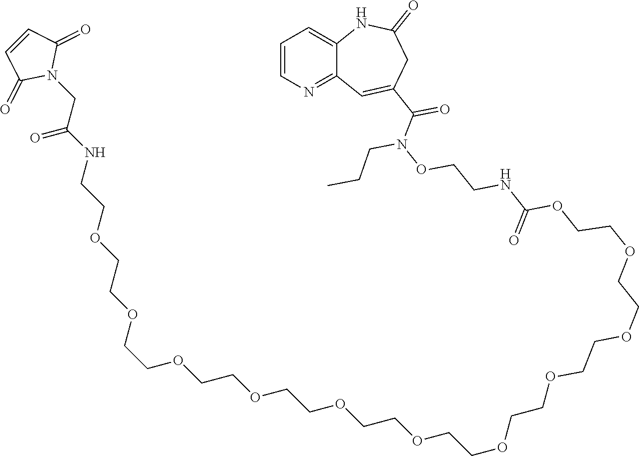 [SMILES: CCCN(OCCNC(=O)OCCOCCOCCOCCOCCOCCOCCOCCOCCOCCOCCNC(=O)CN1C(=O)C=CC1=O)C(=O)C1=Cc2ncccc2NC(=O)C1] | 969.1 |
| CL-8 | 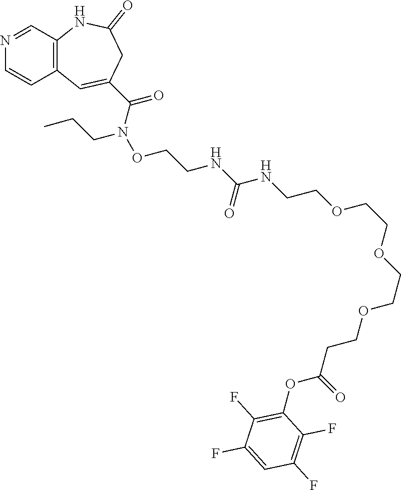 [SMILES: CCCN(OCCNC(=O)NCCOCCOCCOCCC(=O)Oc1c(F)c(F)cc(F)c1F)C(=O)C1=Cc2ccncc2NC(=O)C1] | 699.7 |

### Aza-Benzazepine Immunoconjugates

Immune-stimulating antibody conjugates, i.e. immunoconjugates, direct TLR7/8 agonists into tumors to activate tumor-infiltrating myeloid cells and initiate a broad innate and adaptive anti-tumor immune response (Ackerman, et al., (2021) Nature Cancer 2:18-33.

Exemplary embodiments of immunoconjugates comprise an antibody covalently attached to one or more aza-benzazepine moieties by a linker, and having Formula I:

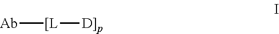

or a pharmaceutically acceptable salt thereof, wherein:
Ab is an antibody;
p is an integer from 1 to 8;
L is the linker;
D is the aza-benzazepine moiety having the formula:

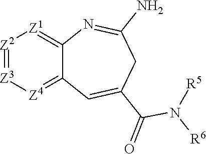

Z¹ is selected from CR¹ and N;
Z² is selected from CR² and N;
Z³ is selected from CR³ and N;
Z⁴ is selected from CR⁴ and N;
where one or two of Z¹, Z², Z³, and Z⁴ are N;
R¹, R², R³, R⁴, R⁵, and R⁶ are independently selected from the group consisting of H, C(═O), C(═O)N(R⁵), O, N(R⁵), S, S(O)₂, S(O)₂N(R⁵), C₁-C₁₂ alkyl, C₂-C₆ alkenyl, C₂-C₆ alkynyl, C₃-C₁₂ carbocyclyl, C₆-C₂₀ aryl, C₂-C₉ heterocyclyl, and C₁-C₂₀ heteroaryl, each of which are independently and optionally substituted with one or more groups selected from:
—(C₁-C₁₂ alkyldiyl)-N(R⁷)—*;
—(C₁-C₁₂ alkyldiyl)-N(R⁷)₂;
—(C₁-C₁₂ alkyldiyl)-OR⁷;
—(C₃-C₁₂ carbocyclyl);
—(C₃-C₁₂ carbocyclyl)-*;
—(C₃-C₁₂ carbocyclyl)-(C₁-C₁₂ alkyldiyl)-NR⁷—*;
—(C₃-C₁₂ carbocyclyl)-(C₁-C₁₂ alkyldiyl)-N(R⁷)₂;
—(C₃-C₁₂ carbocyclyl)-NR⁷—C(═NR⁷)NR⁷—*;
—(C₆-C₂₀ aryl);
—(C₆-C₂₀ aryldiyl)-*;
—(C₆-C₂₀ aryldiyl)-N(R⁷)—*;
—(C₆-C₂₀ aryldiyl)-(C₁-C₁₂ alkyldiyl)-N(R⁷)—*;
—(C₆-C₂₀ aryldiyl)-(C₁-C₁₂ alkyldiyl)-(C₂-C₂₀ heterocyclyldiyl)-*;
—(C₆-C₂₀ aryldiyl)-(C₁-C₁₂ alkyldiyl)-N(R⁷)₂;
—(C₆-C₂₀ aryldiyl)-(C₁-C₁₂ alkyldiyl)-NR⁷—C(═NR^{7a})N(R⁷)—*;
—(C₂-C₂₀ heterocyclyl);
—(C₂-C₂₀ heterocyclyl)-*;
—(C₂-C₉ heterocyclyl)-(C₁-C₁₂ alkyldiyl)-NR⁷—*;
—(C₂-C₉ heterocyclyl)-(C₁-C₁₂ alkyldiyl)-N(R⁷)₂;
—(C₂-C₉ heterocyclyl)-C(═O)—(C₁-C₁₂ alkyldiyl)-N(R⁷)—*;
—(C₂-C₉ heterocyclyl)-NR⁷—C(═NR^{7a})NR⁷—*;
—(C₂-C₉ heterocyclyl)-NR⁷—(C₆-C₂₀ aryldiyl)-(C₁-C₁₂ alkyldiyl)-N(R⁷)—*;
—(C₂-C₉ heterocyclyl)-(C₆-C₂₀ aryldiyl)-*;
—(C₁-C₂₀ heteroaryl);
—(C₁-C₂₀ heteroaryl)-*;
—(C₁-C₂₀ heteroaryl)-(C₁-C₁₂ alkyldiyl)-N(R⁷)—*;
—(C₁-C₂₀ heteroaryl)-(C₁-C₁₂ alkyldiyl)-N(R⁷)₂;
—(C₁-C₂₀ heteroaryl)-NR⁷—C(═NR^{7a})N(R⁷)—*;
—(C₁-C₂₀ heteroaryl)-N(R⁷)C(═O)—(C₁-C₁₂ alkyldiyl)-N(R⁷)—*;
—C(═O)—*;
—C(═O)—(C₁-C₁₂ alkyldiyl)-N(R⁷)—*;
—C(═O)—(C₂-C₂₀ heterocyclyldiyl)-*;
—C(═O)N(R⁷)₂;
—C(═O)N(R⁷)—*;
—C(═O)N(R⁷)—(C₁-C₁₂ alkyldiyl)-N(R⁷)C(═O)R⁷;
—C(═O)N(R⁷)—(C₁-C₁₂ alkyldiyl)-N(R⁷)C(═O)N(R⁷)₂;
—C(═O)NR⁷—(C₁-C₁₂ alkyldiyl)-N(R⁷)CO₂R⁷;
—C(═O)NR⁵—(C₁-C₁₂ alkyldiyl)-N(R⁵⁷)C(═NR^{57a})N(R⁵⁷)₂;
—C(═O)NR⁵—(C₁-C₁₂ alkyldiyl)-NR⁵⁷C(═NR^{7a})R⁷;
—C(═O)NR⁵—(C₁-C₈ alkyldiyl)-NR⁷(C₂-C₅ heteroaryl);
—C(═O)NR⁷—(C₁-C₂₀ heteroaryldiyl)-N(R⁷)—*;
—C(═O)NR⁷—(C₁-C₂₀ heteroaryldiyl)-*;
—C(═O)NR⁷—(C₁-C₂₀ heteroaryldiyl)-(C₁-C₁₂ alkyldiyl)-N(R⁷)₂;
—C(═O)NR⁷—(C₁-C₂₀ heteroaryldiyl)-(C₂-C₂₀ heterocyclyldiyl)-C(═O)NR⁷—(C₁-C₁₂ alkyldiyl)-NR⁷—*;
—N(R⁷)₂;
—N(R⁷)—*;
—N(R⁷)C(═O)R⁷;
—N(R⁷)C(═O)—*;
—N(R⁷)C(═O)N(R⁷)₂;
—N(R⁷)C(═O)N(R⁷)—*;
—N(R⁷)CO₂R⁷;
—NR⁷C(═NR^{7a})N(R⁷)₂;
—NR⁷C(═NR^{7a})N(R⁷)—*;
—NR⁷C(═NR^{7a})R⁷;
—N(R⁷)C(═O)—(C₁-C₁₂ alkyldiyl)-N(R⁷)—*;
—N(R⁷)—(C₂-C₅ heteroaryl);
—N(R⁷)—S(═O)₂—(C₁-C₁₂ alkyl);
—O—(C₁-C₁₂ alkyl);
—O—(C₁-C₁₂ alkyldiyl)-N(R⁷)₂;
—O—(C₁-C₁₂ alkyldiyl)-N(R⁷)—*;
—O—C(═O)N(R⁷)₂;
—O—C(═O)N(R⁷)—*;
—O—(R⁷)—*;
—OR⁷;
—S(═O)₂—(C₂-C₂₀ heterocyclyldiyl)-*;
—S(═O)₂—(C₂-C₂₀ heterocyclyldiyl)-(C₁-C₁₂ alkyldiyl)-N(R⁷)₂;
—S(═O)₂—(C₂-C₂₀ heterocyclyldiyl)-(C₁-C₁₂ alkyldiyl)-NR⁷—*; and
—S(═O)₂—(C₂-C₂₀ heterocyclyldiyl)-(C₁-C₁₂ alkyldiyl)-OH;
or R⁵ and R⁶ together form a 5- or 6-membered heterocyclyl ring;
R⁷ is independently selected from the group consisting of H, C₆-C₂₀ aryl, C₃-C₁₂ carbocyclyl, C₆-C₂₀ aryldiyl, C₁-C₁₂ alkyl, and C₁-C₁₂ alkyldiyl, or two R⁵ groups together form a 5- or 6-membered heterocyclyl ring;
R^{7a} is selected from the group consisting of C₆-C₂₀ aryl and C₁-C₂₀ heteroaryl; where the asterisk * indicates the attachment site of L, and where one of R¹, R², R³, R⁴, R⁵ and R⁶ is attached to L; and
alkyl, alkyldiyl, alkenyl, alkenyldiyl, alkynyl, alkynyldiyl, aryl, aryldiyl, carbocyclyl, carbocyclyldiyl, heterocyclyl, heterocyclyldiyl, heteroaryl, and heteroaryldiyl are independently and optionally substituted with one or more groups independently selected from F, Cl, Br, I, —CN, —CH₃, —CH₂CH₃, —CH═CH₂, —C═CH, —C—CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —O(CH₂CH₂O)r-(CH₂)ₘCO₂H, —O(CH₂CH₂O)~H, —OCH₂F, —OCHF₂, —OCF₃, —OP(O)(OH)₂, —S(O)₂N(CH₃)₂, —SCH₃, —S(O)₂CH₃, and —S(O)₃H.

An exemplary embodiment of the immunoconjugate of Formula I includes wherein L is selected from the group consisting of:
—C(═O)-PEG-;
—C(═O)-PEG-C(═O)N(R⁸)—(C₁-C₁₂ alkyldiyl)-C(═O)-Gluc-;
—C(═O)-PEG-O—;
—C(═O)-PEG-O—C(═O)—;
—C(═O)-PEG-C(═O)—;
—C(═O)-PEG-C(═O)-PEP-;
—C(═O)-PEG-N(R⁸)—;
—C(═O)-PEG-N(R⁸)—C(═O)—;
—C(═O)-PEG-N(R⁸)-PEG-C(═O)-PEP-;
—C(═O)-PEG-N⁺(R⁸)₂-PEG-C(═O)-PEP-;
—C(═O)-PEG-C(═O)-PEP-N(R⁸)—(C₁-C₁₂ alkyldiyl)-;
—C(═O)-PEG-C(═O)-PEP-N(R⁸)—(C₁-C₁₂ alkyldiyl)N(R⁸)C(═O)—(C₂-C₅ monoheterocyclyldiyl)-;
—C(═O)-PEG-SS-(C₁-C₁₂ alkyldiyl)-OC(═O)—;
—C(═O)-PEG-SS-(C₁-C₁₂ alkyldiyl)-C(═O)—;
—C(═O)—(C₁-C₁₂ alkyldiyl)-C(═O)-PEP-;
—C(═O)—(C₁-C₁₂ alkyldiyl)-C(═O)-PEP-N(R⁸)—(C₁-C₁₂ alkyldiyl)-;
—C(═O)—(C₁-C₁₂ alkyldiyl)-C(═O)-PEP-N(R⁸)—(C₁-C₁₂ alkyldiyl)-N(R⁸)—C(═O);
—C(═O)—(C₁-C₁₂ alkyldiyl)-C(═O)-PEP-N(R⁸)—(C₁-C₁₂ alkyldiyl)-N(R⁸)C(═O)—(C₂-C₅ monoheterocyclyldiyl)-;
-succinimidyl-(CH₂)ₘ—C(═O)N(R⁸)-PEG-;
-succinimidyl-(CH₂)ₘ—C(═O)N(R⁸)-PEG-C(═O)N(R⁸)—(C₁-C₁₂ alkyldiyl)-C(═O)-Gluc-;
-succinimidyl-(CH₂)ₘ—C(═O)N(R⁸)-PEG-O—;
-succinimidyl-(CH₂)ₘ—C(═O)N(R⁸)-PEG-O—C(═O)—;
-succinimidyl-(CH₂)ₘ—C(═O)N(R⁸)-PEG-C(═O)—;
-succinimidyl-(CH₂)ₘ—C(═O)N(R⁸)-PEG-N(R⁸)—;
-succinimidyl-(CH₂)ₘ—C(═O)N(R⁸)-PEG-N(R⁸)—C(═O)—;
-succinimidyl-(CH₂)ₘ—C(═O)N(R⁸)-PEG-C(═O)-PEP-;
-succinimidyl-(CH₂)ₘ—C(═O)N(R⁸)-PEG-SS-(C₁-C₁₂ alkyldiyl)-OC(═O)—;
-succinimidyl-(CH₂)ₘ—C(═O)-PEP-N(R⁸)—(C₁-C₁₂ alkyldiyl)-;
-succinimidyl-(CH₂)ₘ—C(═O)-PEP-N(R⁸)—(C₁-C₁₂ alkyldiyl)N(R⁸)C(═O)—; and
-succinimidyl-(CH₂)ₘ—C(═O)-PEP-N(R⁸)—(C₁-C₁₂ alkyldiyl)N(R⁸)C(═O)—(C₂-C₅ monoheterocyclyldiyl)-;
R⁸ is independently H or C₁-C₆ alkyl;
PEG has the formula: —(CH₂CH₂O)ₙ—(CH₂)ₘ—; m is an integer from 1 to 5, and n is an integer from 1 to 50;
Gluc has the formula:

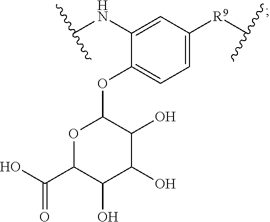

PEP has the formula:

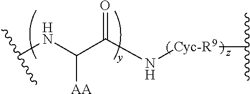

where AA is independently selected from a natural or unnatural amino acid side chain, or one or more of AA, and an adjacent nitrogen atom form a 5-membered ring proline amino acid, and the wavy line indicates a point of attachment;
Cyc is selected from C₆-C₂₀ aryldiyl and C₁-C₂₀ heteroaryldiyl, optionally substituted with one or more groups selected from F, Cl, NO₂, —OH, —OCH₃, and a glucuronic acid having the structure:

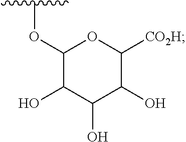 [SMILES: *OC1OC(C(=O)O)C(O)C(O)C1O]

R⁹ is selected from the group consisting of —CH(R¹⁰)O—, —CH₂—, —CH₂N(R¹⁰)—, and —CH(R¹⁰)O—C(═O)—, where R¹⁰ is selected from H, C₁-C₆ alkyl, C(═O)—C₁-C₆ alkyl, and —C(═O)N(R¹)₂, where R¹¹ is independently selected from the group consisting of H, C₁-C₁₂ alkyl, and —(CH₂CH₂O)ₙ—(CH₂)ₘ—OH, where m is an integer from 1 to 5, and n is an integer from 2 to 50, or two R¹¹ groups together form a 5- or 6-membered heterocyclyl ring;
y is an integer from 2 to 12;
z is 0 or 1.

An exemplary embodiment of the immunoconjugate of Formula I includes wherein Z¹ is N.

An exemplary embodiment of the immunoconjugate of Formula I includes wherein Z² is N.

An exemplary embodiment of the immunoconjugate of Formula I includes wherein Z³ is N.

An exemplary embodiment of the immunoconjugate of Formula I includes wherein Z⁴ is N.

An exemplary embodiment of the immunoconjugate of Formula I includes wherein R⁵ and R⁶ are independently selected from C₁-C₈ alkyl, —O—(C₁-C₁₂ alkyl), —(C₁-C₁₂ alkyldiyl)-OR⁵, —(C₁-C₈ alkyldiyl)-N(R⁵)CO₂R⁵, —(C₁-C₁₂ alkyl)-OC(O)N(R⁵)₂, —O—(C₁-C₁₂ alkyl)-N(R⁵)CO₂R⁵, and —O—(C₁-C₁₂ alkyl)-OC(O)N(R⁵)₂.

An exemplary embodiment of the immunoconjugate of Formula I includes wherein R⁵ is C₁-C₈ alkyl and R⁶ is —O—(C₁-C₁₂ alkyl).

An exemplary embodiment of the immunoconjugate of Formula I includes wherein R⁵ is —CH₂CH₂CH₃ and R⁶ is selected from —CH₂CH₂CH₂NHCO₂(t-Bu), —OCH₂CH₂NHCO₂(cyclobutyl), and —CH₂CH₂CH₂NHCO₂(cyclobutyl).

An exemplary embodiment of the immunoconjugate of Formula I includes wherein R⁵ and R⁶ are each independently selected from —CH₂CH₂CH₃, —OCH₂CH₃, —OCH₂CF₃, —CH₂CH₂CF₃, —OCH₂CH₂OH, and —CH₂CH₂CH₂OH.

An exemplary embodiment of the immunoconjugate of Formula I includes wherein R⁵ is —CH₂CH₂CH₃ and R⁶ is —OCH₂CH₃.

An exemplary embodiment of the immunoconjugate of Formula I includes wherein R⁶ is selected from the group consisting of:

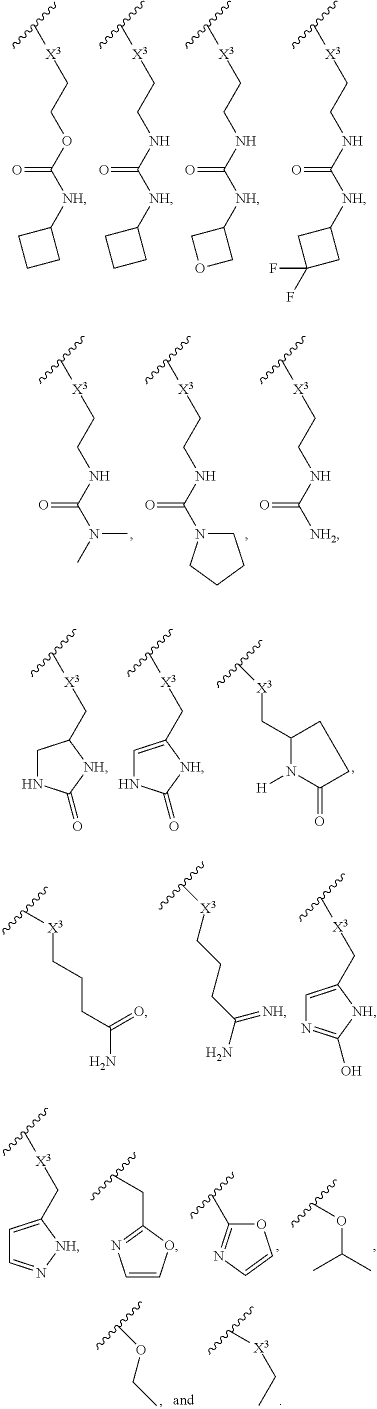

An exemplary embodiment of the immunoconjugate of Formula I includes where R¹ is attached to L.

An exemplary embodiment of the immunoconjugate of Formula I includes where R² is attached to L.

An exemplary embodiment of the immunoconjugate of Formula I includes where R³ is attached to L.

An exemplary embodiment of the immunoconjugate of Formula I includes where R⁴ is attached to L.

An exemplary embodiment of the immunoconjugate of Formula I includes where R⁵ or R⁶ is attached to L.

An exemplary embodiment of the immunoconjugate of Formula I includes wherein L is —C(═O)-PEG- or —C(═O)-PEG-C(═O)—.

An exemplary embodiment of the immunoconjugate of Formula I includes wherein L is attached to a cysteine thiol of the antibody.

An exemplary embodiment of the immunoconjugate of Formula I includes wherein for the PEG, m is 1 or 2, and n is an integer from 2 to 10, or wherein n is 10.

An exemplary embodiment of the immunoconjugate of Formula I includes wherein L comprises PEP and PEP is a dipeptide and has the formula:

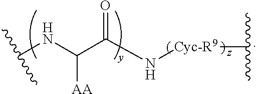

An exemplary embodiment of the immunoconjugate of Formula I includes wherein AA is independently selected from H, —CH₃, —CH(CH₃)₂, —CH₂(C₆H₅), —CH₂CH₂CH₂CH₂NH₂, —CH₂CH₂CH₂NHC(NH)NH₂, —CHCH(CH₃)CH₃, —CH₂SO₃H, and —CH₂CH₂CH₂NHC(O)NH₂; or two AA form a 5-membered ring proline amino acid.

An exemplary embodiment of the immunoconjugate of Formula I includes wherein PEP is a dipeptide and has the formula:

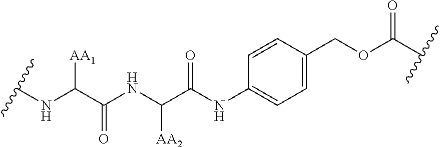

wherein AA₁ and AA₂ are independently selected from a side chain of a naturally-occurring amino acid.

An exemplary embodiment of the immunoconjugate of Formula I includes wherein AA₁ is —CH(CH₃)₂, and AA₂ is —CH₂CH₂CH₂NHC(O)NH₂.

An exemplary embodiment of the immunoconjugate of Formula I includes wherein AA₁ and AA₂ are independently selected from GlcNAc aspartic acid, —CH₂SO₃H, and —CH₂OPO₃H.

An exemplary embodiment of the immunoconjugate of Formula I includes wherein PEP is a tripeptide and has the formula:

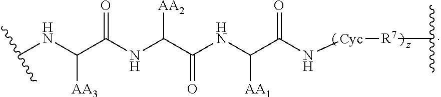

An exemplary embodiment of the immunoconjugate of Formula I includes wherein PEP is a tetrapeptide and has the formula:

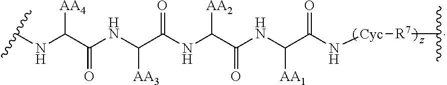

An exemplary embodiment of the immunoconjugate of Formula I includes wherein
AA₁ is selected from the group consisting of Abu, Ala, and Val;
AA₂ is selected from the group consisting of Nle(O-Bzl), Oic and Pro;
AA₃ is selected from the group consisting of Ala and Met(O)₂; and
AA₄ is selected from the group consisting of Oic, Arg(NO₂), Bpa, and Nle(O-Bzl).

An exemplary embodiment of the immunoconjugate of Formula I includes wherein L comprises PEP and PEP is selected from the group consisting of Ala-Pro-Val, Asn-Pro-Val, Ala-Ala-Val, Ala-Ala-Pro-Ala, Ala-Ala-Pro-Val, and Ala-Ala-Pro-Nva.

An exemplary embodiment of the immunoconjugate of Formula I includes wherein L comprises PEP and PEP is selected from the structures:

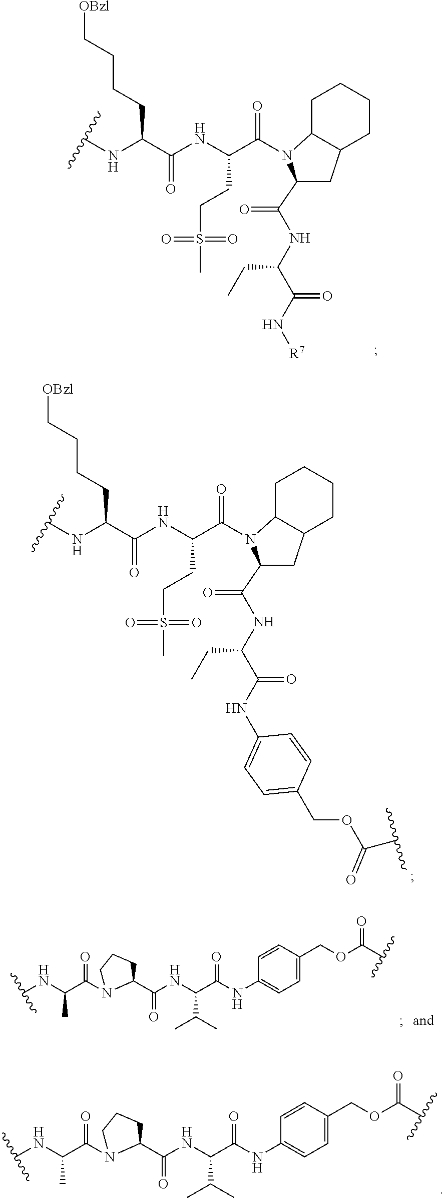

An exemplary embodiment of the immunoconjugate of Formula I includes wherein L is selected from the structures:

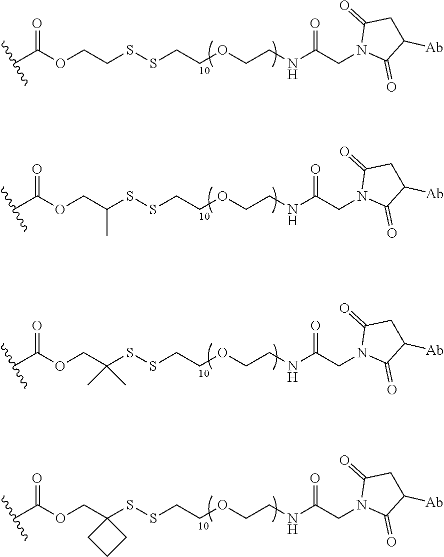

where the wavy line indicates the attachment to one of R¹, R², R³, R⁴, R⁵, and R⁶.

The invention includes all reasonable combinations, and permutations of the features, of the Formula I embodiments.

In certain embodiments, the immunoconjugate compounds of the invention include those with immunostimulatory activity. The immunoconjugates of the invention selectively deliver an effective dose of a aza-benzazepine (azaBz) drug or metabolite to tumor tissue, whereby greater selectivity (i.e., a lower efficacious dose) may be achieved while increasing the therapeutic index (“therapeutic window”) relative to unconjugated azaBz.

Each immunoconjugate of Tables 3a, 3b, 3c was prepared according to the methods of Example 201, purified by HPLC, and characterized by mass spectroscopy.

TABLE 3a
Aza-benzazepine Immunoconjugates (IC)

| | | | | cDC |
|---|---|---|---|---|
| | | | | Activation |
| Immuno- | | | | (IL 12p70 |
| conjugate | azaBzL | Antigen | | Secretion) - |
| IC No. | Table 2a | Antibody | DAR | EC₅₀ (nM) |
| IC-1 | azaBzL-4 | HER2 | 3.5 | 0.8 |
| | | trastuzumab | | |
| IC-2 | azaBzL-5 | HER2 | 3.2 | 0.6 |
| | | trastuzumab | | |
| IC-3 | azaBzL-6 | HER2 | 3.8 | 0.07 |
| | | trastuzumab | | |
| IC-4 | azaBzL-7 | HER2 | 3.4 | 0.1 |
| | | trastuzumab | | |
| IC-5 | azaBzL-2 | HER2 | 3.2, 3.3. | N/A |
| | | trastuzumab | | |
| IC-6 | azaBzL-1 | TROP2 | 3.8 | 0.7 |
| IC-7 | azaBzL-1 | PD-L1 | 3.1, 3.6 | 0.7 |
| IC-8 | azaBzL-1 | bezlotoxumab | 3.5 | ND |
| IC-9 | azaBzL-1 | HER2 | 3.3 | 1.4 |
| | | trastuzumab | | |
| IC-10 | azaBzL-9 | HER2 | 3.5 | N/A |
| | | trastuzumab | | |
| IC-11 | azaBzL-10 | HER2 | 3.5 | 0.6 |
| | | trastuzumab | | |
| IC-12 | azaBzL-11 | HER2 | 3.6 | N/A |
| | | trastuzumab | | |
| IC-13 | azaBzL-13 | HER2 | 3.5 | ND |
| | | trastuzumab | | |
| IC-14 | azaBzL-14 | HER2 | 3.6 | N/A |
| | | trastuzumab | | |

N/A = no calculable EC50,
ND = not measured

TABLE 3b
Aza-benzazepine Immunoconjugates (IC)

| | | | | cDC |
|---|---|---|---|---|
| | | | | Activation |
| Immuno- | | | | (IL 12p70 |
| conjugate | azaBzL | Antigen | | Secretion) - |
| IC No. | Table 2b | Antibody | DAR | EC₅₀ (nM) |
| IC-15 | azaBzL-1 | CEA.3-G1f | 3.8 | N/A |
| | | CEACAM5 | | |
| | | SEQ ID NO: 1, 6 | | |
| IC-16 | azaBzL-4 | CEA.3-G1f | 3.8, 4.0 | N/A |
| | | CEACAM5 | | |
| | | SEQ ID NO: 1, 6 | | |
| IC-17 | azaBzL-5 | CEA.3-G1f | 4.0 | N/A |
| | | CEACAM5 | | |
| | | SEQ ID NO: 1, 6 | | |
| IC-18 | azaBzL-21 | HER2 | 4.0 | N/A |
| | | trastuzumab | | |
| IC-19 | azaBzL-10 | CEA.3-G1f | 4.0 | 0.78 |
| | | CEACAM5 | | |
| | | SEQ ID NO: 1, 6 | | |
| IC-20 | azaBzL-11 | CEA.3-G1f | 3.5 | N/A |
| | | CEACAM5 | | |
| | | SEQ ID NO: 1, 6 | | |
| IC-21 | azaBzL-21 | CEA.3-G1f | 3.6 | N/A |
| | | CEACAM5 | | |
| | | SEQ ID NO: 1, 6 | | |
| IC-22 | azaBzL-5 | CEA.6-G1f | 3.5 | N/A |
| | | CEACAM5 | | |
| | | SEQ ID NO: 2, 7 | | |
| IC-23 | azaBzL-17 | HER2 | 2.6 | 1.1 |
| | | trastuzumab | | |
| IC-24 | azaBzL-26 | HER2 | 2.8 | N/A |
| | | trastuzumab | | |
| IC-25 | azaBzL-4 | CEA.6-G1f | 3.7 | ND |
| | | CEACAM5 | | |
| IC-26 | azaBzL-28 | HER2 | 3.0 | 1.0 |
| | | trastuzumab | | |
| IC-27 | azaBzL-29 | HER2 | 2.9 | 3.0 |
| | | trastuzumab | | |
| IC-28 | azaBzL-30 | HER2 | 2.8 | 0.6 |
| | | trastuzumab | | |
| IC-29 | azaBzL-149 | HER2 | 2.8 | ND |
| | | trastuzumab | | |
| IC-30 | azaBzL-33 | HER2 | 2.4 | ND |
| | | trastuzumab | | |
| IC-31 | azaBzL-34 | HER2 | 2.6 | N/A |
| | | trastuzumab | | |
| IC-32 | azaBzL-35 | HER2 | 2.6 | N/A |
| | | trastuzumab | | |
| IC-33 | azaBzL-32 | HER2 | 2.5 | N/A |
| | | trastuzumab | | |
| IC-34 | azaBzL-31 | HER2 | 4.0 | N/A |
| | | trastuzumab | | |
| IC-35 | azaBzL-27 | HER2 | 3.7 | ND |
| | | trastuzumab | | |
| IC-36 | azaBzL-24 | HER2 | 3.6 | ND |
| | | trastuzumab | | |
| IC-37 | azaBzL-25 | HER2 | 3.3 | ND |
| | | trastuzumab | | |
| IC-38 | azaBzL-1 | HER2 | 4.0 | ND |
| | | Tras-G1f-N297A | | |
| IC-39 | azaBzL-1 | HER2 | 3.9 | ND |
| | | Tras-G1f-FEA | | |
| IC-40 | azaBzL-1 | HER2 | 4.0 | ND |
| | | Tras-G1f-STR | | |
| IC-41 | azaBzL-20 | CEA.3-G1f | 3.6 | N/A |
| | | CEACAM5 | | |
| | | SEQ ID NO: 1, 6 | | |
| IC-42 | azaBzL-27 | CEA.3-G1f | 4.1 | N/A |
| | | CEACAM5 | | |
| | | SEQ ID NO: 1, 6 | | |
| IC-43 | azaBzL-24 | CEA.3-G1f | 4.0 | 3.5 |
| | | CEACAM5 | | |
| | | SEQ ID NO: 1, 6 | | |
| IC-44 | azaBzL-36 | HER2 | 2.8 | ND |
| | | trastuzumab | | |
| IC-45 | azaBzL-37 | HER2 | 3.9 | 0.5 |
| | | trastuzumab | | |
| IC-46 | azaBzL-43 | HER2 | 3.9 | ND |
| | | trastuzumab | | |
| IC-47 | azaBzL-42 | HER2 | 3.9, 4.1 | 0.8 |
| | | trastuzumab | | |
| IC-48 | azaBzL-37 | CEA.3-G1f | 3.7 | 1.1 |
| | | CEACAM5 | | |
| | | SEQ ID NO: 1, 6 | | |
| IC-49 | azaBzL-39 | CEA.3-G1f | 3.7, 3.9 | 1.8 |
| | | CEACAM5 | | |
| | | SEQ ID NO: 1, 6 | | |
| IC-50 | azaBzL-40 | CEA.3-G1f | 3.7 | N/A |
| | | CEACAM5 | | |
| | | SEQ ID NO: 1, 6 | | |
| IC-51 | azaBzL-42 | CEA.3-G1f | 3.6, 3.9 | 1.1 |
| | | CEACAM5 | | |
| | | SEQ ID NO: 1, 6 | | |
| IC-52 | azaBzL-43 | CEA.3-G1f | 3.6 | N/A |
| | | CEACAM5 | | |
| | | SEQ ID NO: 1, 6 | | |
| IC-53 | azaBzL-44 | CEA.3-G1f | 3.9 | N/A |
| | | CEACAM5 | | |
| | | SEQ ID NO: 1, 6 | | |
| IC-54 | azaBzL-41 | CEA.3-G1f | 3.2, 4.0 | 1.9 |
| | | CEACAM5 | | |
| | | SEQ ID NO: 1, 6 | | |
| IC-55 | azaBzL-50 | Imject | 1.6 | ND |
| | | Mariculture KLH | | |
| IC-56 | azaBzL-38 | HER2 | 2.5 | 0.7 |
| | | trastuzumab | | |
| IC-57 | azaBzL-45 | HER2 | 2.9 | 0.5 |
| | | trastuzumab | | |
| IC-58 | azaBzL-46 | HER2 | 2.3 | ND |
| | | trastuzumab | | |
| IC-59 | azaBzL-47 | HER2 | 2.5 | ND |
| | | trastuzumab | | |
| IC-60 | azaBzL-48 | HER2 | 2.7 | ND |
| | | trastuzumab | | |
| IC-61 | azaBzL-49 | HER2 | 2.7 | ND |
| | | trastuzumab | | |
| IC-62 | azaBzL-51 | HER2 | 2.8 | 0.7 |
| | | trastuzumab | | |
| IC-63 | azaBzL-52 | HER2 | 2.6 | 0.5 |
| | | trastuzumab | | |
| IC-64 | azaBzL-53 | HER2 | 2.5 | 0.5 |
| | | trastuzumab | | |
| IC-65 | azaBzL-50 | BSA monomer | 1.2, 1.4 | ND |
| IC-66 | azaBzL-54 | CEA.3-G1f | 4.1 | 1.9 |
| | | CEACAM5 | | |
| | | SEQ ID NO: 1, 6 | | |
| IC-67 | azaBzL-55 | CEA.3-G1f | 3.8 | 2.1 |
| | | CEACAM5 | | |
| | | SEQ ID NO: 1, 6 | | |
| IC-68 | azaBzL-56 | CEA.3-G1f | 3.9 | 1.7 |
| | | CEACAM5 | | |
| | | SEQ ID NO: 1, 6 | | |
| IC-69 | azaBzL-57 | CEA.3-G1f | 3.7 | N/A |
| | | CEACAM5 | | |
| | | SEQ ID NO: 1, 6 | | |
| IC-70 | azaBzL-150 | CEA.3-G1f | 3.8 | 1.0 |
| | | CEACAM5 | | |
| | | SEQ ID NO: 1, 6 | | |
| IC-71 | azaBzL-58 | CEA.3-G1f | 3.7 | 2.2 |
| | | CEACAM5 | | |
| | | SEQ ID NO: 1, 6 | | |
| IC-72 | azaBzL-59 | CEA.3-G1f | 3.7, 4.0 | 1.4 |
| | | CEACAM5 | | |
| | | SEQ ID NO: 1, 6 | | |
| IC-73 | azaBzL-60 | CEA.3-G1f | 3.6 | N/A |
| | | CEACAM5 | | |
| | | SEQ ID NO: 1, 6 | | |
| IC-74 | azaBzL-61 | CEA.3-G1f | 3.6 | N/A |
| | | CEACAM5 | | |
| | | SEQ ID NO: 1, 6 | | |
| IC-75 | azaBzL-62 | CEA.3-G1f | 3.8 | N/A |
| | | CEACAM5 | | |
| | | SEQ ID NO: 1, 6 | | |
| IC-76 | azaBzL-73 | CEA.3-G1f | 3.8 | NA |
| | | CEACAM5 | | |
| | | SEQ ID NO: 1, 6 | | |
| IC-77 | azaBzL-63 | CEA.3-G1f | 3.6 | N/A |
| | | CEACAM5 | | |
| | | SEQ ID NO: 1, 6 | | |
| IC-78 | azaBzL-64 | CEA.3-G1f | 3.6 | N/A |
| | | CEACAM5 | | |
| | | SEQ ID NO: 1, 6 | | |
| IC-79 | azaBzL-65 | CEA.3-G1f | 3.8 | N/A |
| | | CEACAM5 | | |
| | | SEQ ID NO: 1, 6 | | |
| IC-80 | azaBzL-66 | CEA.3-G1f | 3.7 | N/A |
| | | CEACAM5 | | |
| | | SEQ ID NO: 1, 6 | | |
| IC-81 | azaBzL-67 | CEA.3-G1f | 3.7 | N/A |
| | | CEACAM5 | | |
| | | SEQ ID NO: 1, 6 | | |
| IC-82 | azaBzL-68 | CEA.3-G1f | 3.7 | N/A |
| | | CEACAM5 | | |
| | | SEQ ID NO: 1, 6 | | |
| IC-83 | azaBzL-69 | CEA.3-G1f | 3.8 | 3.4 |
| | | CEACAM5 | | |
| | | SEQ ID NO: 1, 6 | | |
| IC-84 | azaBzL-70 | CEA.3-G1f | 3.8 | N/A |
| | | CEACAM5 | | |
| | | SEQ ID NO: 1, 6 | | |
| IC-85 | azaBzL-71 | CEA.3-G1f | 3.7 | 2.7 |
| | | CEACAM5 | | |
| | | SEQ ID NO: 1, 6 | | |
| IC-86 | azaBzL-72 | CEA.3-G1f | 3.7, 4.0 | 2.3 |
| | | CEACAM5 | | |
| | | SEQ ID NO: 1, 6 | | |
| IC-87 | azaBzL-74 | CEA.3-G1f | 3.8 | N/A |
| | | CEACAM5 | | |
| | | SEQ ID NO: 1, 6 | | |
| IC-88 | azaBzL-75 | CEA.3-G1f | 3.8 | 4.2 |
| | | CEACAM5 | | |
| | | SEQ ID NO: 1, 6 | | |
| IC-89 | azaBzL-77 | CEA.3-G1f | 3.5 | 2.3 |
| | | CEACAM5 | | |
| | | SEQ ID NO: 1, 6 | | |
| IC-90 | azaBzL-78 | CEA.3-G1f | 3.9 | N/A |
| | | CEACAM5 | | |
| | | SEQ ID NO: 1, 6 | | |
| IC-91 | azaBzL-83 | CEA.3-G1f | 3.8 | N/A |
| | | CEACAM5 | | |
| | | SEQ ID NO: 1, 6 | | |
| IC-92 | azaBzL-84 | CEA.3-G1f | 3.8 | 1.8 |
| | | CEACAM5 | | |
| | | SEQ ID NO: 1, 6 | | |
| IC-93 | azaBzL-86 | CEA.3-G1f | 3.7 | 2.9 |
| | | CEACAM5 | | |
| | | SEQ ID NO: 1, 6 | | |
| IC-94 | azaBzL-87 | CEA.3-G1f | 3.7, 4.0 | 1.8 |
| | | CEACAM5 | | |
| | | SEQ ID NO: 1, 6 | | |
| IC-95 | azaBzL-76 | CEA.3-G1f | 3.5 | N/A |
| | | CEACAM5 | | |
| | | SEQ ID NO: 1, 6 | | |
| IC-96 | azaBzL-92 | CEA.3-G1f | 3.7 | N/A |
| | | CEACAM5 | | |
| | | SEQ ID NO: 1, 6 | | |
| IC-97 | azaBzL-93 | CEA.3-G1f | 3.9 | ND |
| | | CEACAM5 | | |
| | | SEQ ID NO: 1, 6 | | |
| IC-98 | azaBzL-94 | CEA.3-G1f | 3.9 | ND |
| | | CEACAM5 | | |
| | | SEQ ID NO: 1, 6 | | |
| IC-99 | azaBzL-95 | CEA.3-G1f | 3.3 | ND |
| | | CEACAM5 | | |
| | | SEQ ID NO: 1, 6 | | |
| IC-100 | azaBzL-85 | CEA.3-G1f | 3.7, 4.1 | 1.2 |
| | | CEACAM5 | | |
| | | SEQ ID NO: 1, 6 | | |
| IC-101 | azaBzL-88 | CEA.3-G1f | 3.7 | N/A |
| | | CEACAM5 | | |
| | | SEQ ID NO: 1, 6 | | |
| IC-102 | azaBzL-89 | CEA.3-Gf | 3.8 | N/A |
| | | CEACAM5 | | |
| | | SEQ ID NO: 1, 6 | | |
| IC-103 | azaBzL-96 | CEA.3-G1f | 3.6 | N/A |
| | | CEACAM5 | | |
| | | SEQ ID NO: 1, 6 | | |
| IC-104 | azaBzL-97 | CEA.3-G1f | 3.4 | N/A |
| | | CEACAM5 | | |
| | | SEQ ID NO: 1, 6 | | |
| IC-105 | azaBzL-98 | CEA.3-G1f | 3.5 | N/A |
| | | CEACAM5 | | |
| | | SEQ ID NO: 1, 6 | | |
| IC-106 | azaBzL-99 | CEA.3-G1f | 3.7 | N/A |
| | | CEACAM5 | | |
| | | SEQ ID NO: 1, 6 | | |
| IC-107 | azaBzL-79 | CEA.3-G1f | 3.8 | N/A |
| | | CEACAM5 | | |
| | | SEQ ID NO: 1, 6 | | |
| IC-108 | azaBzL-80 | CEA.3-G1f | 3.8 | N/A |
| | | CEACAM5 | | |
| | | SEQ ID NO: 1, 6 | | |
| IC-109 | azaBzL-81 | CEA.3-G1f | 3.8 | 1.9 |
| | | CEACAM5 | | |
| | | SEQ ID NO: 1, 6 | | |
| IC-110 | azaBzL-82 | CEA.3-G1f | 3.8, 4.1 | 1.3 |
| | | CEACAM5 | | |
| | | SEQ ID NO: 1, 6 | | |
| IC-111 | azaBzL-90 | CEA.3-G1f | 3.6, 3.9 | 1.6 |
| | | CEACAM5 | | |
| | | SEQ ID NO: 1, 6 | | |
| IC-112 | azaBzL-91 | CEA.3-G1f | 4.0 | N/A |
| | | CEACAM5 | | |
| | | SEQ ID NO: 1, 6 | | |
| IC-113 | azaBzL-104 | CEA.3-G1f | 3.5 | 3.2 |
| | | CEACAM5 | | |
| | | SEQ ID NO: 1, 6 | | |
| IC-114 | azaBzL-105 | CEA.3-G1f | 3.7 | ND |
| | | CEACAM5 | | |
| | | SEQ ID NO: 1, 6 | | |
| IC-115 | azaBzL-106 | CEA.3-G1f | 3.4 | 3.2 |
| | | CEACAM5 | | |
| | | SEQ ID NO: 1, 6 | | |
| IC-116 | azaBzL-107 | CEA.3-G1f | 3.9 | ND |
| | | CEACAM5 | | |
| | | SEQ ID NO: 1, 6 | | |
| IC-117 | azaBzL-108 | CEA.3-G1f | 3.8 | ND |
| | | CEACAM5 | | |
| | | SEQ ID NO: 1, 6 | | |
| IC-118 | azaBzL-100 | CEA.3-G1f | 3.5 | ND |
| | | CEACAM5 | | |
| | | SEQ ID NO: 1, 6 | | |
| IC-119 | azaBzL-101 | CEA.3-G1f | 3.6 | ND |
| | | CEACAM5 | | |
| | | SEQ ID NO: 1, 6 | | |
| IC-120 | azaBzL-102 | CEA.3-G1f | 3.6 | ND |
| | | CEACAM5 | | |
| | | SEQ ID NO: 1, 6 | | |
| IC-121 | azaBzL-103 | CEA.3-G1f | 3.4 | N/A |
| | | CEACAM5 | | |
| | | SEQ ID NO: 1, 6 | | |
| IC-122 | azaBzL-50 | BSA monomer | 5.3 | ND |
| IC-123 | azaBzL-17 | CEA.3-G1f | 2.0 | 2.4 |
| | | CEACAM5 | | |
| | | SEQ ID NO: 1, 6 | | |
| IC-124 | azaBzL-17 | CEA.9-G1fhL2 | 2.5 | ND |
| | | SEQ ID NO: 3, 8 | | |
| IC-125 | azaBzL-32 | CEA.9-G1fhL2 | 2.5 | ND |
| | | SEQ ID NO: 3, 8 | | |
| IC-126 | azaBzL-38 | CEA.9-G1fhL2 | 2.5 | ND |
| | | SEQ ID NO: 3, 8 | | |
| IC-127 | azaBzL-51 | CEA.9-G1fhL2 | 2.8 | ND |
| | | SEQ ID NO: 3, 8 | | |
| IC-128 | azaBzL-52 | CEA.9-G1fhL2 | 2.5 | ND |
| | | SEQ ID NO: 3, 8 | | |
| IC-129 | azaBzL 53- | CEA.9-G1fhL2 | 2.8 | ND |
| | | SEQ ID NO: 3, 8 | | |
| IC-130 | azaBzL-82 | CEA.3-G1f-G236A | 4.2 | 3.8 |
| | | SEQ ID NO: 4, 6 | | |
| IC-131 | azaBzL-82 | CEA.3-G1f-SDIE | 4.2 | 1.6 |
| | | SEQ ID NO: 5, 9 | | |
| IC-132 | azaBzL-82 | CEA.3-G1fnf | 4.1 | 2.3 |
| | | SEQ ID NO: 1, 6 | | |
| IC-133 | azaBzL-41 | HER2 | 3.5 | 0.6 |
| | | trastuzumab | | |
| IC-134 | azaBzL-59 | HER2 | 3.2 | 0.7 |
| | | trastuzumab | | |
| IC-135 | azaBzL-72 | HER2 | 3.4 | 0.7 |
| | | trastuzumab | | |
| IC-136 | azaBzL-116 | HER2 | 3.0 | ND |
| | | trastuzumab | | |
| IC-137 | azaBzL-117 | HER2 | 2.9 | ND |
| | | trastuzumab | | |
| IC-138 | azaBzL-119 | HER2 | 2.9 | ND |
| | | trastuzumab | | |
| IC-139 | azaBzL-121 | HER2 | 2.9 | ND |
| | | trastuzumab | | |
| IC-140 | azaBzL-112 | HER2 | 3.3 | 1.8 |
| | | trastuzumab | | |
| IC-141 | azaBzL-113 | HER2 | 3.3 | 1.6 |
| | | trastuzumab | | |
| IC-142 | azaBzL-114 | HER2 | 3.2 | 1.5 |
| | | trastuzumab | | |
| IC-143 | azaBzL-118 | HER2 | 3.2 | 0.4 |
| | | trastuzumab | | |
| IC-144 | azaBzL-120 | HER2 | 2.5 | ND |
| | | trastuzumab | | |
| IC-145 | azaBzL-122 | HER2 | 2.5 | ND |
| | | trastuzumab | | |
| IC-146 | azaBzL-123 | HER2 | 2.5 | ND |
| | | trastuzumab | | |
| IC-147 | azaBzL-124 | HER2 | 2.4 | 0.6 |
| | | trastuzumab | | |
| IC-148 | azaBzL-125 | HER2 | 2.5 | ND |
| | | trastuzumab | | |
| IC-149 | azaBzL-126 | HER2 | 2.6 | ND |
| | | trastuzumab | | |
| IC-150 | azaBzL-127 | HER2 | 2.6 | 5.4 |
| | | trastuzumab | | |
| IC-151 | azaBzL-129 | HER2 | 2.7 | ND |
| | | trastuzumab | | |
| IC-152 | azaBzL-39 | HER2 | 4.1 | 0.7 |
| | | trastuzumab | | |
| IC-153 | azaBzL-57 | HER2 | 4.0 | 1.4 |
| | | trastuzumab | | |
| IC-154 | azaBzL-76 | HER2 | 3.9 | 1.7 |
| | | trastuzumab | | |
| IC-155 | azaBzL-79 | HER2 | 3.9 | 0.6 |
| | | trastuzumab | | |
| IC-156 | azaBzL-87 | HER2 | 3.8 | ND |
| | | trastuzumab | | |
| IC-157 | azaBzL-103 | HER2 | 4.0 | 1.5 |
| | | trastuzumab | | |
| IC-158 | azaBzL-104 | HER2 | 3.5 | 1.5 |
| | | trastuzumab | | |
| IC-159 | azaBzL-109 | HER2 | 3.4 | 0.8 |
| | | trastuzumab | | |
| IC-160 | azaBzL-110 | HER2 | 4.0 | 0.5 |
| | | trastuzumab | | |
| IC-161 | azaBzL-111 | HER2 | 3.7 | 2.2 |
| | | trastuzumab | | |
| IC-162 | azaBzL-148 | HER2 | 2.9, 2.3 | 1.7 |
| | | trastuzumab | | |

N/A = no calculable EC50,
ND = not measured

TABLE 3c
Comparator Immunoconjugates (CIC)

| | | | | cDC Activation |
|---|---|---|---|---|
| Comparator | Comparator | | | (IL 12p70 |
| Immunocon- | compound | Antigen | | Secretion) - |
| jugate No. | Table 2c | Antibody | DAR | EC₅₀ (nM) |
| CIC-1 | C-1 | HER2 | 2.4 | 1.6 (n = 5) |
| | | trastuzumab | | |

Secreted cytokine levels in supernatants were determined using a LegendPlex cytokine bead array kit. Immunoconjugates (IC) of Tables 3a and 3b induce secretion of cytokine TNFα (alpha), relevant to mounting an immune response to cancer and demonstrate the activation of myeloid cells when exposed to antigen-expressing tumor cells such as HER2. The aza-benzazepine immunoconjugates of Tables 3a and 3b stimulated higher levels of TNFα than the comparator immunoconjugate CIC-1. Notably, the aza-benzazepine payload represents a more efficient payload providing increased activity, while decreasing molecular weight and hydrophobicity. Naked antibody does not induce myeloid activation, demonstrating the dependence on the TLR7/8 activating payload.

Drug loading is represented by p, the number of aza-benzazepine (azaBz) moieties per antibody in an immunoconjugate of Formula I, and as measured (DAR) in the exemplary Immunoconjugates of Table 3a. Drug (azaBz) loading may range from 1 to about 8 drug moieties (D) per antibody. Immunoconjugates of Formula I include mixtures or collections of antibodies conjugated with a range of drug moieties, from 1 to about 8. In some embodiments, the number of drug moieties that can be conjugated to an antibody is limited by the number of reactive or available amino acid side chain residues such as lysine and cysteine. In some embodiments, free cysteine residues are introduced into the antibody amino acid sequence by the methods described herein. In such aspects, p may be 1, 2, 3, 4, 5, 6, 7, or 8, and ranges thereof, such as from 1 to 8 or from 2 to 5. In any such aspect, p and n are equal (i.e., p=n=1, 2, 3, 4, 5, 6, 7, or 8, or some range there between). Exemplary immunoconjugates of Formula I include, but are not limited to, antibodies that have 1, 2, 3, or 4 engineered cysteine amino acids (Lyon, R. et al. (2012) Methods in Enzym. 502:123-138). In some embodiments, one or more free cysteine residues are already present in an antibody forming intra-chain and inter-chain disulfide bonds (native disulfide groups), without the use of engineering, in which case the existing free, reduced cysteine residues may be used to conjugate the antibody to a drug. In some embodiments, an antibody is exposed to reducing conditions prior to conjugation of the antibody in order to generate one or more free cysteine residues.

For some immunoconjugates, p may be limited by the number of attachment sites on the antibody. For example, where the attachment is a cysteine thiol, as in certain exemplary embodiments described herein, an antibody may have only one or a limited number of cysteine thiol groups, or may have only one or a limited number of sufficiently reactive thiol groups, to which the drug may be attached. In other embodiments, one or more lysine amino groups in the antibody may be available and reactive for conjugation with a azaBz-linker compound of Formula II. In certain embodiments, higher drug loading, e.g. p>5, may cause aggregation, insolubility, toxicity, or loss of cellular permeability of certain antibody-drug conjugates. In certain embodiments, the average drug loading for an immunoconjugate ranges from 1 to about 8; from about 2 to about 6; or from about 3 to about 5. In certain embodiments, an antibody is subjected to denaturing conditions to reveal reactive nucleophilic groups such as lysine or cysteine.

The loading (drug/antibody ratio) of an immunoconjugate may be controlled in different ways, and for example, by: (i) limiting the molar excess of the azaBz-linker intermediate compound relative to antibody, (ii) limiting the conjugation reaction time or temperature, and (iii) partial or limiting reductive denaturing conditions for optimized antibody reactivity.

It is to be understood that where more than one nucleophilic group of the antibody reacts with a drug, then the resulting product is a mixture of immunoconjugate compounds with a distribution of one or more drug moieties attached to an antibody. The average number of drugs per antibody may be calculated from the mixture by a dual ELISA antibody assay, which is specific for antibody and specific for the drug. Individual immunoconjugate molecules may be identified in the mixture by mass spectroscopy and separated by HPLC, e.g. hydrophobic interaction chromatography (see, e.g., McDonagh et al. (2006) Prot. Engr. Design & Selection 19(7):299-307; Hamblett et al. (2004) Clin. Cancer Res. 10:7063-7070; Hamblett, K. J., et al. “Effect of drug loading on the pharmacology, pharmacokinetics, and toxicity of an anti-CD30 antibody-drug conjugate,” Abstract No. 624, American Association for Cancer Research, 2004 Annual Meeting, Mar. 27-31, 2004, Proceedings of the AACR, Volume 45, March 2004; Alley, S. C., et al. “Controlling the location of drug attachment in antibody-drug conjugates,” Abstract No. 627, American Association for Cancer Research, 2004 Annual Meeting, March 27-31, 2004, Proceedings of the AACR, Volume 45, March 2004). In certain embodiments, a homogeneous immunoconjugate with a single loading value may be isolated from the conjugation mixture by electrophoresis or chromatography.

Assessment of Immunoconjugate Activity In Vitro may be conducted according to the methods of Example 203.

### Compositions of Immunoconjugates

The invention provides a composition, e.g., a pharmaceutically or pharmacologically acceptable composition or formulation, comprising a plurality of immunoconjugates as described herein and optionally a carrier therefor, e.g., a pharmaceutically or pharmacologically acceptable carrier. The immunoconjugates can be the same or different in the composition, i.e., the composition can comprise immunoconjugates that have the same number of adjuvants linked to the same positions on the antibody construct and/or immunoconjugates that have the same number of aza-benzazepine (azaBz) adjuvants linked to different positions on the antibody construct, that have different numbers of azaBz adjuvants linked to the same positions on the antibody construct, or that have different numbers of azaBz adjuvants linked to different positions on the antibody construct.

In an exemplary embodiment, a composition comprising the immunoconjugate compounds comprises a mixture of the immunoconjugate compounds, wherein the average drug (aza-Bz) loading per antibody (DAR) in the mixture of immunoconjugate compounds is about 2 to about 5.

A composition of immunoconjugates of the invention can have an average adjuvant to antibody construct ratio (DAR) of about 0.4 to about 10. A skilled artisan will recognize that the number of aza-benzazepine adjuvants conjugated to the antibody construct may vary from immunoconjugate to immunoconjugate in a composition comprising multiple immunoconjugates of the invention and thus the adjuvant to antibody construct (e.g., antibody) ratio can be measured as an average which may be referred to as the drug to antibody ratio (DAR). The adjuvant to antibody construct (e.g., antibody) ratio can be assessed by any suitable means, many of which are known in the art, including conventional means such as mass spectrometry, ELISA assay, and HPLC. The quantitative distribution of immunoconjugates in a composition in terms of p may also be determined. In some instances, separation, purification, and characterization of homogeneous immunoconjugates where p is a certain value from immunoconjugates with other drug loadings may be achieved by means such as reverse phase HPLC or electrophoresis.

In some embodiments, the composition further comprises one or more pharmaceutically or pharmacologically acceptable excipients. For example, the immunoconjugates of the invention can be formulated for parenteral administration, such as IV administration or administration into a body cavity or lumen of an organ. Alternatively, the immunoconjugates can be injected intra-tumorally. Compositions for injection will commonly comprise a solution of the immunoconjugate dissolved in a pharmaceutically acceptable carrier. Among the acceptable vehicles and solvents that can be employed are water and an isotonic solution of one or more salts such as sodium chloride, e.g., Ringer's solution. In addition, sterile fixed oils can conventionally be employed as a solvent or suspending medium. For this purpose, any bland fixed oil can be employed, including synthetic monoglycerides or diglycerides. In addition, fatty acids such as oleic acid can likewise be used in the preparation of injectables. These compositions desirably are sterile and generally free of undesirable matter. These compositions can be sterilized by conventional, well known sterilization techniques. The compositions can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like.

The composition can contain any suitable concentration of the immunoconjugate. The concentration of the immunoconjugate in the composition can vary widely, and will be selected primarily based on fluid volumes, viscosities, body weight, and the like, in accordance with the particular mode of administration selected and the patient's needs. In certain embodiments, the concentration of an immunoconjugate in a solution formulation for injection will range from about 0.1% (w/w) to about 10% (w/w).

Method of Treating Cancer with Immunoconjugates

The invention provides a method for treating cancer. The method includes administering a therapeutically effective amount of an immunoconjugate as described herein (e.g., as a composition as described herein) to a subject in need thereof, e.g., a subject that has cancer and is in need of treatment for the cancer. The method includes administering a therapeutically effective amount of an immunoconjugate (IC) selected from Table 3a.

It is contemplated that the immunoconjugate of the present invention may be used to treat various hyperproliferative diseases or disorders, e.g. characterized by the overexpression of a tumor antigen. Exemplary hyperproliferative disorders include benign or malignant solid tumors and hematological disorders such as leukemia and lymphoid malignancies.

In another aspect, an immunoconjugate for use as a medicament is provided. In certain embodiments, the invention provides an immunoconjugate for use in a method of treating an individual comprising administering to the individual an effective amount of the immunoconjugate. In one such embodiment, the method further comprises administering to the individual an effective amount of at least one additional therapeutic agent, e.g., as described herein.

In a further aspect, the invention provides for the use of an immunoconjugate in the manufacture or preparation of a medicament. In one embodiment, the medicament is for treatment of cancer, the method comprising administering to an individual having cancer an effective amount of the medicament. In one such embodiment, the method further comprises administering to the individual an effective amount of at least one additional therapeutic agent, e.g., as described herein.

Carcinomas are malignancies that originate in the epithelial tissues. Epithelial cells cover the external surface of the body, line the internal cavities, and form the lining of glandular tissues. Examples of carcinomas include, but are not limited to, adenocarcinoma (cancer that begins in glandular (secretory) cells such as cancers of the breast, pancreas, lung, prostate, stomach, gastroesophageal junction, and colon) adrenocortical carcinoma; hepatocellular carcinoma; renal cell carcinoma; ovarian carcinoma; carcinoma in situ; ductal carcinoma; carcinoma of the breast; basal cell carcinoma; squamous cell carcinoma; transitional cell carcinoma; colon carcinoma; nasopharyngeal carcinoma; multilocular cystic renal cell carcinoma; oat cell carcinoma; large cell lung carcinoma; small cell lung carcinoma; non-small cell lung carcinoma; and the like. Carcinomas may be found in prostrate, pancreas, colon, brain (usually as secondary metastases), lung, breast, and skin. In some embodiments, methods for treating non-small cell lung carcinoma include administering an immunoconjugate containing an antibody construct that is capable of binding a tumor-associated antigen.

Soft tissue tumors are a highly diverse group of rare tumors that are derived from connective tissue. Examples of soft tissue tumors include, but are not limited to, alveolar soft part sarcoma; angiomatoid fibrous histiocytoma; chondromyoxid fibroma; skeletal chondrosarcoma; extraskeletal myxoid chondrosarcoma; clear cell sarcoma; desmoplastic small round-cell tumor; dermatofibrosarcoma protuberans; endometrial stromal tumor; Ewing's sarcoma; fibromatosis (Desmoid); infantile fibrosarcoma; gastrointestinal stromal tumor; bone giant cell tumor; tenosynovial giant cell tumor; inflammatory myofibroblastic tumor; uterine leiomyoma; leiomyosarcoma; lipoblastoma; typical lipoma; spindle cell or pleomorphic lipoma; atypical lipoma; chondroid lipoma; well-differentiated liposarcoma; myxoid/round cell liposarcoma; pleomorphic liposarcoma; myxoid malignant fibrous histiocytoma; high-grade malignant fibrous histiocytoma; myxofibrosarcoma; malignant peripheral nerve sheath tumor; mesothelioma; neuroblastoma; osteochondroma; osteosarcoma; primitive neuroectodermal tumor; alveolar rhabdomyosarcoma; embryonal rhabdomyosarcoma; benign or malignant schwannoma; synovial sarcoma; Evan's tumor; nodular fasciitis; desmoid-type fibromatosis; solitary fibrous tumor; dermatofibrosarcoma protuberans (DFSP); angiosarcoma; epithelioid hemangioendothelioma; tenosynovial giant cell tumor (TGCT); pigmented villonodular synovitis (PVNS); fibrous dysplasia; myxofibrosarcoma; fibrosarcoma; synovial sarcoma; malignant peripheral nerve sheath tumor; neurofibroma; pleomorphic adenoma of soft tissue; and neoplasias derived from fibroblasts, myofibroblasts, histiocytes, vascular cells/endothelial cells, and nerve sheath cells.

A sarcoma is a rare type of cancer that arises in cells of mesenchymal origin, e.g., in bone or in the soft tissues of the body, including cartilage, fat, muscle, blood vessels, fibrous tissue, or other connective or supportive tissue. Different types of sarcoma are based on where the cancer forms. For example, osteosarcoma forms in bone, liposarcoma forms in fat, and rhabdomyosarcoma forms in muscle. Examples of sarcomas include, but are not limited to, primitive neuroectodermal tumor (PNET) of the thoracopulmonary region (Askin's tumor); sarcoma botryoides; chondrosarcoma; malignant hemangioendothelioma; malignant schwannoma; osteosarcoma; and soft tissue sarcomas (e.g., alveolar soft part sarcoma; angiosarcoma; cystosarcoma phyllodesdermatofibrosarcoma protuberans (DFSP); desmoid tumor; desmoplastic small round cell tumor; epithelioid sarcoma; extraskeletal chondrosarcoma; extraskeletal osteosarcoma; fibrosarcoma; gastrointestinal stromal tumor (GIST); hemangiopericytoma; hemangiosarcoma (more commonly referred to as “angiosarcoma”); Kaposi's sarcoma; leiomyosarcoma; liposarcoma; lymphangiosarcoma; malignant peripheral nerve sheath tumor (MPNST); neurofibrosarcoma; synovial sarcoma; and undifferentiated pleomorphic sarcoma).

A teratoma is a type of germ cell tumor that may contain several different types of tissue (e.g., can include tissues derived from any and/or all of the three germ layers: endoderm, mesoderm, and ectoderm), including, for example, hair, muscle, and bone. Teratomas occur most often in the ovaries in women, the testicles in men, and the tailbone in children.

Melanoma is a form of cancer that begins in melanocytes (cells that make the pigment melanin). Melanoma may begin in a mole (skin melanoma), but can also begin in other pigmented tissues, such as in the eye or in the intestines.

Merkel cell carcinoma is a rare type of skin cancer that usually appears as a flesh-colored or bluish-red nodule on the face, head or neck. Merkel cell carcinoma is also called neuroendocrine carcinoma of the skin. In some embodiments, methods for treating Merkel cell carcinoma include administering an immunoconjugate containing an antibody construct that is capable of binding, for example, CEA (e.g., labetuzumab, biosimilars thereof, or biobetters thereof). In some embodiments, the Merkel cell carcinoma has metastasized when administration occurs.

Leukemias are cancers that start in blood-forming tissue, such as the bone marrow, and cause large numbers of abnormal blood cells to be produced and enter the bloodstream. For example, leukemias can originate in bone marrow-derived cells that normally mature in the bloodstream. Leukemias are named for how quickly the disease develops and progresses (e.g., acute versus chronic) and for the type of white blood cell that is affected (e.g., myeloid versus lymphoid). Myeloid leukemias are also called myelogenous or myeloblastic leukemias. Lymphoid leukemias are also called lymphoblastic or lymphocytic leukemia. Lymphoid leukemia cells may collect in the lymph nodes, which can become swollen. Examples of leukemias include, but are not limited to, Acute myeloid leukemia (AML), Acute lymphoblastic leukemia (ALL), Chronic myeloid leukemia (CML), and Chronic lymphocytic leukemia (CLL).

Lymphomas are cancers that begin in cells of the immune system. For example, lymphomas can originate in bone marrow-derived cells that normally mature in the lymphatic system. There are two basic categories of lymphomas. One category of lymphoma is Hodgkin lymphoma (HL), which is marked by the presence of a type of cell called the Reed-Sternberg cell. There are currently 6 recognized types of HL. Examples of Hodgkin lymphomas include nodular sclerosis classical Hodgkin lymphoma (CHL), mixed cellularity CHL, lymphocyte-depletion CHL, lymphocyte-rich CHL, and nodular lymphocyte predominant HL.

The other category of lymphoma is non-Hodgkin lymphomas (NHL), which includes a large, diverse group of cancers of immune system cells. Non-Hodgkin lymphomas can be further divided into cancers that have an indolent (slow-growing) course and those that have an aggressive (fast-growing) course. There are currently 61 recognized types of NHL. Examples of non-Hodgkin lymphomas include, but are not limited to, AIDS-related Lymphomas, anaplastic large-cell lymphoma, angioimmunoblastic lymphoma, blastic NK-cell lymphoma, Burkitt's lymphoma, Burkitt-like lymphoma (small non-cleaved cell lymphoma), chronic lymphocytic leukemia/small lymphocytic lymphoma, cutaneous T-Cell lymphoma, diffuse large B-Cell lymphoma, enteropathy-type T-Cell lymphoma, follicular lymphoma, hepatosplenic gamma-delta T-Cell lymphomas, T-Cell leukemias, lymphoblastic lymphoma, mantle cell lymphoma, marginal zone lymphoma, nasal T-Cell lymphoma, pediatric lymphoma, peripheral T-Cell lymphomas, primary central nervous system lymphoma, transformed lymphomas, treatment-related T-Cell lymphomas, and Waldenstrom's macroglobulinemia.

Brain cancers include any cancer of the brain tissues. Examples of brain cancers include, but are not limited to, gliomas (e.g., glioblastomas, astrocytomas, oligodendrogliomas, ependymomas, and the like), meningiomas, pituitary adenomas, and vestibular schwannomas, primitive neuroectodermal tumors (medulloblastomas).

Immunoconjugates of the invention can be used either alone or in combination with other agents in a therapy. For instance, an immunoconjugate may be co-administered with at least one additional therapeutic agent, such as a chemotherapeutic agent. Such combination therapies encompass combined administration (where two or more therapeutic agents are included in the same or separate formulations), and separate administration, in which case, administration of the immunoconjugate can occur prior to, simultaneously, and/or following, administration of the additional therapeutic agent and/or adjuvant. Immunoconjugates can also be used in combination with radiation therapy.

The immunoconjugates of the invention (and any additional therapeutic agent) can be administered by any suitable means, including oral, parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

The immunoconjugate is administered to a subject in need thereof in any therapeutically effective amount using any suitable dosing regimen, such as the dosing regimens utilized for labetuzumab, biosimilars thereof, and biobetters thereof. For example, the methods can include administering the immunoconjugate to provide a dose of from about 100 ng/kg to about 50 mg/kg to the subject. The immunoconjugate dose can range from about 5 mg/kg to about 50 mg/kg, from about 10 μg/kg to about 5 mg/kg, or from about 100 μg/kg to about 1 mg/kg. The immunoconjugate dose can be about 100, 200, 300, 400, or 500 μg/kg. The immunoconjugate dose can be about 1, 2, 3, 4, 5, 6, 7, 8, 9, or 10 mg/kg. The immunoconjugate dose can also be outside of these ranges, depending on the particular conjugate as well as the type and severity of the cancer being treated. Frequency of administration can range from a single dose to multiple doses per week, or more frequently. In some embodiments, the immunoconjugate is administered from about once per month to about five times per week. In some embodiments, the immunoconjugate is administered once per week.

In another aspect, the invention provides a method for preventing cancer. The method comprises administering a therapeutically effective amount of an immunoconjugate (e.g., as a composition as described above) to a subject. In certain embodiments, the subject is susceptible to a certain cancer to be prevented.

Some embodiments of the invention provide methods for treating cancer as described above, wherein the cancer is breast cancer. Breast cancer can originate from different areas in the breast, and a number of different types of breast cancer have been characterized. For example, the immunoconjugates of the invention can be used for treating ductal carcinoma in situ; invasive ductal carcinoma (e.g., tubular carcinoma; medullary carcinoma; mucinous carcinoma; papillary carcinoma; or cribriform carcinoma of the breast); lobular carcinoma in situ; invasive lobular carcinoma; inflammatory breast cancer; and other forms of breast cancer such as triple negative (test negative for estrogen receptors, progesterone receptors, and excess HER2 protein) breast cancer. In some embodiments, methods for treating breast cancer include administering an immunoconjugate containing an antibody construct that is capable of binding a tumor-associated antigen (TAA), or tumors over-expressing a TAA

In some embodiments, the cancer is susceptible to a pro-inflammatory response induced by TLR7 and/or TLR8.

In some embodiments, a therapeutically effective amount of an immunoconjugate is administered to a patient in need to treat cervical cancer, endometrial cancer, ovarian cancer, prostate cancer, pancreatic cancer, esophageal cancer, bladder cancer, urinary tract cancer, urothelial carcinoma, lung cancer, non-small cell lung cancer, Merkel cell carcinoma, colon cancer, colorectal cancer, gastric cancer, or breast cancer. The Merkel cell carcinoma cancer may be metastatic Merkel cell carcinoma. The breast cancer may be triple-negative breast cancer. The esophageal cancer may be gastroesophageal junction adenocarcinoma.

### EXAMPLES

### Example 3 Synthesis of 2-amino-4-((2-((tert-butoxycarbonyl)amino)ethoxy)(propyl)carbamoyl)-3H-pyrido[4,3-b]azepine-8-carboxylic acid, azaBz-3

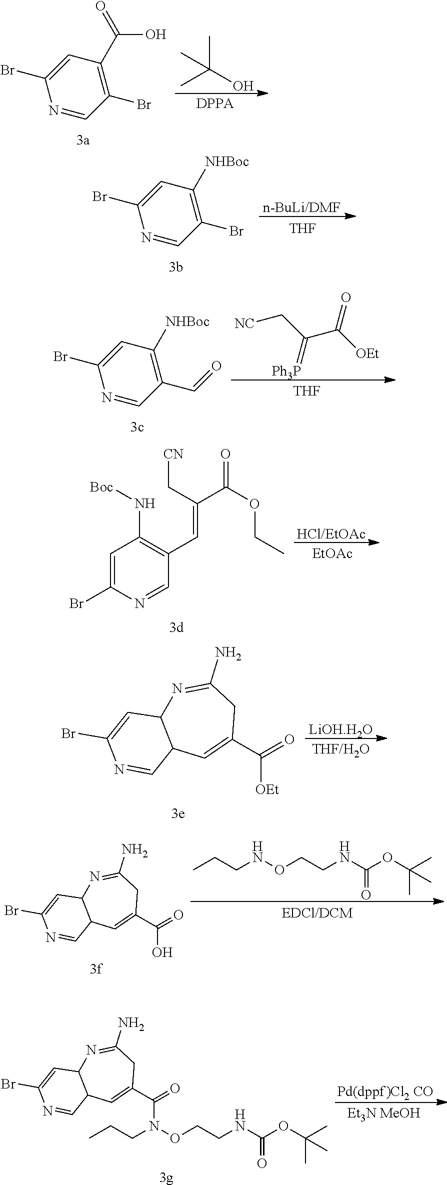

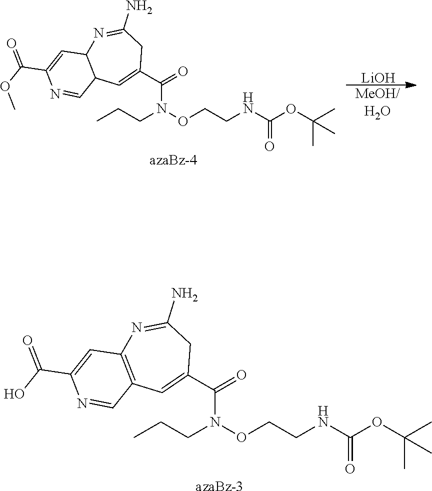

### Preparation of tert-butyl (2,5-dibromopyridin-4-yl)carbamate, 3b

To a solution of 2,5-dibromopyridine-4-carboxylic acid, 3a (25.0 g, 89.0 mmol, 1 eq) and Et₃N (27.0 g, 267 mmol, 37.2 mL, 3 eq) in tert-butanol (200 mL) was added diphenylphosphoryl azide, DPPA (49.0 g, 178 mmol, 38.6 mL, 2 eq), and then stirred at 80° C. for 12 hr to effect Curtius rearrangement by addition of tert-butanol to the intermediate isocyanate. The mixture was concentrated under reduced pressure. The residue was purified by flash silica gel chromatography (ISCO®; 120 g SepaFlash® Silica Flash Column, Eluent of 0~25% Ethyl acetate/Petroleum ether gradient @100 mL/min) to afford 3b (25 g, 71.02 mmol, 79.80% yield) as a white solid. ¹H NMR (CDCl₃, 400 MHz) δ 8.49 (s, 1H), 8.43 (s, 1H), 1.65 (s, 9H). LC/MS [M+H] 350.9 (calculated); LC/MS [M+H] 350.9 (observed).

### Preparation of tert-butyl (2-bromo-5-formylpyridin-4-yl)carbamate, 3c

To a solution of 3b (10 g, 28.4 mmol, 1 eq) in THE (150 mL) was added n-BuLi (2.5 M, 27.3 mL, 2.4 eq) at −78° C. under N₂, and the mixture was stirred for 0.5 hr at this temperature. Then DMF (10.4 g, 142 mmol, 10.9 mL, 5 eq) was added at −78° C. and stirred for 0.5 hr. The mixture was quenched with aqueous NH₄Cl, diluted with water 50 mL and extracted with EtOAc (50 mL×3). The combined organic layers were washed with brine (20 mL×2), dried over Na₂SO₄, filtered and concentrated under reduced pressure to give a residue. The residue was purified by flash silica gel chromatography (ISCO®; 25 g SepaFlash® Silica Flash Column, Eluent of 0~40% Ethyl acetate/Petroleum ether gradient @80 mL/min) to afford 3c (4.5 g, 14.94 mmol, 52.60% yield) as a white solid. ¹H NMR (CDCl₃, 400 MHz) δ 9.93 (s, 1H), 8.62 (s, 1H), 8.51 (s, 1H), 1.55 (s, 9H).

### 3 Preparation of ethyl (E)-3-(6-bromo-4-((tert-butoxycarbonyl)amino)pyridin-3-yl)-2-(cyanomethyl)acrylate, 3d

A solution of 3c (4.5 g, 14.9 mmol, 1 eq) and ethyl 3-cyano-2-(triphenyl-λ5-phosphanylidene)propanoate (6.37 g, 16.4 mmol, 1.1 eq) in DCM (50 mL) was stirred at 30° C. for 2 hr. The mixture was concentrated under reduced pressure. The residue was purified by flash silica gel chromatography (ISCO®; 25 g SepaFlash® Silica Flash Column, Eluent of 0~50% Ethyl acetate/Petroleum ether gradient @80 mL/min) to afford 3d (5 g, 12.2 mmol, 81.56% yield) as a yellow solid. ¹H NMR (CDCl₃, 400 MHz) δ 8.44 (s, 1H), 8.07 (s, 1H), 7.68 (s, 1H), 6.48-6.43 (m, 1H), 4.41 (q, J=7.2 Hz, 2H), 3.40 (s, 2H), 1.55 (s, 9H), 1.43 (t, J=7.2 Hz, 3H). LC/MS [M+H] 410.01 (calculated); LC/MS [M+H] 410.1 (observed).

### Preparation of ethyl 2-amino-8-bromo-3H-pyrido[4,3-b]azepine-4-carboxylate, 3e

To a solution of 3d (5 g, 12.2 mmol, 1 eq) in EtOAc (10 mL) was added HCl/EtOAc (4 M, 22.2 mL, 7.29 eq), and then stirred at 25° C. for 16 hr. The mixture was filtered and concentrated under reduced pressure to afford 3e (4 g, 11.54 mmol, 94.69% yield, HCl) as a yellow solid. ¹H NMR (MeOD, 400 MHz) δ 8.62 (s, 1H), 7.96 (s, 1H), 7.61 (s, 1H), 4.36 (q, J=7.2 Hz, 2H), 3.65 (s, 2H), 1.38 (t, J=7.2 Hz, 3H). LC/MS [M+H] 310.01 (calculated); LC/MS [M+H] 310.1 (observed).

### Preparation of 2-amino-8-bromo-3H-pyrido[4,3-b]azepine-4-carboxylic acid, 3f

To a solution of 3e (2 g, 5.77 mmol, 1 eq, HCl) in THE (15 mL) and water (5 mL) was added lithium hydroxide hydrate, LiOH·H2O (484 mg, 11.5 mmol, 2 eq), and then stirred at 45° C. for 1 hr. The mixture was quenched with 1N HCl until pH=2~3, and extracted with DCM/i-prOH (10 mL×3, 3/1). The organic layers were washed with brine (10 mL×2) dried over Na₂SO₄ filtered and concentrated under reduced pressure to give 3f (800 mg, 2.84 mmol, 49.15% yield) as a white solid. LC/MS [M+H] 282.0 (calculated); LC/MS [M+H] 282.1 (observed).

### Preparation of tert-butyl (2-((2-amino-8-bromo-N-propyl-3H-pyrido[4,3-b]azepine-4-carboxamido)oxy)ethyl)carbamate, 3g

To a solution of 3f (800 mg, 2.84 mmol, 1 eq) in DCM (8 mL) and DMA (3 mL) were added methanesulfonic acid (273 mg, 2.84 mmol, 202 μL, 1 eq), tert-butyl N-[2-(propylaminooxy)ethyl]carbamate (743 mg, 3.40 mmol, 1.2 eq) and EDCI (2.17 g, 11.3 mmol, 4 eq), the mixture stirred at 25° C. for 1 hr. The reaction mixture was diluted with water (20 mL), and extracted with EtOAc (20 mL×3). The combined organic layers were washed with brine (15 mL×2), dried over Na₂SO₄, filtered and concentrated under reduced pressure to give a residue. The residue was purified by flash silica gel chromatography (ISCO®; 20 g SepaFlash® Silica Flash Column, Eluent of 0-100% Ethyl acetate/Petroleum ether gradient at 45 mL/min) to afford 3g (800 mg, 1.66 mmol, 58.48% yield) as brown oil. LC/MS [M+H] 482.1 (calculated); LC/MS [M+H] 482.2 (observed).

### Preparation of methyl 2-amino-4-[2-(tert-butoxycarbonylamino)ethoxy-propyl-carbamoyl]-3H-pyrido[4,3-b]azepine-8-carboxylate, azaBz-4

A mixture of 3g (450 mg, 933 umol, 1 eq), Pd(dppf)Cl₂ (68.3 mg, 93.3 umol, 0.1 eq) and Et₃N (283 mg, 2.80 mmol, 390 μL, 3 eq) in MeOH (15 mL) was degassed and purged with CO for 3 times, and then stirred at 80° C. for 16 hr (50 PSI). The mixture was filtered and concentrated under reduced pressure. The residue was purified by prep-HPLC (neutral condition column: Waters Xbridge Prep OBD C18 150*40 mm*10 um; mobile phase: [water(NH₄HCO₃)-ACN];B %: 10%-40%,8 min) to afford azaBz-4 (100 mg, 216.68 umol, 23.23% yield) as a white solid. ¹H NMR (MeOD, 400 MHz) δ 8.57 (s, 1H), 7.80 (s, 1H), 7.36 (s, 1H), 3.96 (s, 3H), 3.91 (t, J=5.2 Hz, 2H), 3.72 (t, J=7.2 Hz, 2H), 3.23 (t, J=5.2 Hz, 2H), 3.00 (s, 2H), 1.76 (sxt, J=7.2 Hz, 2H), 1.32 (s, 9H), 0.98 (t, J=7.2 Hz, 3H). LC/MS [M+H] 462.2 (calculated); LC/MS [M+H] 462.3 (observed).

### Preparation of azaBz-3

To a solution of azaBz-4 (200 mg, 433 umol, 1 eq) in THE (1 mL) and Water (0.3 mL) was added LiOH·H2O (21.8 mg, 520 umol, 1.2 eq), and then stirred at 25° C. for 1 hr. The mixture was filtered and purified by prep-HPLC (neutral condition column: Waters Xbridge Prep OBD C18 150*40 mm*10 um; mobile phase: [water(NH4HCO3)-ACN];B %: 5%-35%,8 min) to afford azaBz-3 (20 mg, 44.69 umol, 10.31% yield) as a white solid. ¹H NMR (MeOD, 400 MHz) δ 8.57 (s, 1H), 7.80 (s, 1H), 7.37 (s, 1H), 3.90 (t, J=5.2 Hz, 2H), 3.73 (t, J=7.2 Hz, 2H), 3.23 (t, J=5.2 Hz, 2H), 3.10 (s, 1H), 1.82-1.71 (m, 2H), 1.33 (s, 9H), 0.98 (t, J=7.2 Hz, 3H). LC/MS [M+H] 448.2 (calculated); LC/MS [M+H] 448.2 (observed).

### Example 6 Synthesis of 2-amino-8-(2-(aminomethyl)pyrimidin-5-yl)-N-ethoxy-N-propyl-3H-pyrido[4,3-b]azepine-4-carboxamide, azaBz-6

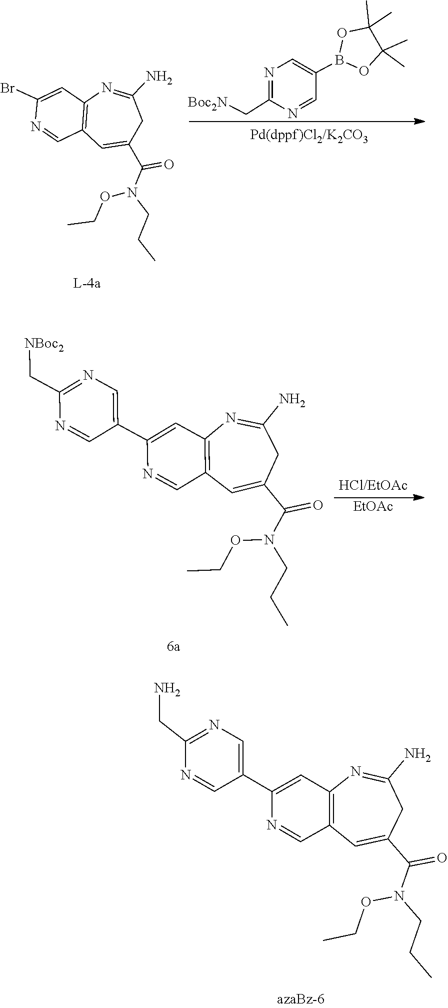

### Preparation of tert-butyl N-[[5-[2-amino-4-[ethoxy(propyl)carbamoyl]-3H-pyrido[4,3-b]azepin-8-yl]pyrimidin-2-yl]methyl]-N-tertbutoxycarbonyl-carbamate, 6a

A mixture of 2-amino-8-bromo-N-ethoxy-N-propyl-3H-pyrido[4,3-b]azepine-4-carboxamide, L-4a (200 mg, 545 umol, 1 eq), tert-Butyl N-tert-butoxycarbonyl-N-[[5-(4,4,5,5-tetramethyl-1,3,2-dioxaborolan-2-yl)pyrimidin-2-yl]methyl]carbamate (285 mg, 654 umol, 1.2 eq), K₂CO₃ (151 mg, 1.09 mmol, 2 eq), and Pd(dppf)Cl₂ (19.9 mg, 27.23 umol, 0.05 eq) in dioxane (6 mL) and H₂O (1.2 mL) was degassed and purged with N₂ for 3 times, and then the mixture was stirred at 95° C. for 2 hr under N₂ atmosphere. The reaction mixture was poured into H₂O (10 mL), and extracted with EtOAc (10 mL×3). The combined organic layers were washed with brine (10 mL), dried over Na₂SO₄, filtered and concentrated under reduced pressure to give a residue. The residue was purified by column chromatography (SiO2, Petroleum ether/Ethyl acetate=I1/0 to 0/1) to afford 6a (320 mg, 537 umol, 98.6% yield) as a yellow solid. LC/MS [M+H] 596.3 (calculated); LC/MS [M+H] 596.4 (observed).

### Preparation of azaBz-6

To a solution of 6a (600 mg, 1.01 mmol, 1 eq) in EtOAc (12 mL) was added HCl/EtOAc (20 mL). The mixture was stirred at 25° C. for 2 hr. The reaction mixture was concentrated under reduced pressure to give a residue, the residue was purified by prep-HPLC column: Phenomenex Luna 80*30 mm*3 um; mobile phase: [water(TFA)-ACN];B %: 1%-20%,8 min to afford azaBz-6 (350 mg, 885 umol, 87.87% yield TFA) as yellow solid. ¹H NMR (MeOD, 400 MHz) δ 9.51 (s, 2H), 8.96 (s, 1H), 8.10 (s, 1H), 7.52 (s, 1H), 4.50 (s, 2H), 3.97 (q, J=7.2 Hz, 2H), 3.76 (t, J=7.2 Hz, 2H), 3.57 (s, 2H), 1.83-1.70 (m, 2H), 1.21 (t, J=7.2 Hz. 3H), 1.00 (t, J=7.2 Hz, 3H). LC/MS [M+H] 396.2 (calculated); LC/MS [M+H] 396.0 (observed).

### Example L-1 Synthesis of 2-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]ethyl N-[2-[(2-amino-3H-pyrido[3,4-b]azepine-4-carbonyl)-propyl-amino]oxyethyl]carbamate, azaBzL-1

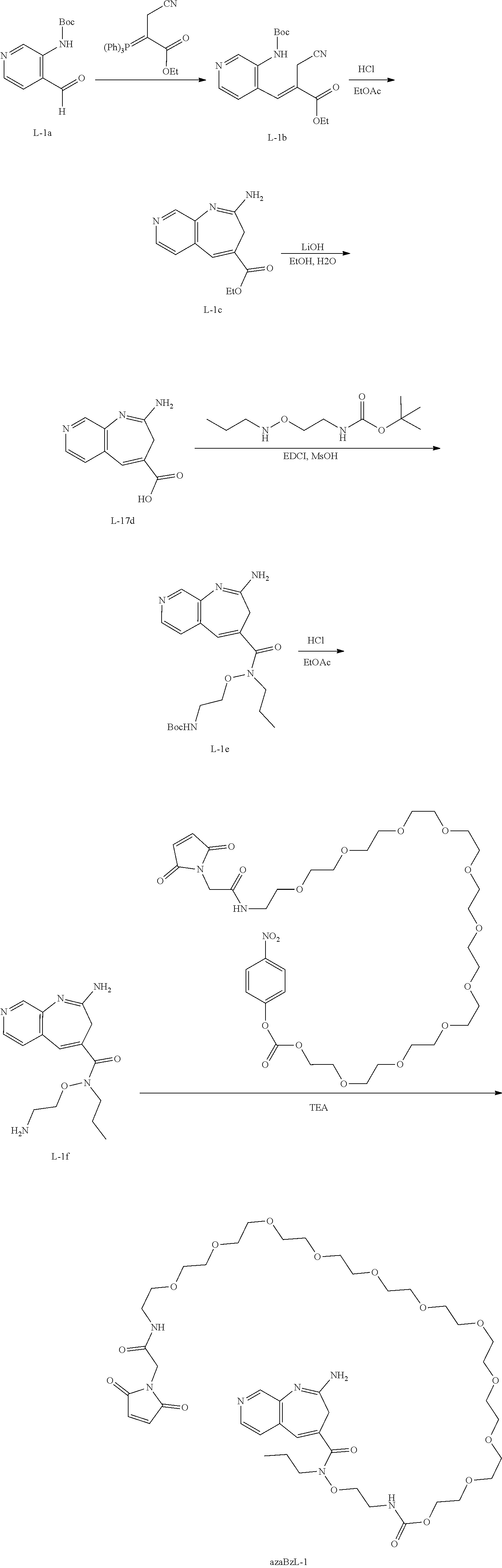

### Preparation of ethyl (E)-3-[3-(tert-butoxycarbonylamino)-4-pyridyl]-2-(cyanomethyl) prop-2-enoate, L-1b

To a solution of tert-butyl N-(4-formyl-3-pyridyl)carbamate, L-1a (500 mg, 2.25 mmol, 1.0 eq) in THE (5 mL) was added ethyl 3-cyano-2-(triphenyl-λ⁵-phosphanylidene)propanoate (871 mg, 2.25 mmol, 1.0 eq). The mixture was stirred at 55° C. for 1 h. The reaction mixture was quenched by addition of H₂O (5 mL), and then extracted with EtOAc (20 mL×3). The combined organic layers were washed with brine (5 mL×3), dried over Na₂SO₄, filtered and concentrated under reduced pressure to give a residue. The residue was purified by column chromatography (SiO₂, Petroleum ether/Ethyl acetate=1/0 to 0/1) to give L-1b (2.8 g, crude) as yellow oil. LC/MS [M+H] 332.2 (calculated); LC/MS [M+H] 332.0 (observed).

### Preparation of ethyl 2-amino-3H-pyrido[3,4-b]azepine-4-carboxylate, L-1c

To a solution of L-1b (2.6 g, 7.85 mmol, 1.0 eq) in EtOAc (1 mL) was added HCl/EtOAc (4 M, 20 mL, 10.2 eq). The mixture was stirred at 25° C. for 12 h, then was stirred at 50° C. for another 2 h. The reaction mixture was filtered. Then the filter cake was dried under reduced pressure to give a residue. Compound L-1c (980 mg, 4.24 mmol, 54.0% yield) was obtained as yellow oil. ¹H NMR (400 MHz, DMSO-d₆) δ10.27 (s, 1H), 9.27 (s, 1H), 8.72 (s, 1H), 8.55 (d, J=5.2 Hz, 1H), 7.88 (s, 1H), 7.74 (d, J=5.2 Hz, 1H), 4.29 (q, J=7.2 Hz, 2H), 3.57 (s, 2H), 1.32 (t, J=7.2 Hz, 3H). LC/MS [M+H] 232.1 (calculated); LC/MS [M+H] 232.2 (observed).

### Preparation of 2-amino-3H-pyrido[3,4-b]azepine-4-carboxylic acid, L-1d

To a solution of L-1c (480 mg, 2.08 mmol, 1.0 eq) in H₂O (1 mL) and EtOH (5 mL) was added LiOH (149 mg, 6.23 mmol, 3.0 eq). The mixture was stirred at 50° C. for 2 h. The reaction mixture was quenched with 2.5N HCl at 0° C. until pH=5-6, the result mixture was concentrated under reduced pressure to remove EtOH. The solid was filtered and the filter cake was dried under reduced pressure to give a residue, L-1d (250 mg, 1.23 mmol, 59.2% yield) obtained as a white solid. ¹H NMR (400 MHz, DMSO-d₆) δ8.68 (s, 1H), 8.56 (d, J=5.6 Hz, 1H), 7.86 (s, 1H), 7.79 (d, J=5.6 Hz, 1H). LC/MS [M+H] 204.1 (calculated); LC/MS [M+H] 204.0 (observed).

### Preparation of tert-butyl N-[2-[(2-amino-3H-pyrido[3,4-b]azepine-4-carbonyl)-propyl-amino]oxyethyl]carbamate, L-1e

To a solution of L-1d (250 mg, 1.23 mmol, 1.0 eq) and tert-butyl N-[2-(propylaminooxy)ethyl]carbamate (537 mg, 2.46 mmol, 2.0 eq) in DCM (7.5 mL) and DMA (1.5 mL) was added MsOH (236 mg, 2.46 mmol, 175 μL, 2.0 eq) and EDCI (943 mg, 4.92 mmol, 4.0 eq). The mixture was stirred at 25° C. for 2 h. The mixture was concentrated to remove DCM and the residue was diluted with water (30 mL). Then the pH of the aqueous phase was adjusted to 8-9 with aq·Na₂CO₃ at 0° C. Then extracted with EtOAc (10 ml×3). The organic layer was washed with brine, dried over Na₂SO₄, filtered and concentrated. The residue was purified by column chromatography (SiO₂, Petroleum ether/Ethyl acetate=I1/0 to 3/1) to give L-1e (420 mg, 1.04 mmol, 84.6% yield) as a white solid. ¹H NMR (400 MHz, MeOD) δ8.37 (s, 1H), 8.08 (d, J=5.2 Hz, 1H), 7.37 (d, J=5.4 Hz, 1H), 7.20 (s, 1H), 4.61 (s, 1H), 3.91 (t, J=5.2 Hz, 2H), 3.72 (t, J=7.2 Hz, 2H), 3.23 (t, J=5.2 Hz, 2H), 2.96 (s, 2H), 1.76 (sxt, J=7.6 Hz, 2H), 1.34 (s, 9H), 0.98 (t, J=7.6 Hz, 3H). LC/MS [M+H] 404.2 (calculated); LC/MS [M+H] 404.2 (observed).

### Preparation of 2-amino-N-(2-aminoethoxy)-N-propyl-3H-pyrido[3,4-b]azepine-4-carboxamide, L-1f

To a solution of L-1e (55 mg, 136 umol, 1.0 eq) in EtOAc (1 mL) was added HCl/EtOAc (4 M, 10 mL, 293.0 eq), and then stirred at 25° C. for 1 h. The reaction mixture was concentrated under reduced pressure to give a residue, L-1f (51.2 mg, crude, 2HCl) obtained as a white solid. LC/MS [M+H] 304.2 (calculated); LC/MS [M+H] 304.2 (observed).

### Preparation of azaBzL-1

To a solution of L-1f (50 mg, 94.1 umol, 1.0 eq, 2TFA) in DMF (1 mL) was added DIEA (60.8 mg, 470 umol, 81.9 uL 5.0 eq) and 2-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]ethyl (4-nitrophenyl) carbonate (75.6 mg, 94.1 umol, 1.0 eq), and then stirred at 25° C. for 1 h. The mixture was quenched with TFA until pH=~6. Then the mixture was filtered purified by prep-HPLC (column: Phenomenex Luna 80*30 mm*3 um; mobile phase: [water(TFA)-ACN];B %: 5%-35%,8 min) to give azaBzL-1 (40 mg, 41.3 umol, 43.9% yield) as yellow oil. ¹H NMR (400 MHz, MeOD-d₄) δ8.68 (s, 1H), 8.54 (d, J=5.6 Hz, 1H), 7.63 (d, J=5.2 Hz, 1H), 7.38 (s, 1H), 6.89 (s, 2H), 4.17 (s, 2H), 3.97 (t, J=4.8 Hz, 2H), 3.89-3.81 (m, 2H), 3.75 (t, J=7.2 Hz, 2H), 3.71-3.57 (m, 38H), 3.54 (t, J=5.6 Hz, 2H), 3.52-3.48 (m, 2H), 3.45 (s, 2H), 3.38 (q, J=5.2 Hz, 2H), 1.85-1.73 (m, 2H), 1.00 (t, J=7.6 Hz, 3H). LC/MS [M+H] 968.5 (calculated); LC/MS [M+H] 968.5 (observed).

### Example L-3 Synthesis of 2-amino-4-(((40-(2,5-dioxo-2,5-dihydro-1H-pyrrol-1-yl)-4,39-dioxo-5,8,11,14,17,20,23,26,29,32,35-undecaoxa-3,38-diazatetracontyl)oxy)(propyl)carbamoyl)-3H-pyrido[4,3-b]azepine-8-sulfonic acid, azaBzL-3

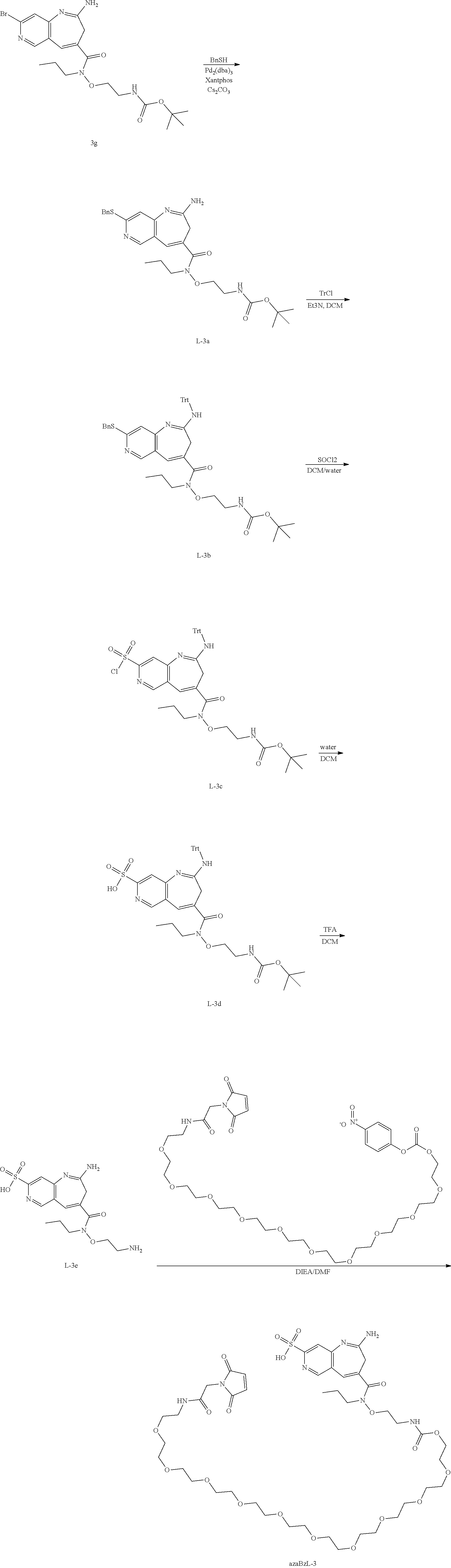

### Preparation of tert-butyl (2-((2-amino-8-(benzylthio)-N-propyl-3H-pyrido[4,3-b]azepine-4-carboxamido)oxy)ethyl)carbamate, L-3a

A mixture of tert-butyl N-[2-[(2-amino-8-bromo-3H-pyrido[4,3-b]azepine-4-carbonyl)-propyl-amino]oxyethyl]carbamate, 3g (400 mg, 829 umol, 1 eq), phenylmethanethiol (680 mg, 5.47 mmol, 642 μL, 6.60 eq), (1E,4E)-1,5-diphenylpenta-1,4-dien-3-one; palladium, Pd₂(dba)₃ (75.9 mg, 82.9 umol, 0.1 eq), diisopropylethylamine, DIEA (214 mg, 1.66 mmol, 289 μL, 2 eq) and (5-diphenylphosphanyl-9,9-dimethyl-xanthen-4-yl)-diphenyl-phosphane, Xantphos (96.0 mg, 166 umol, 0.2 eq) in dioxane (5 mL) was degassed and purged with N₂ for 3 times, and then stirred at 110° C. for 2 h under N₂ atmosphere. The mixture was filtered and the filtration was concentrated under reduced pressure. The residue was diluted with H₂O 50 mL at 0° C., and then extracted with EtOAc (60 mL×3). The combined organic layers were washed with brine (50 mL), dried over Na₂SO₄, filtered and concentrated under reduced pressure to give a residue. The residue was purified by flash silica gel chromatography (ISCO®; 80 g SepaFlash® Silica Flash Column, Eluent of 0~100%0 Ethyl acetate/Petroleum ether gradient at 80 mL/min) to afford L-3a (350 mg, 665.83 umol, 80.2900 yield) as a yellow solid. ¹H NMR (MeOD, 400 MHz) δ 8.37 (s, 1H), 7.41 (d, J=7.2 Hz, 2H), 7.32-7.25 (m, 3H), 7.24-7.19 (m, 1H), 6.92 (s, 1H), 4.37 (s, 2H), 3.89 (t, J=5.2 Hz, 2H), 3.71 (t, J=7.2 Hz, 2H), 3.23 (t, J=5.2 Hz, 2H), 3.00-2.93 (m, 2H), 1.75 (m, 2H), 1.33 (s, 9H), 0.97 (t, J=7.6 Hz, 3H). LC/MS [M+H] 526.2 (calculated); LC/MS [M+H] 526.3 (observed).

### Preparation of tert-butyl (2-((2-amino-8-(benzylthio)-N-propyl-3H-pyrido[4,3-b]azepine-4-carboxamido)oxy)ethyl)carbamate, L-3b

To a solution of L-3a (300 mg, 571 umol, 1 eq) in DCM (10 mL) were added TrtCl (318 mg, 1.14 mmol, 2 eq) and Et₃N (231 mg, 2.28 mmol, 318 μL, 4 eq) at 25° C., and then stirred at 50° C. for 16 hr. The reaction mixture was diluted with water 20 mL and extracted with DCM (50 mL*3). The combined organic layers were washed with brine (20 mL), dried over Na₂SO₄, filtered and concentrated under reduced pressure to give a residue to afford the compound L-3b (350 mg, 456 umol, 79.86% yield) as a yellow solid. LC/MS [M+H] 768.4 (calculated); LC/MS [M+H] 768.3 (observed).

### Preparation of tert-butyl (2-((8-(chlorosulfonyl)-N-propyl-2-(tritylamino)-3H-pyrido[4,3-b]azepine-4-carboxamido)oxy)ethyl)carbamate, L-3c

To a solution of L-3b (300 mg, 391 umol, 1 eq) in DCM (5 mL) and water (2 mL) was added sulfuryl chloride (211 mg, 1.56 mmol, 156 μL, 4 eq), and then stirred at 0° C. for 1 hr. The reaction mixture was quenched by addition saturated NaHCO₃ 10 mL at 0° C., extracted with DCM (15 mL×3). The combined organic layers were washed with brined (10 mL), dried over Na₂SO₄, filtered and concentrated under reduced pressure to give a residue. The crude product L-3c (300 mg, crude) as yellow solid was used into the next step without further purification. LC/MS [M+H] 744.2 (calculated); LC/MS [M+H] 744.3 (observed).

### Preparation of 4-((2-((tert-butoxycarbonyl)amino)ethoxy)(propyl)carbamoyl)-2-(tritylamino)-3H-pyrido[4,3-b]azepine-8-sulfonic acid, L-3d

A solution of L-3c (300 mg, 403 umol, 1 eq) in water (3 mL) and MeCN (5 mL) was stirred at 100° C. for 0.5 hr. The mixture was concentrated under reduced pressure. The crude L-1d (270 mg, 372 umol, 92.23% yield) as yellow solid was used into the next step without further purification. LC/MS [M+H] 726.3 (calculated); LC/MS [M+H] 726.3 (observed).

### Preparation of 2-amino-4-[2-aminoethoxy(propyl)carbamoyl]-3H-pyrido[4,3-b]azepine-8-sulfonic acid, L-3e

A solution of L-3d (200 mg, 276 umol, 1 eq) in DCM (3 mL) and TFA (1 mL) was stirred at 25° C. for 4 hr. The mixture was concentrated under reduced pressure. The residue was diluted with water (10 mL) and extracted with MTBE (5 mL) to remove the excess TFA, the aqueous layer was concentrated to give a crude product. The crude product L-3e (100 mg, crude) as yellow solid was used into the next step without further purification. LC/MS [M+H] 384.1 (calculated); LC/MS [M+H] 384.2 (observed).

### Preparation of azaBzL-3

To a solution of L-3e (50 mg, 130 umol, 1 eq TFA) and DIEA (67.4 mg, 522 umol, 90.9 uL, 4 eq) in DMF (0.5 mL) was added 2-[2-[2-[2-[2-[2-[2-[2-[2-[2-[2-[[2-(2,5-dioxopyrrol-1 -yl)acetyl]amino]ethoxy]ethoxy]ethoxy]ethoxy]ethoxy]ethoxy]ethoxy]ethoxy]ethoxy]ethoxy]ethyl (4-nitrophenyl) carbonate (105 mg, 130 umol, 1 eq) at 0° C., and then stirred at 0° C. for 0.5 hr. The mixture was filtered and purified by prep-HPLC (TFA condition; column: Phenomenex Luna 80*30 mm*3 um; mobile phase: [water(TFA)-ACN];B %: 5%-25%,8 min) to afford azaBzL-3 (15 mg, 14.31 umol, 10.97% yield) as a white solid. ¹H NMR (MeOD, 400 MHz) δ 8.79 (s, 1H), 7.90 (s, 1H), 7.51 (s, 1H), 6.91 (s, 2H), 4.19 (s, 2H), 3.99 (br t, J=4.6 Hz, 2H), 3.87 (br d, J=4.0 Hz, 2H), 3.77 (br t, J=7.2 Hz, 2H), 3.67-3.61 (m, 38H), 3.58-3.48 (m, 6H), 3.42-3.38 (m, 2H), 1.83-1.75 (m, 2H), 1.05-1.00 (m, 3H). LC/MS [M+H] 1048.4 (calculated); LC/MS [M+H] 1048.5 (observed).

### Example L-4 Synthesis of 2-amino-8-(2-(38-(2,5-dioxo-2,5-dihydro-1H-pyrrol-1-yl)-3,37-dioxo-6,9,12,15,18,21,24,27,30,33-decaoxa-2,36-diazaoctatriacontyl)pyrimidin-5-yl)-N-ethoxy-N-propyl-3H-pyrido[4,3-b]azepine-4-carboxamide, azaBzL-4

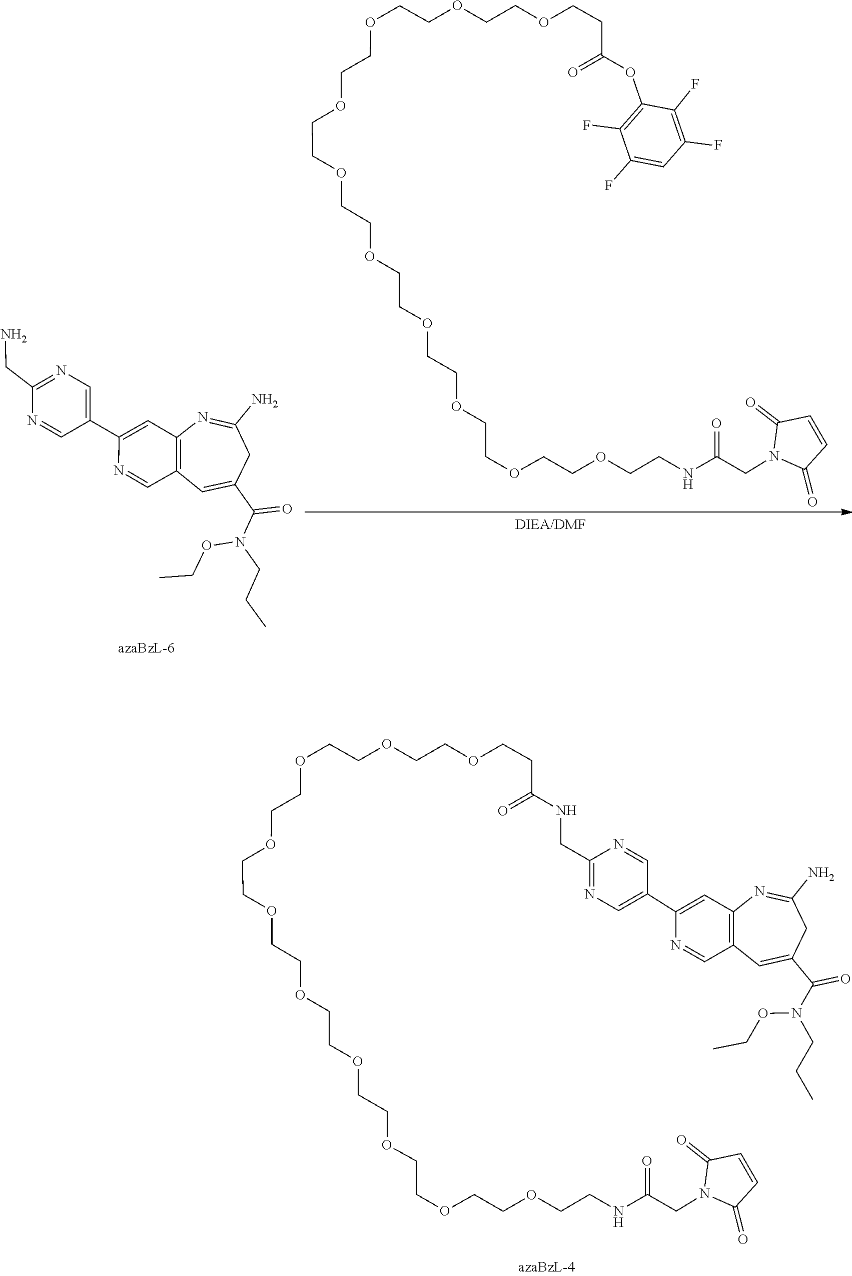 [SMILES: CCCN(OCC)C(=O)C1=Cc2cnc(-c3cnc(CN)nc3)cc2N=C(N)C1 ; O=C(CN1C(=O)C=CC1=O)NCCOCCOCCOCCOCCOCCOCCOCCOCCOCCOCCC(=O)Oc1c(F)c(F)cc(F)c1F ; CCCN(OCC)C(=O)C1=Cc2cnc(-c3cnc(CNC(=O)CCOCCOCCOCCOCCOCCOCCOCCOCCOCCOCCNC(=O)CN4C(=O)C=CC4=O)nc3)cc2N=C(N)C1]

To a mixture of 2-amino-8-[2-(aminomethyl)pyrimidin-5-yl]-N-ethoxy-N-propyl-3H-pyrido[4,3-b]azepine-4-carboxamide, azaBz-6 (100 mg, 196 umol, 1 eq, TFA) and DIEA (101 mg, 785 umol, 137 μL, 4 eq) in DMF (1 mL) was added (2,3,5,6-tetrafluorophenyl) 3-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]propanoate (320 mg, 393 umol, 2 eq), and the mixture was stirred at 25° C. for 2 hr under N₂ atmosphere. The reaction mixture was filtered and purified by prep-HPLC (Water-ACN condition). column: Waters Xbridge BEH C18 100*25 mm*5 um; mobile phase: [Water-ACN];B %: 5%-35%,20 min to afford azaBzL-4 (33.3 mg, 31.89 umol, 16.25% yield) as a yellow solid. ¹H NMR (MeOD, 400 MHz) δ 9.36 (s, 2H), 8.59 (s, 1H), 7.56 (s, 1H), 7.32 (s, 1H), 6.87 (s, 1H), 4.67 (s, 2H), 4.15 (s, 2H), 3.95-3.85 (m, 2H), 3.75-3.65 (m, 4H), 3.64-3.49 (m, 38H), 3.35-3.32 (m, 2H), 3.02 (s, 2H), 2.62-2.55 (m, 2H), 1.77-1.71 (m, 2H), 1.21-1.12 (m, 3H), 1.02-0.95 (m, 3H). LC/MS [M+H] 1044.3 (calculated); LC/MS [M+H] 1044.5 (observed).

### Example L-5 Synthesis of 2-amino-N8-(1-(2,5-dioxo-2,5-dihydro-1H-pyrrol-1-yl)-2-oxo-6,9,12,15,18,21,24,27,30,33-decaoxa-3-azapentatriacontan-35-yl)-N4-ethoxy-N4-propyl-3H-pyrido[4,3-b]azepine-4,8-dicarboxamide, azaBzL-5

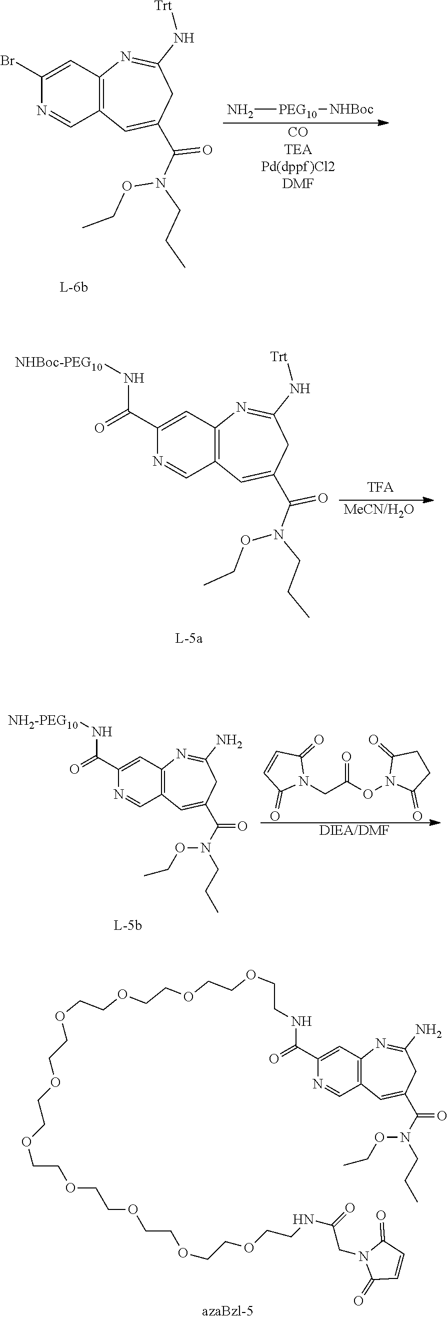

### Preparation of tert-butyl (1-(4-(ethoxy(propyl)carbamoyl)-2-(tritylamino)-3H-pyrido[4,3-b]azepin-8-yl)-1-oxo-5,8,11,14,17,20,23,26,29,32-decaoxa-2-azatetratriacontan-34-yl)carbamate, L-5a

A mixture of tert-butyl N-[2-[2-[2-[2-[2-[2-[2-[2-[2-[2-(2-aminoethoxy)ethoxy]ethoxy]ethoxy]ethoxy]ethoxy]ethoxy]ethoxy]ethoxy]ethoxy]ethyl]carbamate, NH₂-PEG₁₀-NHBoc (355 mg, 591 umol, 1.2 eq), 8-bromo-N-ethoxy-N-propyl-2-(tritylamino)-3H-pyrido[4,3-b]azepine-4-carboxamide, L-6b (300 mg, 492.16 umol, 1 eq), triethylamine, TEA (249 mg, 2.46 mmol, 343 μL, 5 eq), Pd(dppf)Cl₂ (72.02 mg, 98.4 umol, 0.2 eq) in DMF (8 mL) was degassed and purged with carbon monoxide, CO for 3 times, and then stirred at 80° C. for 16 hr under CO (50Psi) atmosphere. The reaction mixture was diluted with water (100 mL) and extracted with EtOAc (100 mL×3). The combined organic layers were washed with brine (100 mL), dried over Na₂SO₄, filtered and concentrated under reduced pressure to give a residue. The residue was purified by column chromatography (SiO2, Petroleum ether/Ethyl acetate=0/1 to 1/1) to afford L-5a (530 mg, 458 umol, 93.04% yield) was obtained as a yellow oil. LC/MS [M+H] 1157.6 (calculated); LC/MS [M+H] 1157.6 (observed).

### Preparation of 2-amino-N8-(32-amino-3,6,9,12,15,18,21,24,27,30-decaoxadotriacontyl)-N4-ethoxy-N4-propyl-3H-pyrido[4,3-b]azepine-4,8-dicarboxamide, L-5b

A mixture of L-5a (50 mg, 43.2 umol, 1 eq) and TFA (98.5 mg, 864 umol, 63.9 uL, 20 eq) in DCM (1 mL) was degassed and purged with N₂ for 3 times, and then stirred at 40° C. for 16 hr under N₂ atmosphere. The reaction mixture was concentrated under reduced pressure to give L-5b (40 mg, crude), used in the next step without further purification as yellow oil. LC/MS [M+H] 815.5 (calculated); LC/MS [M+H] 815.4 (observed).

### Preparation of azaBzL-5

To a solution of L-5b (35 mg, 37.68 umol, 1 eq, TFA) and 2,5-dioxopyrrolidin-1-yl 2-(2,5-dioxo-2,5-dihydro-1H-pyrrol-1-yl)acetate (9.50 mg, 37.7 umol, 1 eq) in DMF (0.2 mL) was added DIEA (19.5 mg, 151 umol, 26.3 uL, 4 eq), and then stirred at 25° C. for 1 hr. The reaction mixture was filtered and purified by prep-HPLC (TFA condition). column: Phenomenex Luna 80*30 mm*3 um; mobile phase: [water(TFA)-ACN];B %: 10%-40%,8 min. to give azaBzL-5 (20 mg, 21.01 umol, 55.76% yield) as a white solid. ¹H NMR (MeOD, 400 MHz) δ 8.79 (s, 1H), 8.01 (s, 1H), 7.50 (s, 1H), 6.87 (s, 2H) 4.14 (s, 2H), 3.92-3.98 (m, 2H), 3.77-3.72 (m, 2H), 3.7-3.65 (m, 2H), 3.63-3.57 (m, 38H), 3.55-3.48 (m, 2H), 3.47-3.44 (m, 2H), 3.41-3.32 (m, 2H), 1.78-1.72 (m, 2H), 1.18 (t, J=7.2 Hz, 3H), 0.98 (t, J=7.2 Hz, 3H). LC/MS [M+H] 952.5 (calculated); LC/MS [M+H] 952.5 (observed).

### Example L-6 Synthesis of 2-amino-8-(N-(1-(2,5-dioxo-2,5-dihydro-1H-pyrrol-1-yl)-2-oxo-6,9,12,15,18,21,24,27,30,33-decaoxa-3-azapentatriacontan-35-yl)sulfamoyl)-N-ethoxy-N-propyl-3H-pyrido[4,3-b]azepine-4-carboxamide, azaBzL-6

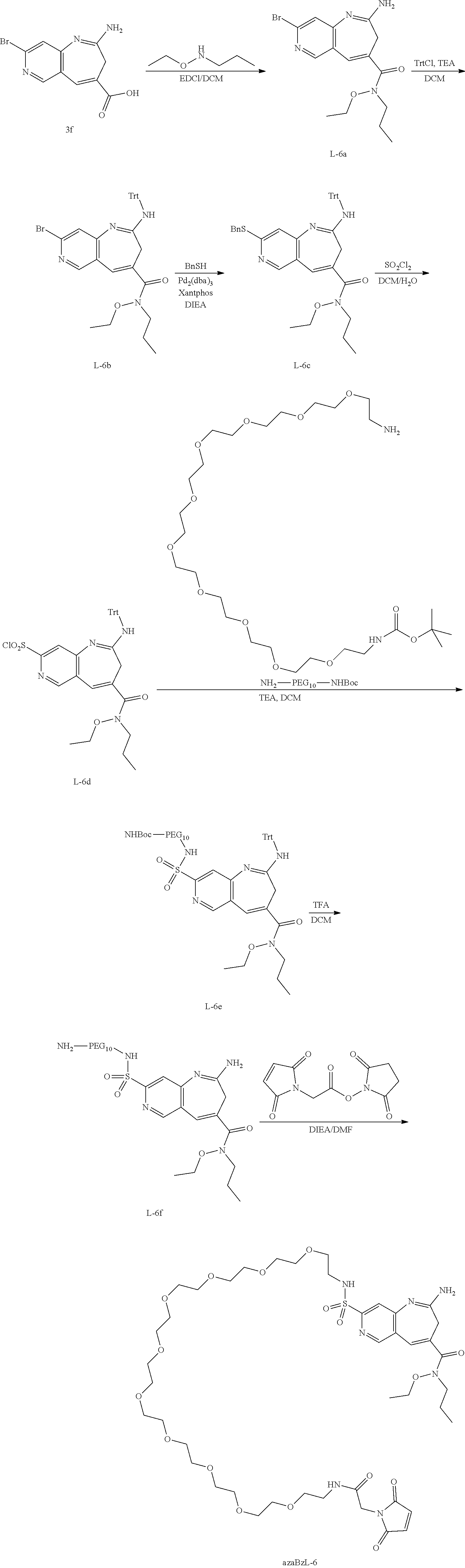

### Preparation of 2-amino-8-bromo-N-ethoxy-N-propyl-3H-pyrido[4,3-b]azepine-4-carboxamide, L-6a

To a solution of 2-amino-8-bromo-3H-pyrido[4,3-b]azepine-4-carboxylic acid, 3f (3 g, 10.6 mmol, 1 eq) in DCM (40 mL) and DMA (10 mL) were added methanesulfonic acid (1.02 g, 10.6 mmol, 0.757 mL, 1 eq), N-ethoxypropan-1-amine (1.29 g, 9.25 mmol, 0.9 eq, HCl) and 1-ethyl-3-(3-dimethylaminopropyl)carbodiimide hydrochloride, EDCI, CAS Reg. No. 1892-57-5 (8.15 g, 42.5 mmol, 4 eq), the mixture was stirred at 25° C. for 1 hr. The reaction mixture was diluted with water (80 mL) and extracted with DCM (50 mL×3). The combined organic layers were washed with brine (20 mL), dried over Na₂SO₄, filtered and concentrated under reduced pressure to give a residue. The crude product was triturated with MTBE (10 mL) at 25° C. for 10 min to afford L-6a (3.2 g, 8.71 mmol, 81.9% yield) as a white solid. LC/MS [M+H] 367.1 (calculated); LC/MS [M+H] 367.0 (observed).

### Preparation of 8-bromo-N-ethoxy-N-propyl-2-(tritylamino)-3H-pyrido[4,3-b]azepine-4-carboxamide, L-6b

To a solution of L-6a (700 mg, 1.91 mmol, 1 eq) in DCM (20 mL) were added triphenylmethylchloride, trityl chloride, TrtCl (1.33 g, 4.77 mmol, 2.5 eq) and triethylamine, Et₃N (964 mg, 9.53 mmol, 1.33 mL, 5 eq), and then stirred at 50° C. for 16 hr. The reaction mixture was diluted with water (20 mL) and extracted with DCM (20 mL×3). The combined organic layers were washed with brine (20 mL), dried over Na₂SO₄, filtered and concentrated under reduced pressure to give a residue. The residue was purified by flash silica gel chromatography (ISCO®; 40 g SepaFlash® Silica Flash Column, Eluent of 0~60% Ethyl acetate/Petroleum ether gradient @50 mL/min) to afford L-6b (1 g, 1.64 mmol, 86.07% yield) as a white solid. LC/MS [M+H] 609.2 (calculated); LC/MS [M+H] 609.0 (observed).

### Preparation of 8-(benzylthio)-N-ethoxy-N-propyl-2-(tritylamino)-3H-pyrido[4,3-b]azepine-4-carboxamide, L-6c

To a solution of L-6b (600 mg, 0.986 mmol, 1 eq) and phenylmethanethiol, BnSH (306 mg, 2.46 mmol, 0.289 mL, 2.5 eq) in dioxane (30 mL) were added (1E,4E)-1,5-diphenylpenta-1,4-dien-3-one; palladium, Pd₂(dba)₃ (90.3 mg, 98.6 umol, 0.1 eq), DIEA (255 mg, 1.97 mmol, 0.343 mL, 2 eq) and (5-diphenylphosphanyl-9,9-dimethyl-xanthen-4-yl)-diphenyl-phosphane, Xantphos, CAS Reg. No. 161265-03-8 (114 mg, 0.197 mmol, 0.2 eq), the mixture was stirred at 110° C. for 2 hr under N₂. The reaction mixture was quenched by addition of water (50 mL) at 0° C., and then extracted with EtOAc (50 mL×3). The combined organic layers were washed with brine (30 mL), dried over Na₂SO₄, filtered and concentrated under reduced pressure to give a residue. The residue was purified by flash silica gel chromatography (ISCO®; 40 g SepaFlash® Silica Flash Column, Eluent of 0~80% Ethyl acetate/Petroleum ether gradient @70 mL/min) to afford L-6c (500 mg, 0.767 mmol, 77.8% yield) as a yellow solid. LC/MS [M+H] 653.3 (calculated); LC/MS [M+H] 653.0 (observed).

### Preparation of 4-(ethoxy(propyl)carbamoyl)-2-(tritylamino)-3H-pyrido[4,3-b]azepine-8-sulfonyl chloride, L-6d

To a solution of L-6c (380 mg, 0.582 mmol, 1 eq) in DCM (2 mL) and water (0.3 mL) was added sulfuryl chloride, SO₂Cl₂ (393 mg, 2.91 mmol, 0.291 mL, 5 eq) at 0° C., then stirred at 25° C. for 1 hr. The reaction mixture was quenched by addition saturated aqueous NaHCO₃ (15 mL) at 0° C., and then extracted with DCM (20 mL×3). The combined organic layers were washed with brine (20 mL), dried over Na₂SO₄, filtered and concentrated under reduced pressure to give crude L-6d (400 mg, crude) as yellow oil, used in the next step without further purification. LC/MS [M+H] 629.2 (calculated); LC/MS [M+H] 629.0 (observed).

### Preparation of tert-butyl N-[2-[2-[2-[2-[2-[2-[2-[2-[2-[2-[2-[[4-[ethoxy(propyl)carbamoyl]-2-(tritylamino)-3H-pyrido[4,3-b]azepin-8 -yl]sulfonylamino]ethoxy]ethoxy]ethoxy]ethoxy]ethoxy]ethoxy]ethoxy]ethoxy]ethoxy]ethoxy]ethyl]carbamate, L-6e

To a solution of tert-butyl N-[2-[2-[2-[2-[2-[2-[2-[2-[2-[2-(2-aminoethoxy)ethoxy]ethoxy]ethoxy]ethoxy]ethoxy]ethoxy]ethoxy]ethoxy]ethoxy]ethyl]carbamate, NH₂-PEG₁₀-NHBoc (458 mg, 0.76 mmol, 1.2 eq) and Et₃N (322 mg, 3.18 mmol, 0.442 mL, 5 eq) in DCM (8 mL) was added L-6d (400 mg, 0.64 mmol, 1 eq) at 0° C., then stirred at 25° C. for 0.5 hr. The mixture was filtered and concentrated under reduced pressure. The residue was purified by flash silica gel chromatography (ISCO®; 20 g SepaFlash® Silica Flash Column, Eluent of 0-100% Ethyl acetate/Petroleum ether gradient @50 mL/min) to afford L-6e (500 mg, 0.419 mmol, 65.90% yield) as colorless oil. LC/MS [M+H] 1193.6 (calculated); LC/MS [M+H] 1193.3 (observed).

### Preparation of 2-amino-8-(N-(32-amino-3,6,9,12,15,18,21,24,27,30-decaoxadotriacontyl)sulfamoyl)-N-ethoxy-N-propyl-3H-pyrido[4,3-b]azepine-4-carboxamide, L-6f

To a solution of L-6e (100 mg, 83.8 umol, 1 eq) in DCM (0.3 mL) was added TFA (191 mg, 1.68 mmol, 124 μL, 20 eq), and then stirred at 50° C. for 0.5 hr. The mixture was filtered and concentrated under reduced pressure to give, L-6f (100 mg, crude) as yellow oil, used in the next step without further purification. LC/MS [M+H] 851.4 (calculated); LC/MS [M+H] 851.2 (observed).

### Preparation of azaBzL-6

To a solution of L-6f (100 mg, 104 umol, 1 eq, TFA) in DMF (0.3 mL) were added diisopropylethylamine, DIEA (53.6 mg, 414 umol, 72.2 uL, 4 eq) and (2,5-dioxopyrrolidin-1-yl) 2-(2,5-dioxopyrrol-1-yl)acetate (26.13 mg, 104 umol, 1 eq) at 0° C., then stirred at 25° C. for 0.5 hr. The mixture was filtered and purified by prep-HPLC (TFA condition; column: Phenomenex Luna 80*30 mm*3 um; mobile phase: [water(TFA)-ACN];B %: 1%-30%,8 min) to afford azaBzL-6 (53 mg, 53.6 umol, 51.8% yield) as colorless oil. ¹H NMR (MeOD, 400 MHz) δ 8.86 (s, 1H), 7.94 (s, 1H), 7.52 (s, 1H), 6.89 (s, 2H), 4.17 (s, 2H), 3.98 (q, J=7.2 Hz, 2H), 3.76 (t, J=7.2 Hz, 2H), 3.66-3.59 (m, 32H), 3.56-3.47 (m, 1OH), 3.37 (td, J=4.8, 9.6 Hz, 4H), 1.78 (m, 2H), 1.20 (t, J=7.2 Hz, 3H), 1.00 (t, J=7.6 Hz, 3H). LC/MS [M+H] 988.5 (calculated); LC/MS [M+H] 988.2 (observed).

### Example L-7 Synthesis of 2-amino-8-(N-(1-(2,5-dioxo-2,5-dihydro-1H-pyrrol-1-yl)-2-oxo-6,9,12,15,18,21,24,27,30,33-decaoxa-3-azapentatriacontan-35-yl)-N-methylsulfamoyl)-N-ethoxy-N-propyl-3H-pyrido[4,3-b]azepine-4-carboxamide, azaBzL-7

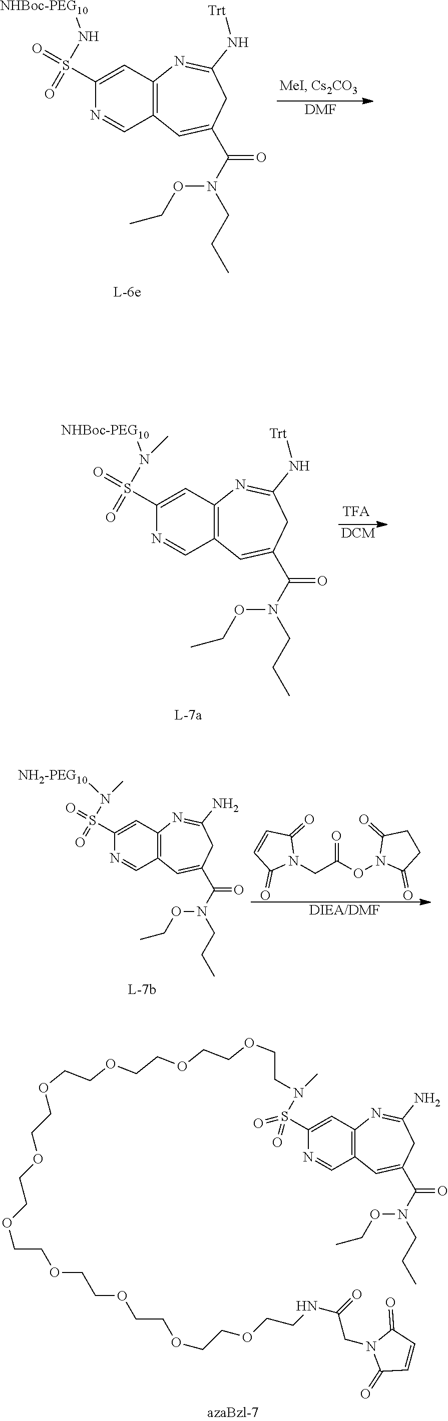

### Preparation of tert-butyl (2-((4-(ethoxy(propyl)carbamoyl)-2-(tritylamino)-3H-pyrido[4,3-b]azepin-8-yl)sulfonyl)-5,8,11,14,17,20,23,26,29,32-decaoxa-2-azatetratriacontan-34-yl)carbamate, L-7a

To a solution of tert-butyl N-[2-[2-[2-[2-[2-[2-[2-[2-[2-[2-[2-[[4-[ethoxy(propyl)carbamoyl]-2-(tritylamino)-3H-pyrido[4,3-b]azepin-8-yl]sulfonylamino]ethoxy]ethoxy]ethoxy]ethoxy]ethoxy]ethoxy]ethoxy]ethoxy]ethoxy]ethoxy]ethyl]carbamate, L-6e (200 mg, 168 umol, 1 eq) in MeCN (1 mL) were added cesium carbonate, Cs₂CO₃ (109 mg, 0.335 mmol, 2 eq) and methyl iodide, Mel (28.5 mg, 201 umol, 12.5 uL, 1.2 eq), the mixture was stirred at 25° C. for 1 hr. The resulting mixture was filtered and concentrated under reduced pressure to afford L-7a (200 mg, crude) as colorless oil. ¹H NMR (MeOD, 400 MHz) δ 8.44 (s, 1H), 7.35-7.31 (m, 7H), 7.25-7.16 (m, 9H), 6.91 (s, 1H), 4.61 (s, 1H), 4.05-3.99 (m, 2H), 3.79 (t, J=6.8 Hz, 2H), 3.64-3.48 (m, 42H), 3.23-3.19 (m, 2H), 3.04 (s, 2H), 2.89 (s, 3H), 1.83-1.77 (m, 2H), 1.43 (s, 9H), 1.24 (t, J=7.2 Hz, 3H), 1.01 (t, J=7.6 Hz, 3H). LC/MS [M+H] 1207.6 (calculated); LC/MS [M+H] 1207.8 (observed).

### Preparation of 2-amino-8-(N-(32-amino-3,6,9,12,15,18,21,24,27,30-decaoxadotriacontyl)-N-methylsulfamoyl)-N-ethoxy-N-propyl-3H-pyrido[4,3-b]azepine-4-carboxamide, L-7b

To a solution of L-7a (140 mg, 116 umol, 1 eq) in DCM (0.3 mL) was added TFA (264 mg, 2.32 mmol, 172 μL, 20 eq), and then stirred at 50° C. for 1 hr. The mixture was filtered and concentrated under reduced pressure to afford L-7b (100 mg, crude) as a yellow solid. LC/MS [M+H] 865.5 (calculated); LC/MS [M+H] 865.5 (observed).

### Preparation of azaBzL-7

To a solution of L-7b (100 mg, 102 umol, 1 eq, TFA) and DIEA (52.8 mg, 409 umol, 71.2 uL, 4 eq) in DMF (0.5 mL) was added (2,5-dioxopyrrolidin-1-yl) 2-(2,5-dioxopyrrol-1-yl)acetate (25.8 mg, 102 umol, 1 eq) at 0° C., then stirred at 25° C. for 0.5 hr. The mixture was filtered and purified by prep-HPLC (TFA condition column: Phenomenex Luna 80*30 mm*3 um; mobile phase: [water(TFA)-ACN];B %: 5%-35%,8 min to afford azaBzL-7 (45 mg, 44.90 umol, 43.96% yield) as yellow oil. ¹H NMR (MeOD, 400 MHz) δ 8.83 (s, 1H), 7.87 (s, 1H), 7.51 (s, 1H), 6.89 (s, 2H), 4.17 (s, 2H), 3.98 (q, J=7.2 Hz, 2H), 3.78-3.73 (m, 2H), 3.66-3.58 (m, 34H), 3.56-3.51 (m, 4H), 3.51-3.46 (m, 7H), 3.37 (br t, J=5.2 Hz, 2H), 3.06 (s, 2H), 1.78 (m, 2H), 1.20 (t, J=7.2 Hz, 3H), 1.00 (t, J=7.6 Hz, 3H). LC/MS [M+H] 1002.5 (calculated); LC/MS [M+H] 1002.2 (observed).

### Example L-8 Synthesis of 2-amino-N8-(1-(2,5-dioxo-2,5-dihydro-1H-pyrrol-1-yl)-2-oxo-6,9,12,15,18,21,24,27,30,33-decaoxa-3-azapentatriacontan-35-yl)-N4-ethoxy-N8-methyl-N4-propyl-3H-pyrido[4,3-b]azepine-4,8-dicarboxamide, azaBzL-8

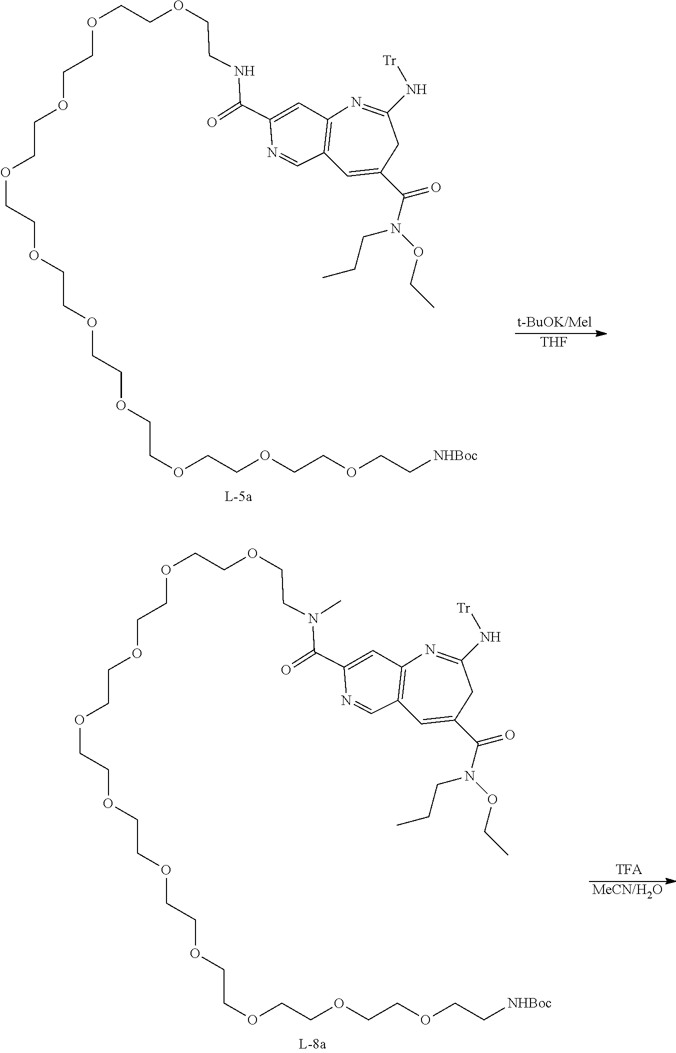

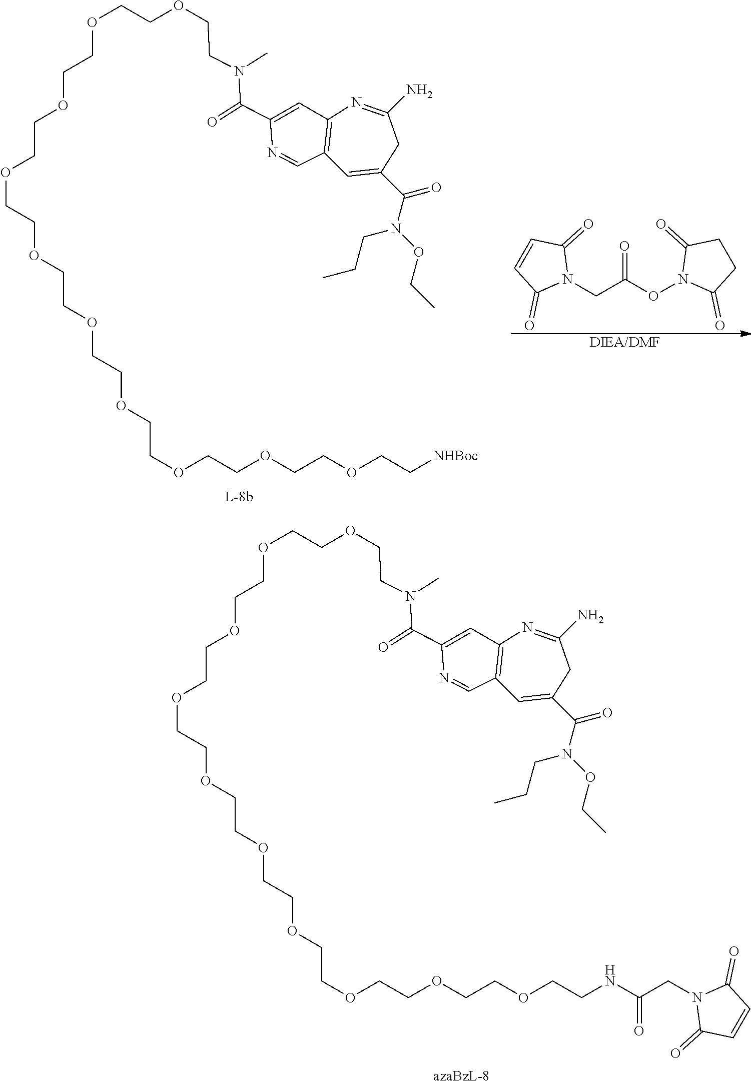 [SMILES: CCCN(OCC)C(=O)C1=Cc2cnc(C(=O)N(C)CCOCCOCCOCCOCCOCCOCCOCCOCCOCCOCCNC(=O)OC(C)(C)C)cc2N=C(N)C1 ; O=C(CN1C(=O)C=CC1=O)ON1C(=O)CCC1=O ; "DIEA/DMF" ; CCCN(OCC)C(=O)C1=Cc2cnc(C(=O)N(C)CCOCCOCCOCCOCCOCCOCCOCCOCCOCCOCCNC(=O)CN3C(=O)C=CC3=O)cc2N=C(N)C1]

### Preparation of tert-butyl N-[2-[2-[2-[2-[2-[2-[2-[2-[2-[2-[2-[[4-[ethoxy(propyl)carbamoyl]-2-(tritylamino)-3H-pyrido[4,3-b]azepine-8-carbonyl]-methyl-amino]ethoxy]ethoxy]ethoxy]ethoxy]ethoxy]ethoxy]ethoxy]ethoxy]ethoxy]ethoxy]ethyl]carbamate, L-8a

To a solution of tert-butyl N-[2-[2-[2-[2-[2-[2-[2-[2-[2-[2-[2-[[4-[ethoxy(propyl)carbamoyl]-2-(tritylamino)-3H-pyrido[4,3-b]azepine-8-carbonyl]amino]ethoxy]ethoxy]ethoxy]ethoxy]ethoxy]ethoxy]ethoxy]ethoxy]ethoxy]ethoxy]ethyl]carbamate, L-5a (160 mg, 138 umol, 1 eq) in THE (2 mL) was added t-BuOK (1 M, 276 μL, 2 eq) and Mel (19.6 mg, 138 umol, 8.61 uL, 1 eq) at 25° C. for 2 hr. The mixture was filtered and purified by prep-HPLC (TFA condition; column: Phenomenex Luna 80*30 mm*3 um; mobile phase: [water(TFA)-ACN];B %: 35%-65%,8 min) to afford L-6a (80 mg, 68.3 umol, 49.40% yield) as colorless oil. LC/MS [M+H] 1171.7 (calculated); LC/MS [M+H] 1171.3 (observed).

### Preparation of 2-amino-N8-(32-amino-3,6,9,12,15,18,21,24,27,30-decaoxadotriacontyl)-N4-ethoxy-N8-methyl-N4-propyl-3H-pyrido[4,3-b]azepine-4,8-dicarboxamide, L-8b

To a solution of L-8a (70 mg, 59.8 umol, 1 eq) in DCM (0.2 mL) was added TFA (68.1 mg, 598 umol, 44.2 uL, 10 eq), then the mixture was stirred at 50° C. for 0.5 hr. The mixture was filtered and concentrated under reduced pressure. The residue was purified by prep-HPLC (TFA condition; column: Phenomenex Luna 80*30 mm*3 um; mobile phase: [water(TFA)-ACN];B %: 1%-28%, 8 min) to afford L-8b (30 mg, 31.8 umol, 53.24% yield, TFA) as yellow oil. ¹H NMR (DMSO-d₆, 400 MHz,) δ 8.52 (s, 1H), 8.51-8.46 (m, 1H), 7.72 (s, 1H), 7.68-7.49 (m, 2H), 7.21 (s, 1H), 3.86 (q, J=6.8 Hz, 2H), 3.65-.0.48 (m, 44H), 3.04-2.97 (m, 4H), 2.90 (s, 3H), 1.72-1.63 (m, 2H), 1.07 (t, J=7.2 Hz, 3H), 0.96-0.91 (m, 3H). LC/MS [M+H] 829.5 (calculated); LC/MS [M+H] 829.5 (observed).

### Preparation of azaBzL-8

To a solution of L-8b (20 mg, 24.1 umol, 1 eq) in DMF (0.5 mL) were added DIEA (12.5 mg, 96.5 umol, 16.8 uL, 4 eq) and 2,5-dioxopyrrolidin-1-yl 2-(2,5-dioxo-2,5-dihydro-1H-pyrrol-1-yl)acetate (6.08 mg, 24.1 umol, 1 eq), then stirred at 25° C. for 0.5 hr. The mixture was filtered and purified by prep-HPLC (TFA condition; column: Phenomenex Luna 80*30 mm*3 um; mobile phase: [water(TFA)-ACN];B %: 5%-35%, 8 min) to afford azaBzL-8 (15 mg, 15.5 umol, 64.4% yield) as yellow oil. ¹H NMR (MeOD, 400 MHz) δ 8.80 (s, 1H), 8.16 (s, 1H), 7.52 (s, 1H), 6.91 (s, 2H), 4.19 (s, 2H), 4.02-3.94 (m, 2H), 3.77 (t, J=7.2 Hz, 2H), 3.72 (m, 2H), 3.69-3.61 (m, 36H), 3.56 (m, 2H), 3.44-3.36 (m, 6H), 3.17 (s, 3H), 1.79 (m, 2H), 1.23-1.17 (m, 3H), 1.05-0.99 (m, 3H). LC/MS [M+H] 966.5 (calculated); LC/MS [M+H] 966.2 (observed).

### Example L-9 Synthesis of 2-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]ethyl N-[2-[(6-amino-7H-pyrido[3,2-b]azepine-8-carbonyl)-propyl-amino]oxyethyl]carbamate, azaBzL-9

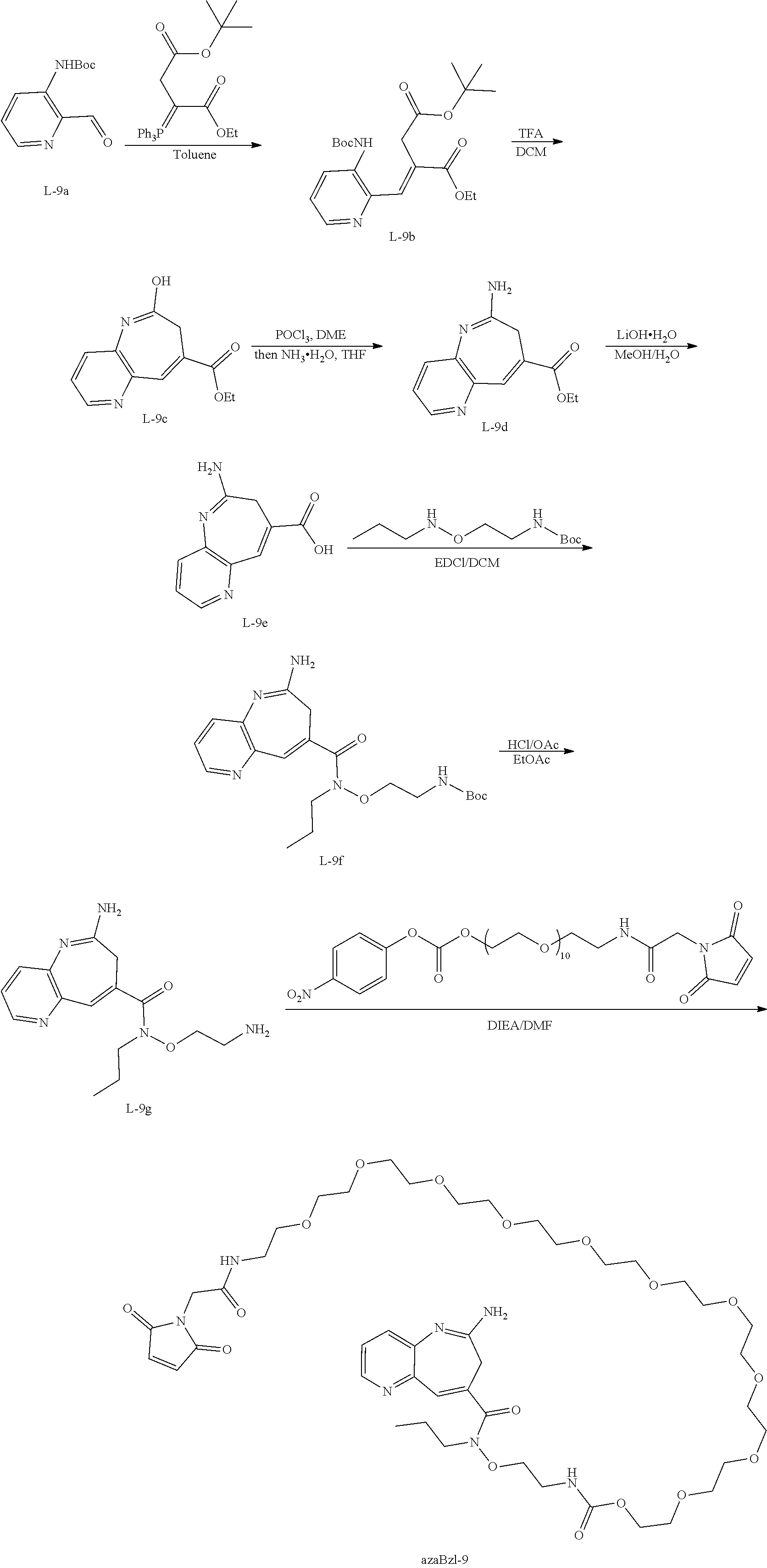

### Preparation of 4-(tert-butyl) 1-ethyl (E)-2-((3-((tert-butoxycarbonyl)amino)pyridin-2-yl)methylene)succinate, L-9b

A solution of tert-butyl (2-formylpyridin-3-yl)carbamate, L-9a (1 g, 4.50 mmol, 1 eq) and O4-tert-butyl O1-ethyl 2-(triphenyl-λ5-phosphanylidene)butanedioate (2.50 g, 5.40 mmol, 1.2 eq) in DCM (5 mL) was stirred at 50° C. for 2 hrs. The mixture was filtered and concentrated under reduced pressure. The residue was purified by flash silica gel chromatography (ISCO®; 40 g SepaFlash® Silica Flash Column, Eluent of 0-30% Ethyl acetate/Petroleum ether gradient @60 mL/min) to afford L-9b (350 mg, 861 umol, 19.1% yield) as colorless oil. ¹H NMR (CDCl₃, 400 MHz) δ 8.36-8.31 (m, 2H), 7.76 (s, 1H), 7.25 (dd, J=4.8, 8.4 Hz, 1H), 6.72 (s, 1H), 4.32 (q, J=7.2 Hz, 2H), 3.70 (s, 2H), 1.54 (s, 9H), 1.42 (s, 9H), 1.36 (t, J=7.2 Hz, 3H). LC/MS [M+H] 407.2 (calculated); LC/MS [M+H] 407.2 (observed).

### Preparation of ethyl 6-hydroxy-7H-pyrido[3,2-b]azepine-8-carboxylate, L-9c

To a solution of L-9b (350 mg, 861 umol, 1 eq) in DCM (5 mL) was added TFA (982 mg, 8.61 mmol, 638 μL, 10 eq) and stirred at 50° C. for 3 hrs. The mixture was concentrated under reduced pressure to afford L-9c (300 mg, crude) as a yellow solid. ¹H NMR (CDCl₃, 400 MHz) δ 8.78 (s, 1H), 8.65 (dd, J=1.2, 4.8 Hz, 1H), 8.05 (s, 1H), 7.64-7.60 (m, 1H), 7.53 (dd, J=4.4, 8.4 Hz, 1H), 4.36 (q, J=7.2 Hz, 2H), 3.43 (s, 2H), 1.39 (t, J=7.2 Hz, 3H). LC/MS [M+H] 233.0 (calculated); LC/MS [M+H] 233.2 (observed).

### Preparation of ethyl 6-amino-7H-pyrido[3,2-b]azepine-8-carboxylate, L-9d

The compound L-9c (700 mg, 3.01 mmol, 1 eq) was dissolved in POCl₃ (6.93 g, 45.2 mmol, 4.21 mL, 15 eq) and the mixture was stirred at 90° C. for 16 hrs under N₂. The mixture was concentrated under reduced pressure. Then the residue was dissolved in MeCN (20 mL), the solution was added NH₃·H₂O (27.30 g, 234 mmol, 30 mL, 30% purity, 65.1 eq) and then stirred at 25° C. for 0.5 hr. The reaction mixture was extracted with EtOAc (30 mL×3). The combined organic layers were washed with brine (20 mL×2), dried over [Na₂SO₄], filtered and concentrated under reduced pressure to give L-9d (600 mg, 2.59 mmol, 72.2% yield) as a brown solid. LC/MS [M+H] 232.1 (calculated); LC/MS [M+H] 232.1 (observed).

### Preparation of 6-amino-7H-pyrido[3,2-b]azepine-8-carboxylic acid, L-9e

To a solution of L-9d (600 mg, 2.59 mmol, 1 eq) in MeOH (1 mL) and water (0.3 mL) was added lithium hydroxide hydrate, LiOH·H₂O (327 mg, 7.78 mmol, 3 eq), and then stirred at 25° C. for 2 hr. The pH of the mixture was adjusted to 5-6 with 1N HCl, then the precipitate was filtered and the solid was concentrated under reduced pressure to afford L-9e (300 mg, 1.48 mmol, 56.9% yield) as a brown solid. LC/MS [M+H] 204.1 (calculated); LC/MS [M+H] 204.2 (observed).

### Preparation of tert-butyl (2-((6-amino-N-propyl-7H-pyrido[3,2-b]azepine-8-carboxamido)oxy)ethyl)carbamate, L-9f

To a solution of L-9e (240 mg, 1.18 mmol, 1 eq) and tert-butyl N-[2-(propylaminooxy)ethyl]carbamate (335 mg, 1.54 mmol, 1.3 eq) in DCM (3 mL) and dimethylacetamide, DMA (2 mL) were added methane sulfonic acid (170 mg, 1.77 mmol, 127 L, 1.5 eq) and EDCI (906 mg, 4.72 mmol, 4 eq), and then stirred at 25° C. for 1 hr. The reaction mixture was diluted with water 10 mL and extracted with EtOAc (15 mL×3). The combined organic layers were washed with brine (10 mL×2), dried over [Na₂SO₄], filtered and concentrated under reduced pressure to give a residue. The residue was purified by flash silica gel chromatography (ISCO®; 25 g SepaFlash® Silica Flash Column, Eluent of 0~100% Ethyl acetate/Petroleum ether to 50% Ethyl acetate/MeOH gradient @45 mL/min) to afford L-9f (300 mg, 743 mol, 62.9% yield) as brown oil. LC/MS [M+H] 404.2 (calculated); LC/MS [M+H] 404.3 (observed).

### Preparation of 6-amino-N-(2-aminoethoxy)-N-propyl-7H-pyrido[3,2-b]azepine-8-carboxamide, L-9g

To a solution of L-9f (200 mg, 496 mol, 1 eq) in dioxane (1 mL) was added HCl/dioxane (4 M, 4.00 mL, 32 eq), and then stirred at 25° C. for 0.5 hr. The mixture was concentrated under reduced pressure to afford the crude product L-9g (200 mg, crude, HCl) as a brown solid. LC/MS [M+H] 304.2 (calculated); LC/MS [M+H] 304.2 (observed).

### Preparation of azaBzL-9

To a solution of L-9g (150 mg, 441 mol, 1 eq, HCl) and diisopropylethylamine, DIEA (285 mg, 2.21 mmol, 384 μL, 5 eq) in DMF (1 mL) was added 2-[2-[2-[2-[2-[2-[2-[2-[2-[2-[2-[[2-(2,5-dioxopyrrol-1 -yl)acetyl]amino]ethoxy]ethoxy]ethoxy]ethoxy]ethoxy]ethoxy]ethoxy]ethoxy]ethoxy]ethoxy]ethyl (4-nitrophenyl) carbonate (248 mg, 309 mol, 0.7 eq), and then stirred at 25° C. for 1 hr. The mixture was filtered and the filtrate was purified by prep-HPLC (TFA condition; column: Phenomenex Luna C18 75*30 mm*3 um; mobile phase: [H₂O (0.1% TFA)-ACN]; gradient: 10%-40% B over 8.0 min) to afford azaBzL-9 (50 mg, 51.6 mol, 11.7% yield) as a yellow oil. ¹H NMR (MeOD, 400 MHz) δ 8.66 (dd, J=1.2, 4.4 Hz, 1H), 7.89 (d, J=8.0 Hz, 1H), 7.58 (dd, J=4.4, 8.4 Hz, 1H), 7.37 (s, 1H), 6.89 (s, 2H), 4.17 (s, 2H), 3.99 (t, J=5.2 Hz, 2H), 3.94-3.88 (m, 2H), 3.77 (t, J=7.2 Hz, 2H), 3.66-3.59 (m, 38H), 3.56-3.52 (m, 2H), 3.52-3.49 (m, 2H), 3.47 (s, 2H), 3.41-3.35 (m, 2H), 1.85-1.75 (m, 2H), 1.00 (t, J=7.2 Hz, 3H). LC/MS [M+H] 968.5 (calculated); LC/MS [M+H] 968.3 (observed).

### Example L-10 Synthesis of 6-amino-3-[2-[[3-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]propanoylamino]methyl]pyrimidin-5-yl]-N-ethoxy-N-propyl-7H-pyrido[3,2-b]azepine-8-carboxamide, azaBzL-10

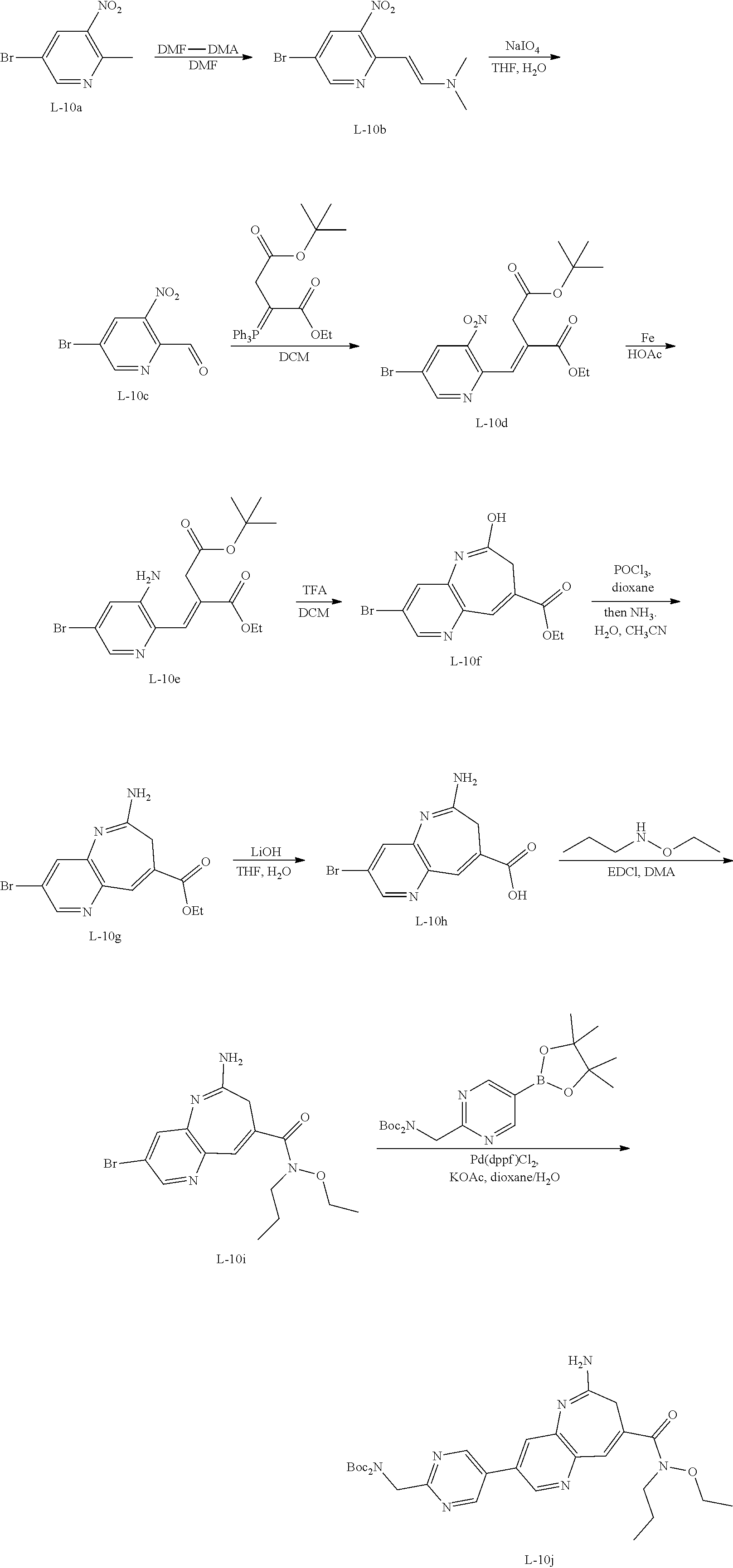

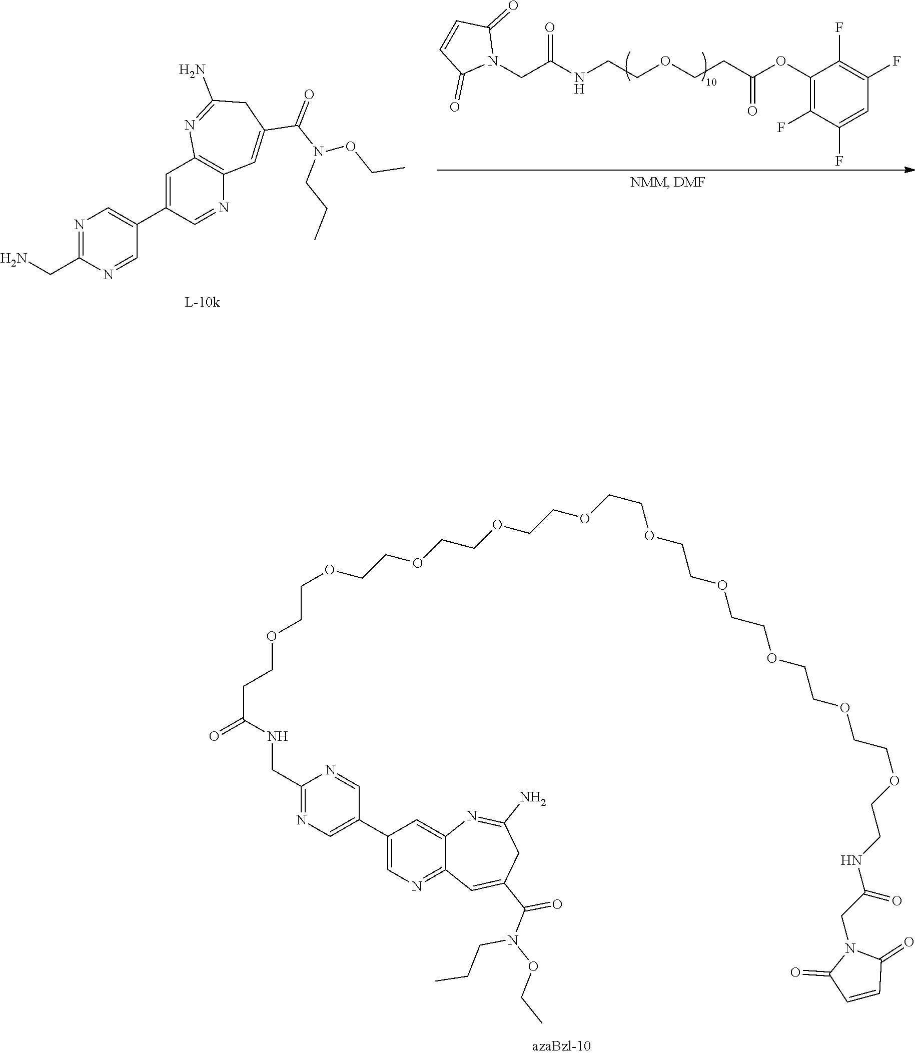

### Preparation of(E)-2-(5-bromo-3-nitro-2-pyridyl)-N,N-dimethyl-ethenamine, L-10b

To a solution of 5-bromo-2-methyl-3-nitro-pyridine, L-10a (150 g, 691 mmol, 1 eq) in DMF (750 mL) was added DMF-DMA (165 g, 1.38 mol, 184 mL, 2 eq) at 25° C., and then heated to 90° C. and stirred at 90° C. for 1 hr. The reaction mixture was cooled to 25° C. and concentrated under reduced pressure to give a residue. The residue was diluted with H₂O (1000 mL) at 0° C. and the solid was filtered and the filter cake was dried under reduced pressure to give L-10b (181 g, 665 mmol, 96.2% yield) as a red solid. ¹H NMR (DMSO-d₆, 400 MHz) δ 8.44 (d, J=2.4 Hz, 1H), 8.34 (d, J=2.4 Hz, 1H), 8.14 (d, J=12.4 Hz, 1H), 6.07 (d, J=12.4 Hz, 1H), 3.04 (s, 6H).

### Preparation of 5-bromo-3-nitro-pyridine-2-carbaldehyde, L-10c

To a solution of L-10b (90 g, 331 mmol, 1 eq) in THF (750 mL) and H₂O (750 mL) was added NaIO₄ (156 g, 728 mmol, 40.3 mL, 2.2 eq) in portions at 0° C., and then warmed to 25° C. and stirred at 25° C. for 1 hr. The reaction mixture was filtered and the filtrate was diluted with H₂O (500 mL) and extracted with EtOAc (500 mL×3). The combined organic layers were washed with sat. Na₂S₂O₃ (150 mL×3), brine (150 mL×3), dried over Na₂SO₄, filtered and concentrated under reduced pressure to give a residue. The residue was purified by column chromatography (SiO₂, Petroleum ether/Ethyl acetate=I1/0 to 1/1) to give L-10c (54.3 g, 235 mmol, 71.1% yield) as a brown solid. ¹H NMR (CDCl₃, 400 MHz) δ 10.22 (s, 1H), 9.04 (d, J=1.6 Hz, 1H), 8.38 (d, J=1.6 Hz, 1H). LC/MS [M+H] 230.9 (calculated); LC/MS [M+H] 231.0 (observed).

### Preparation of O4-tert-butyl O1-ethyl (2E)-2-[(5-bromo-3-nitro-2-pyridyl)methylene]butanedioate, L-10d

To a solution of L-10c (103 g, 446 mmol, 1 eq) in DCM (1500 mL) was added O4-tert-butyl O1-ethyl 2-(triphenyl-phosphanylidene)butanedioate (186 g, 401 mmol, 0.9 eq) in portions at 25° C. under N₂, and then heated to 50° C. and stirred for 4 hr. The reaction mixture was cooled to 25° C. and concentrated under reduced pressure to give a residue. The residue was purified by column chromatography (SiO₂, Petroleum ether/Ethyl acetate=I1/0 to 1/1) to give L-10d (150.8 g, 363 mmol, 81.5% yield) as a light yellow solid. ¹H NMR (CDCl₃, 400 MHz) δ 8.89 (d, J=2.0 Hz, 1H), 8.48 (d, J=2.0 Hz, 1H), 8.01 (s, 1H), 4.33 (q, J=7.2 Hz, 2H), 3.75 (s, 2H), 1.42 (s, 9H), 1.36 (t, J=7.2 Hz, 3H). LC/MS [M+Na] 437.0 (calculated); LC/MS [M+Na]436.9 (observed).

### Preparation of O4-tert-butyl O1-ethyl (2E)-2-[(3-amino-5-bromo-2-pyridyl)methylene]butanedioate, L-10e

To a solution of L-10d (30.1 g, 72.5 mmol, 1 eq) in AcOH (500 mL) was added iron filings, Fe (20.2 g, 362 mmol, 5 eq) in portions at 25° C. under N₂, and then stirred at 25° C. for 2 hrs. The reaction mixture was filtered and the filtrate was concentrated under reduced pressure to give a residue. The residue was diluted with H₂O (500 mL) and adjusted pH=8-9 with sat. NaHCO₃ at 0° C. The mixture was filtered and the filtrate was extracted with EtOAc (400 mL×3). The combined organic layers were washed with brine (200 mL), dried over Na₂SO₄, filtered and concentrated under reduced pressure to give L-10e (27.3 g, 71 mmol, 97.9% yield) as a yellow solid. ¹H NMR (MeOD, 400 MHz) δ 7.92 (d, J=2.0 Hz, 1H), 7.71 (s, 1H), 7.30 (d, J=2.0 Hz, 1H), 4.28 (q, J=7.2 Hz, 2H), 3.80 (s, 2H), 1.42 (s, 9H), 1.34 (t, J=7.2 Hz, 3H). LC/MS [M+H] 385.1 (calculated); LC/MS [M+H] 385.0 (observed).

### Preparation of methyl 8-(dipropylcarbamoyl)-6-(tritylamino)-7H-pyrido[3,2-b]azepine-3-carboxylate, L-10f

To a solution of L-10e (82.0 g, 213 mmol, 1 eq) in DCM (900 mL) was added TFA (243 g, 2.13 mol, 158 mL, 10 eq) at 25° C., and then heated to 50° C. and stirred for 2 hrs. The reaction mixture was cooled to 25° C. and concentrated under reduced pressure to give a residue. The residue was poured into ice-water (1000 mL) at 0° C. and adjusted pH=8-9 with sat. NaHCO₃ at 0° C. The aqueous phase was extracted with EtOAc (500 mL×3). The combined organic layers were washed with brine (100 mL), dried over Na₂SO₄, filtered and concentrated under reduced pressure to give L-10f (74.0 g, crude) as a yellow solid. ¹H NMR (DMSO-d₆, 400 MHz) δ 10.64 (s, 1H), 8.58 (d, J=2.0 Hz, 1H), 7.79 (d, J=2.0 Hz, 1H), 7.67 (s, 1H), 4.26 (q, J=7.2 Hz, 2H), 3.24 (s, 2H), 1.29 (t, J=7.2 Hz, 3H). LC/MS [M+H] 310.9 (calculated); LC/MS [M+H] 310.9 (observed).

### Preparation of ethyl 6-amino-3-bromo-7H-pyrido[3,2-b]azepine-8-carboxylate, L-10g

To a solution of L-1Of (10.1 g, 32.5 mmol, 1 eq) in dioxane (100 mL) was added POCl₃ (24.9 g, 162 mmol, 15.1 mL, 5 eq) at 25° C., and then heated to 120° C. and stirred for 6 hrs. The reaction mixture was cooled to 0° C. and added to NH₃·H₂O (275 g, 1.96 mol, 303 mL, 25% purity, 58.3 eq) in CH₃CN (40 mL), then stirred at 0° C. for 1 hr. The reaction mixture was filtered and the filter cake was dried under reduced pressure as pure product. The filtrate was extracted with EtOAc (150 mL×3). The combined organic layers were washed with brine (100 mL×3), dried over Na₂SO₄, filtered and concentrated under reduced pressure to give a residue. The residue was purified by column chromatography (SiO₂, Petroleum ether/Ethyl acetate=I/O to Ethyl acetate/Methanol=10:1) to give L-10g (9.1 g, 29.3 mmol, 87.1% yield) as a brown solid. ¹H NMR (DMSO-d₆, 400 MHz) δ 8.34 (d, J=2.0 Hz, 1H), 7.66 (s, 1H), 7.62 (d, J=2.0 Hz, 1H), 7.33 (s, 2H), 4.25 (q, J=7.2 Hz, 2H), 2.96 (s, 2H), 1.30 (t, J=7.2 Hz, 3H). LC/MS [M+H] 310.0 (calculated); LC/MS [M+H] 309.9 (observed).

### Preparation of 6-amino-3-bromo-7H-pyrido[3,2-b]azepine-8-carboxylic acid, L-10h

To a solution of L-10g (27.0 g, 87.1 mmol, 1 eq) in THE (300 mL) was added a solution of LiOH·H₂O (11.0 g, 261 mmol, 3 eq) in H₂O (100 mL) at 0° C., and then warmed to 25° C. and stirred for 2 hrs. The reaction mixture was cooled to 0° C. and adjusted pH=5-6 with 1 N HCl at 0° C., and then concentrated under reduced pressure to remove THF. The solid precipitate was filtered and the filter cake was dried under reduced pressure to give L-10h (24.1 g, 85.5 mmol, 98.1% yield) as a brown solid. ¹H NMR (DMSO-d₆, 400 MHz) δ 8.32 (d, J=2.0 Hz, 1H), 7.61 (s, 1H), 7.60 (d, J=2.0 Hz, 1H), 7.28 (s, 2H), 2.94 (s, 2H). LC/MS [M+H] 282.0 (calculated); LC/MS [M+H] 281.9 (observed).

### Preparation of 6-amino-3-bromo-N-ethoxy-N-propyl-7H-pyrido[3,2-b]azepine-8-carboxamide, L-10i

To a solution of L-10h (12.0 g, 42.5 mmol, 1 eq), N-ethoxypropan-1-amine (5.94 g, 42.5 mmol, 1 eq, HCl) and MsOH (4.09 g, 42.5 mmol, 3.04 mL, 1 eq) in DCM (120 mL) and DMA (12 mL) was added EDCI (24.5 g, 128 mmol, 3 eq) at 0° C., and then warmed to 25° C. and stirred for 1 hr. The reaction mixture was concentrated under reduced pressure to remove DCM. The residue was quenched by addition of H₂O (50 mL) at 0° C., and then adjusted pH=8-9 with aq. NaHCO₃ at 0° C. and extracted with EtOAc (30 mL*3). The combined organic layers were washed with brine (30 mL×3), dried over Na₂SO₄, filtered and concentrated under reduced pressure to give a residue. The residue was purified by column chromatography (SiO₂, Petroleum ether/Ethyl acetate=I1/0 to Ethyl acetate/Methanol=10:1) to give L-10i (15 g, 41.0 mmol, 96.3% yield) as a brown solid. ¹H NMR (DMSO-d₆, 400 MHz) δ 8.28 (d, J=2.0 Hz, 1H), 7.57 (d, J=2.0 Hz, 1H), 7.24 (s, 1H), 7.17 (s, 1H), 7.02 (s, 1H), 3.85 (q, J=7.2 Hz, 2H), 3.62 (t, J=7.2 Hz, 2H), 2.84 (s, 2H), 1.69-1.59 (m, 2H), 1.05 (t, J=7.2 Hz, 3H), 0.90 (t, J=7.2 Hz, 3H). LC/MS [M+H] 367.1 (calculated); LC/MS [M+H] 367.0 (observed).

### Preparation of tert-butyl N-[[5-[6-amino-8-[ethoxy(propyl)carbamoyl]-7H-pyrido[3,2-b]azepin-3-yl]pyrimidin-2-yl]methyl]-N-tert-butoxycarbonyl-carbamate, L-10j

To a solution of tert-butyl N-tert-butoxycarbonyl-N-[[5-(4,4,5,5-tetramethyl-1,3,2-dioxaborolan-2-yl)pyrimidin-2-yl]methyl]carbamate (180 mg, 414 mol, 0.8 eq) in dioxane (2 mL) were added L-10i (190 mg, 517 mol, 1 eq) and a solution of K₂CO₃ (143 mg, 1.03 mmol, 2 eq) in water (0.2 mL), then Pd(dppf)Cl₂ (37.9 mg, 51.7 mol, 0.1 eq) was added and stirred at 100° C. for 1 hr under N₂. The mixture was concentrated under reduced pressure. The residue was purified by prep-HPLC (TFA condition; column: Phenomenex Luna 80*30 mm*3 um; mobile phase: [water(TFA)-ACN]; gradient: 5%-35% B over 8 min) to afford L-10j (160 mg, 268 mol, 51.9% yield) as a yellow solid. LC/MS [M+H] 596.3 (calculated); LC/MS [M+H] 596.3 (observed).

### Preparation of 6-amino-3-(2-(aminomethyl)pyrimidin-5-yl)-N-ethoxy-N-propyl-7H-pyrido[3,2-b]azepine-8-carboxamide, L-10k

To a solution of L-10j (160 mg, 269 mol, 1 eq) in EtOAc (2 mL) was added HCl/EtOAc (4 M, 5 mL, 74.5 eq), and then stirred at 25° C. for 1 hr. The mixture was concentrated under reduced pressure to afford L-1Ok (100 mg, 231 mol, 86.2% yield, HCl) as a brown solid. LC/MS [M+H] 396.2 (calculated); LC/MS [M+H] 396.2 (observed).

### Preparation of azaBzL-10

To a solution of L-10k (100 mg, 232 mol, 1 eq, HCl) in DMF (1 mL) were added N-methylmorpholine, NMM (46.8 mg, 463 mol, 50.9 μL, 2 eq) and (2,3,5,6-tetrafluorophenyl) 3-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]propanoate (245 mg, 301 mol, 1.3 eq) at 0° C., and then stirred at 0° C. for 0.5 hr. The reaction mixture was quenched with TFA until pH=5-6, and then purified by prep-HPLC (TFA condition; column: Phenomenex Luna C18 75*30 mm*3 um; mobile phase: [H₂O (0.1% TFA)-ACN]; gradient: 10%-40% B over 8.0 min) to afford azaBzL-10 (50 mg, 47.8 mol, 20.6% yield) as yellow oil. ¹H NMR (MeOD, 400 MHz) δ 9.17 (s, 2H), 9.06 (d, J=2.0 Hz, 1H), 8.14 (d, J=2.0 Hz, 1H), 7.49 (s, 1H), 6.91 (s, 2H), 4.72 (s, 2H), 4.18 (s, 2H), 3.81 (q, J=7.2 Hz, 2H), 3.83-3.75 (m, 4H), 3.65-3.60 (m, 36H), 3.54-3.52 (m, 4H), 3.38-3.36 (m, 2H), 2.62 (t, J=6.0 Hz, 2H), 1.84-1.78 (m, 2H), 1.24 (t, J=7.2 Hz, 3H), 1.03 (t, J=7.2 Hz, 3H). LC/MS [M+H] 1044.5 (calculated); LC/MS [M+H] 1044.3 (observed).

### Example L-17 Synthesis of (2,3,5,6-tetrafluorophenyl) 3-[2-[2-[2-[2-[2-[2-[2-[2-[2-[3-[[5-[6-amino-8-[ethoxy(propyl)carbamoyl]-7H-pyrido[3,2-b]azepin-3-yl]pyrimidin-2-yl]methylamino]-3-oxo-propoxy]ethoxy]ethoxy]ethoxy]ethoxy]ethoxy]ethoxy]ethoxy]ethoxy]ethoxy]propanoate, azaBzL-17

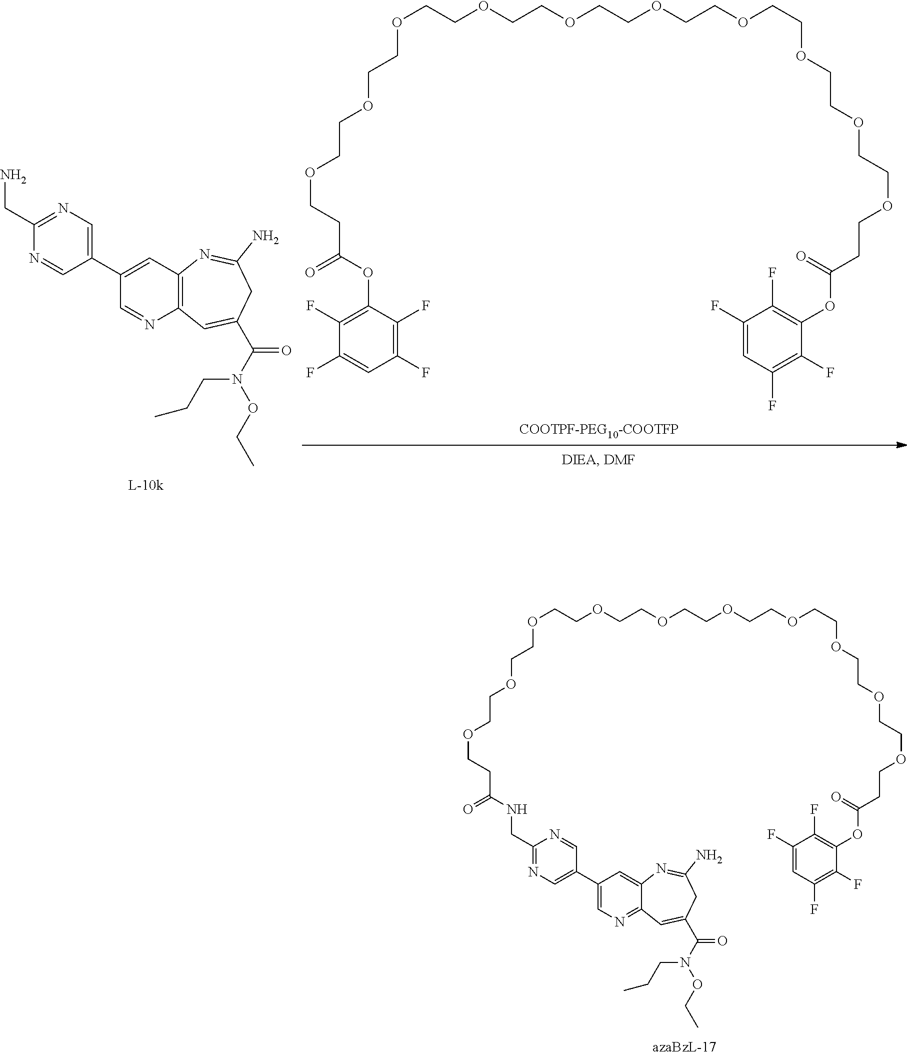

To a solution of(2,3,5,6-tetrafluorophenyl) 3-[2-[2-[2-[2-[2-[2-[2-[2-[2-[3-oxo-3-(2,3,5,6-tetrafluorophenoxy)propoxy]ethoxy]ethoxy]ethoxy]ethoxy]ethoxy]ethoxy]ethoxy]ethoxy]ethoxy]propanoate (97.3 mg, 0.114 mmol, 2.5 eq) in DMF (0.5 mL) were added a solution of 6 -amino-3-[2-(aminomethyl)pyrimidin-5-yl]-N-ethoxy-N-propyl-7H-pyrido[3,2-b]azepine-8-carboxamide, L-10k (18 mg, 0.0455 mmol, 1 eq) and DIEA (35.3 mg, 0.273 mmol, 0.476 mL, 6 eq) in DMF (0.5 mL) at 0° C., then the mixture was stirred at 25° C. for 1 hr. The reaction mixture was purified by prep-HPLC (TFA condition; column: Phenomenex Luna C18 75*30 mm*3 um; mobile phase: [H2O (0.1% TFA)-ACN]; gradient: 20%-50% B over 8.0 min) to afford azaBzL-17 (12 mg, 0.111 mmol, 24.3% yield) as colorless oil. ¹H NMR (MeOD, 400 MHz) δ 9.16 (s, 2H), 9.03 (d, J=2.0 Hz, 1H), 8.12 (d, J=2.0 Hz, 1H), 7.47-7.38 (m, 2H), 4.72 (d, J=5.6 Hz, 2H), 4.02 (q, J=7.2 Hz, 2H), 3.90-3.86 (m, 2H), 3.83-3.77 (m, 4H), 3.68-3.60 (m, 36H), 3.54 (s, 2H), 2.99 (t, J=6.0 Hz, 2H), 2.62 (t, J=6.0 Hz, 2H), 1.85-1.76 (m, 2H), 1.24 (t, J=7.2 Hz, 3H), 1.01 (t, J=7.2 Hz, 3H). LC/MS [M+H] 1084.5 (calculated); LC/MS [M+H] 1084.7 (observed).

### Example L-20 Synthesis of 2-amino-N-((40-(2,5-dimethylene-2,5-dihydro-1H-pyrrol-1-yl)-4,39-dioxo-8,11,14,17,20,23,26,29,32,35-decaoxa-3,5,38-triazatetracontyl)oxy)-N-propyl-3H-pyrido[3,4-b]azepine-4-carboxamide, azaBzL-20

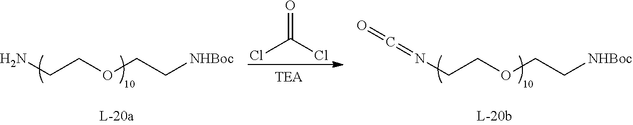

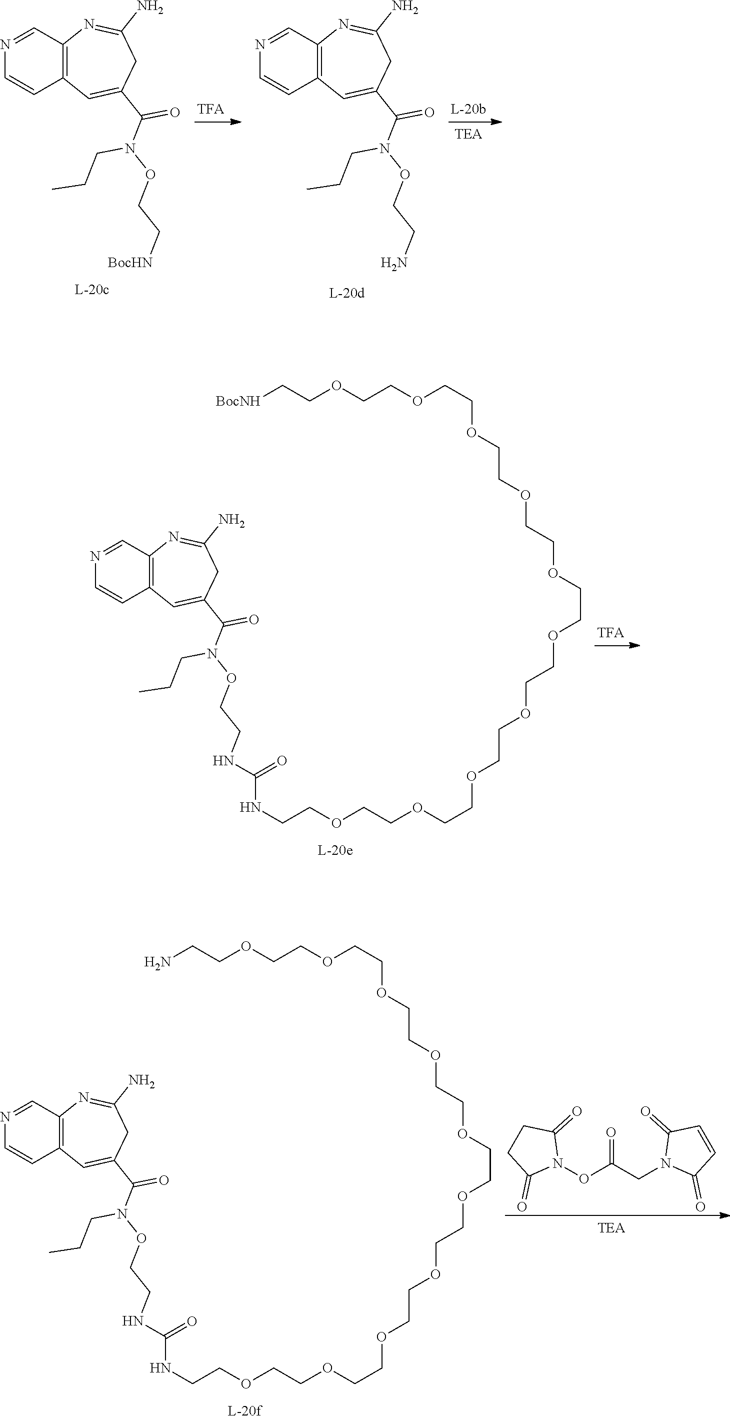

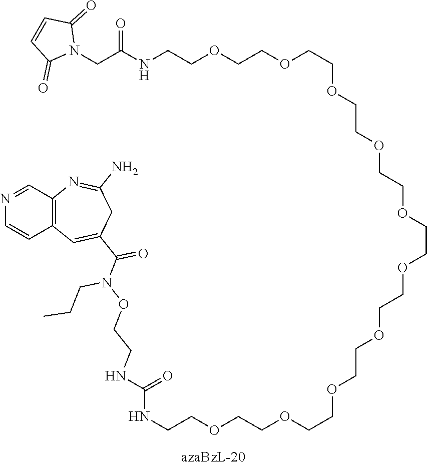 [SMILES: CCCN(OCCNC(=O)NCCOCCOCCOCCOCCOCCOCCOCCOCCOCCOCCNC(=O)CN1C(=O)C=CC1=O)C(=O)C1=Cc2ccncc2N=C(N)C1]

### Preparation of tert-butyl (32-isocyanato-3,6,9,12,15,18,21,24,27,30-decaoxadotriacontyl)carbamate, L-20b

To a solution of tert-butyl (32-amino-3,6,9,12,15,18,21,24,27,30-decaoxadotriacontyl)carbamate, L-20a (0.15 g, 0.25 mmol, 1 eq) in DCM was added TEA (0.348 ml, 2.5 mmol, 10 eq), followed by phosgene (0.892 ml as a 1.4 M solution in toluene, 0.25 mmol, 1 eq). The reaction mixture was monitored by LCMS, concentrated, and purified by reverse phase HPLC to give L-20b (78 mg, 0.125 mmol, 50%). LC/MS [M+H] 627.37 (calculated); LC/MS [M+H] 627.64 (observed).

### Preparation of 2-amino-N-(2-aminoethoxy)-N-propyl-3H-pyrido[3,4-b]azepine-4-carboxamide, L-20d

tert-Butyl (2-((2-amino-N-propyl-3H-pyrido[3,4-b]azepine-4-carboxamido)oxy)ethyl)carbamate, L-20c (6.1 mg, 0.015 mmol, 1 eq) was suspended in minimal TFA. After 15 minutes, the reaction mixture was concentrated to give crude, L-20d (12.7 mg, 0.031 mmol, 100%). LC/MS [M+H] 304.18 (calculated); LC/MS [M+H] 304.28 (observed).

### Preparation of tert-butyl (39-(2-amino-3H-pyrido[3,4-b]azepine-4-carbonyl)-34-oxo-3,6,9,12,15,18,21,24,27,30,38-undecaoxa-33,35,39-triazadotetracontyl)carbamate, L-20e

To a mixture of L-20d (37.8 mg, 0.124 mmol, 1 eq) and 2Am4CBza-L-18b (78 mg, 0.124 mmol, 1 eq) in DMF was added TEA (0.17 ml, 1.24 mmol, 10 eq). The reaction was stirred at room temperature, then diluted with water and purified by reverse phase HPLC to give L-20e (48 mg, 0.052 mmol, 41%). LC/MS [M+H] 930.54 (calculated); LC/MS [M+H] 930.54 (observed).

### Preparation of 2-amino-N-((37-amino-4-oxo-8,11,14,17,20,23,26,29,32,35-decaoxa-3,5-diazaheptatriacontyl)oxy)-N-propyl-3H-pyrido[3,4-b]azepine-4-carboxamide, L-20f

L-20e (48 mg, 0.052 mmol, 1 eq) was dissolved in minimal TFA. After 15 minutes, the reaction mixture was concentrated to give L-20f as a TFA salt (0.053 g, 0.050 mmol, 96%). LC/MS [M+H] 830.49 (calculated); LC/MS [M+H] 830.76 (observed).

### Preparation of azaBzL-20

To a solution of L-20f (0.053 g, 0.050 mmol, 1 eq) in DMF (0.5 ml) was added TEA (0.09 ml, 0.64 mmol, 12.8 eq) followed by 2,5-dioxopyrrolidin-1-yl 2-(2,5-dioxo-2,5-dihydro-1H-pyrrol-1-yl)acetate (0.016 g, 0.064 mmol, 1.28 eq). The reaction mixture was concentrated, diluted with 1% TFA in water, and purified by reverse phase HPLC to give azaBzL-20 (38.5 mg, 0.040 mmol, 80%). LC/MS [M+H] 967.50 (calculated); LC/MS [M+H] 967.80 (observed).

Alternatively, azaBzL-20 may be synthesized as follows:

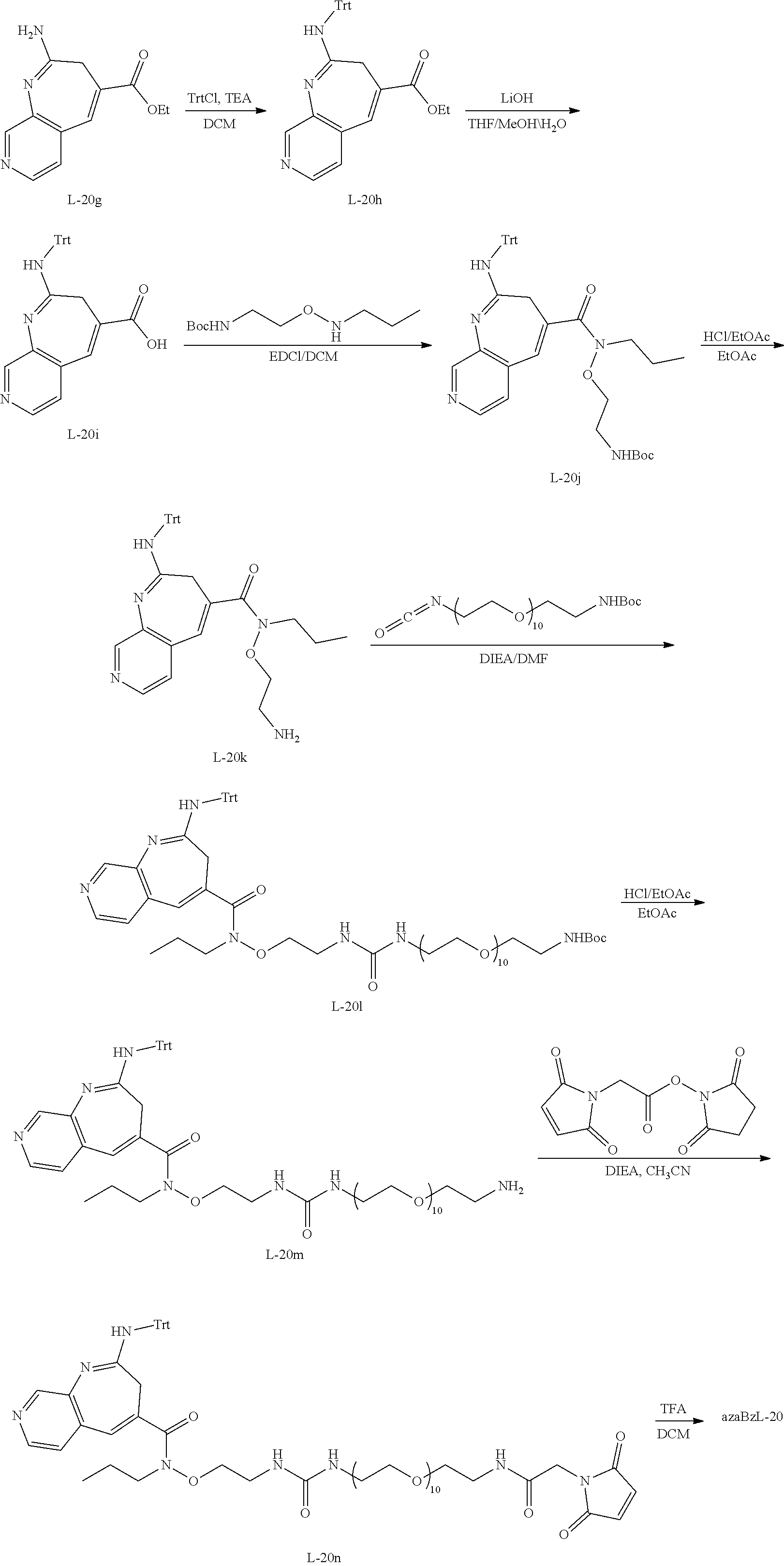

### Preparation of ethyl 2-(tritylamino)-3H-pyrido[3,4-b]azepine-4-carboxylate, L-20h

To a solution of ethyl 2-amino-3H-pyrido[3,4-b]azepine-4-carboxylate, L-20g (4 g, 17.3 mmol, 1 eq) in DCM (50 mL) was added Et₃N (3.50 g, 34.5 mmol, 4.82 mL, 2 eq) and TrtCl (12.0 g, 43.2 mmol, 2.5 eq), and then stirred at 50° C. for 16 hrs. The mixture was filtered and washed with EtOAc (50 mL) and the filtrate was concentrated. The residue was purified by flash silica gel chromatography (ISCO®; 80 g SepaFlash® Silica Flash Column, Eluent of 0~40% Ethyl acetate/MeOH gradient @50 mL/min) to give L-20h (5.2 g, 10.9 mmol, 63.4% yield) as a light yellow solid. LC/MS [M+H] 474.2 (calculated); LC/MS [M+H] 474.1 (observed).

### Preparation of 2-(tritylamino)-3H-pyrido[3,4-b]azepine-4-carboxylic acid, L-20i

To a solution of L-20h (4.5 g, 9.50 mmol, 1 eq) in THE (30 mL) and MeOH (15 mL) was added the solution of LiOH·H₂O (1.20 g, 28.51 mmol, 3 eq) in H₂O (10 mL), and then stirred at 60° C. for 2 hrs. The mixture was concentrated to removed THE and MeOH, and the suspension was filtered and the filter cake was concentrated to give L-20i (3.5 g, 7.86 mmol, 82.68% yield) as a yellow solid. ¹H NMR (MeOD, 400 MHz) δ 7.84 (d, J=5.2 Hz, 1H), 7.64 (s, 1H), 7.43 (s, 1H), 7.35-7.12 (m, 16H), 2.93 (s, 2H). LC/MS [M+H] 446.2 (calculated); LC/MS [M+H] 446.1 (observed).

### Preparation of tert-butyl N-[2-[propyl-[2-(tritylamino)-3H-pyrido[3,4-b]azepine-4-carbonyl]amino]oxyethyl]carbamate, L-20j

To a solution of L-20i (3.5 g, 7.86 mmol, 1 eq) in DMA (8 mL) and DCM (40 mL) were added methanesulfonic acid (755 mg, 7.86 mmol, 561 μL, 1 eq), tert-butyl N-[2-(propylaminooxy)ethyl]carbamate (1.71 g, 7.86 mmol, 1 eq) and EDCI (4.52 g, 23.57 mmol, 3 eq), then stirred at 0° C. for 0.5 hr. The mixture was concentrated to remove DCM, and adjusted to pH=10 with aq. Na₂CO₃, extracted with DCM (50 mL×3). The combined organic phase was washed with brine (50 mL), dried by Na₂SO₄ and concentrated to give L-20j (4.5 g, 6.97 mmol, 88.70% yield) as light yellow oil. ¹H NMR (MeOD, 400 MHz) δ 7.92-7.82 (m, 1H), 7.67 (s, 1H), 7.41-7.32 (m, 6H), 7.29-7.13 (m, 11H), 3.97 (t, J=5.2 Hz, 2H), 3.77 (t, J=6.8 Hz, 2H), 3.28-3.26 (m, 2H), 3.06 (s, 2H), 1.89-1.73 (m, 2H), 1.32 (s, 9H), 1.00 (t, J=7.2 Hz, 3H). LC/MS [M+H] 646.3 (calculated); LC/MS [M+H] 646.2 (observed).

### Preparation of N-(2-aminoethoxy)-N-propyl-2-(tritylamino)-3H-pyrido[3,4-b]azepine-4-carboxamide, L-20k

To a solution of L-20j (2.7 g, 4.18 mmol, 1 eq) in EtOAc (20 mL) was added HCl/EtOAc (4 M, 52.26 mL, 50 eq), and then stirred at 25° C. for 0.5 hr. The mixture was concentrated to give crude L-20k (2.5 g, crude, HCl) as a light yellow solid. LC/MS [M+H] 546.3 (calculated); LC/MS [M+H] 546.2 (observed).

### Preparation of tert-butyl N-[2-[2-[2-[2-[2-[2-[2-[2-[2-[2-[2-[2-[propyl-[2-(tritylamino)-3H-pyrido[3,4-b]azepine-4-carbonyl]amino]oxyethylcarbamoylamino]ethoxy]ethoxy]ethoxy]ethoxy]ethoxy]ethoxy]ethoxy]ethoxy]ethoxy]ethoxy]ethyl]carbamate, L-201

To a solution of N-(2-aminoethoxy)-N-propyl-2-(tritylamino)-3H-pyrido[3,4-b]azepine-4-carboxamide (2 g, 3.44 mmol, 1 eq, HCl) in DMF (15 mL) were added DIEA (888 mg, 6.87 mmol, 1.20 mL, 2 eq) and tert-butyl N-[2-[2-[2-[2-[2-[2-[2-[2-[2-[2-(2-isocyanatoethoxy)ethoxy]ethoxy]ethoxy]ethoxy]ethoxy]ethoxy]ethoxy]ethoxy]ethoxy]ethyl]carbamate, L-20b (2.05 g, 3.26 mmol, 0.95 eq), an then stirred at 0° C. for 1 hr. The mixture was adjusted pH=5 with TFA at 0° C. and filtered. The filtrate was purified by prep-HPLC (column: Phenomenex luna C18 (250*70 mm, 15 um); mobile phase: [H₂O (0.10% TFA)-ACN]; gradient: 30%-60% B over 20.0 min) to give L-201 (3.7 g, 3.16 mmol, 91.8% yield) as yellow oil. ¹H NMR (MeOD, 400 MHz) δ 8.13 (d, J=6.0 Hz, 1H), 7.87-7.81 (m, 2H), 7.36-7.33 (m, 7H), 7.30-7.15 (m, 9H), 4.02-4.01 (m, 2H), 3.80 (t, J=7.2 Hz, 2H), 3.72-3.52 (m, 39H), 3.50-3.44 (m, 2H), 3.41-3.33 (m, 4H), 3.25-3.17 (m, 4H), 3.05 (t, J=5.2 Hz, 2H), 1.88-1.75 (m, 2H), 1.43 (s, 9H), 1.03 (t, J=7.2 Hz, 3H). LC/MS [M+H] 1172.6 (calculated); LC/MS [M+H] 1172.9 (observed).

### Preparation of N-[2-[2-[2-[2-[2-[2-[2-[2-[2-[2-[2-(2-aminoethoxy)ethoxy]ethoxy]ethoxy]ethoxy]ethoxy]ethoxy]ethoxy]ethoxy]ethoxy]ethylcarbamoylamino]ethoxy]-N-propyl-2-(tritylamino)-3H-pyrido[3,4-b]azepine-4-carboxamide, L-20m

To a solution of L-201 (1.5 g, 1.28 mmol, 1 eq) in EtOAc (20 mL) was added HCl/EtOAc (4 M, 15.9 mL, 50 eq), and then stirred at 25° C. for 0.5 hr. The mixture was concentrated to give crude L-20m (1.5 g, crude, HCl) as a light yellow solid. ¹H NMR (MeOD, 400 MHz,) δ 8.23 (d, J=6.0 Hz, 1H), 7.95 (d, J=6.0 Hz, 1H), 7.90 (s, 1H), 7.49-7.20 (m, 16H), 4.05 (t, J=5.2 Hz, 2H), 3.87-3.76 (m, 4H), 3.74-3.56 (m, 38H), 3.44 (t, J=5.2 Hz, 2H), 3.38 (t, J=5.2 Hz, 2H), 3.18-3.16 (m, 4H), 1.89-1.78 (m, 2H), 1.04 (t, J=7.2 Hz, 3H). LC/MS [M+H] 1072.6 (calculated); LC/MS [M+H] 1072.4 (observed).

### Preparation of N-[2-[2-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]eth ylcarbamoylamino]ethoxy]-N-propyl-2-(tritylamino)-3H-pyrido[3,4-b]azepine-4-carboxamide, L-20n

To a solution of L-20m (1.5 g, 1.35 mmol, 1 eq, HCl) in CH₃CN (30 mL) were added DIEA (874 mg, 6.76 mmol, 1.18 mL, 5 eq) and (2,5-dioxopyrrolidin-1-yl) 2-(2,5-dioxopyrrol-1-yl)acetate (307 mg, 1.22 mmol, 0.9 eq), and then stirred at 0° C. for 0.5 hr. The mixture was adjusted pH=4 with TFA and then concentrated to remove CH₃CN. The crude L-20n (1.6 g, crude) was obtained as yellow oil. LC/MS [M+H] 1209.6 (calculated); LC/MS [M+H] 1209.9 (observed).

### Preparation of azaBzL-20

To a solution of L-20n (2.2 g, 1.82 mmol, 1 eq) in DCM (20 mL) was added TFA (4.15 g, 36.3 mmol, 2.70 mL, 20 eq), and then stirred at 50° C. for 16 hrs. The mixture was concentrated to remove DCM and TFA. The residue was purified by prep-HPLC (column: Welch Ultimate XB-Diol 250*50*10 um; mobile phase: [Heptane-EtOH]; gradient: 5%-95% B over 25.0 min) to give azaBzL-20 (858.6 mg, 887.84 mol, 48.81% yield) as light orange oil. ¹H NMR (MeOD, 400 MHz,) δ 8.66 (s, 1H), 8.53 (d, J=5.2 Hz, 1H), 7.61 (d, J=5.2 Hz, 1H), 7.39 (s, 1H), 6.89 (s, 2H), 4.17 (s, 2H), 3.95 (t, J=5.2 Hz, 2H), 3.82-3.73 (m, 2H), 3.70-3.52 (m, 38H), 3.46 (s, 2H), 3.40-3.38 (m, 6H), 3.11 (t, J=5.2 Hz, 2H), 1.85-1.69 (m, 2H), 1.00 (t, J=7.2 Hz, 3H). LC/MS [M+H] 967.5 (calculated); LC/MS [M+H] 967.5 (observed).

### Example L-28 Synthesis of (2,3,5,6-tetrafluorophenyl) 3-[2-[2-[2-[2-[2-[2-[2-[2-[2-[3-[[5-[6-amino-8-(dipropylcarbamoyl)-7H-pyrido[3,2-b]azepin-3-yl]pyrimidin-2-yl]methylamino]-3-oxo-propoxy]ethoxy]ethoxy]ethoxy]ethoxy]ethoxy]ethoxy]ethoxy]ethoxy]ethoxy]propanoate, azaBzL-28

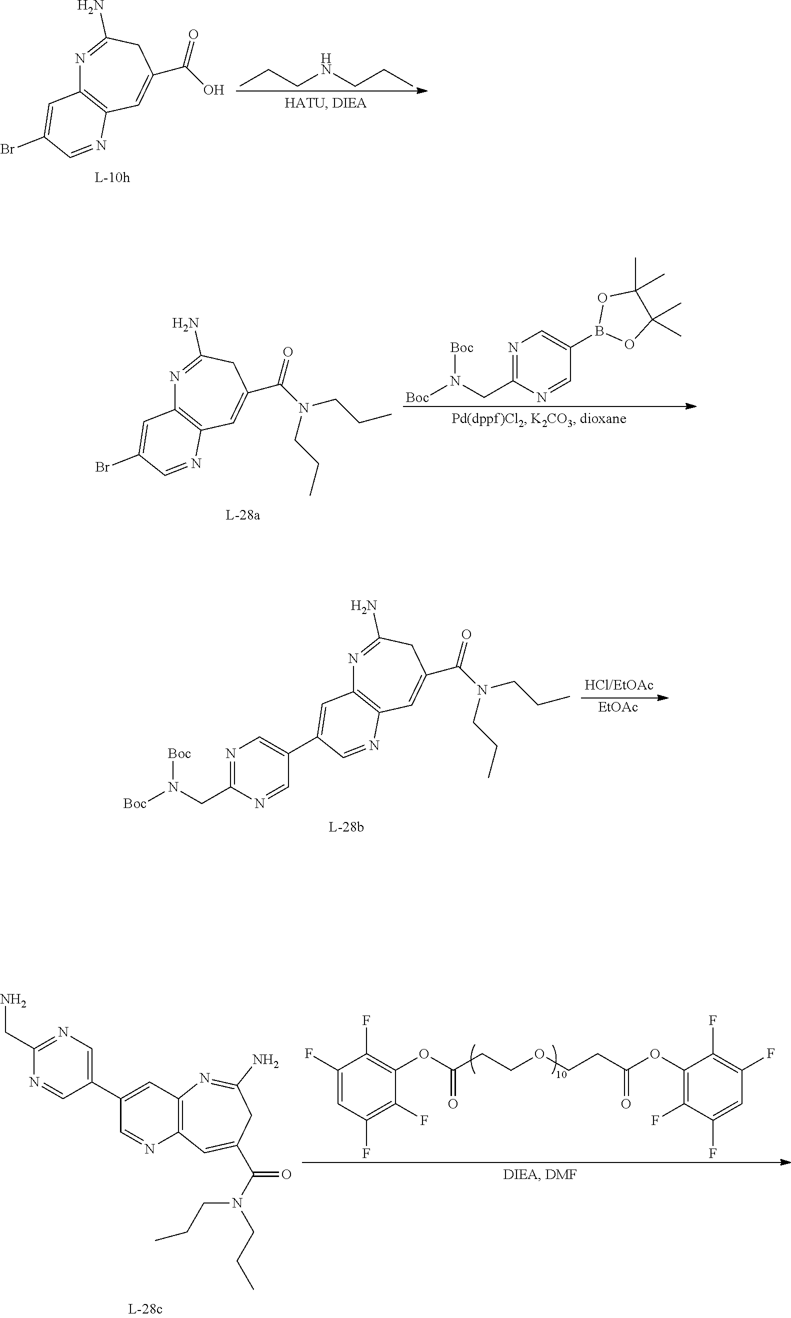

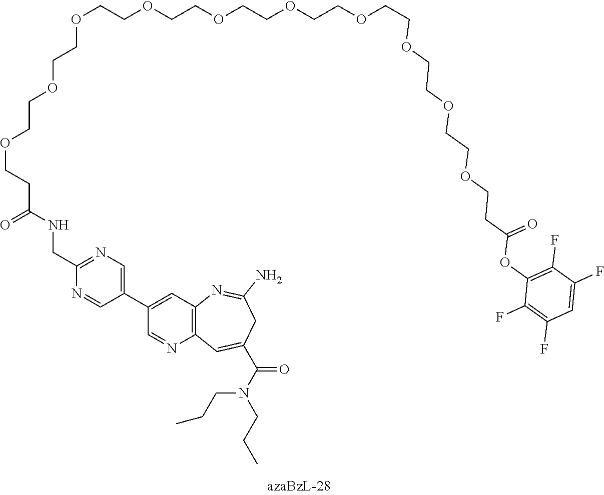 [SMILES: CCCN(CCC)C(=O)C1=Cc2ncc(-c3cnc(CNC(=O)CCOCCOCCOCCOCCOCCOCCOCCOCCOCCOCCC(=O)Oc4c(F)c(F)cc(F)c4F)nc3)cc2N=C(N)C1]

### Preparation of 6-amino-3-bromo-N,N-dipropyl-7H-pyrido[3,2-b]azepine-8-carboxamide, L-28a

To a solution of 6-amino-3-bromo-7H-pyrido[3,2-b]azepine-8-carboxylic acid, L-10h (0.6 g, 2.13 mmol, 1 eq) in DMF (10 mL) were added HATU (889 mg, 2.34 mmol, 1.1 eq), N-propylpropan-1-amine (430 mg, 4.25 mmol, 586 μL, 2 eq) and DIEA (824 mg, 6.38 mmol, 1.11 mL, 3 eq), and then stirred at 0° C. for 0.5 hr. The mixture was diluted with ice water (50 mL) and extracted with EtOAc (30 mL×3). The combined organic layers were washed with brine (20 mL), dried over Na₂SO₄, filtered and concentrated under reduced pressure to give a residue. The residue was purified by flash silica gel chromatography (ISCO®; 20 g SepaFlash® Silica Flash Column, Eluent of 0-100% Ethyl acetate/Petroleum ether to EtOAc=5/1 gradient @60 mL/min) to give L-28a (0.53 g, 1.45 mmol, 68.2% yield) as yellow oil. ¹H NMR (MeOD, 400 MHz) δ 8.31 (d, J=2.0 Hz, 1H), 7.71 (d, J=2.0 Hz, 1H), 6.84 (s, 1H), 3.51-3.38 (m, 4H), 3.30 (s, 2H), 1.78-1.56 (m, 4H), 1.08-0.77 (m, 6H). LC/MS [M+H] 365.1 (calculated); LC/MS [M+H] 365.0 (observed).

### Preparation of tert-butyl N-[[5-[6-amino-8-(dipropylcarbamoyl)-7H-pyrido[3,2-b]azepin-3-yl]pyrimidin-2-yl]methyl]-N-tert-butoxycarbonyl-carbamate, L-28b

To a mixture L-28a (0.43 g, 1.18 mmol, 1 eq), tert-butyl N-tert-butoxycarbonyl-N-[[5-(4,4,5,5-tetramethyl-1,3,2-dioxaborolan-2-yl)pyrimidin-2-yl]methyl]carbamate (615 mg, 1.41 mmol, 1.2 eq) in dioxane (3 mL) and H₂O (0.3 mL) were added K₂CO₃ (325.41 mg, 2.35 mmol, 2 eq) and Pd(dppf)Cl₂ (86.1 mg, 117.7 mol, 0.1 eq) under N₂, and then stirred at 100° C. for 2 hrs under N₂ atmosphere. The mixture was filtered and concentrated to give a residue. The residue was purified by flash silica gel chromatography (ISCO®; 20 g SepaFlash® Silica Flash Column, Eluent of 0~100% Ethyl acetate/Petroleum ether to EtOAc/MeOH=5/1 gradient @75 mL/min) to afford L-28b (0.6 g, 1.01 mmol, 85.8% yield) as a light yellow solid. ¹H NMR (MeOD, 400 MHz) δ 9.11 (s, 2H), 8.63 (d, J=2.0 Hz, 1H), 7.85 (d, J=2.0 Hz, 1H), 6.94 (s, 1H), 5.07 (s, 2H), 3.45 (t, J=7.2 Hz, 4H), 3.32 (s, 2H), 1.79-1.61 (m, 4H), 1.45 (s, 18H), 1.09-0.78 (m, 6H). LC/MS [M+H] 594.3 (calculated); LC/MS [M+H] 594.4 (observed).

### Preparation of 6-amino-3-[2-(aminomethyl)pyrimidin-5-yl]-N,N-dipropyl-7H-pyrido[3,2-b]azepine-8-carboxamide, L-28c

To a solution of L-28b (100 mg, 168 mol, 1 eq) in EtOAc (2 mL) was added HCl/EtOAc (4 M, 2.11 mL, 50 eq), and then stirred at 25° C. for 0.5 hr. The mixture was concentrated to L-28c (70 mg, 162 mol, 96.6% yield, HCl) as an off-white solid. ¹H NMR (MeOD, 400 MHz) δ 9.30 (s, 2H), 9.06 (d, J=2.0 Hz, 1H), 8.26 (d, J=2.0 Hz, 1H), 7.12 (s, 1H), 4.52 (s, 2H), 3.61-3.42 (m, 6H), 1.84-1.67 (m, 4H), 1.11-0.87 (m, 6H). LC/MS [M+H] 394.2 (calculated); LC/MS [M+H] 394.1 (observed).

### Preparation of azaBzL-28

A mixture of L-28c (50 mg, 116 mol, 1 eq, HCl) and DIEA (45.0 mg, 348 mol, 60.7 L, 3 eq) in DMF (1 mL) was added to the solution of (2,3,5,6-tetrafluorophenyl) 3-[2-[2-[2-[2-[2-[2-[2-[2-[2-[3-oxo-3-(2,3,5,6-tetrafluorophenoxy)propoxy]ethoxy]ethoxy]ethoxy]ethoxy]ethoxy]ethoxy]ethoxy]ethoxy]ethoxy]propanoate (119 mg, 139 mol, 1.2 eq) in DMF (1 mL), and then was stirred at 0° C. for 0.5 hr. The mixture was adjusted pH=5 with TFA and filtered. The filtrate was purified by prep-HPLC (column: Phenomenex Luna C18 75*30 mm*3 um; mobile phase: [H₂O (0.10% TFA)-ACN]; gradient: 25%-45% B over 8.0 min) to give azaBzL-28 (64.5 mg, 49.2 mol, 42.3% yield, 2TFA) as light yellow oil. ¹H NMR (MeOD, 400 MHz) δ 9.17 (s, 2H), 9.04 (d, J=2.0 Hz, 1H), 8.15 (d, J=2.0 Hz, 1H), 7.48-7.40 (m, 1H), 7.11 (s, 1H), 4.72 (s, 2H), 3.88 (t, J=6.0 Hz, 2H), 3.82 (t, J=6.0 Hz, 2H), 3.71-3.59 (m, 36H), 3.58-3.43 (m, 6H), 2.99 (t, J=6.0 Hz, 2H), 2.62 (t, J=6.0 Hz, 2H), 1.82-1.67 (m, 4H), 1.00-0.97 (m, 6H). LC/MS [M+H] 1082.5 (calculated); LC/MS [M+H] 1082.4 (observed).

Example L-32 Synthesis of (2,3,5,6-tetrafluorophenyl) 3-[2-[2-[2-[2-[2-[2-[2-[2-[2-[3-[3-[[6-amino-8-[ethoxy(propyl)carbamoyl]-7H-pyrido[3,2-b]azepine-3-carbonyl]amino]-7,8-dihydro-5H-1,6-naphthyridin-6-yl]-3-oxo-propoxy]ethoxy]ethoxy]ethoxy]ethoxy]ethoxy]ethoxy]ethoxy]ethoxy]ethoxy]propanoate, azaBzL-32

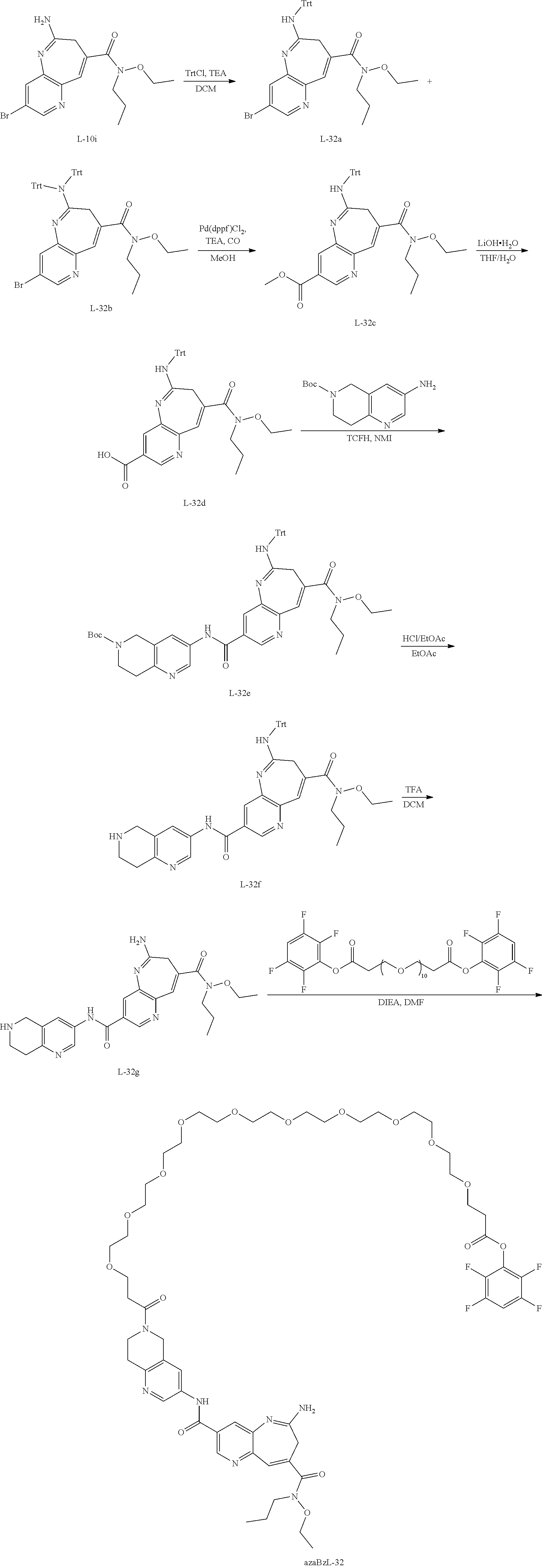

### Preparation of 3-bromo-N-ethoxy-N-propyl-6-(tritylamino)-7H-pyrido[3,2-b]azepine-8-carboxamide and 3-bromo-6-(ditritylamino)-N-ethoxy-N-propyl-7H-pyrido[3,2-b]azepine-8-carboxamide, L-32a

To a solution of 6-amino-3-bromo-N-ethoxy-N-propyl-7H-pyrido[3,2-b]azepine-8-carboxamide, L-10i (25.0 g, 68.1 mmol, 1 eq) and TEA (20.7 g, 204 mmol, 28.4 mL, 3 eq) in DCM (500 mL) was added trityl chloride, TrtCl (28.5 g, 102 mmol, 1.5 eq) at 25° C. under N₂, and then heated to 50° C. and stirred for 12 hrs. The reaction mixture was quenched by addition of H₂O (300 mL) at 0° C., and then extracted with DCM (150 mL×3). The combined organic layers were washed with brine (50 mL), dried over Na₂SO₄, filtered and concentrated under reduced pressure to give a residue. The residue was purified by column chromatography (SiO₂, Petroleum ether/Ethyl acetate=I1/0 to 1/1) to give L-32a (11.3 g, 18.5 mmol, 27.2% yield) as a yellow solid and 3-bromo-6-(ditritylamino)-N-ethoxy-N-propyl-7H-pyrido[3,2-b]azepine-8-carboxamide, L-32b (8.8 g, 10.3 mmol, 15.1% yield) as a yellow solid. LC/MS [M+H] 609.2 (calculated); LC/MS [M+H] 609.1 (observed). LC/MS [M+H] 851.3 (calculated); LC/MS [M+H] 851.3 (observed).

### Preparation of methyl 8-[ethoxy(propyl)carbamoyl]-6-(tritylamino)-7H-pyrido[3,2-b]azepine-3-carboxylate, L-32c

A mixture of L-32a (5.60 g, 9.19 mmol, 1 eq), Pd(dppf)Cl₂ (672 mg, 918 mol, 0.1 eq) and TEA (2.79 g, 27.6 mmol, 3.84 mL, 3 eq) in MeOH (50 mL) was degassed and purged with CO for 3 times, and then heated to 80° C. and stirred for 16 hrs under CO atmosphere (50 psi). The reaction mixture was cooled to 25° C. and filtered and the filter cake was dried under reduced pressure as pure product. The filtrate was concentrated under reduced pressure to give a residue. The residue was purified by column chromatography (SiO₂, Petroleum ether/Ethyl acetate=I/O to 1/1) to give L-32c (3.50 g, 5.95 mmol, 64.7% yield) as a brown solid. ¹H NMR (CDCl₃, 400 MHz) δ 8.78 (s, 1H), 7.50 (s, 2H), 7.35-7.28 (m, 6H), 7.26-7.15 (m, 9H), 6.33 (s, 1H), 3.98-3.96 (m, 2H), 3.90 (s, 3H), 3.76 (t, J=6.8 Hz, 2H), 2.83 (s, 2H), 1.78-1.75 (m, 2H), 1.28 (t, J=6.8 Hz, 3H), 0.98 (t, J=6.8 Hz, 3H). LC/MS [M+H] 589.3 (calculated); LC/MS [M+H] 589.2 (observed).

### Preparation of methyl 8-[ethoxy(propyl)carbamoyl]-6-(tritylamino)-7H-pyrido[3,2-b]azepine-3-carboxylic acid, L-32d

To a solution of L-32c (2.75 g, 4.67 mmol, 1 eq) in THE (30 mL) was added a solution of LiOH·H₂O (588 mg, 14.0 mmol, 3 eq) in H₂O (10 mL) at 0° C., and then warmed to 25° C. and stirred for 2 hrs. The reaction mixture was cooled to 0° C. and diluted with H₂O (30 mL) and adjusted to pH=5 with 2 N HCl at 0° C., concentrated under reduced pressure to remove THF. The mixture was filtered and the filter cake was dried under reduced pressure to give L-32d (2.80 g, crude) as a light yellow solid. ¹H NMR (DMSO-d₆, 400 MHz) δ 8.57 (s, 1H), 8.39 (s, 1H), 7.33-7.20 (m, 12H), 7.19-7.14 (m, 4H), 7.12 (s, 1H), 3.96 (q, J=7.2 Hz, 2H), 3.70 (t, J=6.8 Hz, 2H), 3.03 (s, 2H), 1.77-1.64 (m, 2H), 1.14 (t, J=6.8 Hz, 3H), 0.94 (t, J=7.2 Hz, 3H). LC/MS [M+H] 575.3 (calculated); LC/MS [M+H] 575.3 (observed).

### Preparation of tert-butyl 3-[[8-[ethoxy(propyl)carbamoyl]-6-(tritylamino)-7H-pyrido[3,2-b]azepine-3-carbonyl]amino]-7,8-dihydro-5H-1,6-naphthyridine-6-carboxylate, L-32e

To a solution of L-32d (0.95 g, 1.65 mmol, 1 eq) in acetonitrile, ACN (10 mL) were added N-methylimidazole, NMI (1.36 g, 16.5 mmol, 1.32 mL, 10 eq) and tert-butyl 3-amino-7,8-dihydro-5H-1,6-naphthyridine-6-carboxylate (618 mg, 2.48 mmol, 1.5 eq), then added TCFH (1.86 g, 6.61 mmol, 4 eq). The mixture was stirred at 25° C. for 2 hrs, quenched by addition of H₂O (20 mL) at 0° C., and then extracted with EtOAc (30 mL×3). The combined organic layers were washed with brine (20 mL×2), dried over Na₂SO₄, filtered and concentrated under reduced pressure to give a residue. The residue was purified by flash silica gel chromatography (ISCO®; 20 g SepaFlash® Silica Flash Column, Eluent of 70% Ethyl acetate/Petroleum ether gradient @80 mL/min) to give L-32e (2.3 g, crude) was obtained as a yellow oil. LC/MS [M+H] 806.4 (calculated); LC/MS [M+H] 806.3 (observed).

### Preparation of N8-ethoxy-N8-propyl-N3-(5,6,7,8-tetrahydro-1,6-naphthyridin-3-yl)-6-(tritylamino)-7H-pyrido[3,2-b]azepine-3,8-dicarboxamide, L-32f

To a solution of L-32e (2.30 g, 2.85 mmol, 1 eq) in EtOAc (20 mL) was added HCl/EtOAc (4 M, 10.7 mL, 15 eq), and then stirred at 25° C. for 2 hrs. The reaction mixture was concentrated under reduced pressure to give a residue. The residue was purified by prep-HPLC (column: Welch Xtimate C18 250*70 mm*0 um; mobile phase: [H₂O (0.1% TFA)-ACN]; gradient: 25%-55% B over 20.0 min) to give L-32e (1 g, 1.42 mmol, 49.7% yield) as a yellow solid. ¹H NMR (MeOD, 400 MHz) δ 8.81 (s, 1H), 8.71 (d, J=2.0 Hz, 1H), 8.23 (d, J=2.0 Hz, 1H), 7.66 (d, J=2.0 Hz, 1H), 7.45-7.22 (m, 16H), 4.48 (s, 2H), 4.05 (q, J=7.2 Hz, 2H), 3.81 (t, J=6.8 Hz, 2H), 3.65 (t, J=6.4 Hz, 2H), 3.23 (t, J=6.4 Hz, 4H), 1.88-1.77 (m, 2H), 1.26 (t, J=7.2 Hz, 3H), 1.04 (t, J=7.2 Hz, 3H). LC/MS [M+H] 706.3 (calculated); LC/MS [M+H] 706.4 (observed).

### Preparation of 6-amino-N8-ethoxy-N8-propyl-N3-(5,6,7,8-tetrahydro-1,6-naphthyridin-3-yl)-7H-pyrido[3,2-b]azepine-3,8-dicarboxamide, L-32g

To a solution of L-32f (0.30 g, 425 mol, 1 eq) in DCM (3 mL) was added TFA (969 mg, 8.50 mmol, 631 μL, 20 eq), and then heated to 50° C. and stirred for 16 hrs. The reaction mixture was concentrated under reduced pressure to give a residue. The residue was purified by prep-HPLC (column: Phenomenex Luna C18 75*30 mm*3 um; mobile phase: [H₂O (0.1% TFA)-ACN]; gradient: 1%-30% B over 8.0 min) to give L-32g (0.18 g, 388 mol, 91.4% yield) as a white solid. ¹H NMR (MeOD, 400 MHz) δ 9.15 (s, 1H), 8.80 (s, 1H), 8.35 (s, 1H), 8.28 (s, 1H), 7.45 (s, 1H), 4.49 (s, 2H), 4.10 (q, J=7.2 Hz, 2H), 3.77 (t, J=7.2 Hz, 2H), 3.66 (t, J=6.4 Hz, 2H), 3.51 (s, 2H), 3.23 (t, J=6.4 Hz, 2H), 1.84-1.74 (m, 2H), 1.21 (t, J=7.2 Hz, 3H), 1.01 (t, J=7.2 Hz, 3H). LC/MS [M+H] 464.2 (calculated); LC/MS [M+H] 464.1 (observed).

### Preparation of azaBzL-32

To a solution of (2,3,5,6-tetrafluorophenyl) 3-[2-[2-[2-[2-[2-[2-[2-[2-[2-[3-oxo-3-(2,3,5,6-tetrafluorophenoxy)propoxy]ethoxy]ethoxy]ethoxy]ethoxy]ethoxy]ethoxy]ethoxy]ethoxy]ethoxy]propanoate (295 mg, 345 mol, 2 eq) in DMF (2 mL) was added dropwise the solution of DIEA (22.3 mg, 173 mol, 30.1 μL, 1 eq) and L-32g (0.08 g, 173 mol, 1 eq) in DMF (2 mL), and then stirred at 0° C. for 1 hr. The reaction mixture was adjusted pH=6 with TFA and then purified by prep-HPLC (column: Phenomenex Luna C18 75*30 mm*3 um; mobile phase: [H₂O (0.1% TFA)-ACN]; gradient: 15%-45% B over 8.0 min) to give azaBzL-32 (0.04 g, 34.2 mol, 19.8% yield, 98.5% purity) as a colorless oil. ¹H NMR (MeOD, 400 MHz) δ 9.20 (s, 1H), 8.95 (s, 1H), 8.41-8.22 (m, 2H), 7.50-7.36 (m, 2H), 4.03-3.95 (m, 4H), 3.85 (t, J=6.0 Hz, 2H), 3.82-3.74 (m, 4H), 3.64-3.56 (m, 34H), 3.53-3.51 (m, 5H), 3.39-3.37 (m, 1H), 3.18 (t, J=6.0 Hz, 1H), 3.08-3.01 (m, 1H), 2.96 (t, J=6.0 Hz, 2H), 2.83-2.77 (m, 2H), 1.83-1.74 (m, 2H), 1.21 (t, J=7.2 Hz, 3H), 1.01 (t, J=7 Hz, 3H). LC/MS [M+H] 1152.5 (calculated); LC/MS [M+H] 1152.6 (observed).

### Example L-37 Synthesis of 6-amino-3-[3-[3-[[3-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]propanoylamino]methyl]azetidin-1-yl]sulfonylphenyl]-N-ethoxy-N-propyl-7H-pyrido[3,2-b]azepine-8-carboxamide, azaBzL-37

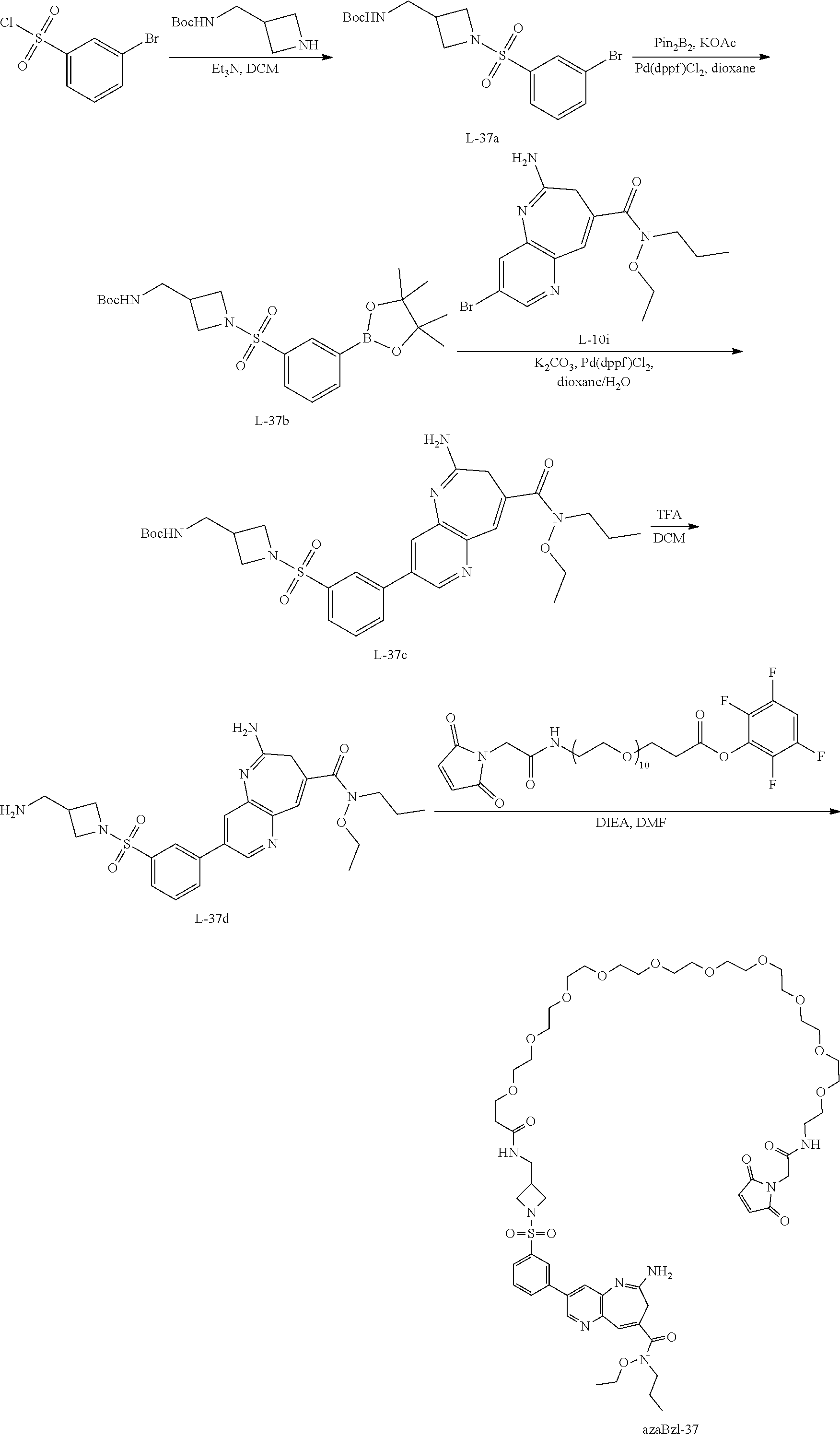

### Preparation of tert-butyl N-[[1-(3-bromophenyl)sulfonylazetidin-3-yl]methyl]carbamate, L-37a

To a solution of tert-butyl N-(azetidin-3-ylmethyl)carbamate;hydrochloride (10.5 g, 46.9 mmol, 1 eq) in DCM (25 mL) were added triethylamine, Et₃N (9.50 g, 93.9 mmol, 13.1 mL, 2 eq) and 3-bromobenzenesulfonyl chloride (12.0 g, 46.9 mmol, 6.77 mL, 1 eq) at 0° C., and then stirred at 25° C. for 1 hr. The residue was poured into water (15 mL). The aqueous phase was extracted with DCM (20 mL×3). The combined organic phase was washed with brine (10 mL×3), dried with anhydrous Na₂SO₄, filtered and concentrated by vacuum. The crude product was triturated with MTBE (30 mL) at 0° C. for 15 min. to give L-37a (14.0 g, 34.5 mmol, 73.6% yield) as a white solid. LC/MS [M+Na] 427.0 (calculated); LC/MS [M+Na] 427.0 (observed).

### Preparation of tert-butyl N-[[1-[3-(4,4,5,5-tetramethyl-1,3,2-dioxaborolan-2-yl)phenyl]sulfonylazetidin-3-yl]methyl]carbamate, L-37b

A mixture of L-37a (3.00 g, 7.40 mmol, 1 eq), KOAc (1.45 g, 14.8 mmol, 2 eq), Pin₂B₂ (2.44 g, 9.62 mmol, 1.3 eq) and Pd(dppf)Cl₂ (270 mg, 370 mol, 0.05 eq) in dioxane (30 mL) was degassed and purged with N₂ for 3 times at 25° C., and heated to 95° C. stirred for 2 hrs under N₂ atmosphere. The reaction mixture was concentrated by vacuum. The residue was purified by flash silica gel chromatography (biotage®; 40g SepaFlash® Silica Flash Column, Eluent of 0~35% Ethyl acetate/Petroleum ether gradient @80 mL/min) to give L-37b (3.30 g, 7.29 mmol, 98.6% yield) was obtained as a yellow oil. ¹H NMR (CDCl₃, 400 MHz) δ 8.26 (s, 1H), 8.06 (d, J=7.2 Hz, 1H), 7.91 (d, J=8.0 Hz, 1H), 7.57 (t, J=8.0 Hz, 1H), 3.82 (t, J=8.0 Hz 2H), 3.55-3.46 (m, 2H), 3.16 (t, J=6.0 Hz, 2H), 2.65-2.55 (m, 1H), 1.36 (s, 9H), 1.27 (s, 12H). LC/MS [M+Na] 475.2 (calculated); LC/MS [M+Na] 475.1 (observed).

### Preparation of tert-butyl N-[[1-[3-[6-amino-8-[ethoxy(propyl)carbamoyl]-7H-pyrido[3,2-b]azepin-3-yl]phenyl]sulfonylazetidin-3-yl]methyl]carbamate, L-37c

To a solution of L-37c (312 mg, 689 mol, 1.3 eq) in dioxane (5.00 mL) and H₂O (0.5 mL) were added K₂CO₃ (147 mg, 1.06 mmol, 2 eq), 6-amino-3-bromo-N-ethoxy-N-propyl-7H-pyrido[3,2-b]azepine-8-carboxamide, L-10i (195 mg, 531 mol, 1 eq) and Pd(dppf)Cl₂ (19.4 mg, 26.5 mol, 0.05 eq) at 25° C. The solution was degassed and purged with N₂ for 3 times, then heated to 95° C. and stirred for 2 hrs under N₂ atmosphere. The reaction mixture was cooled to 25° C., filtered and the filtrate was concentrated under reduced pressure to give a residue. The residue was purified by flash silica gel chromatography (biotage®; 4g SepaFlash® Silica Flash Column, Eluent of 0~40% Ethyl acetate/Petroleum ether gradient@60 mL/min) to give L-37c (300 mg, 489 mol, 92.3% yield) as brown oil. ¹H NMR (CDCl₃, 400 MHz) δ 8.78 (s, 1H), 8.12 (s, 1H), 7.90-7.97 (m, 3H), 7.65-7.77 (m, 2H), 3.98 (q, J=7.2 Hz, 2H), 3.92-3.84 (m, 2H), 3.75 (t, J=7.2 Hz, 2H), 3.62-3.55 (m, 2H), 3.21-3.11 (m, 2H), 3.10 (s, 2H), 2.72-2.59 (m, 1H), 1.78-1.75 (m, 2H), 1.39 (s, 9H), 1.31-1.28 (m, 3H), 0.99 (t, J=7.6, 3H). LC/MS [M+H] 613.3 (calculated); LC/MS [M+H] 613.2 (observed).

### Preparation of 6-amino-3-[3-[3-(aminomethyl)azetidin-1-yl]sulfonylphenyl]-N-ethoxy-N-propyl-7H-pyrido[3,2-b]azepine-8-carboxamide, L-37d

To a solution of L-37c (0.25 g, 408 mol, 1 eq) in DCM (1 mL) was added TFA (465 mg, 4.08 mmol, 303 μL, 10 eq), and then heated to 50° C. and stirred for 1 hr. The reaction mixture was cooled to 25° C. and concentrated by vacuum. The residue was purified by prep-HPLC (column: Phenomenex luna C18 250*50 mm*15 um; mobile phase: [H₂O (0.1% TFA)-ACN]; gradient: 5%-30% B over 10.0 min) to give L-37d (100 mg, 195 mol, 47.8% yield) was obtained as a white solid. LC/MS [M+H] 513.2 (calculated); LC/MS [M+H] 513.2 (observed).

### Preparation of azaBzL-37

To a solution of L-37d (50.0 mg, 97.5 mol, 1 eq) in DMF (0.5 mL) were added DIEA (37.8 mg, 293 mol, 50.9 μL, 3 eq) and (2,3,5,6-tetrafluorophenyl) 3-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]propanoate (67.5 mg, 82.9 mol, 0.85 eq) at 0° C., and then stirred at 25° C. for 1 hr. The reaction mixture was acidified to pH-6 with TFA and filtered. The filtrate was purified by prep-HPLC (column: Phenomenex luna C18 100*40 mm*3 um; mobile phase: [H₂O (0.10% TFA)-ACN]; gradient: 10%-45% B over 8.0 min) to give azaBzL-37 (33.0 mg, 28.4 mol, 29.1% yield) as colorless oil. ¹H NMR (MeOD, 400 MHz) δ 9.05 (d, J=2.0 Hz, 1H), 8.19-8.16 (m, 3H), 7.98 (d, J=8.0 Hz, 1H), 7.89 (t, J=8.0 Hz, 1H), 7.48 (s, 1H), 6.89 (s, 2H), 4.16 (s, 2H), 4.05-3.99 (m, 2H), 3.89 (t, J=8.0 Hz, 2H), 3.79-3.75 (m, 2H), 3.65-3.59 (m, 38H), 3.58-3.56 (m, 2H), 3.55-3.53 (m, 4H), 3.36 (s, 2H), 3.15 (d, J= 6.4 Hz, 2H), 2.73-2.64 (m, 1H), 2.29 (t, J=6.0 Hz, 2H), 1.84-1.75 (m, 2H), 1.23 (t, J=7.2 Hz, 3H), 1.01 (t, J=7.2 Hz, 3H). LC/MS [M+H] 1161.5 (calculated); LC/MS [M+H] 1161.7 (observed).

### Example L-38 Synthesis of (2,3,5,6-tetrafluorophenyl) 3-[2-[2-[2-[2-[2-[2-[2-[2-[2-[3-[[1-[3-[6-amino-8-[ethoxy(propyl)carbamoyl]-7H-pyrido[3,2-b]azepin-3-yl]phenyl]sulfonylazetidin-3-yl]methylamino]-3-oxo-propoxy]ethoxy]ethoxy]ethoxy]ethoxy]ethoxy]ethoxy]ethoxy]ethoxy]ethoxy]propanoate, azaBzL-38

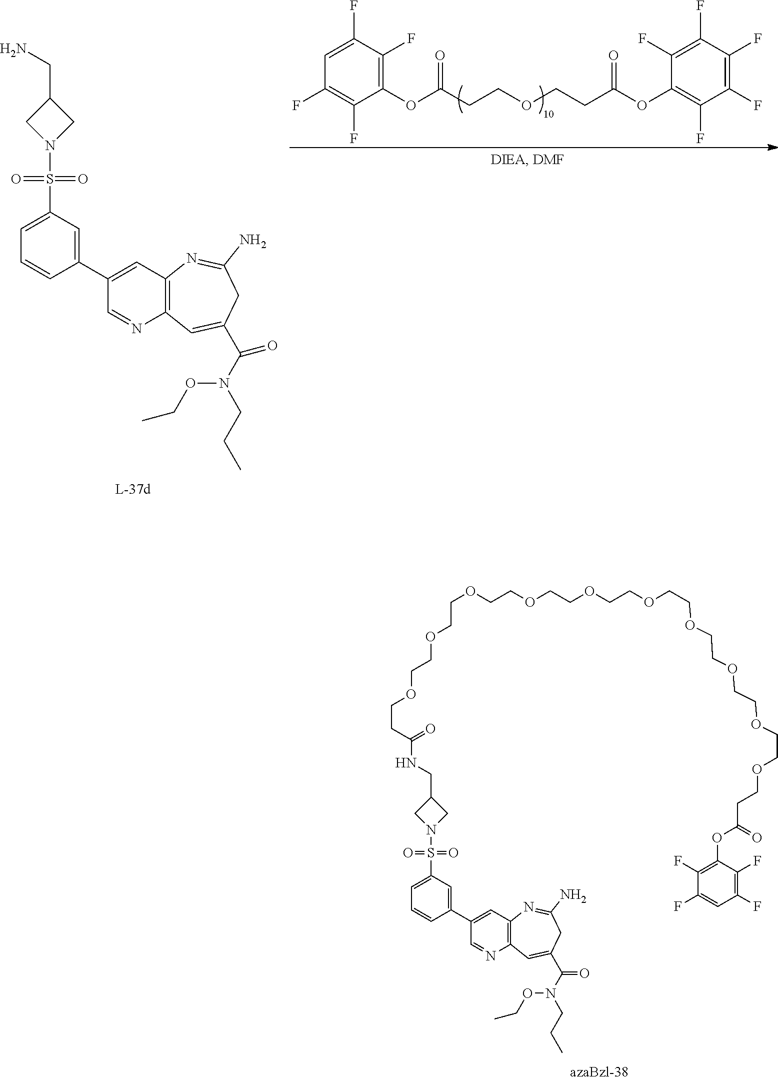

To a solution of(2,3,5,6-tetrafluorophenyl) 3-[2-[2-[2-[2-[2-[2-[2-[2-[2-[3-oxo-3-(2,3,5,6-tetrafluorophenoxy)propoxy]ethoxy]ethoxy]ethoxy]ethoxy]ethoxy]ethoxy]ethoxy]ethoxy]ethoxy]propanoate (140 mg, 164 mol, 1.2 eq) in DMF (1 mL) were added DIEA (52.9 mg, 409 mol, 71.3 μL, 3 eq) and 6-amino-3-[3-[3-(aminomethyl)azetidin-1-yl]sulfonylphenyl]-N-ethoxy-N-propyl-7H-pyrido[3,2-b]azepine-8-carboxamide, L-37d (70 mg, 136 mol, 1 eq) at 0° C., and then stirred at 25° C. for 1 hr. The reaction mixture was acidified by TFA to Ph-6 and filtered. The filtrate was purified by prep-HPLC (column: Phenomenex Luna C18 80*30 mm*3 um; mobile phase: [H₂O (0.1% TFA)-ACN]; gradient: 30%-60% B over 8.0 min). (30.0 mg, 24.9 mol, 18.3% yield) to give azaBzL-38 as colorless oil. ¹H NMR (MeOD, 400 MHz) δ 9.04 (d, J=2.0 Hz, 1H), 8.18-8.16 (m, 3H), 7.98 (d, J=8.0 Hz, 1H), 7.89 (t, J=8.0 Hz, 1H), 7.48 (s, 1H), 7.44-7.37 (m, 1H), 4.01 (q, J=7.2 Hz, 2H), 3.91-3.87 (m, 2H), 3.87-3.84 (m, 2H), 3.77 (t, J=7.2 Hz, 2H), 3.64-3.59 (m, 36H), 3.57-3.55 (m, 2H), 3.54-3.51 (m, 4H), 3.14 (d, J=6.4 Hz, 2H), 2.97 (t, J=6.00 Hz, 2H), 2.74-2.64 (m, 1H), 2.28 (t, J=6.00 Hz, 2H), 1.82-1.75 (m, 2H), 1.22 (t, J=7.2 Hz, 3H), 1.02 (t, J=7.2 Hz, 3H). LC/MS [M+H] 1201.5 (calculated); LC/MS [M+H] 1201.4 (observed).

### Example L-39 Synthesis of 6-amino-3-[(3S)-3-[3-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]propanoylamino]piperidine-1-carbonyl]-N-ethoxy-N-propyl-7H-pyrido[3,2-b]azepine-8-carboxamide, azaBzL-39

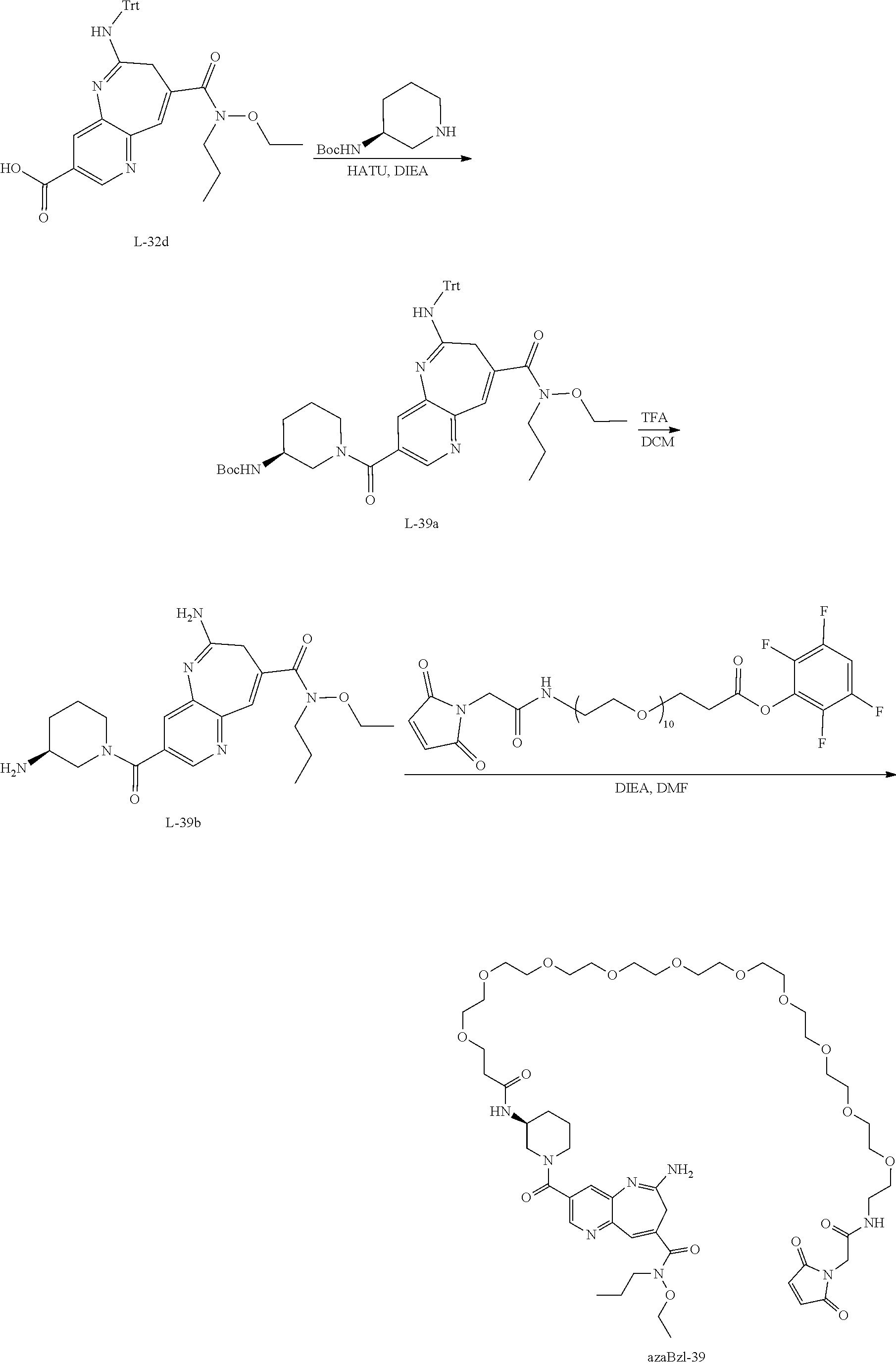

### Preparation of tert-butyl N-[(3S)-1-[8-[ethoxy(propyl)carbamoyl]-6-(tritylamino)-7H-pyrido[3,2-b]azepine-3-carbonyl]-3-piperidyl]carbamate, L-39a

To a mixture of 8-[ethoxy(propyl)carbamoyl]-6-(tritylamino)-7H-pyrido[3,2-b]azepine-3-carboxylic acid, L-32d (300 mg, 522 mol, 1 eq) and tert-butyl N-[(3S)-3-piperidyl]carbamate (125 mg, 627 mol, 1.2 eq) in MeCN (8 mL) was added NMI (171 mg, 2.09 mmol, 167 μL, 4 eq) and chloro-N,N,N′,N′-tetramethylformamidinium hexafluorophosphate, TCFH, CAS Reg. No. 207915-99-9 (219 mg, 783 mol, 1.5 eq), and then stirred at 25° C. for 1 hr. Alternatively, HATU can be used as the coupling reagent. The reaction mixture was poured into water (10 mL). The aqueous phase was extracted with ethyl acetate (10 mL×3). The combined organic phase was washed with brine (8 mL×3), dried with anhydrous Na₂SO₄, filtered and concentrated in vacuum. The residue was purified by flash silica gel chromatography (biotage®; 12g SepaFlash® Silica Flash Column, Eluent of 0-45% Ethyl acetate/Petroleum ether gradient @80 mL/min) to give L-39a (350 mg, 462 mol, 88.6% yield) was obtained as yellow oil. LC/MS [M+H] 757.4 (calculated); LC/MS [M+H] 757.4 (observed).

### Preparation of 6-amino-3-[(3S)-3-aminopiperidine-1-carbonyl]-N-ethoxy-N-propyl-7H-pyrido[3,2-b]azepine-8-carboxamide, L-39b

To a solution of L-39a (350 mg, 462 mol, 1 eq) in DCM (7 mL) was added TFA (1.58 g, 13.8 mmol, 1.03 mL, 30 eq), then heated to 50° C. and stirred for 2 hrs. The reaction mixture was concentrated in vacuum. The crude product was purified by re-crystallization from MTBE (15 mL) at 0° C. to give L-39b (160 mg, 386 mol, 83.5% yield) as a yellow solid. LC/MS [M+H] 415.2 (calculated); LC/MS [M+H] 415.3 (observed).

### Preparation of azaBzL-39

To a mixture of L-39b (60.0 mg, 145 mol, 1 eq) in DMF (2 mL) was added DIEA (56.1 mg, 434 mol, 75.6 μL, 3 eq) and (2,3,5,6-tetrafluorophenyl) 3-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]propanoate (118 mg, 145 mol, 1 eq) at 0° C., and then stirred at 25° C. for 0.5 hr. The pH of the reaction solution was adjusted to about 9 by adding TFA and purified by prep-HPLC (column: Phenomenex Luna C18 75*30 mm*3 um; mobile phase: [H₂O (0.1% TFA)-ACN]; gradient: 5%-45% B over 8.0 min) to give azaBzL-39 (35.0 mg, 32.9 mol, 22.7% yield) as colorless oil. ¹H NMR (DMSO-d₆, 400 MHz) δ 9.28 (s, 1H), 8.64 (s, 1H), 8.21 (t, J=5.2 Hz, 1H), 7.93 (d, J=7.2 Hz, 1H), 7.79 (s, 1H), 7.21 (s, 1H), 7.08 (s, 2H), 4.01 (s, 2H), 3.90 (q, J=7.2 Hz, 2H), 3.64 (t, J=7.2 Hz, 2H), 3.62-3.58 (m, 1H), 3.52-3.47 (m, 38H), 3.45-3.43 (m, 4H), 3.43-3.42 (m, 2H), 3.41-3.40 (m, 2H), 3.21-3.17 (m, 2H), 2.36-2.29 (m, 2H), 1.89-1.77 (m, 2H), 1.68-1.63 (m, 2H), 1.57-1.45 (m, 2H), 1.09 (t, J=7.2 Hz, 3H), 0.91 (t, J=7.2 Hz, 3H). LC/MS [M+H] 1063.5 (calculated); LC/MS [M+H] 1063.4 (observed).

### Example L-41 Synthesis of 6-amino-3-[(3R)-3-[3-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]propanoylamino] piperidine-1-carbonyl]-N-ethoxy-N-propyl-7H-pyrido[3,2-b]azepine-8-carboxamide, azaBzL-41

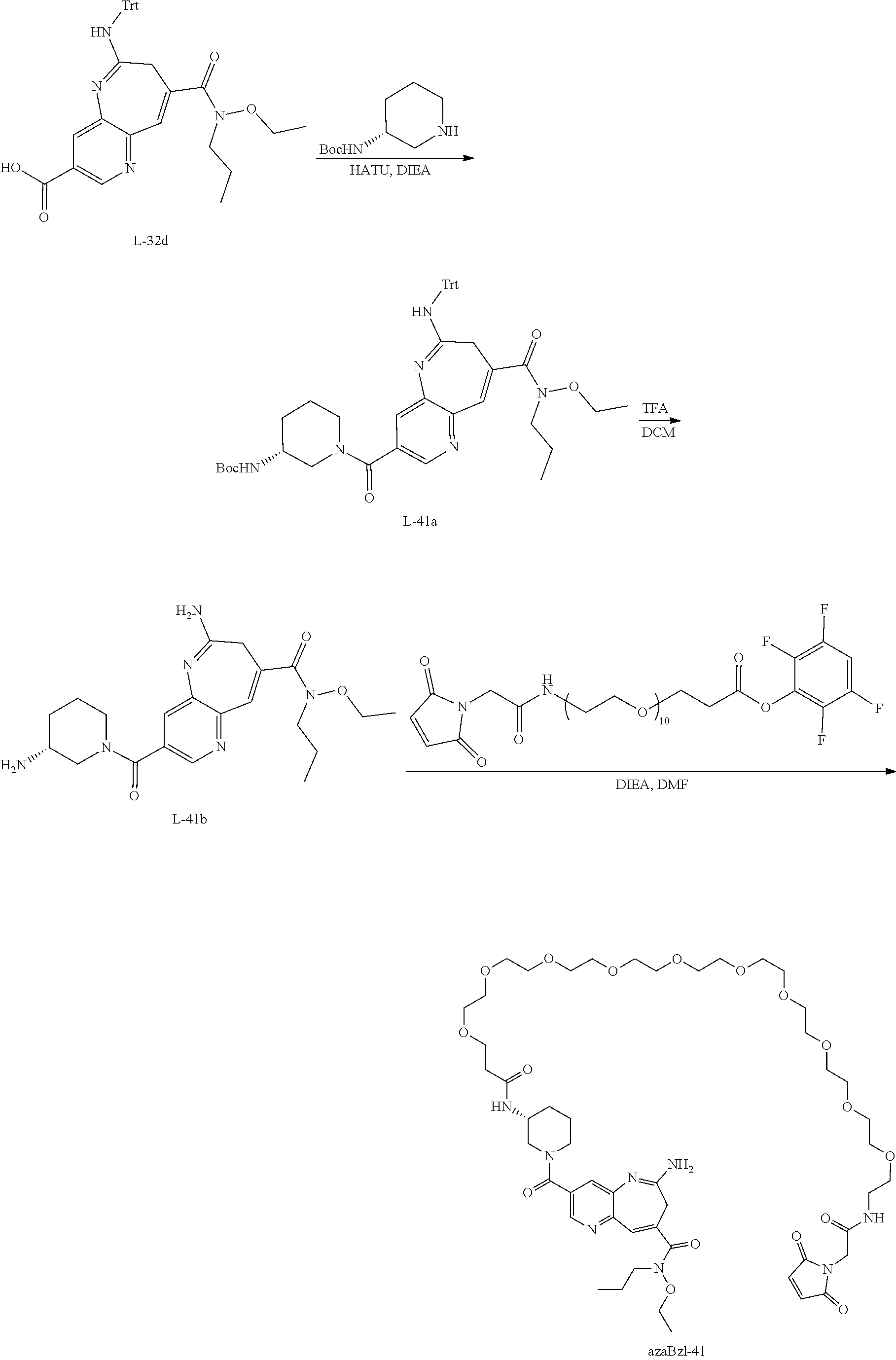

### Preparation of tert-butyl N-[(3R)-1-[8-[ethoxy(propyl)carbamoyl]-6-(tritylamino)-7H-pyrido[3,2-b]azepine-3-carbonyl]-3-piperidyl]carbamate, L-41a

To a solution of 8-[ethoxy(propyl)carbamoyl]-6-(tritylamino)-7H-pyrido[3,2-b]azepine-3-carboxylic acid, L-32d (300 mg, 522 mol, 1 eq) in DMF (5.00 mL) were added HATU (297 mg, 783 mol, 1.5 eq), DIEA (134 mg, 1.04 mmol, 181 μL, 2 eq) and tert-butyl N-[(3R)-3-piperidyl]carbamate (125 mg, 626 mol, 1.2 eq) at 0° C., then warmed to 25° C. and stirred for 1 hr. The reaction mixture was diluted with ice water 5 mL and extracted with ethyl acetate 15 mL (5 mL×3). The organic layers were washed with water (5 mL) and brine (5 mL), dried over Na₂SO₄, filtered and concentrated under reduced pressure to give a residue. The residue was purified by flash silica gel chromatography (ISCO®; 12 g SepaFlash® Silica Flash Column, Eluent of 0-100% Ethyl acetate/Petroleum ether gradient @60 mL/min) to obtain L-41a (443 mg, crude) as yellow oil. LC/MS [M+H] 757.4 (calculated); LC/MS [M+H] 757.2 (observed).

### Preparation of 6-amino-3-[(3R)-3-aminopiperidine-1-carbonyl]-N-ethoxy-N-propyl-7H-pyrido[3,2-b]azepine-8-carboxamide, L-41b

To a solution of L-41a (443 mg, 585 mol, 1 eq) in DCM (5 mL) was added TFA (1.33 g, 11.7 mmol, 869 μL, 20 eq) at 25° C., and then heated to 50° C. and stirred for 12 hrs. After that, the reaction mixture was concentrated under reduced pressure to give a residue. The residue was triturated with MTBE (5 mL) at 25° C. for 20 min, filtered and the filter cake was concentrated under reduced pressure to obtain L-41b (285 mg, crude) as a red brown solid. ¹H NMR (MeOD, 400 MHz,) δ 8.7 (s, 1H), 7.90 (d, J=1.6 Hz, 1H), 7.41 (s, 1H), 3.98 (q, J=7.2 Hz, 2H), 3.80-3.67 (m, 4H), 3.50 (s, 2H), 3.45-3.36 (m, 2H), 2.23-2.14 (m, 1H), 1.88-1.86 (m, 2H), 1.83-1.64 (m, 4H), 1.20 (t, J=7.2 Hz, 3H), 1.01 (t, J=7.2 Hz, 3H). LC/MS [M+H] 415.2 (calculated); LC/MS [M+H] 415.2 (observed).

### Preparation of azaBzL-41

To a solution of L-41b (48 mg, 90.8 mol, 1 eq, TFA salt) in DMF (1.5 mL) were added DIEA (35.2 mg, 272 mol, 47.4 μL, 3 eq) and (2,3,5,6-tetrafluorophenyl) 3-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]propanoate (74.0 mg, 90.8 mol, 1 eq) at 0° C., and then stirred at 0° C. for 0.5 hr. The reaction mixture was adjusted pH=6 with TFA at 0° C., and then purified by prep-HPLC (column: Phenomenex luna C18 100*40 mm*3 um; mobile phase: [H2O (0.1% TFA)-ACN];gradient: 5%-40% B over 8.0 min) to give azaBzL-41 (20 mg, 18.8 mol, 20.7% yield) as colorless oil. ¹H NMR (DMSO-d₆, 400 MHz) δ 8.52 (s, 1H), 7.96-7.90 (m, 1H), 7.68 (d, J=1.0 Hz, 1H), 7.63 (d, J=7.2 Hz, 1H), 7.19 (s, 1H), 7.01 (s, 2H), 4.03 (s, 2H), 3.92 (q, J=7.0 Hz, 2H), 3.75-3.71 (m, 1H), 3.66 (t, J=7.0 Hz, 3H), 3.62-3.57 (m, 2H), 3.55-3.49 (m, 36H), 3.44 (t, J=5.9 Hz, 2H), 3.31 (d, J=1.1 Hz, 2H), 3.26-3.20 (m, 6H), 2.35-2.31 (m, 2H), 1.92-1.74 (m, 2H), 1.73-1.66 (m, 2H), 1.50-1.49 (m, 2H), 1.12 (t, J=7.0 Hz, 3H), 0.94 (t, J=7.5 Hz, 3H). LC/MS [M+H] 1063.5 (calculated); LC/MS [M+H] 1063.4 (observed).

### Example L-42 Synthesis of 2-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]ethyl N-[[1-[6-amino-8-[ethoxy(propyl)carbamoyl]-7H-pyrido[3,2-b]azepine-3-carbonyl]azetidin-3-yl]methyl]carbamate, azaBzL-42

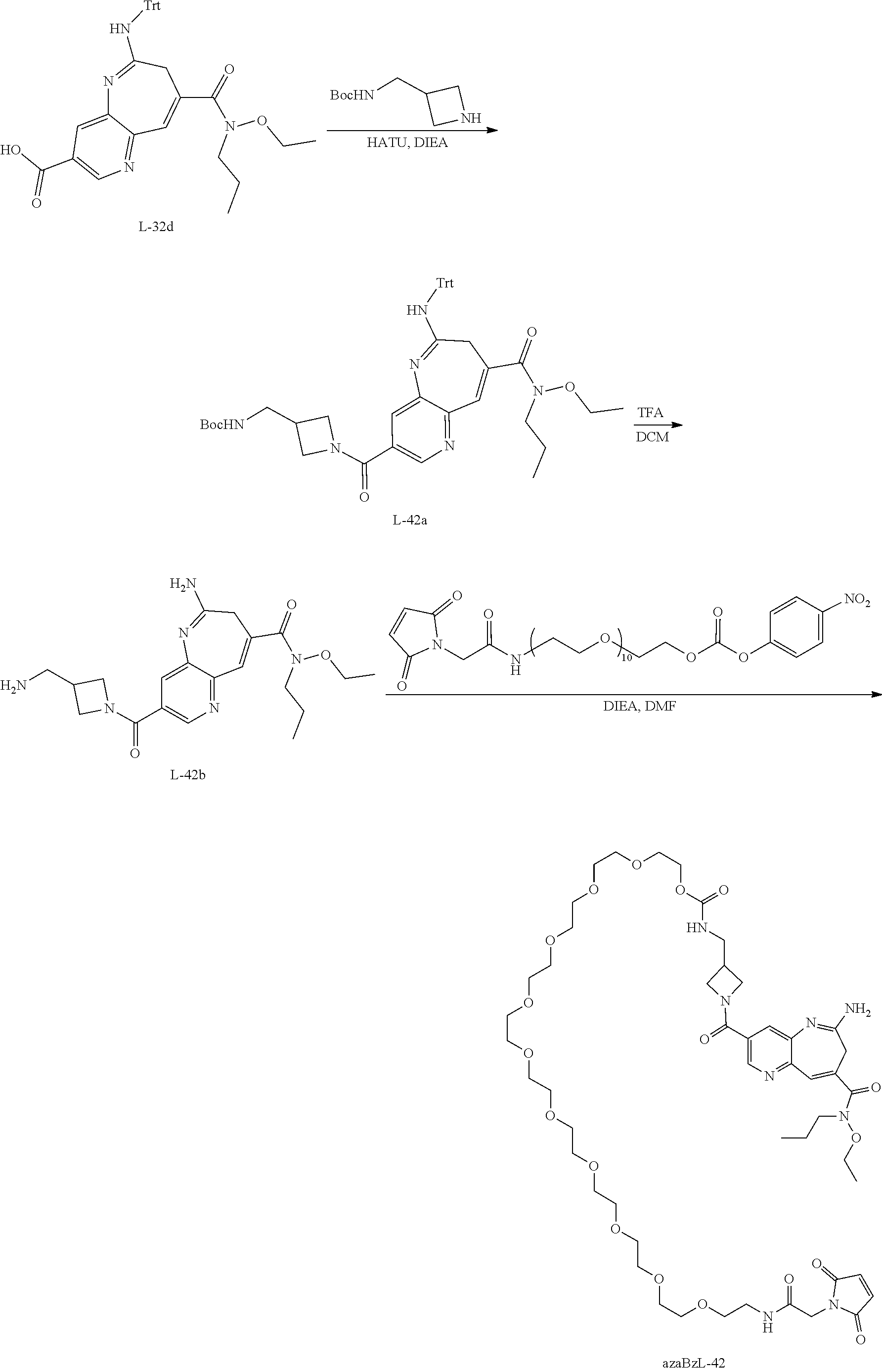

### Preparation of tert-butyl N-[[1-[8-[ethoxy(propyl)carbamoyl]-6-(tritylamino)-7H-pyrido[3,2-b]azepine-3-carbonyl]azetidin-3-yl]methyl]carbamate, L-42a

A mixture of 8-[ethoxy(propyl)carbamoyl]-6-(tritylamino)-7H-pyrido[3,2-b]azepine-3-carboxylic acid, L-32d (450 mg, 783 mol, 1 eq), HATU (298 mg, 783 mol, 1 eq) and DIEA (304 mg, 2.35 mmol, 409 μL, 3 eq) in DMF (4 mL) was added tert-butyl N-(azetidin-3-ylmethyl)carbamate (190 mg, 1.02 mmol, 1.3 eq), and then stirred at 25° C. for 1 hr. The reaction mixture was diluted with H₂O (30 mL), then extracted with EtOAc (10 ml×3). The organic layer was washed with brine, dried over Na₂SO₄, filtered and concentrated. The residue was purified by flash silica gel chromatography (ISCO®; 12 g SepaFlash® Silica Flash Column, Eluent of 0-100% Ethyl acetate/Petroleum ether ether gradient @80 mL/min) to give L-42a (463 mg, 623 mol, 79.6% yield) as a light yellow solid. ¹H NMR (MeOD, 400 MHz,) δ 8.35 (d, J=2.0 Hz, 1H), 7.36-7.32 (m, 6H), 7.29-7.16 (m, 10H), 7.03 (d, J=2.0 Hz, 1H), 4.32 (t, J=8.8 Hz, 1H), 4.18 (t, J=9.2 Hz, 1H), 4.01 (q, J=7.2 Hz, 2H), 3.98-3.93 (m, 1H), 3.86 (dd, J=5.2, 10.4 Hz, 1H), 3.80 (t, J=6.8 Hz, 2H), 3.28 (d, J=6.8 Hz, 2H), 3.01-2.98 (m, 2H), 2.88-2.85 (m, 1H), 1.85-1.76 (m, 2H), 1.41 (s, 9H), 1.24-1.20 (m, 3H), 1.01 (t, J=7.2 Hz, 3H). LC/MS [M+H] 743.4 (calculated); LC/MS [M+H] 743.3 (observed).

### Preparation of 6-amino-3-[3-(aminomethyl)azetidine-1-carbonyl]-N-ethoxy-N-propyl-7H-pyrido[3,2-b]azepine-8-carboxamide, L-42b

To a solution of L-42a (360 mg, 485 mol, 1 eq) in DCM (2 mL) was added TFA (1.11 g, 9.69 mmol, 720 μL, 20 eq), and then stirred at 50° C. for 12 hrs. The reaction mixture was cooled to 25° C., concentrated under reduced pressure. The residue was purified by prep-HPLC (column: Phenomenex Luna C18 75*30 mm*3 um; mobile phase: [H₂O (0.1% TFA)-ACN]; gradient: 5%-35% B over 8.0 min) to give L-42b (180 mg, 350 mol, 72.2% yield, TFA) as a light yellow solid. ¹H NMR (MeOD, 400 MHz,) δ 8.85 (d, J=1.6 Hz, 1H), 8.07 (d, J=1.6 Hz, 1H), 7.40 (s, 1H), 4.62 (t, J=8.8 Hz, 1H), 4.39 (t, J=9.6 Hz, 1H), 4.31-4.24 (m, 1H), 4.05-3.94 (m, 3H), 3.76 (t, J=7.2 Hz, 2H), 3.48 (s, 2H), 3.31-3.29 (m, 2H), 3.12-3.02 (m, 1H), 1.82-1.73 (m, 2H), 1.20 (t, J=7.2 Hz, 3H), 1.01 (t, J=7.2 Hz, 3H). LC/MS [M+H] 401.2 (calculated); LC/MS [M+H] 401.2 (observed).

### Preparation of azaBzL-42

To a solution of L-42b (45 mg, 87.5 mol, 1 eq, TFA) in DMF (1 mL) were added DIEA (33.9 mg, 262 mol, 45.7 μL, 3 eq) and 2-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]ethyl (4-nitrophenyl)carbonate (70.3 mg, 87.5 mol, 1 eq) under N₂ at 0° C., and then warmed to 25° C. and stirred for 1 h. The pH of the mixture was adjusted to 6 with TFA, and the reaction mixture was concentrated under reduced pressure to give a residue. The residue was purified by prep-HPLC (column: Phenomenex Luna C18 75*30 mm*3 um; mobile phase: [H₂O (0.1% TFA)-ACN]; gradient: 20%-40% B over 8.0 min) to give azaBzL-42 (53.0 mg, 44.9 mol, 51.4% yield, TFA) as light yellow oil. ¹H NMR (MeOD, 400 MHz,) δ 8.87 (d, J=1.6 Hz, 1H), 8.05 (s, 1H), 7.42 (s, 1H), 6.89 (s, 2H), 4.52 (t, J=8.8 Hz, 1H), 4.28 (t, J=9.6 Hz, 1H), 4.21-4.13 (m, 5H), 4.02-3.94 (m, 3H), 3.76 (t, J=7.2 Hz, 2H), 3.68-3.61 (m, 38H), 3.56-3.53 (m, 2H), 3.50 (s, 2H), 3.42-3.36 (m, 4H), 2.97-2.88 (m, 1H), 1.83-1.73 (m, 2H), 1.21 (t, J=7.2 Hz, 3H), 1.00 (t, J=7.2 Hz, 3H). LC/MS [M+H] 1065.5 (calculated); LC/MS [M+H] 1065.4 (observed).

### Example L-51 Synthesis of (2,3,5,6-tetrafluorophenyl) 3-[2-[2-[2-[2-[2-[2-[2-[2-[2-[3-[[5-[[6-amino-8-[ethoxy(propyl)carbamoyl]-7H-pyrido[3,2-b]azepine-3-carbonyl]amino]-3-pyridyl]methylamino]-3-oxo-propoxy]ethoxy]ethoxy]ethoxy]ethoxy]ethoxy]ethoxy]ethoxy]ethoxy]ethoxy]propanoate, azaBzL-51

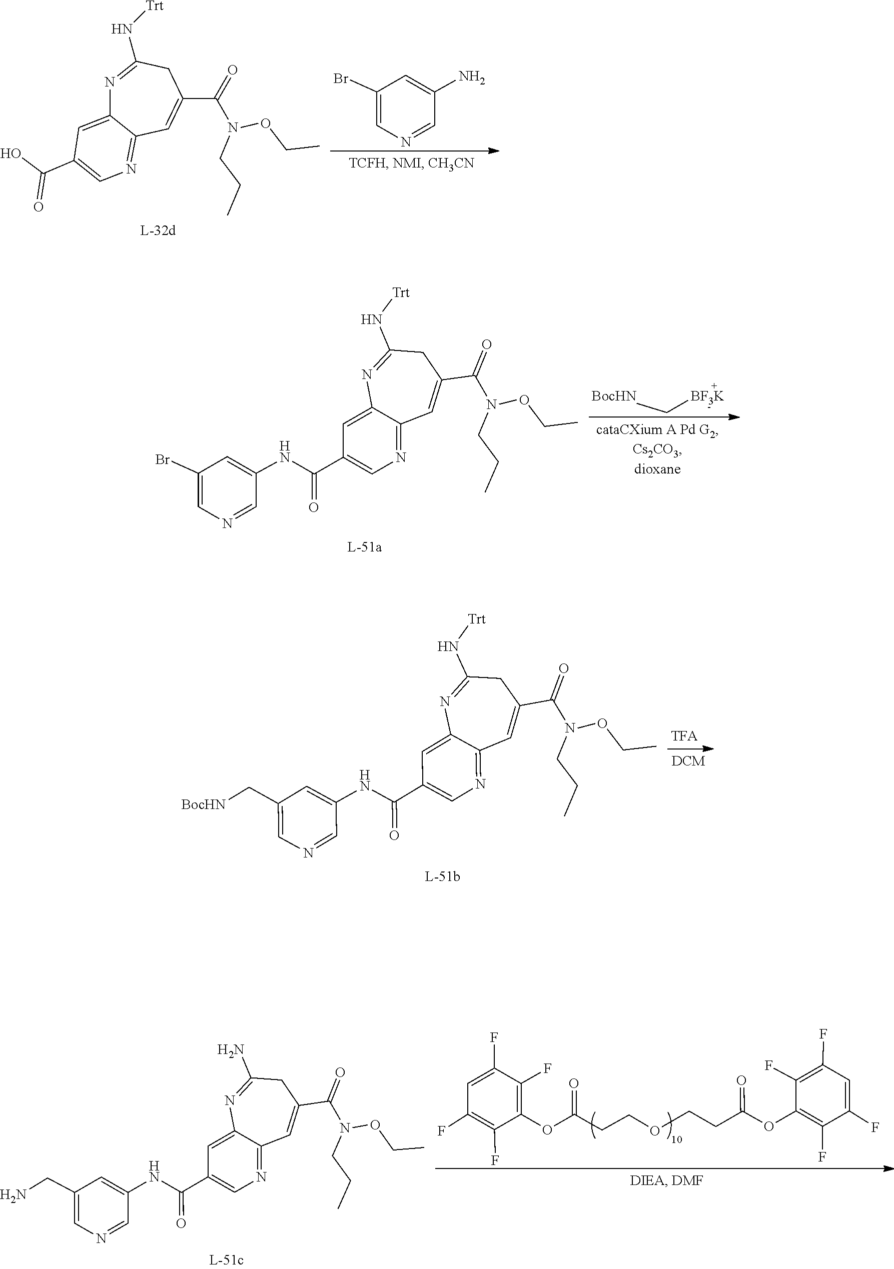

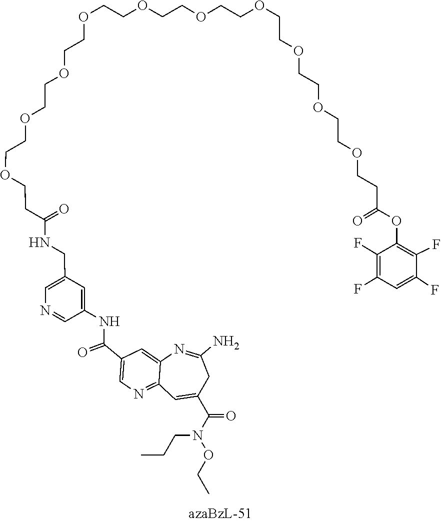 [SMILES: CCCN(OCC)C(=O)C1=Cc2ncc(C(=O)Nc3cncc(CNC(=O)CCOCCOCCOCCOCCOCCOCCOCCOCCOCCOCCC(=O)Oc4c(F)c(F)cc(F)c4F)c3)cc2N=C(N)C1]

### Preparation of N3-(5-bromo-3-pyridyl)-N8-ethoxy-N8-propyl-6-(tritylamino)-7H-pyrido[3,2-b]azepine-3,8-dicarboxamide, L-51a

To a solution of 8-[ethoxy(propyl)carbamoyl]-6-(tritylamino)-7H-pyrido[3,2-b]azepine-3-carboxylic acid, L-32d (1.00 g, 1.74 mmol, 1 eq), 5-bromopyridin-3-amine (452 mg, 2.61 mmol, 1.5 eq) and NMI (429 mg, 5.22 mmol, 416 μL, 3 eq) in CH₃CN (10 mL) was added TCFH (732 mg, 2.61 mmol, 1.5 eq) at 0° C., and then warmed to 20° C. and stirred at 20° C. for 0.5 hr. The reaction mixture was concentrated under reduced pressure to give a residue. The residue was diluted with H₂O (30 mL) and extracted with EtOAc (30 mL×3). The combined organic layers were washed with brine (10 mL), dried over Na₂SO₄, filtered and concentrated under reduced pressure to give a residue. The residue was purified by column chromatography (SiO₂, Petroleum ether/Ethyl acetate=I1/0 to 0/1) to give L-51a (1.00 g, 1.34 mmol, 77.2% yield) as a yellow solid. LC/MS [M+H] 729.2 (calculated); LC/MS [M+H] 729.2 (observed).

### Preparation of tert-butyl N-[[5-[[8-[ethoxy(propyl)carbamoyl]-6-(tritylamino)-7H-pyrido[3,2-b]azepine-3-carbonyl]amino]-3-pyridyl]methyl]carbamate, L-51b

A mixture of L-51a (1.00 g, 1.37 mmol, 1 eq), potassium; (tert-butoxycarbonylamino)methyl-trifluoro-boranide (357 mg, 1.51 mmol, 1.1 eq), [2-(2-aminophenyl)phenyl]-chloro-palladium;bis(1-adamantyl)-butyl-phosphane (91.6 mg, 137 mol, 0.1 eq) and Cs₂CO₃ (893 mg, 2.74 mmol, 2 eq) in dioxane (10 mL) and H₂O (2 mL) was degassed and purged with N₂ for 3 times, and then stirred at 110° C. for 1 hr under N₂ atmosphere.

The reaction mixture was cooled to 20° C., filtered and concentrated under reduced pressure to give a residue. The residue was purified by column chromatography (SiO₂, Petroleum ether/Ethyl acetate=I1/0 to 0/1) to give L-51b (1.00 g, 1.28 mmol, 93.6% yield) as a yellow solid. ¹H NMR (CDCl₃, 400 MHz) δ 8.73 (s, 1H), 8.61 (s, 1H), 8.34 (s, 1H), 8.20 (s, 1H), 7.98 (s, 1H), 7.54 (s, 1H), 7.35-7.28 (m, 6H), 7.26-7.16 (m, 9H), 6.42 (s, 1H), 5.02 (s, 1H), 4.36 (d, J=5.6 Hz, 2H), 3.98 (q, J=7.2 Hz, 2H), 3.77 (t, J=7.2 Hz, 2H), 2.86 (s, 2H), 1.83-1.76 (m, 2H), 1.47 (s, 9H), 1.32-1.26 (m, 3H), 0.99 (t, J=7.2 Hz, 3H). LC/MS [M+H] 780.4 (calculated); LC/MS [M+H] 780.3 (observed).

### Preparation of 6-amino-N3-[5-(aminomethyl)-3-pyridyl]-N8-ethoxy-N8-propyl-7H-pyrido[3,2-b]azepine-3,8-dicarboxamide, L-51c

To a solution of L-51b (0.6 g, 769 mol, 1 eq) in DCM (10 mL) was added TFA (2.63 g, 23.1 mmol, 1.71 mL, 30 eq) at 25° C. under N₂, and then heated to 50° C. and stirred at 50° C. for 2 hrs. The reaction mixture was cooled to 25° C., filtered and concentrated under reduced pressure to give a residue. The residue was triturated with MTBE (10 mL) at 25° C. for 10 min, the mixture was filtered and the filter cake was dried under reduced pressure to afford L-51c (0.5 g, 611 mol, 79.4% yield, 95.2% purity, 3TFA) as a light yellow solid. ¹H NMR (MeOD, 400 MHz) δ 9.17 (d, J=1.2 Hz, 1H), 8.91 (d, J=1.2 Hz, 1H), 8.59 (s, 1H), 8.48 (s, 1H), 8.39 (d, J=1.2 Hz, 1H), 7.45 (s, 1H), 4.26 (s, 2H), 4.00 (q, J=7.2 Hz, 2H), 3.77 (t, J=7.2 Hz, 2H), 3.52 (s, 2H), 1.86-1.72 (m, 2H), 1.21 (t, J=7.2 Hz, 3H), 1.02 (t, J=7.2 Hz, 3H). LC/MS [M+H] 438.2 (calculated); LC/MS [M+H] 438.2 (observed).

### Preparation of azaBzL-51

To a solution of L-51c (0.1 g, 128 mol, 1 eq, 3TFA) in DMF (1 mL) were added DIEA (66.3 mg, 513 mol, 89.4 μL, 4 eq) and (2,3,5,6-tetrafluorophenyl) 3-[2-[2-[2-[2-[2-[2-[2-[2-[2-[3-oxo-3-(2,3,5,6-tetrafluorophenoxy)propoxy]ethoxy]ethoxy]ethoxy]ethoxy]ethoxy]ethoxy]ethoxy]ethoxy]ethoxy]propanoate (274 mg, 321 mol, 2.5 eq) at 0° C., and then stirred at 0° C. for 0.5 hr. The reaction mixture was adjusted to pH=5-6 with TFA at 0° C. and purified by prep-HPLC (column: Phenomenex Luna C18 75*30 mm*3 um; mobile phase: [H₂O (0.1% TFA)-ACN]; gradient: 20%-50% B over 8.0 min) to give azaBzL-51 (70.7 mg, 47.8 mol, 37.2% yield, 91.5% purity, 2TFA) as yellow oil. ¹H NMR (MeOD, 400 MHz) δ 9.21 (d, J=2.0 Hz, 1H), 9.18 (d, J=2.0 Hz, 1H), 8.51 (s, 1H), 8.47 (s, 1H), 8.38 (d, J=2.0 Hz, 1H), 7.50-7.37 (m, 2H), 4.62-4.53 (m, 2H), 4.00 (q, J=6.8 Hz, 2H), 3.85 (t, J=6.0 Hz, 2H), 3.82-3.73 (m, 4H), 3.67-3.55 (m, 36H), 3.52 (s, 2H), 2.96 (t, J=6.0 Hz, 2H), 2.54 (t, J=6.0 Hz, 2H), 1.83-1.74 (m, 2H), 1.21 (t, J=7.2 Hz, 3H), 1.01 (t, J=7.2 Hz, 3H). LC/MS [M+H] 1126.5 (calculated); LC/MS [M+H] 1126.5 (observed).

### Example L-52 Synthesis of (2,3,5,6-tetrafluorophenyl) 3-[2-[2-[2-[2-[2-[2-[2-[2-[2-[3-[3-[[6-amino-8-(dipropylcarbamoyl)-7H-pyrido[3,2-b]azepine-3-carbonyl]amino]-7,8-dihydro-5H-1,6-naphthyridin-6-yl]-3-oxo-propoxy]ethoxy]ethoxy]ethoxy]ethoxy]ethoxy]ethoxy]ethoxy]ethoxy]ethoxy]propanoate, azaBzL-52

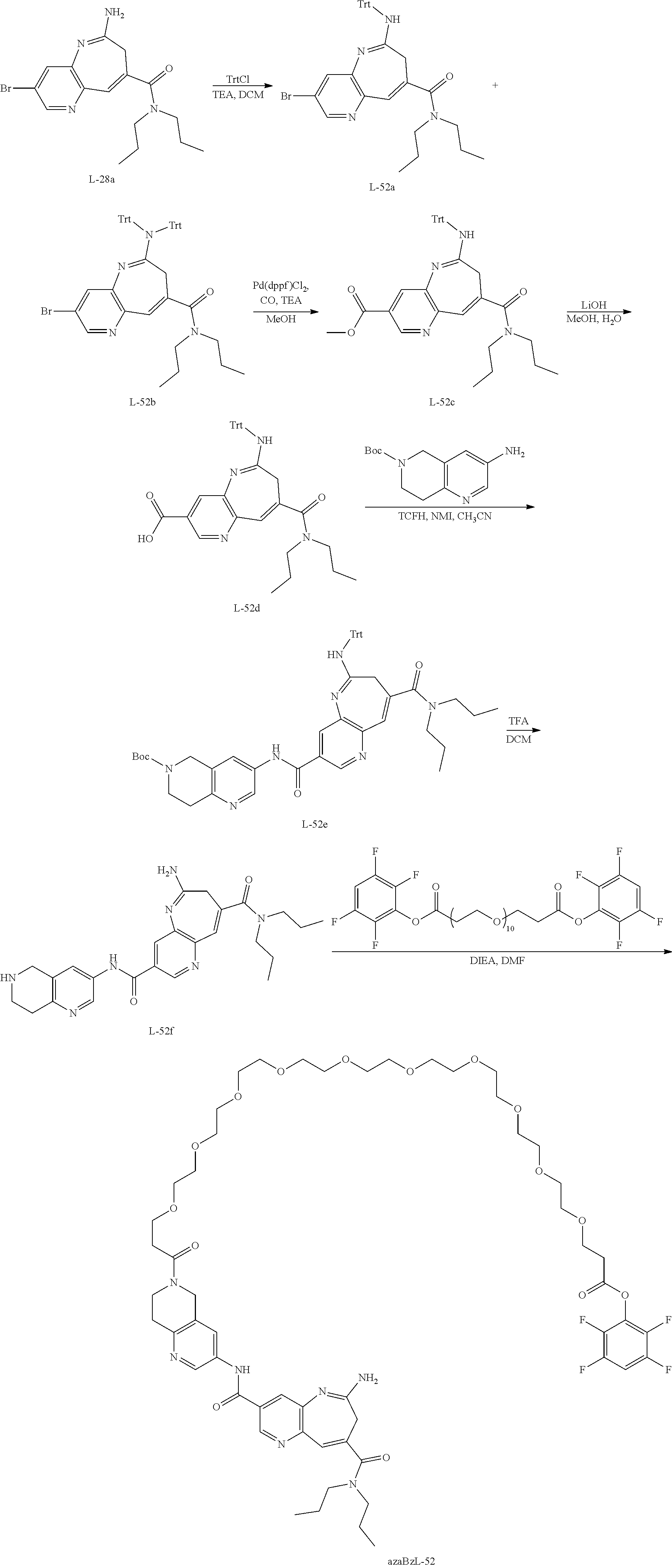

### Preparation of 3-bromo-N,N-dipropyl-6-(tritylamino)-7H-pyrido[3,2-b]azepine-8-carboxamide, L-52a

To a solution of 6-amino-3-bromo-N,N-dipropyl-7H-pyrido[3,2-b]azepine-8-carboxamide, L-28a (10.0 g, 27.4 mmol, 1 eq) and TEA (11.1 g, 110 mmol, 15.2 mL, 4 eq) in DCM (200 mL) was added TrtCl (19.1 g, 68.4 mmol, 2.5 eq) at 25° C., and then heated to 50° C. and stirred at 50° C. for 12 hrs. The reaction mixture was quenched by addition of H₂O (300 mL) at 0° C., and extracted with DCM (150 mL×3). The combined organic layers were washed with brine (50 mL), dried over Na₂SO₄, filtered and concentrated under reduced pressure to give a residue. The residue was purified by column chromatography (SiO₂, Petroleum ether/Ethyl acetate=I1/0 to 1/1) to give L-52a (6.00 g, 9.88 mmol, 36.1% yield) and 3-bromo-6-(ditritylamino)-N,N-dipropyl-7H-pyrido[3,2-b]azepine-8-carboxamide, L-52b (2.5 g, 2.94 mmol, 10.7% yield) as yellow solids. LC/MS [M+H] 607.2 (calculated); LC/MS [M+H] 607.2 (observed). LC/MS [M+H] 849.3 (calculated); LC/MS [M+H] 849.3 (observed).

### Preparation of methyl 8-(dipropylcarbamoyl)-6-(tritylamino)-7H-pyrido[3,2-b]azepine-3-carboxylate, L-52c

A mixture of L-52a (4.60 g, 7.57 mmol, 1 eq), Pd(dppf)Cl₂ (554 mg, 757 mol, 0.1 eq) and TEA (2.30 g, 22.7 mmol, 3.16 mL, 3 eq) in MeOH (50 mL) was degassed and purged with CO for 3 times, and then heated to 80° C. and stirred at 80° C. for 16 hrs under CO atmosphere(50 psi). The reaction mixture was concentrated under reduced pressure to give a residue. The residue was purified by column chromatography (SiO₂, Petroleum ether/Ethyl acetate=I1/0 to 1/1) to give L-52c (3.50 g, 5.97 mmol, 78.8% yield) as a brown solid. ¹H NMR (CDCl₃, 400 MHz) δ 8.75 (d, J=2.0 Hz, 1H), 7.49 (s, 1H), 7.37-7.31 (m, 5H), 7.26-7.15 (m, 1OH), 6.91 (s, 1H), 6.30 (s, 1H), 3.90 (s, 3H), 3.69-3.36 (m, 4H), 2.77 (s, 2H), 1.74-1.62 (m, 4H), 1.00-0.85 (m, 6H). LC/MS [M+H] 587.3 (calculated); LC/MS [M+H] 587.3 (observed).

### Preparation of 8-(dipropylcarbamoyl)-6-(tritylamino)-7H-pyrido[3,2-b]azepine-3-carboxylic acid, L-52d

To a solution of L-52c (2.50 g, 4.26 mmol, 1 eq) in MeOH (30 mL) was added a solution of LiOH·H₂O (1.07 g, 25.6 mmol, 6 eq) in H₂O (10 mL) at 25° C., and then heated to 80° C. and stirred at 80° C. for 20 hr. The reaction mixture was filtered. The filtrate was cooled to 0° C. and diluted with H₂O (30 mL) and adjusted to pH=5 with 2 N HCl at 0° C., concentrated under reduced pressure to remove MeOH. The mixture was filtered and the filter cake was dried under reduced pressure to afford L-52d (671.5 mg, 1.17 mmol, 27.5% yield) as a light yellow solid. ¹H NMR (DMSO-d₆, 400 MHz) δ 8.56 (d, J=2.0 Hz, 1H), 8.40 (s, 1H), 7.33-7.28 (m, 6H), 7.27-7.20 (m, 7H), 7.19-7.13 (m, 3H), 6.77 (s, 1H), 3.45-3.37 (m, 4H), 2.99 (s, 2H), 1.69-1.56 (m, 4H), 1.04-0.75 (m, 6H). LC/MS [M+H] 573.3 (calculated); LC/MS [M+H] 573.3 (observed).

### Preparation of tert-butyl 3-[[8-(dipropylcarbamoyl)-6-(tritylamino)-7H-pyrido[3,2-b]azepine-3-carbonyl]amino]-7,8-dihydro-5H-1,6-naphthyridine-6-carboxylate, L-52e

To a solution of L-52d (310 mg, 541 mol, 1 eq) and tert-butyl 3-amino-7,8-dihydro-5H-1,6-naphthyridine-6-carboxylate (148 mg, 595 mol, 1.1 eq) in MeCN (5 mL) were added NMI (222 mg, 2.71 mmol, 215 μL, 5 eq) and TCFH (303 mg, 1.08 mmol, 2 eq), and then stirred at 20° C. for 0.5 hr. The reaction mixture was concentrated under reduced pressure, and the residue was diluted with ice-water (w/w=1/1) (10 mL). The aqueous phase was extracted with ethyl acetate (10 mL×3). The combined organic phase was washed with brine (10 mL), dried with anhydrous Na₂SO₄, filtered and concentrated in vacuum. The residue was purified by flash silica gel chromatography (ISCO®; 12 g SepaFlash® Silica Flash Column, Eluent of 0~100% Ethyl acetate/Petroleum ether gradient @40 mL/min) to give L-52e (0.38 g, 472 mol, 87.3% yield) as a yellow solid. LC/MS [M+H] 804.4 (calculated); LC/MS [M+H] 804.5 (observed).

### Preparation of 6-amino-N8,N8-dipropyl-N3-(5,6,7,8-tetrahydro-1,6-naphthyridin-3-yl)-7H-pyrido[3,2-b]azepine-3,8-dicarboxamide, L-52f

To a solution of L-52e (0.38 g, 472 mol, 1 eq) in DCM (10 mL) was added TFA (1.35 g, 11.8 mmol, 877 μL, 25 eq), and then stirred at 50° C. for 15 hrs. The mixture was concentrated under reduced pressure to afford L-52f (0.25 g, 434 mol, 91.9% yield, TFA) as yellow oil. LC/MS [M+H] 462.3 (calculated); LC/MS [M+H] 462.3 (observed).

### Preparation of azaBzL-52

To a solution of (2,3,5,6-tetrafluorophenyl) 3-[2-[2-[2-[2-[2-[2-[2-[2-[2-[3-oxo-3-(2,3,5,6-tetrafluorophenoxy)propoxy]ethoxy]ethoxy]ethoxy]ethoxy]ethoxy]ethoxy]ethoxy]ethoxy]ethoxy]propanoate (266 mg, 311 mol, 2.5 eq) in DMF (1 mL) was added a solution of DIPEA (64.3 mg, 498 mol, 86.7 μL, 4 eq) and L-52f (0.1 g, 124 mol, 1 eq, 3TFA) in DMF (1 mL) at 0° C., and then stirred at 0° C. for 0.5 hr. The reaction mixture was adjusted to pH=5-6 with TFA at 0° C. and purified by prep-HPLC (column: Phenomenex Luna C18 75*30 mm*3 um; mobile phase: [H₂O (0.1% TFA)-ACN]; gradient: 20%-50% B over 8.0 min) to give azaBzL-52 (35.4 mg, 25.7 mol, 20.6% yield, 2TFA) as yellow oil. ¹H NMR (MeOD, 400 MHz) δ 9.21-9.19 (m, 1H), 8.98 (s, 1H), 8.40-8.36 (m, 1H), 8.35-8.27 (m, 1H), 7.49-7.37 (m, 1H), 7.10 (s, 1H), 4.05-3.95 (m, 2H), 3.90-3.83 (m, 2H), 3.80-3.78 (m, 3H), 3.66-3.56 (m, 38H), 3.50-3.45 (m, 4H), 3.40-3.36 (m, 2H), 3.19 (t, J=5.6 Hz, 1H), 3.06 (t, J=5.6 Hz, 1H), 2.97 (t, J=6.0 Hz, 2H), 2.85-2.74 (m, 2H), 1.76-1.67 (m, 4H), 1.05-0.85 (m, 6H). LC/MS [M+H] 1150.5 (calculated); LC/MS [M+H] 1150.7 (observed).

### Example L-53 Synthesis of (2,3,5,6-tetrafluorophenyl) 3-[2-[2-[2-[2-[2-[2-[2-[2-[2-[3-[[5-[[6-amino-8-(dipropylcarbamoyl)-7H-pyrido[3,2-b]azepine-3-carbonyl]amino]-3-pyridyl]methylamino]-3-oxo-propoxy]ethoxy]ethoxy]ethoxy]ethoxy]ethoxy]ethoxy]ethoxy]ethoxy]ethoxy]propanoate, azaBzL-53

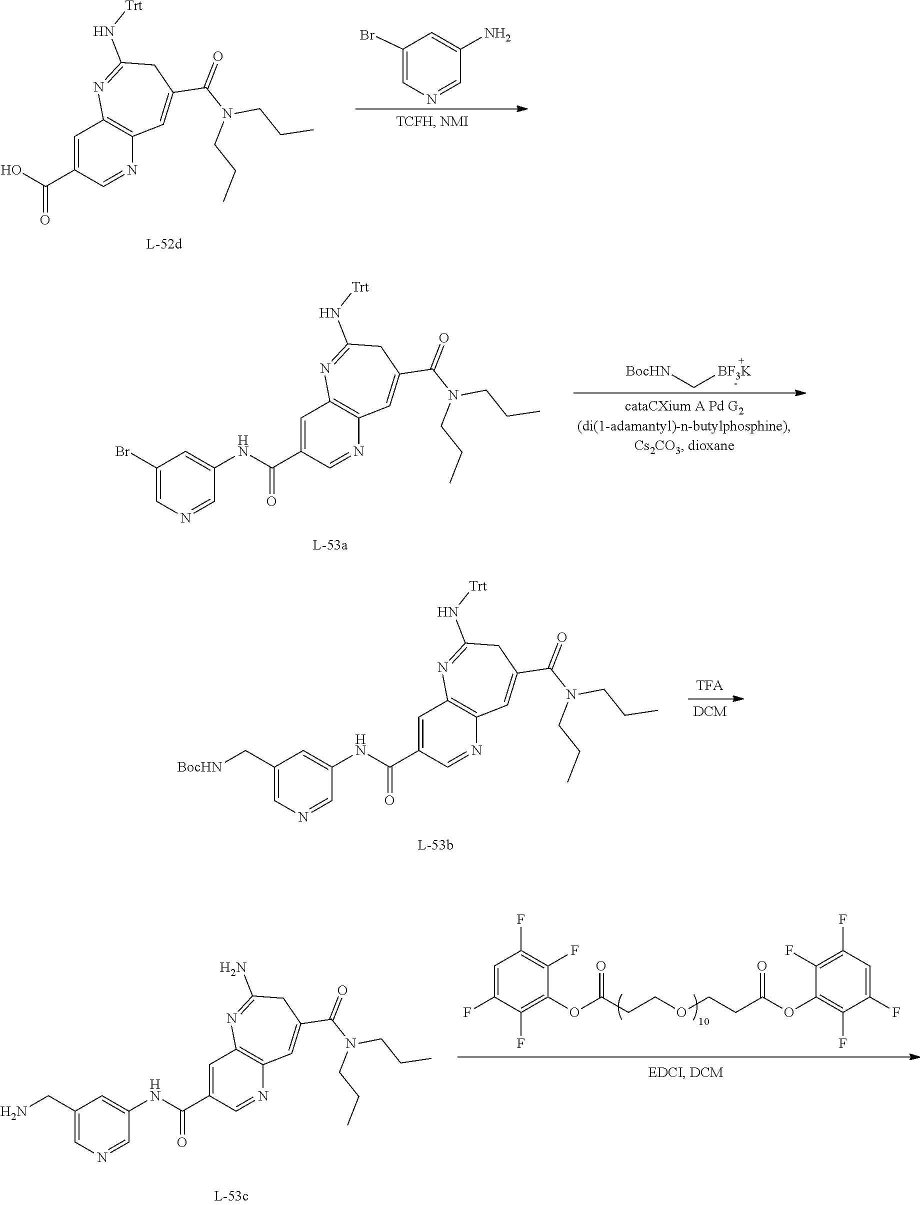

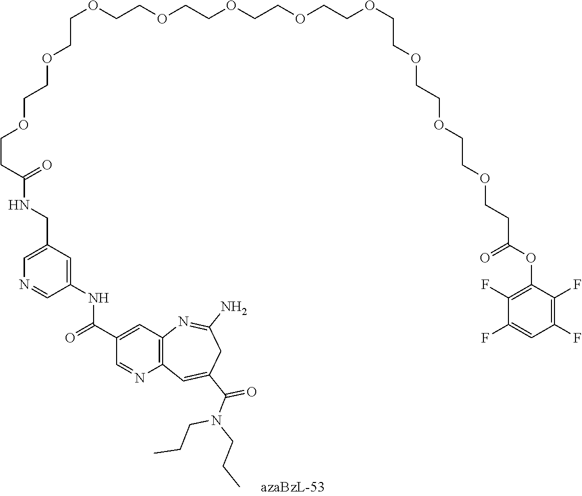 [SMILES: CCCN(CCC)C(=O)C1=Cc2ncc(C(=O)Nc3cncc(CNC(=O)CCOCCOCCOCCOCCOCCOCCOCCOCCOCCOCCC(=O)Oc4c(F)c(F)cc(F)c4F)c3)cc2N=C(N)C1]

### Preparation of N3-(5-bromo-3-pyridyl)-N8,N8-dipropyl-6-(tritylamino)-7H-pyrido[3,2-b]azepine-3,8-dicarboxamide, L-53a

To a solution of 8-(dipropylcarbamoyl)-6-(tritylamino)-7H-pyrido[3,2-b]azepine-3-carboxylic acid, L-52d (450 mg, 785 mol, 1 eq) and 5-bromopyridin-3-amine (149 mg, 864 mol, 1.1 eq) in MeCN (5 mL) were added NMI (322 mg, 3.93 mmol, 313 μL, 5 eq) and TCFH (440 mg, 1.57 mmol, 2 eq), and then stirred at 20° C. for 0.5 hr. The reaction mixture was concentrated under reduced pressure to give a residue, and the residue was diluted with ice-water (w/w=1/1) (10 mL). The aqueous phase was extracted with ethyl acetate (10 mL×3). The combined organic phase was washed with brine (10 mL), dried with anhydrous Na₂SO₄, filtered and concentrated in vacuum. The residue was purified by flash silica gel chromatography (ISCO®; 12 g SepaFlash® Silica Flash Column, Eluent of 0-100% Ethyl acetate/Petroleum ether gradient @40 mL/min) to give L-53a (410 mg, 563 mol, 71.7% yield) as a yellow solid. ¹H NMR (MeOD, 400 MHz) δ 8.66 (d, J=2.0 Hz, 1H), 8.48 (d, J=2.0 Hz, 1H), 8.41 (t, J=2.0 Hz, 1H), 8.30 (d, J=2.0 Hz, 1H), 7.34 (d, J=2.0 Hz, 1H), 7.30-7.28 (m, 6H), 7.15-7.10 (m, 6H), 7.10-7.05 (m, 3H), 6.79 (s, 1H), 3.43-3.38 (m, 4H), 2.88 (s, 2H), 1.68-1.58 (m, 4H), 0.94-0.82 (m, 6H). LC/MS [M+H] 727.2 (calculated); LC/MS [M+H] 727.4 (observed).

### Preparation of tert-butyl N-[[5-[[8-(dipropylcarbamoyl)-6-(tritylamino)-7H-pyrido[3,2-b]azepine-3-carbonyl]amino]-3-pyridyl]methyl]carbamate, L-53b

A mixture of L-53a (360 mg, 494 mol, 1 eq), potassium; (tert-butoxycarbonylamino)methyl-trifluoro-boranuide (129 mg, 544 mol, 1.1 eq), [2-(2-aminophenyl)phenyl]-chloro-palladium;bis(1-adamantyl)-butyl-phosphane (33.1 mg, 49.4 mol, 0.1 eq), Cs₂CO₃ (322 mg, 989 mol, 2 eq) in dioxane (5 mL) and H₂O (1 mL) was degassed and purged with N₂ for 3 times, and then stirred at 110° C. for 15 hrs under N₂ atmosphere. The mixture was concentrated under reduced pressure. The residue was purified by flash silica gel chromatography (ISCO®; 12 g SepaFlash® Silica Flash Column, Eluent of 0~100% Ethyl acetate/Petroleum ether gradient @40 mL/min) to give L-53b (0.35 g, 445 mol, 90.9% yield) as a yellow solid. ¹H NMR (MeOD, 400 MHz) δ 8.75 (s, 1H), 8.60 (d, J=2.0 Hz, 1H), 8.26 (s, 1H), 8.16 (s, 1H), 7.46 (s, 1H), 7.45-7.40 (m, 6H), 7.27-7.18 (m, 9H), 6.91 (s, 1H), 4.33 (s, 2H), 3.55-3.50 (m, 4H), 2.99 (s, 2H), 1.78-1.72 (m, 4H), 1.49 (s, 9H), 1.02-0.95 (m, 6H). LC/MS [M+H] 778.4 (calculated); LC/MS [M+H] 778.3 (observed).

### Preparation of 6-amino-N3-[5-(aminomethyl)-3-pyridyl]-N8,N8-dipropyl-7H-pyrido[3,2-b]azepine-3,8-dicarboxamide, L-53c

To a mixture of L-53b (0.35 g, 450 mol, 1 eq) in DCM (1 mL) was added TFA (1.54 g, 13.5 mmol, 1.00 mL, 30 eq) at 20° C., and then warm to 50° C. and stirred for 1 hr. The mixture was concentrated. The crude product was purified by re-crystallization from MTBE (10 mL) at 20 C to give L-53c (0.3 g, crude, TFA) was obtained as a yellow solid. ¹H NMR (MeOD, 400 MHz) δ 9.17 (d, J=2.0 Hz, 1H), 8.90 (d, J=2.0 Hz, 1H), 8.59 (s, 1H), 8.47 (s, 1H), 8.38 (d, J=2.0 Hz, 1H), 7.10 (s, 1H), 4.26 (s, 2H), 3.57-3.42 (m, 4H), 1.79-1.64 (m, 4H), 1.05-0.86 (m, 6H). LC/MS [M+H] 436.2 (calculated); LC/MS [M+H] 436.3 (observed).

### Preparation of azaBzL-53

To a mixture of L-53c (0.1 g, 182 mol, 1 eq, TFA) in DMF (1 mL) were added DIEA (70.6 mg, 546 mol, 95.1 μL, 3 eq) and (2,3,5,6-tetrafluorophenyl) 3-[2-[2-[2-[2-[2-[2-[2-[2-[2-[3-oxo-3-(2,3,5,6-tetrafluorophenoxy)propoxy]ethoxy]ethoxy]ethoxy]ethoxy]ethoxy]ethoxy]ethoxy]ethoxy]ethoxy]propanoate (389 mg, 455 mol, 2.5 eq) in one portion at 0° C. and stirred at 0° C. for 0.5 hr. The mixture was adjusted pH-6 with TFA and purified by prep-HPLC(column: Phenomenex Luna C18 75*30 mm*3 um; mobile phase: [H₂O (0.1% TFA)-ACN]; gradient: 20%-50% B over 8.0 min) to give azaBzL-53 (63.8 mg, 54.30 mol, 29.8% yield, 95.6% purity) as yellow oil. ¹H NMR (MeOD, 400 MHz) δ 9.24-9.20 (m, 1H), 8.53-8.52 (m, 1H), 8.39 (d, J=1.6 Hz, 1H), 7.47-7.40 (m, 1H), 7.10 (s, 1H), 4.59 (s, 2H), 3.85 (t, J=6.0 Hz, 2H), 3.78 (t, J=6.0 Hz, 2H), 3.63-3.58 (m, 38H), 3.47-3.31 (m, 4H), 2.96 (t, J=6.0 Hz, 2H), 2.55 (t, J=6.0 Hz, 2H), 1.76-1.67 (m, 4H), 0.98-0.93 (m, 6H). LC/MS [M+H] 1124.5 (calculated); LC/MS [M+H] 1124.7 (observed).

### Example L-57 Synthesis of 6-amino-3-[3-[[3-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]propanoylamino]methyl]azetidine-1-carbonyl]-N-ethoxy-N-propyl-7H-pyrido[3,2-b]azepine-8-carboxamide, azaBzL-57

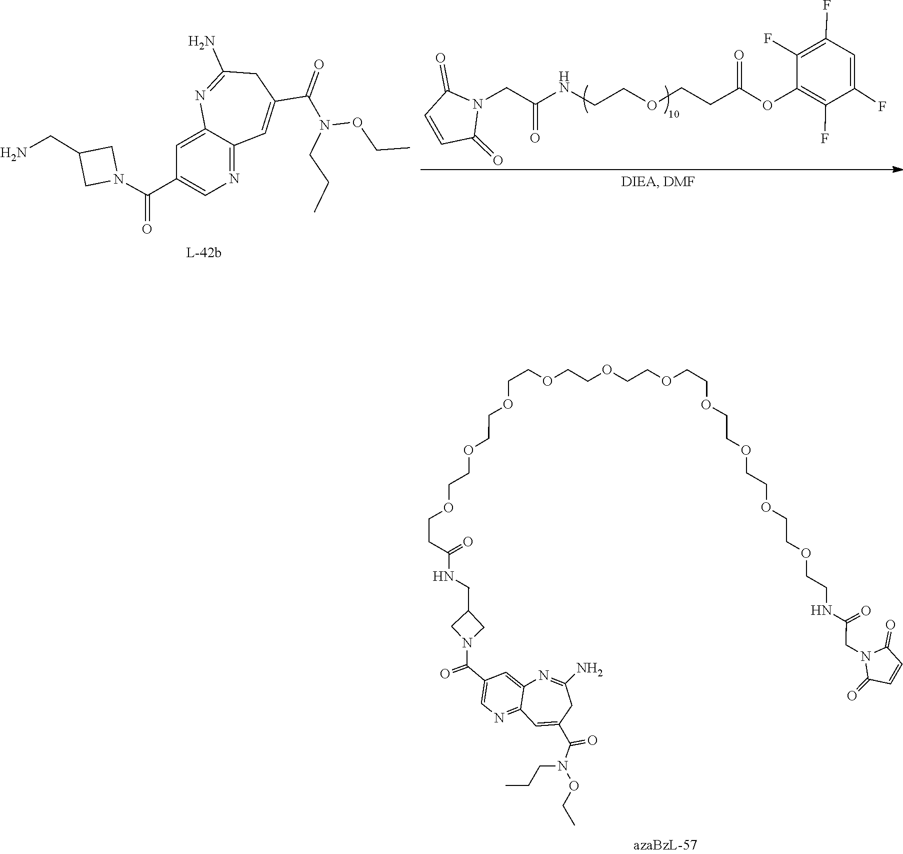

To a solution of 6-amino-3-[3-(aminomethyl)azetidine-1-carbonyl]-N-ethoxy-N-propyl-7H-pyrido[3,2-b]azepine-8-carboxamide, L-42b (65 mg, 126 mol, 1 eq, TFA) in DMF (3 mL) were added DIEA (49.0 mg, 379 mol, 66.0 μL, 3 eq) and (2,3,5,6-tetrafluorophenyl) 3-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]propanoate (103 mg, 126 mol, 1 eq) under N₂ at 0° C., and then warmed to 25° C. and stirred for 1 hr. The mixture was adjusted to pH-6 with TFA, and then concentrated under reduced pressure to give a residue. The residue was purified by prep-HPLC (column: Phenomenex Luna C18 75*30 mm*3 um; mobile phase: [H₂O (0.1% TFA)-ACN]; gradient: 15%-35% B over 8.0 min) to give azaBzL-57 (62 mg, 59.1 mol, 46.7% yield) as light yellow oil. ¹H NMR (MeOD, 400 MHz,) δ 8.88 (d, J=2.0 Hz, 1H), 8.05 (d, J=2.0 Hz, 1H), 7.42 (s, 1H), 6.89 (s, 2H), 4.56-4.46 (m, 1H), 4.33-4.25 (m, 1H), 4.22-4.20 (m, 1H), 4.17 (s, 2H), 4.03-3.95 (m, 3H), 3.78-3.69 (m, 4H), 3.66-3.59 (m, 32H), 3.57-3.52 (m, 6H), 3.51 (s, 2H), 3.50-3.48 (m, 2H), 3.38 (q, J=5.2 Hz, 2H), 3.01-2.91 (m, 1H), 2.45 (t, J=5.6 Hz, 2H), 1.83-1.73 (m, 2H), 1.21 (t, J=7.2 Hz, 3H), 1.00 (t, J=7.2 Hz, 3H). LC/MS [M+H] 1049.5 (calculated); LC/MS [M+H] 1049.4 (observed).

### Example L-59 Synthesis of 6-amino-3-[6-[[3-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]propanoylamino]methyl]-3-pyridyl]-N,N-dipropyl-7H-pyrido[3,2-b]azepine-8-carboxamide, azaBzL-59

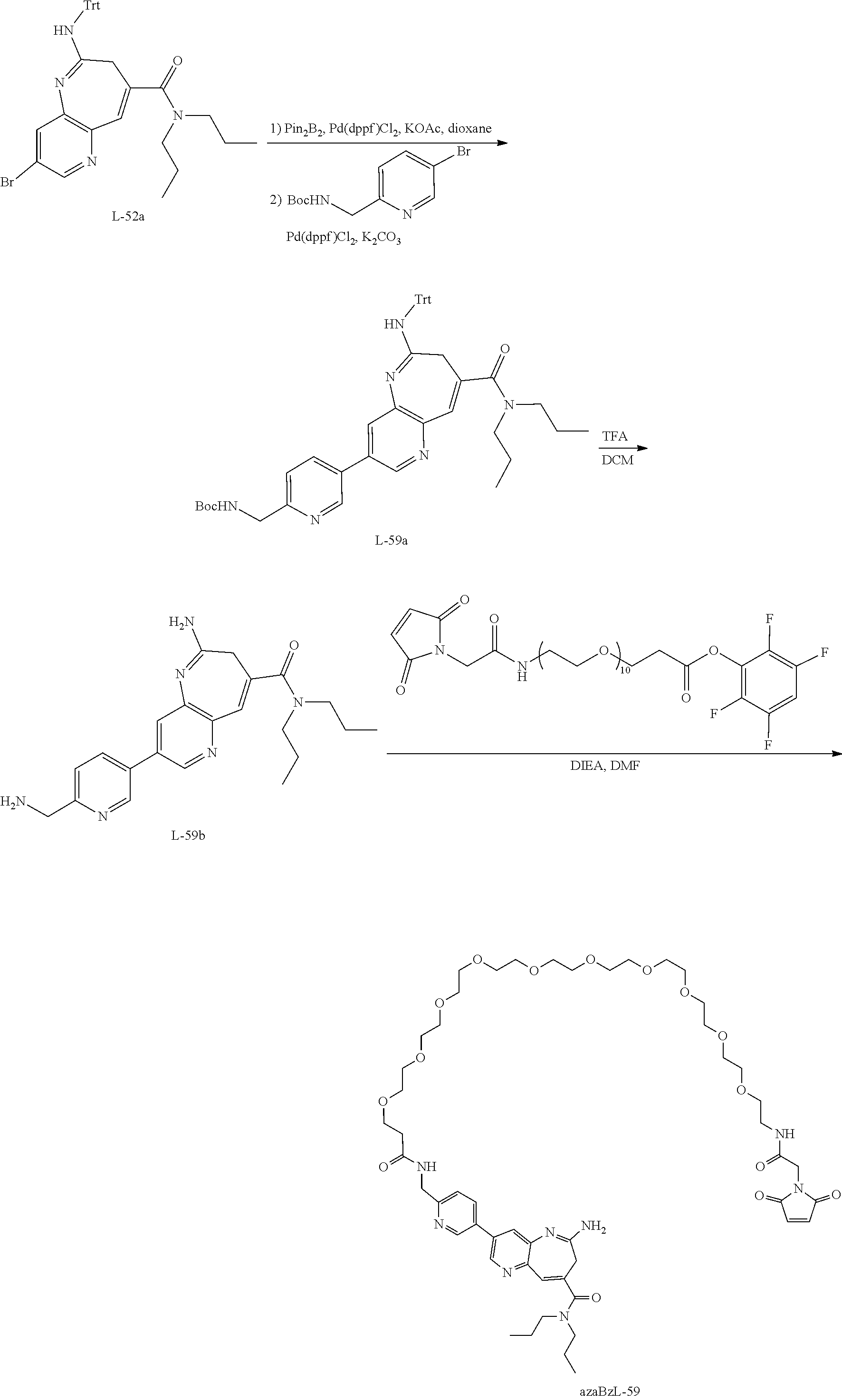

### Preparation of tert-butyl N-[[5-[8-(dipropylcarbamoyl)-6-(tritylamino)-7H-pyrido[3,2-b]azepin-3-yl]-2-pyridyl]methyl]carbamate, L-59a

A mixture of 3-bromo-N,N-dipropyl-6-(tritylamino)-7H-pyrido[3,2-b]azepine-8-carboxamide, L-52a (500 mg, 823 mol, 1 eq) in dioxane (10 mL) were added Pin₂B₂(313 mg, 1.23 mmol, 1.5 eq), KOAc (162 mg, 1.65 mmol, 2 eq) and Pd(dppf)Cl₂ (30.1 mg, 41.2 mol, 0.05 eq), the mixture was degassed and purged with N₂ for 3 times, then heated to 95° C. and stirred at 95° C. for 2 hrs under N₂ atmosphere. The reaction mixture was cooled to room temperature, and then were added tert-butyl N-[(5-bromo-2-pyridyl)methyl]carbamate (211 mg, 733 mol, 1.2 eq), K₂CO₃ (169 mg, 1.22 mmol, 2.0 eq), Pd(dppf)Cl₂ (22.4 mg, 30.6 mol, 0.05 eq) and H₂O (1 mL), the mixture was degassed and purged with N₂ for 3 times, then heated to 95° C. and stirred at 95° C. for another 1 hr under N₂ atmosphere. The reaction mixture was cooled to room temperature then diluted with water and extracted with EtOAc (15 ml×3). The organic layer was washed with brine (10 mL), dried over Na₂SO₄, filtered and concentrated to give L-59a (0.40 g, 544 mol, 89.1% yield) as yellow oil. LC/MS [M+H] 735.4 (calculated); LC/MS [M+H] 735.2 (observed).

### Preparation of 6-amino-3-[6-(aminomethyl)-3-pyridyl]-N,N-dipropyl-7H-pyrido[3,2-b]azepine-8-carboxamide, L-59b

To a solution of L-59a (0.40 g, 544 mol, 1 eq) in DCM (5 mL) was added TFA (1.24 g, 10.9 mmol, 809 μL, 20 eq), and then heated to 50° C. and stirred for 16 hrs. The reaction mixture was cooled to room temperature and concentrated under reduced pressure to give a residue. The residue was purified by prep-HPLC (column: Phenomenex Luna C18 75*30 mm*3 um; mobile phase: [H₂O (0.1% TFA)-ACN]; gradient: 5%-30% B over 8.0 min) to give L-59b (0.15 g, 363 mol, 66.7% yield, 95% purity) as yellow oil. ¹H NMR (MeOD, 400 MHz) δ 9.05 (d, J=2.0 Hz, 1H), 8.98 (d, J=2.0 Hz, 1H), 8.27 (dd, J=2.0, 8.4 Hz, 1H), 8.14 (d, J=2.0 Hz, 1H), 7.63 (d, J=8.4 Hz, 1H), 7.08 (s, 1H), 4.39 (s, 2H), 3.63-3.40 (m, 6H), 1.77-1.67 (m, 4H), 1.03-0.89 (m, 6H). LC/MS [M+H] 393.2 (calculated); LC/MS [M+H] 393.1 (observed).

### Preparation of azaBzL-59

To a solution of L-59b (0.04 g, 102 mol, 1 eq) in DMF (0.5 mL) were added DIEA (26.3 mg, 204 mol, 35.5 μL, 2.0 eq) and (2,3,5,6-tetrafluorophenyl) 3-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]propanoate (83.0 mg, 102 mol, 1 eq), and then stirred at 20° C. for 1 hr. The reaction mixture was adjusted to pH-6 with TFA and purified by prep-HPLC (column: Phenomenex Luna C18 75*30 mm*3 um; mobile phase: [H₂O (0.1% TFA)-ACN]; gradient: 15%-35% B over 8.0 min) to give azaBzL-59 (0.07 g, 65.2 mol, 63.9% yield, 97% purity) as colorless oil. ¹H NMR (MeOD, 400 MHz) δ 9.01 (d, J=2.0 Hz, 1H), 8.95 (d, J=2.0 Hz, 1H), 8.34 (dd, J=2.0, 8.0 Hz, 1H), 8.13 (d, J=2.0 Hz, 1H), 7.72 (d, J=8.0 Hz, 1H), 7.09 (s, 1H), 6.88 (s, 2H), 4.63 (s, 2H), 4.16 (s, 2H), 3.80 (t, J=6.0 Hz, 2H), 3.68-3.64 (m, 4H), 3.64-3.56 (m, 32H), 3.55-3.43 (m, 8H), 3.35 (t, J=5.6 Hz, 2H), 2.58 (t, J=5.6 Hz, 2H), 1.78-1.63 (m, 4H), 1.04-0.87 (m, 6H). LC/MS [M+H] 1041.5 (calculated); LC/MS [M+H] 1041.6 (observed).

### Example L-72 Synthesis of 6-amino-N-ethoxy-N-propyl-3-[rac-(2S)-2-[[3-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]propanoylamino]methyl]morpholine-4-carbonyl]-7H-pyrido[3,2-b]azepine-8-carboxamide, azaBzL-72

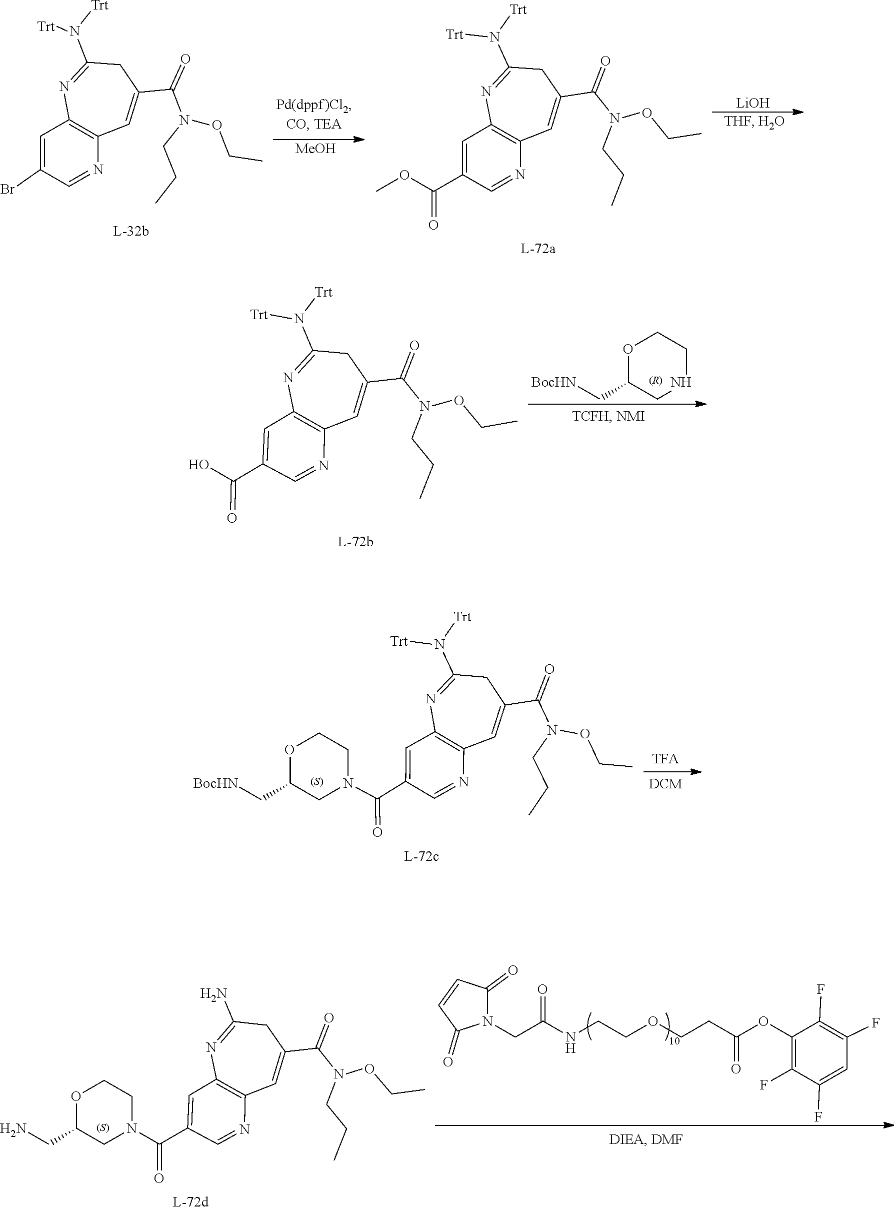

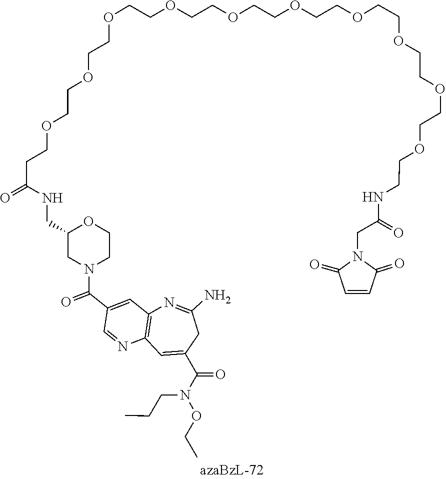 [SMILES: CCCN(OCC)C(=O)C1=Cc2ncc(C(=O)N3CCO[C@@H](CNC(=O)CCOCCOCCOCCOCCOCCOCCOCCOCCOCCOCCNC(=O)CN4C(=O)C=CC4=O)C3)cc2N=C(N)C1]

### Preparation of methyl 6-(ditritylamino)-8-[ethoxy(propyl)carbamoyl]-7H-pyrido[3,2-b]azepine-3-carboxylate, L-72a

A mixture of 3-bromo-6-(ditritylamino)-N-ethoxy-N-propyl-7H-pyrido[3,2-b]azepine-8-carboxamide, L-32b (8.8 g, 10.3 mmol, 1 eq), Pd(dppf)Cl₂ (755 mg, 1.03 mmol, 0.1 eq), TEA (3.14 g, 30.9 mmol, 4.31 mL, 3 eq) in MeOH (150 mL) was degassed and purged with carbon monoxide, CO for 3 times, and then the mixture was heated to 80° C. and stirred for 16 hrs under CO atmosphere (50 psi). The reaction mixture concentrated under reduced pressure to give a residue. The residue was purified by column chromatography (SiO2, Petroleum ether/Ethyl acetate=I1/0 to 1/1) to give L-72a (6 g, 7.01 mmol, 67.9% yield, 97.1% purity) as a yellow solid. LC/MS [M+H] 831.4 (calculated); LC/MS [M+H] 831.3 (observed).

### Preparation of 6-(ditritylamino)-8-[ethoxy(propyl)carbamoyl]-7H-pyrido[3,2-b]azepine-3-carboxylic acid, L-72b

To a solution of L-72a (5.5 g, 6.62 mmol, 1 eq) in THE (50 mL) was added a solution of LiOH·H₂O (833 mg, 19.8 mmol, 3 eq) in H₂O (20 mL) at 0° C., and then warmed to 50° C. and stirred for 8 hrs. The reaction mixture was cooled to 0° C., diluted with H₂O (30 mL) and adjusted to pH=5 with 2 N HCl at 0° C., concentrated under reduced pressure to remove THF. The suspension was filtered and the filter cake was dried under reduced pressure to give L-72b (5.57 g, crude) as a yellow solid. LC/MS [M+H] 817.4 (calculated); LC/MS [M+H] 817.4 (observed).

### Preparation of tert-butyl N-[[rac-(2S)-4-[6-(ditritylamino)-8-[ethoxy(propyl)carbamoyl]-7H-pyrido[3,2-b]azepine-3-carbonyl]morpholin-2-yl]methyl]carbamate, L-72c

To a solution of L-72b (400 mg, 490 mol, 1.0 eq) and tert-butyl N-[[rac-(2R)-morpholin-2-yl]methyl]carbamate (127 mg, 588 mol, 1.2 eq) in MeCN (5 mL) were added N-methylimidazole, NMI (201 mg, 2.45 mmol, 195 μL, 5.0 eq) and TCFH (206 mg, 734 mol, 1.5 eq) at 0° C., and then warmed to 25° C. and stirred for 1 hr. The reaction mixture was quenched by addition H₂O (30 mL) at 0° C., and then extracted with EtOAc (20 mL×3). The combined organic layers were washed with brine (30 mL), dried over Na₂SO₄, filtered and concentrated under reduced pressure to give a residue. The residue was purified by flash silica gel chromatography (Biotage®; 12 g SepaFlash® Silica Flash Column, Eluent of 35~55% Ethyl acetate/Petroleum ether gradient @65 mL/min) to give L-72c (497 mg, 489 mol, 99.9% yield) as a yellow solid. LC/MS [M+H] 1015.5 (calculated); LC/MS [M+H] 1015.4 (observed).

### Preparation of 6-amino-N-ethoxy-N-propyl-3-[rac-(2S)-2-(aminomethyl)morpholine-4-carbonyl]-7H-pyrido[3,2-b]azepine-8-carboxamide, L-72d

To a solution of L-72c (480 mg, 473 mol, 1 eq) in DCM (5 mL) was added TFA (1.48 g, 13.0 mmol, 966 μL, 27.5 eq), and then heated to 50° C. and stirred for 2 hrs. The reaction mixture was concentrated under reduced pressure to remove solvent. The crude product was triturated with MTBE (20 mL) at 25° C. for 10 min. to give L-72d (300 mg, crude, TFA) as a brown solid. ¹H NMR (MeOD, 400 MHz) δ 8.60 (d, J=2.0 Hz, 1H), 7.80 (d, J=2.0 Hz, 1H), 7.30 (s, 1H), 4.57-4.31 (m, 1H), 3.88 (q, J=7.2 Hz, 2H), 3.66 (t, J=7.2 Hz, 2H), 3.62-3.52 (m, 2H), 3.40 (s, 2H), 3.16-2.74 (m, 6H), 1.74-1.63 (m, 2H), 1.10 (t, J=7.2 Hz, 3H), 0.91 (t, J=7.2 Hz, 3H). LC/MS [M+H] 431.2 (calculated); LC/MS [M+H] 431.2 (observed).

### Preparation of azaBzL-72

To a solution of L-72d (50.0 mg, 91.8 mol, 1 eq, TFA) in DMF (1 mL) were added DIEA (35.6 mg, 275 mol, 48.0 μL, 3 eq) and (2,3,5,6-tetrafluorophenyl) 3-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]propanoate (74.8 mg, 91.8 mol, 1 eq) at 0° C., and then warmed to 25° C. and stirred for 1 hr. The reaction mixture was adjusted to pH=5 6 with TFA. The residue was purified by prep-HPLC (column: Phenomenex luna C18 100*40 mm*3 um; mobile phase: [H₂O (0.10% TFA)-ACN]; gradient: 5%-35% B over 8.0 min) to give azaBzL-72 (35 mg, 29.3 mol, 31.9% yield, TFA) as colorless oil. ¹H NMR (MeOD, 400 MHz) δ 8.70 (d, J=2.0 Hz, 1H), 7.86 (d, J=2.0 Hz, 1H), 7.43 (s, 1H), 6.89 (s, 2H), 4.60-4.39 (m, 1H), 4.17 (s, 2H), 3.99 (q, J=7.2 Hz, 2H), 3.76 (t, J=7.2 Hz, 2H), 3.70-3.47 (m, 46H), 3.48-3.30 (m, 2H), 3.25-3.03 (m, 2H), 2.55-2.36 (m, 2H), 1.84-1.73 (m, 2H), 1.21 (t, J=7.2 Hz, 3H), 1.01 (t, J=7.2 Hz, 3H). LC/MS [M+H] 1079.5 (calculated); LC/MS [M+H] 1079.7 (observed).

### Example L-82 Synthesis of 6-amino-3-[1-[2-[2-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]eth ylamino]-2-oxo-ethyl]pyrazol-4-yl]-N-ethoxy-N-propyl-7H-pyrido[3,2-b]azepine-8-carboxamide, azaBzL-82

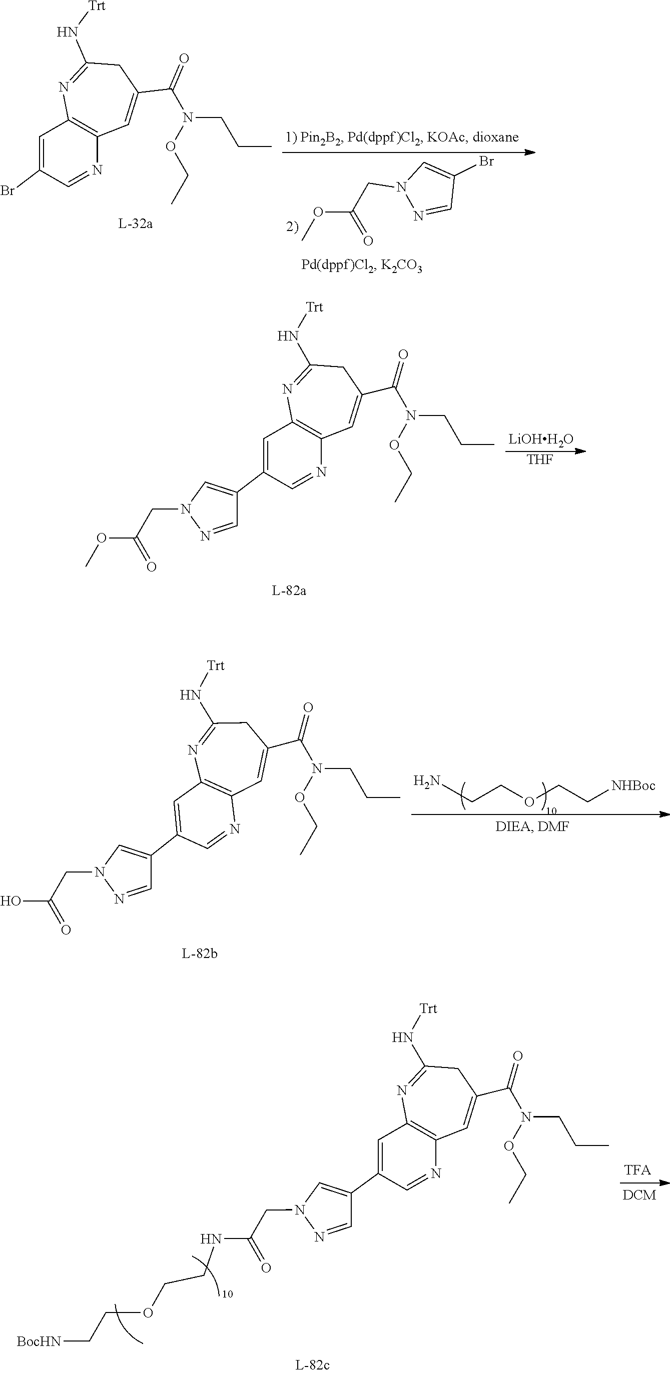

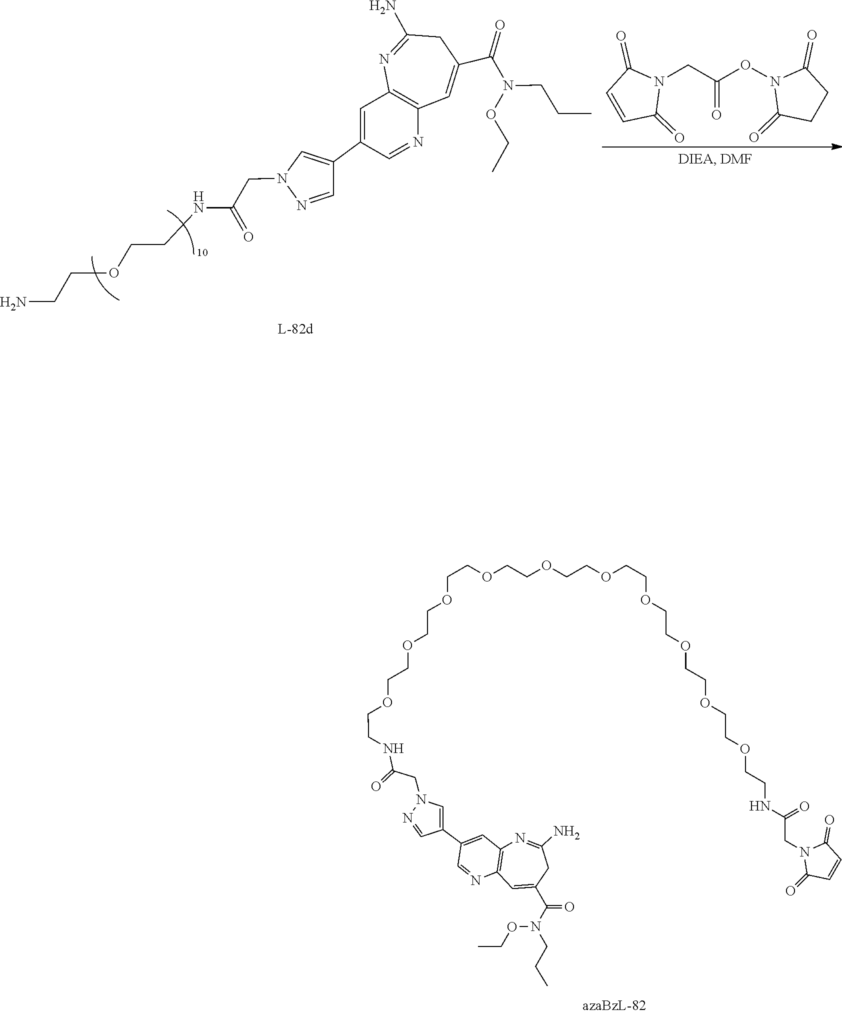

### Preparation of methyl 2-[4-[8-[ethoxy(propyl)carbamoyl]-6-(tritylamino)-7H-pyrido[3,2-b]azepin-3-yl]pyrazol-1-yl]acetate, L-82a

To a solution of 3-bromo-N-ethoxy-N-propyl-6-(tritylamino)-7H-pyrido[3,2-b]azepine-8-carboxamide, L-32a (1.5 g, 2.46 mmol, 1 eq) in dioxane (30 mL) were added Pin₂B₂(937 mg, 3.69 mmol, 1.5 eq), KOAc (483 mg, 4.92 mmol, 2 eq) and Pd(dppf)Cl₂ (180.06 mg, 246.08 mol, 0.1 eq) at 25° C. under N₂, and then heated to 90° C. and stirred for 2 hrs. The reaction mixture was cooled to 25° C. and the added methyl 2-(4-bromopyrazol-1-yl)acetate (200 mg, 913 mol, 1.2 eq), K₂CO₃ (210 mg, 1.52 mmol, 2.0 eq), Pd(dppf)Cl₂ (55.7 mg, 76.1 mol, 0.1 eq) and H₂O (0.7 mL), the reaction mixture was degassed and purged with N₂ for 3 times, heated to 100° C. and stirred for another 1 hr under N₂ atmosphere. The reaction mixture was cooled to 25° C., filtered and concentrated under reduced pressure to give L-82a (500 mg, crude) as black oil. LC/MS [M+H] 669.3 (calculated); LC/MS [M+H] 669.2 (observed).

### Preparation of 2-[4-[8-[ethoxy(propyl)carbamoyl]-6-(tritylamino)-7H-pyrido[3,2-b]azepin-3-yl]pyrazol-1-yl]acetic acid, L-82b

To a solution of L-82a (500 mg, 747 mol, 1 eq) in THE (6 mL) was added a solution of LiOH H₂O (156 mg, 3.74 mmol, 5 eq) in H₂O (2 mL), and then stirred at 25° C. for 1 hr. The reaction mixture was concentrated under reduced pressure to remove THE and adjusted to pH=4-5 with 4N HCl. The precipitate was filtered and the filter cake was collected to afford L-82b (345 mg, 527 mol, 70.5% yield) as a brown solid. ¹H NMR (MeOD, 400 MHz) δ 8.36 (d, J=1.6 Hz, 1H), 7.98 (s, 1H), 7.72 (s, 1H), 7.39-7.32 (m, 6H), 7.28-7.14 (m, 1OH), 7.00 (s, 1H), 4.75 (s, 2H), 4.01 (q, J=6.8 Hz, 2H), 3.80 (t, J=6.8 Hz, 2H), 2.97 (s, 2H), 1.83-1.76 (m, 2H), 1.26-1.23 (m, 3H), 1.01 (t, J=7.2 Hz, 3H). LC/MS [M+H] 655.3 (calculated); LC/MS [M+H] 655.1 (observed).

### Preparation of tert-butyl N-[2-[2-[2-[2-[2-[2-[2-[2-[2-[2-[2-[[2-[4-[8-[ethoxy(propyl)carbamoyl]-6-(tritylamino)-7H-pyrido[3,2-b]azepin-3-yl]pyrazol-1-yl]acetyl]amino]ethoxy]ethoxy]ethoxy]ethoxy]ethoxy]ethoxy]ethoxy]ethoxy]ethoxy]ethoxy]eth yl]carbamate, L-82c

To a solution of L-82b (130 mg, 198 mol, 1 eq) in DMF (2 mL) were added HATU (150 mg, 397 mol, 2 eq), DIEA (76.9 mg, 595 mol, 103 μL, 3 eq) and tert-butyl N-[2-[2-[2-[2-[2-[2-[2-[2-[2-[2-(2-aminoethoxy)ethoxy]ethoxy]ethoxy]ethoxy]ethoxy]ethoxy]ethoxy]ethoxy]ethoxy]ethyl]carbamate (119 mg, 198 mol, 1 eq) at 0° C., and then warmed to 25° C. and stirred for 1 hr. The reaction mixture was diluted with H₂O (5 mL) and extracted with EtOAc (10 mL×3). The combined organic layers were washed with brine (10 mL), dried over Na₂SO₄, filtered and concentrated under reduced pressure to give L-82c (280 mg, crude) as yellow oil. LC/MS [M+H] 1237.7 (calculated); LC/MS [M+H] 1237.3 (observed).

### Preparation of 6-amino-3-[1-[2-[2-[2-[2-[2-[2-[2-[2-[2-[2-[2-(2-aminoethoxy)ethoxy]ethoxy]ethoxy]ethoxy]ethoxy]ethoxy]ethoxy]ethoxy]ethoxy]ethylamino]-2-oxo-ethyl]pyrazol-4-yl]-N-ethoxy-N-propyl-7H-pyrido[3,2-b]azepine-8-carboxamide, L-82d

To a solution of L-82c (280 mg, 226 mol, 1 eq) in DCM (10 mL) was added TFA (515 mg, 4.53 mmol, 336 μL, 20 eq), and then heated to 50° C. and stirred for 1 hr. The reaction mixture was cooled to 25° C. and concentrated under reduced pressure, the residue was diluted with H₂O (5 mL) and extracted with MTBE (5 mL×3). The aqueous phase was lyophilized to afford L-82d (90 mg, 100 mol, 44.4% yield) as yellow oil. ¹H NMR (MeOD, 400 MHz) δ 8.88 (s, 1H), 8.33 (s, 1H), 8.09 (s, 1H), 7.99 (s, 1H), 7.38 (s, 1H), 4.96 (s, 2H), 3.98 (q, J=7.2 Hz, 2H), 3.79-3.74 (m, 4H), 3.73-3.62 (m, 34H), 3.58 (t, J=5.6 Hz, 2H), 3.50-3.41 (m, 4H), 3.28-3.15 (m, 4H), 1.83-1.73 (m, 2H), 1.23-1.19 (m, 3H), 1.01 (t, J=7.2 Hz, 3H). LC/MS [M+H] 895.5 (calculated); LC/MS [M+H] 895.3 (observed).

### Preparation of azaBzL-82

To a solution of L-82d (70 mg, 69.3 mol, 1 eq, TFA) in DMF (1 mL) were added DIEA (26.9 mg, 208 mol, 36.2 μL, 3 eq) and (2,5-dioxopyrrolidin-1-yl) 2-(2,5-dioxopyrrol-1-yl)acetate (17.4 mg, 69.3 mol, 1 eq), and then stirred at 25° C. for 1 hr. The reaction mixture was adjusted to pH=4-5 with TFA and purified by prep-HPLC (column: Phenomenex Luna C18 75*30 mm*3 um; mobile phase: [H₂O (0.1% TFA)-ACN]; gradient: 20%-40% B over 8.0 min) azaBzL-82 (28.1 mg, 27.2 mol, 39.2% yield) as light yellow oil. ¹H NMR (MeOD, 400 MHz) δ 8.91 (d, J=2.0 Hz, 1H), 8.32 (s, 1H), 8.07 (s, 1H), 7.94 (d, J=2.0 Hz, 1H), 7.78 (s, 1H), 6.88 (s, 2H), 4.98 (s, 2H), 4.16 (s, 2H), 3.99 (q, J=7.2 Hz, 2H), 3.77 (t, J=7.2 Hz, 2H), 3.70-3.57 (m, 38H), 3.53 (t, J=5.6 Hz, 2H), 3.49 (s, 2H), 3.45-3.43 (m, 2H), 3.36 (t, J=5.6 Hz, 2H), 1.85-1.73 (m, 2H), 1.22 (t, J=7.2 Hz, 3H), 1.01 (t, J=7.2 Hz, 3H). LC/MS [M+H] 1032.5 (calculated); LC/MS [M+H] 1032.6 (observed).

### Example L-90 Synthesis of 2-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]ethyl N-[rac-(3S)-1-[6-amino-8-[ethoxy(propyl)carbamoyl]-7H-pyrido[3,2-b]azepine-3-carbonyl]pyrrolidin-3-yl]carbamate, azaBzL-90

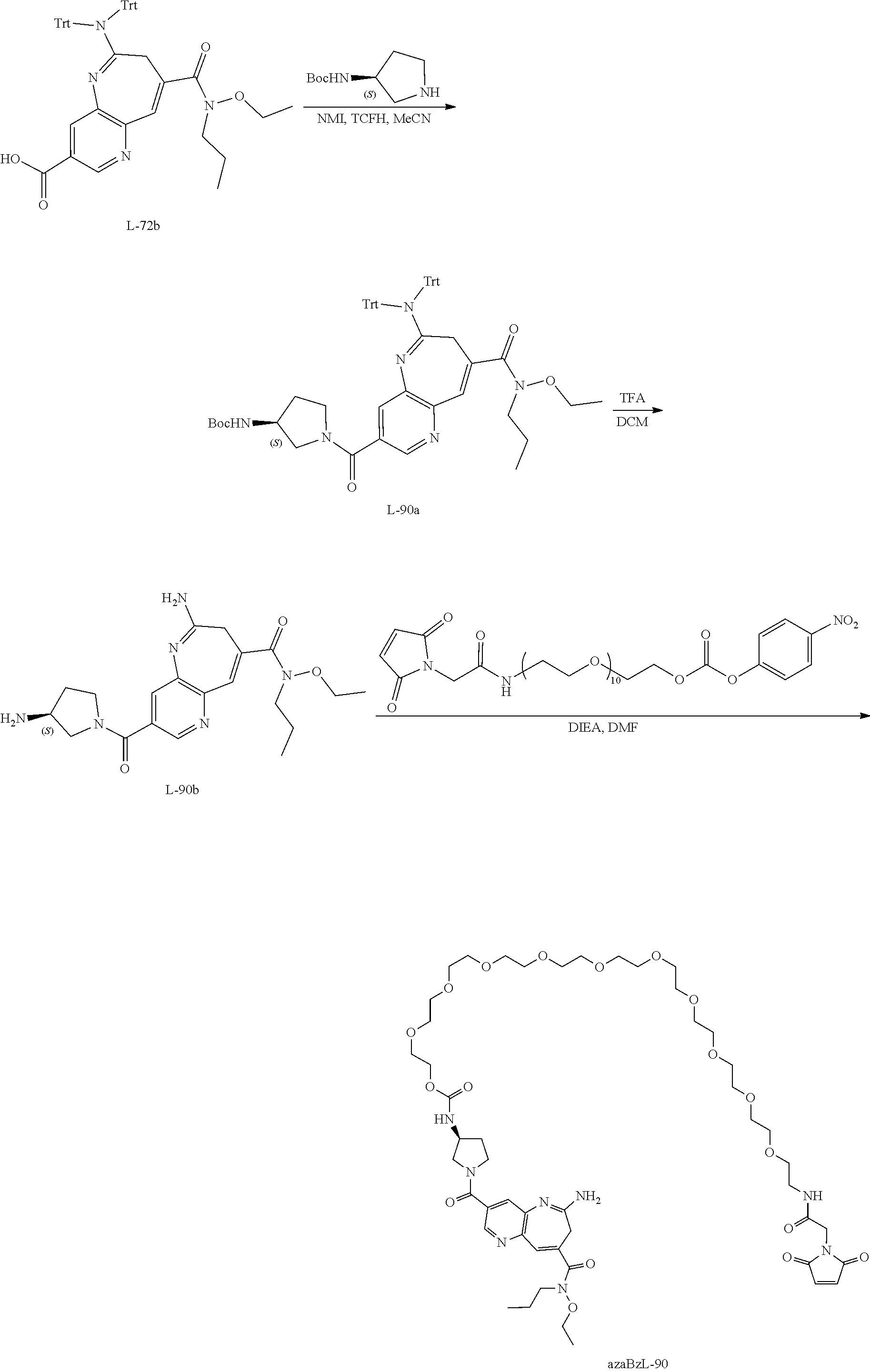

### Preparation of tert-butyl N-[rac-(3S)-1-[6-(ditritylamino)-8-[ethoxy(propyl)carbamoyl]-7H-pyrido[3,2-b]azepine-3-carbonyl]pyrrolidin-3-yl]carbamate, L-90a

To a solution of 6-(ditritylamino)-8-[ethoxy(propyl)carbamoyl]-7H-pyrido[3,2-b]azepine-3-carboxylic acid, L-72b (400 mg, 489 mol, 1 eq) and tert-butyl N-[rac-(3S)-pyrrolidin-3-yl]carbamate (109 mg, 587 mol, 1.2 eq) in MeCN (10 mL) were added NMI (160.7 mg, 1.96 mmol, 156 μL, 4 eq) and TCFH (206 mg, 734 mol, 1.5 eq), and then stirred at 25° C. for 1 hr. The reaction mixture was concentrated under reduced pressure to give a residue. The residue was purified by flash silica gel chromatography (biotage®; 8g SepaFlash® Silica Flash Column, Eluent of 0-60% Ethyl acetate/Petroleum ether gradient @60 mL/min) to give L-90a (500 mg, crude) as brown oil. LC/MS [M+H] 985.5 (calculated); LC/MS [M+H] 985.5 (observed).

### Preparation of 6-amino-N-ethoxy-N-propyl-3-[rac-(3S)-3-aminopyrrolidine-1-carbonyl]-7H-pyrido[3,2-b]azepine-8-carboxamide, L-90b

To a solution of L-90a (500 mg, 507 mol, 1 eq) in DCM (10 mL) was added TFA (1.74 g, 15.2 mmol, 1.13 mL, 30 eq) at 25° C., and then heated to 50° C. and stirred for 2 hrs. The reaction mixture was cooled to 25° C. and concentrated under reduced pressure to give a residue. The residue was triturated with MTBE at 0° C. for 20 min. to give L-90b (300 mg, crude) as brown solid. LC/MS [M+H] 401.2 (calculated); LC/MS [M+H] 401.1 (observed).

### Preparation of azaBzL-90

To a solution of L-90b (60 mg, 127 mol, 1 eq) in DMF (1.5 mL) were added DIEA (49.1 mg, 380 mol, 66.2 μL, 3 eq) and 2-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]ethyl (4-nitrophenyl) carbonate (102 mg, 127 mol, 1 eq) at 0° C., and then warmed to 20° C. and stirred for 1 hr. The reaction mixture was adjusted to 6 with TFA, and purified by prep-HPLC (column: Phenomenex Luna C18 75*30 mm*3 um; mobile phase: [H₂O (0.1% TFA)-ACN]; gradient: 20%-40% B over 8.0 min) to give azaBzL-90 (20 mg, 17.0 mol, 13.3% yield, TFA) as a light yellow solid. ¹H NMR (MeOD, 400 MHz) δ 8.88-8.78 (m, 1H), 7.96 (s, 1H), 7.45 (s, 1H), 6.91 (s, 2H), 4.26-4.21 (m, 1H), 4.19 (s, 2H), 4.17-4.10 (m, 1H), 4.01 (q, J=7.2 Hz, 2H), 3.93-3.86 (m, 1H), 3.85-3.75 (m, 4H), 3.75-3.70 (m, 2H), 3.69-3.60 (m, 36H), 3.59-3.44 (m, 6H), 3.42-3.37 (m, 2H), 2.35-2.20 (m, 1H), 2.09-1.99 (m, 1H), 1.83-1.77 (m, 2H), 1.23 (t, J=7.2 Hz, 3H), 1.03 (t, J=7.2 Hz, 3H). LC/MS [M+H] 1065.5 (calculated); LC/MS [M+H] 1065.4 (observed).

### Example L-148 Synthesis of (2,3,5,6-tetrafluorophenyl) 3-[2-[2-[2-[2-[2-[2-[2-[2-[2-[2-[2-[(2-amino-3H-pyrido[3,4-b]azepine-4-carbonyl)-propyl-amino]oxyethylcarbamoyloxy]ethoxy]ethoxy]ethoxy]ethoxy]ethoxy]ethoxy]ethoxy]ethoxy]ethoxy]ethoxy]propanoate, azaBzL-148

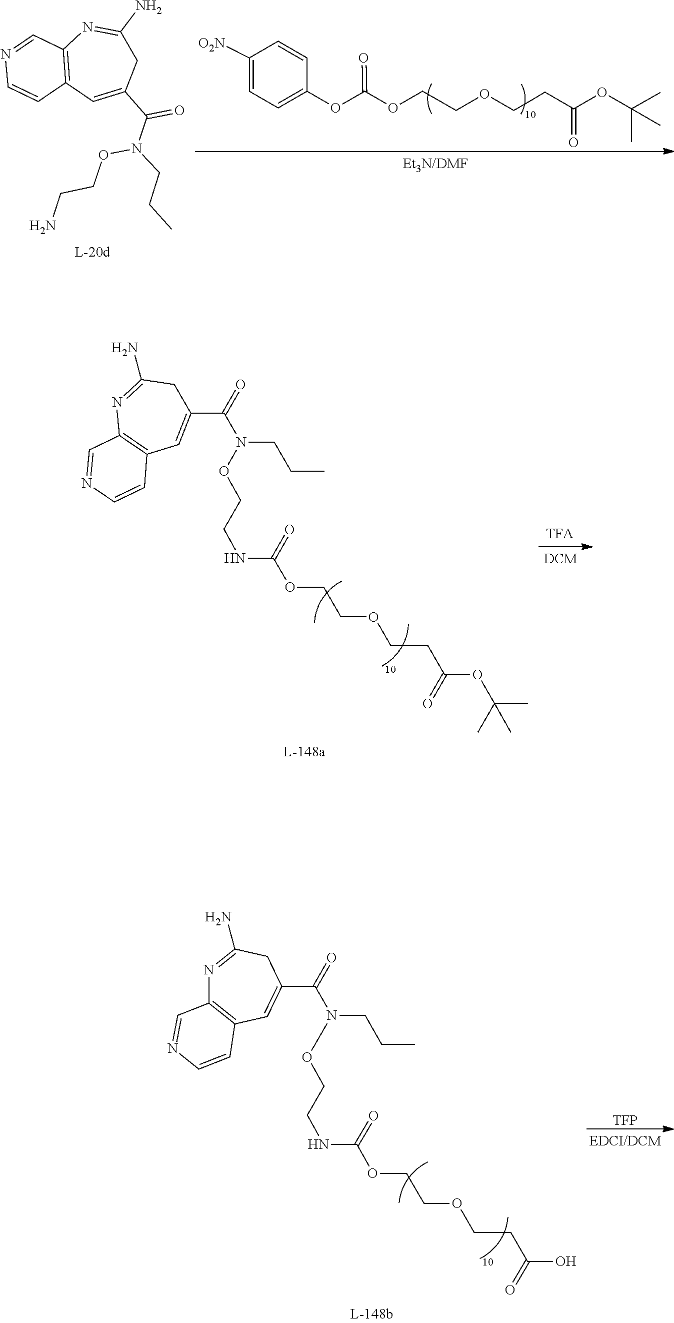

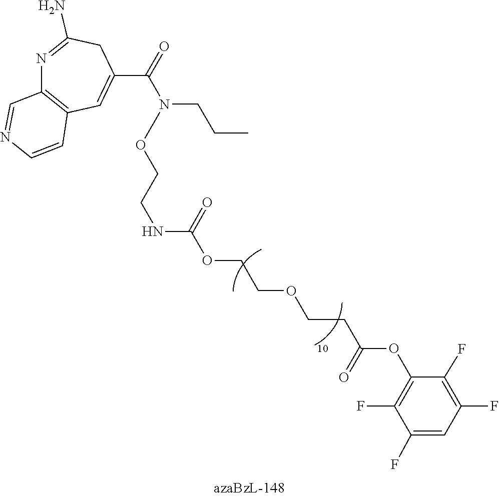

### Preparation of tert-butyl 3-[2-[2-[2-[2-[2-[2-[2-[2-[2-[2-[2-[(2-amino-3H-pyrido[3,4-b]azepine-4-carbonyl)-propyl-amino]oxyethylcarbamoyloxy]ethoxy]ethoxy]ethoxy]ethoxy]ethoxy]ethoxy]ethoxy]ethoxy]ethoxy]ethoxy]propanoate, L-148a

To a solution of 2-amino-N-(2-aminoethoxy)-N-propyl-3H-pyrido[3,4-b]azepine-4-carboxamide, L-20d (210 mg, 617 umol, 1 eq, HCl) in DMF (5 mL) were added DIEA (399 mg, 3.09 mmol, 538 μL, 5 eq) and tert-butyl 3-[2-[2-[2-[2-[2-[2-[2-[2-[2-[2-(4-nitrophenoxy)carbonyloxyethoxy]ethoxy]ethoxy]ethoxy]ethoxy]ethoxy]ethoxy]ethoxy]ethoxy]ethoxy]propanoate (464 mg, 617 umol, 1 eq), and then stirred at 20° C. for 2 hrs. The reaction mixture was filtered to give a residue. The crude product was purified by prep-HPLC (column: Phenomenex Luna 80×30 mm×3 um; mobile phase: [water (TFA)-ACN]; B %: 15%-45%, 8 min) to give L-148a (610 mg, crude) as a yellow oil. LC/MS [M+H] 916.5 (calculated); LC/MS [M+H] 916.5 (observed).

### Preparation of 3-[2-[2-[2-[2-[2-[2-[2-[2-[2-[2-[2-[(2-amino-3H-pyrido[3,4-b]azepine-4-carbonyl)-propyl-amino]oxyethylcarbamoyloxy]ethoxy]ethoxy]ethoxy]ethoxy]ethoxy]ethoxy]ethoxy]ethoxy]ethoxy]ethoxy]propanoic acid, L-148b

To a solution of L-147a (510 mg, 556 umol, 1 eq) in DCM (5 mL) was added trifluoroacetic acid, TFA (1.27 g, 11.1 mmol, 824 μL, 20 eq), and then stirred at 40° C. for 0.5 hr. The reaction mixture was concentrated under reduced pressure. The crude product was purified by prep-HPLC (column: Phenomenex Luna 80×30 mm×3 um; mobile phase: [water (TFA)-ACN]; B %: 5%-35%, 8 min) to give L-148b (220 mg, crude) as yellow oil. LC/MS [M+H] 860.4 (calculated); LC/MS [M+H] 860.3 (observed).

### Preparation of azaBzL-148

To a solution of L-148b (200 mg, 205 umol, 1 eq, TFA) and 2,3,5,6-tetrafluorophenol (170 mg, 1.03 mmol, 5 eq) in DCM (2 mL) and DMA (0.1 mL) was added EDCI (314 mg, 1.64 mmol, 8 eq), and then stirred at 20° C. for 0.5 hr. The reaction mixture was concentrated under reduced pressure to give a residue. The crude product was purified by prep-HPLC (column: Phenomenex Luna 80×30 mm×3 um; mobile phase: [water (TFA)-ACN]; B %: 25%-55%, 8 min). to give azaBzL-148 (90 mg, 89.2 umol, 43.4% yield) as yellow oil. ¹H NMR (MeOD, 400 MHz) δ 8.68 (s, 1H), 8.54 (d, J=4.8 Hz, 1H), 7.63 (d, J=4.8 Hz, 1H), 7.49-7.35 (m, 2H), 3.97 (t, J=4.8 Hz, 2H), 3.92-3.82 (m, 4H), 3.75 (t, J=7.2 Hz, 2H), 3.70-3.56 (m, 40H), 3.51 (d, J=3.6 Hz, 2H), 2.98 (t, J=6.0 Hz, 2H), 1.82-1.71 (m, 2H), 1.00 (t, J=7.2 Hz, 3H). LC/MS [M+H] 1008.4 (calculated); LC/MS [M+H] 1008.2 (observed).

### Example 201 Preparation of Immunoconjugates (IC)

To prepare a lysine-conjugated Immunoconjugate, an antibody is buffer exchanged into a conjugation buffer containing 100 mM boric acid, 50 mM sodium chloride, 1 mM ethylenediaminetetraacetic acid at pH 8.3, using G-25 SEPHADEX™ desalting columns (Sigma-Aldrich, St. Louis, MO) or Zeba™ Spin Desalting Columns (Thermo Fisher Scientific). The eluates are then each adjusted to a concentration of about 1-10 mg/ml using the buffer and then sterile filtered. The antibody is pre-warmed to 20-30° C. and rapidly mixed with 2-20 (e.g., 7-10) molar equivalents of a tetrafluorophenyl (TFP) or sulfonic tetrafluorophenyl (sulfoTFP) ester, aza-benzazepine-linker (azaBz-L) compound of Formula II dissolved in dimethylsulfoxide (DMSO) or dimethylacetamide (DMA) to a concentration of 5 to 20 mM. The reaction is allowed to proceed for about 16 hours at 30° C. and the immunoconjugate (IC) is separated from reactants by running over two successive G-25 desalting columns or Zeba™ Spin Desalting Columns equilibrated in phosphate buffered saline (PBS) at pH 7.2 to provide the Immunoconjugate (IC) of Tables 3a and 3b. Adjuvant-antibody ratio (DAR) is determined by liquid chromatography mass spectrometry analysis using a C4 reverse phase column on an ACQUITY™ UPL C H-class (Waters Corporation, Milford, MA) connected to a XEVO™ G2-XS TOF mass spectrometer (Waters Corporation).

To prepare a cysteine-conjugated Immunoconjugate, an antibody is buffer exchanged into a conjugation buffer containing PBS, pH 7.2 with 2 mM EDTA using Zeba™ Spin Desalting Columns (Thermo Fisher Scientific). The interchain disulfides are reduced using 2-4 molar excess of Tris (2-carboxyethyl) phosphine (TCEP) or dithiothreitol (DTT) at 37° C. for 30 min-2 hours. Excess TCEP or DTT was removed using a Zeba™ Spin Desalting column pre-equilibrated with the conjugation buffer. The concentration of the buffer-exchanged antibody was adjusted to approximately 5 to 20 mg/ml using the conjugation buffer and sterile-filtered. The maleimide-azaBz-L compound is either dissolved in dimethylsulfoxide (DMSO) or dimethylacetamide (DMA) to a concentration of 5 to 20 mM. For conjugation, the antibody is mixed with 10 to 20 molar equivalents of maleimide-azaBz-L. In some instances, additional DMA or DMSO up to 20% (v/v), was added to improve the solubility of the maleimide-azaBz-L in the conjugation buffer. The reaction is allowed to proceed for approximately 30 min to 4 hours at 20° C. The resulting conjugate is purified away from the unreacted maleimide-azaBz-L using two successive Zeba™ Spin Desalting Columns. The columns are pre-equilibrated with phosphate-buffered saline (PBS), pH 7.2. Adjuvant to antibody ratio (DAR) is estimated by liquid chromatography mass spectrometry analysis using a C4 reverse phase column on an ACQUITY™ UPLC H-class (Waters Corporation, Milford, MA) connected to a XEVO™ G2-XS TOF mass spectrometer (Waters Corporation).

For conjugation, the antibody may be dissolved in an aqueous buffer system known in the art that will not adversely impact the stability or antigen-binding specificity of the antibody. Phosphate buffered saline may be used. The azaBz-L compound is dissolved in a solvent system comprising at least one polar aprotic solvent as described elsewhere herein. In some such aspects, azaBz-L is dissolved to a concentration of about 5 mM, about 10 mM, about 20 mM, about 30 mM, about 40 mM or about 50 mM, and ranges thereof such as from about 5 mM to about 50 mM or from about 10 mM to about 30 mM in pH 8 Tris buffer (e.g., 50 mM Tris). In some aspects, the aza-benzazepine-linker intermediate is dissolved in DMSO (dimethylsulfoxide), DMA (dimethylacetamide), acetonitrile, or another suitable dipolar aprotic solvent.

Alternatively in the conjugation reaction, an equivalent excess of azaBz-L solution may be diluted and combined with antibody solution. The azaBz-L solution may suitably be diluted with at least one polar aprotic solvent and at least one polar protic solvent, examples of which include water, methanol, ethanol, n-propanol, and acetic acid. The molar equivalents of azaBz-L intermediate to antibody may be about 1.5:1, about 3:1, about 5:1, about 10:1, about 15:1, or about 20:1, and ranges thereof, such as from about 1.5:1 to about 20:1 from about 1.5:1 to about 15:1, from about 1.5:1 to about 10:1, from about 3:1 to about 15:1, from about 3:1 to about 10:1, from about 5:1 to about 15:1 or from about 5:1 to about 10:1. The reaction may suitably be monitored for completion by methods known in the art, such as LC-MS. The conjugation reaction is typically complete in a range from about 1 hour to about 16 hours. After the reaction is complete, a reagent may be added to the reaction mixture to quench the reaction. If antibody thiol groups are reacting with a thiol-reactive group such as maleimide of the azaBz-L linker intermediate, unreacted antibody thiol groups may be reacted with a capping reagent. An example of a suitable capping reagent is ethylmaleimide.

Following conjugation, the immunoconjugates may be purified and separated from unconjugated reactants and/or conjugate aggregates by purification methods known in the art such as, for example and not limited to, size exclusion chromatography, hydrophobic interaction chromatography, ion exchange chromatography, chromatofocusing, ultrafiltration, centrifugal ultrafiltration, tangential flow filtration, and combinations thereof. For instance, purification may be preceded by diluting the immunoconjugate, such in 20 mM sodium succinate, pH 5. The diluted solution is applied to a cation exchange column followed by washing with, e.g., at least 10 column volumes of 20 mM sodium succinate, pH 5. The conjugate may be suitably eluted with a buffer such as PBS.

### Example 202 HEK Reporter Assay

HEK293 reporter cells expressing human TLR7 or human TLR8 were purchased from Invivogen and vendor protocols were followed for cellular propagation and experimentation. Briefly, cells were grown to 80-85% confluence at 5% CO₂ in DMEM supplemented with 10% FBS, Zeocin, and Blasticidin. Cells were then seeded in 96-well flat plates at 4×10⁴ cells/well with substrate containing HEK detection medium and immunostimulatory molecules. Activity was measured using a plate reader at 620-655 nm wavelength.

### Example 203 Assessment of Immunoconjugate Activity In Vitro

This example shows that Immunoconjugates of the invention are effective at eliciting immune activation, and therefore are useful for the treatment of cancer.
a) Isolation of Human Antigen Presenting Cells: Human myeloid antigen presenting cells (APCs) were negatively selected from human peripheral blood obtained from healthy blood donors (Stanford Blood Center, Palo Alto, California) by density gradient centrifugation using a ROSETTESEP™ Human Monocyte Enrichment Cocktail (Stem Cell Technologies, Vancouver, Canada) containing monoclonal antibodies against CD14, CD16, CD40, CD86, CD123, and HLA-DR. Immature APCs were subsequently purified to >90% purity via negative selection using an EASYSEP™ Human Monocyte Enrichment Kit (Stem Cell Technologies) without CD16 depletion containing monoclonal antibodies against CD14, CD16, CD40, CD86, CD123, and HLA-DR.
b) Myeloid APC Activation Assay: 2×10⁵ APCs are incubated in 96-well plates (Corning, Corning, NY) containing iscove's modified dulbecco's medium, IMDM (Lonza) supplemented with 10% FBS, 100 U/mL penicillin, 100 μg/mL (micrograms per milliliter) streptomycin, 2 mM L-glutamine, sodium pyruvate, non-essential amino acids, and where indicated, various concentrations of unconjugated (naked) antibodies and immunoconjugates (IC) of the invention (as prepared according to the Example above). Cell-free supernatants are analyzed after 18 hours via ELISA to measure TNFα secretion as a readout of a proinflammatory response.
c) PBMC Activation Assay: Human peripheral blood mononuclear cells were isolated from human peripheral blood obtained from healthy blood donors (Stanford Blood Center, Palo Alto, California) by density gradient centrifugation. PBMCs were incubated in 96-well plates (Corning, Corning, NY) in a co-culture with CEA-expressing tumor cells (e.g. MKN-45, HPAF-II) at a 10:1 effector to target cell ratio. Cells were stimulated with various concentrations of unconjugated (naked) antibodies and immunoconjugates of the invention (as prepared according to the Example above). Cell-free supernatants were analyzed by cytokine bead array using a LegendPlex™ kit according to manufacturer's guidelines (BioLegend®, San Diego, CA).
d) Isolation of Human Conventional Dendritic Cells: Human conventional dendritic cells (cDCs) were negatively selected from human peripheral blood obtained from healthy blood donors (Stanford Blood Center, Palo Alto, California) by density gradient centrifugation. Briefly, cells are first enriched by using a ROSETTESEP™ Human CD3 Depletion Cocktail (Stem Cell Technologies, Vancouver, Canada) to remove T cells from the cell preparation. cDCs are then further enriched via negative selection using an EASYSEP™ Human Myeloid DC Enrichment Kit (Stem Cell Technologies).
e) cDC Activation Assay: 8×10⁴ APCs were co-cultured with tumor cells expressing the ISAC target antigen at a 10:1 effector (cDC) to target (tumor cell) ratio. Cells were incubated in 96-well plates (Corning, Corning, NY) containing RPMI-1640 medium supplemented with 10% FBS, and where indicated, various concentrations of the indicated immunoconjugate of the invention (as prepared according to the example above). Following overnight incubation of about 18 hours, cell-free supernatants were collected and analyzed for cytokine secretion (including TNFα) using a BioLegend LEGENDPLEX cytokine bead array.

Activation of myeloid cell types can be measured using various screen assays in addition to the assay described in which different myeloid populations are utilized. These may include the following: monocytes isolated from healthy donor blood, M-CSF differentiated Macrophages, GM-CSF differentiated Macrophages, GM-CSF+IL-4 monocyte-derived Dendritic Cells, conventional Dendritic Cells (cDCs) isolated from healthy donor blood, and myeloid cells polarized to an immunosuppressive state (also referred to as myeloid derived suppressor cells or MDSCs). Examples of MDSC polarized cells include monocytes differentiated toward immunosuppressive state such as M2a M(D (IL4/IL13), M2c M(D (IL10/TGFb), GM-CSF/IL6 MDSCs and tumor-educated monocytes (TEM). TEM differentiation can be performed using tumor-conditioned media (e.g. 786.0, MDA-MB-231, HCC1954). Primary tumor-associated myeloid cells may also include primary cells present in dissociated tumor cell suspensions (Discovery Life Sciences).

Assessment of activation of the described populations of myeloid cells may be performed as a mono-culture or as a co-culture with cells expressing the antigen of interest which the immunoconjugate (IC) may bind to via the CDR region of the antibody. Following incubation for 18-48 hours, activation may be assessed by upregulation of cell surface co-stimulatory molecules using flow cytometry or by measurement of secreted proinflammatory cytokines. For cytokine measurement, cell-free supernatant is harvested and analyzed by cytokine bead array (e.g. LegendPlex from Biolegend) using flow cytometry.

All references, including publications, patent applications, and patents, cited herein are hereby incorporated by reference to the same extent as if each reference were individually and specifically indicated to be incorporated by reference and were set forth in its entirety herein.

1. An immunoconjugate of Formula I comprising an antibody covalently attached to one or more aza-benzazepine moieties by a linker: 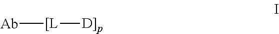
or a pharmaceutically acceptable salt thereof, wherein:
Ab is an antibody;
p is an integer from 1 to 8;
L is the linker;
D is the aza-benzazepine moiety having the formula: 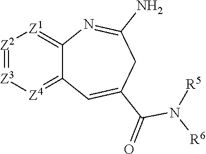
Z¹ is selected from CR¹ and N;
Z² is selected from CR² and N;
Z³ is selected from CR³ and N;
Z⁴ is selected from CR⁴ and N;
where one or two of Z¹, Z², Z³, and Z⁴ are N;
R¹, R², R³, R⁴, R⁵, and R⁶ are independently selected from the group consisting of H, C(═O), C(═O)N(R⁵), O, N(R⁵), S, S(O)₂, S(O)₂N(R⁵), C₁-C₁₂ alkyl, C₂-C₆ alkenyl, C₂-C₆ alkynyl, C₃-C₁₂ carbocyclyl, C₆-C₂₀ aryl, C₂-C₉ heterocyclyl, and C₁-C₂₀ heteroaryl, each of which are independently and optionally substituted with one or more groups selected from:
—(C₁-C₁₂ alkyldiyl)-N(R⁷)—*;
—(C₁-C₁₂ alkyldiyl)-N(R⁷)₂;
—(C₁-C₁₂ alkyldiyl)-OR⁷;
—(C₃-C₁₂ carbocyclyl);
—(C₃-C₁₂ carbocyclyl)-*;
—(C₃-C₁₂ carbocyclyl)-(C₁-C₁₂ alkyldiyl)-NR⁷—*;
—(C₃-C₁₂ carbocyclyl)-(C₁-C₁₂ alkyldiyl)-N(R⁷)₂;
—(C₃-C₁₂ carbocyclyl)-NR⁷—C(═NR⁷)NR⁷—*;
—(C₆-C₂₀ aryl);
—(C₆-C₂₀ aryldiyl)-*;
—(C₆-C₂₀ aryldiyl)-N(R⁷)—*;
—(C₆-C₂₀ aryldiyl)-(C₁-C₁₂ alkyldiyl)-N(R⁷)—*;
—(C₆-C₂₀ aryldiyl)-(C₁-C₁₂ alkyldiyl)-(C₂-C₂₀ heterocyclyldiyl)-*;
—(C₆-C₂₀ aryldiyl)-(C₁-C₁₂ alkyldiyl)-N(R⁷)₂;
—(C₆-C₂₀ aryldiyl)-(C₁-C₁₂ alkyldiyl)-NR⁷—C(═NR^{7a})N(R⁷)—*;
—(C₂-C₂₀ heterocyclyl);
—(C₂-C₂₀ heterocyclyl)-*;
—(C₂-C₉ heterocyclyl)-(C₁-C₁₂ alkyldiyl)-NR⁷—*;
—(C₂-C₉ heterocyclyl)-(C₁-C₁₂ alkyldiyl)-N(R⁷)₂;
—(C₂-C₉ heterocyclyl)-C(═O)—(C₁-C₁₂ alkyldiyl)-N(R⁷)—*;
—(C₂-C₉ heterocyclyl)-NR⁷—C(═NR^{7a})NR⁷—*;
—(C₂-C₉ heterocyclyl)-NR⁷—(C₆-C₂₀ aryldiyl)-(C₁-C₁₂ alkyldiyl)-N(R⁷)—*;
—(C₂-C₉ heterocyclyl)-(C₆-C₂₀ aryldiyl)-*;
—(C₁-C₂₀ heteroaryl);
—(C₁-C₂₀ heteroaryl)-*;
—(C₁-C₂₀ heteroaryl)-(C₁-C₁₂ alkyldiyl)-N(R⁷)—*;
—(C₁-C₂₀ heteroaryl)-(C₁-C₁₂ alkyldiyl)-N(R⁷)₂;
—(C₁-C₂₀ heteroaryl)-NR⁷—C(═NR^{7a})N(R⁷)—*;
—(C₁-C₂₀ heteroaryl)-N(R⁷)C(═O)—(C₁-C₁₂ alkyldiyl)-N(R⁷)—*;
—C(═O)—*;
—C(═O)—(C₁-C₁₂ alkyldiyl)-N(R⁷)—*;
—C(═O)—(C₂-C₂₀ heterocyclyldiyl)-*;
—C(═O)N(R⁷)₂;
—C(═O)N(R⁷)—*;
—C(═O)N(R⁷)—(C₁-C₁₂ alkyldiyl)-N(R⁷)C(═O)R⁷;
—C(═O)N(R⁷)—(C₁-C₁₂ alkyldiyl)-N(R⁷)C(═O)N(R⁷)₂;
—C(═O)NR⁷—(C₁-C₁₂ alkyldiyl)-N(R⁷)CO₂R⁷;
—C(═O)NR⁵—(C₁-C₁₂ alkyldiyl)-N(R⁵⁷)C(═NR^{57a})N(R⁵⁷)₂;
—C(═O)NR⁵—(C₁-C₁₂ alkyldiyl)-NR⁵⁷C(═NR^{7a})R⁷;
—C(═O)NR⁵—(C₁-C₈ alkyldiyl)-NR⁷(C₂-C₅ heteroaryl);
—C(═O)NR⁷—(C₁-C₂₀ heteroaryldiyl)-N(R⁷)—*;
—C(═O)NR⁷—(C₁-C₂₀ heteroaryldiyl)-*;
—C(═O)NR⁷—(C₁-C₂₀ heteroaryldiyl)-(C₁-C₁₂ alkyldiyl)-N(R⁷)₂;
—C(═O)NR⁷—(C₁-C₂₀ heteroaryldiyl)-(C₂-C₂₀ heterocyclyldiyl)-C(═O)NR⁷—(C₁-C₁₂ alkyldiyl)-NR⁷—*;
—N(R⁷)₂;
—N(R⁷)—*;
—N(R⁷)C(═O)R⁷;
—N(R⁷)C(═O)—*;
—N(R⁷)C(═O)N(R⁷)₂;
—N(R⁷)C(═O)N(R⁷)—*;
—N(R⁷)CO₂R⁷;
—NR⁷C(═NR^{7a})N(R⁷)₂;
—NR⁷C(═NR^{7a})N(R⁷)—*;
—NR⁷C(═NR^{7a})R⁷;
—N(R⁷)C(═O)—(C₁-C₁₂ alkyldiyl)-N(R⁷)—*;
—N(R⁷)—(C₂-C₅ heteroaryl);
—N(R⁷)—S(═O)₂—(C₁-C₁₂ alkyl);
—O—(C₁-C₁₂ alkyl);
—O—(C₁-C₁₂ alkyldiyl)-N(R⁷)₂;
—O—(C₁-C₁₂ alkyldiyl)-N(R⁷)—*;
—O—C(═O)N(R⁷)₂;
—O—C(═O)N(R⁷)—*;
—O—(R⁷)—*;
—OR⁷;
—S(═O)₂—(C₂-C₂₀ heterocyclyldiyl)-*;
—S(═O)₂—(C₂-C₂₀ heterocyclyldiyl)-(C₁-C₁₂ alkyldiyl)-N(R⁷)₂;
—S(═O)₂—(C₂-C₂₀ heterocyclyldiyl)-(C₁-C₁₂ alkyldiyl)-NR⁷—*; and
—S(═O)₂—(C₂-C₂₀ heterocyclyldiyl)-(C₁-C₁₂ alkyldiyl)-OH;
or R⁵ and R⁶ together form a 5- or 6-membered heterocyclyl ring;
R⁷ is independently selected from the group consisting of H, C₆-C₂₀ aryl, C₃-C₁₂ carbocyclyl, C₆-C₂₀ aryldiyl, C₁-C₁₂ alkyl, and C₁-C₁₂ alkyldiyl, or two R⁵ groups together form a 5- or 6-membered heterocyclyl ring;
R^{7a} is selected from the group consisting of C₆-C₂₀ aryl and C₁-C₂₀ heteroaryl;
where the asterisk * indicates the attachment site of L, and where one of R¹, R², R³, R⁴, R⁵ and R⁶ is attached to L; and
alkyl, alkyldiyl, alkenyl, alkenyldiyl, alkynyl, alkynyldiyl, aryl, aryldiyl, carbocyclyl, carbocyclyldiyl, heterocyclyl, heterocyclyldiyl, heteroaryl, and heteroaryldiyl are independently and optionally substituted with one or more groups independently selected from F, Cl, Br, I, —CN, —CH₃, —CH₂CH₃, —CH═CH₂, —C═CH, —C═CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —O(CH₂CH₂O)r-(CH₂)ₘCO₂H, —O(CH₂CH₂O)~H, —OCH₂F, —OCHF₂, —OCF₃, —OP(O)(OH)₂, —S(O)₂N(CH₃)₂, —SCH₃, —S(O)₂CH₃, and —S(O)₃H.

2. The immunoconjugate of claim 1 wherein the linker L is a divalent or a branched, trivalent linker.

3. The immunoconjugate of claim 1 wherein the linker L is selected from the group consisting of:
—C(═O)-PEG-;
—C(═O)-PEG-C(═O)N(R⁸)—(C₁-C₁₂ alkyldiyl)-C(═O)-Gluc-;
—C(═O)-PEG-(C₂-C₂₀ heterocyclyldiyl)-;
—C(═O)-PEG-(C₂-C₂₀ heterocyclyldiyl)-(C₁-C₁₂ alkyldiyl)-;
—C(═O)-PEG-O—;
—C(═O)-PEG-O—C(═O)—;
—C(═O)-PEG-C(═O)—;
—C(═O)-PEG-C(═O)-PEP-;
—C(═O)-PEG-N(R⁸)—;
—C(═O)-PEG-N(R⁸)—C(═O)—;
—C(═O)-PEG-N(R⁸)-PEG-C(═O)-PEP-;
—C(═O)-PEG-N⁺(R⁸)₂-PEG-C(═O)-PEP-;
—C(═O)-PEG-C(═O)-PEP-N(R⁸)—(C₁-C₁₂ alkyldiyl)-;
—C(═O)-PEG-C(═O)-PEP-N(R⁸)—(C₁-C₁₂ alkyldiyl)N(R⁸)C(═O)—(C₂-C₅ monoheterocyclyldiyl)-;
—C(═O)-PEG-SS-(C₁-C₁₂ alkyldiyl)-OC(═O)—;
—C(═O)-PEG-SS-(C₁-C₁₂ alkyldiyl)-C(═O)—;
—C(═O)—(C₁-C₁₂ alkyldiyl)-C(═O)-PEP-;
—C(═O)—(C₁-C₁₂ alkyldiyl)-C(═O)-PEP-N(R⁸)—(C₁-C₁₂ alkyldiyl)-;
—C(═O)—(C₁-C₁₂ alkyldiyl)-C(═O)-PEP-N(R⁸)—(C₁-C₁₂ alkyldiyl)-N(R⁸)—C(═O);
—C(═O)—(C₁-C₁₂ alkyldiyl)-C(═O)-PEP-N(R⁸)—(C₁-C₁₂ alkyldiyl)-N(R⁸)C(═O)—(C₂-C₅ monoheterocyclyldiyl)-;
-succinimidyl-(CH₂)ₘ—C(═O)N(R⁸)-PEG-;
-succinimidyl-(CH₂)ₘ—C(═O)N(R⁸)-PEG-C(═O)N(R⁸)—(C₁-C₁₂ alkyldiyl)-C(═O)-Gluc-;
-succinimidyl-(CH₂)ₘ—C(═O)N(R³)—(C₂-C₂₀ heterocyclyldiyl)-;
-succinimidyl-(CH₂)ₘ—C(═O)N(R³)-PEG-(C₂-C₂₀ heterocyclyldiyl)-(C₁-C₁₂ alkyldiyl)-;
-succinimidyl-(CH₂)ₘ—C(═O)N(R⁸)-PEG-O—;
-succinimidyl-(CH₂)ₘ—C(═O)N(R⁸)-PEG-O—C(═O)—;
-succinimidyl-(CH₂)ₘ—C(═O)N(R⁸)-PEG-C(═O)—;
-succinimidyl-(CH₂)ₘ—C(═O)N(R⁸)-PEG-N(R⁸)—;
-succinimidyl-(CH₂)ₘ—C(═O)N(R⁸)-PEG-N(R⁸)—C(═O)—;
-succinimidyl-(CH₂)ₘ—C(═O)N(R⁸)-PEG-C(═O)-PEP-;
-succinimidyl-(CH₂)ₘ—C(═O)N(R⁸)-PEG-SS-(C₁-C₁₂ alkyldiyl)-OC(═O)—;
-succinimidyl-(CH₂)ₘ—C(═O)-PEP-N(R⁸)—(C₁-C₁₂ alkyldiyl)-;
-succinimidyl-(CH₂)ₘ—C(═O)-PEP-N(R⁸)—(C₁-C₁₂ alkyldiyl)N(R⁸)C(═O)—; and
-succinimidyl-(CH₂)ₘ—C(═O)-PEP-N(R⁸)—(C₁-C₁₂ alkyldiyl)N(R⁸)C(═O)—(C₂-C₅ monoheterocyclyldiyl)-;
R⁸ is independently H or C₁-C₆ alkyl;
PEG has the formula: —(CH₂CH₂O)ₙ—(CH₂)ₘ—; m is an integer from 1 to 5, and n is an integer from 1 to 50;
Gluc has the formula: 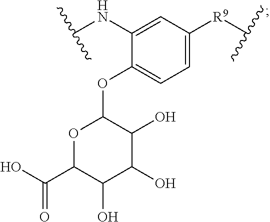
PEP has the formula: 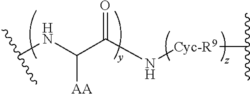
where AA is independently selected from a natural or unnatural amino acid side chain, or one or more of AA, and an adjacent nitrogen atom form a 5-membered ring proline amino acid, and the wavy line indicates a point of attachment;
Cyc is selected from C₆-C₂₀ aryldiyl and C₁-C₂₀ heteroaryldiyl, optionally substituted with one or more groups selected from F, Cl, NO₂, —OH, —OCH₃, and a glucuronic acid having the structure: 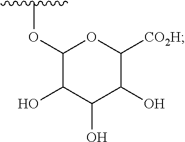 [SMILES: *OC1OC(C(=O)O)C(O)C(O)C1O]
R⁹ is selected from the group consisting of —CH(R¹⁰)O—, —CH₂—, —CH₂N(R¹⁰)—, and —CH(R¹⁰)O—C(═O)—, where R¹⁰ is selected from H, C₁-C₆ alkyl, C(═O)—C₁-C₆ alkyl, and —C(═O)N(R¹)₂, where R¹¹ is independently selected from the group consisting of H, C₁-C₁₂ alkyl, and —(CH₂CH₂O)ₙ—(CH₂)ₘ—OH, where m is an integer from 1 to 5, and n is an integer from 2 to 50, or two R¹¹ groups together form a 5- or 6-membered heterocyclyl ring;
y is an integer from 2 to 12; and
z is 0 or 1.

4. The immunoconjugate of claim 1 wherein the antibody is an immune checkpoint inhibitor.

5. The immunoconjugate of claim 1 wherein the antibody is an antibody construct that has an antigen binding domain that binds an antigen selected from PD-L1, HER2, CEA, and TROP2.

6. The immunoconjugate of claim 5 wherein the antibody is selected from the group consisting of atezolizumab, durvalumab, avelumab, trastuzumab, pertuzumab, margetuximab, HT-19, labetuzumab, and sacituzumab.

7. The immunoconjugate of claim 1 wherein one of Z¹, Z², Z³, and Z⁴ are N.

8. The immunoconjugate of claim 7 wherein Z¹ is N.

9. The immunoconjugate of claim 7 wherein Z² is N.

10. The immunoconjugate of claim 7 wherein Z³ is N.

11. The immunoconjugate of claim 7 wherein Z⁴ is N.

12. The immunoconjugate of claim 1 wherein two of Z¹, Z², Z³, and Z⁴ are N.

13. The immunoconjugate of any one of claims 1 to 12 wherein R⁵ and R⁶ are independently selected from C₁-C₈ alkyl, —O—(C₁-C₁₂ alkyl), —(C₁-C₁₂ alkyldiyl)-OR⁵, —(C₁-C₈ alkyldiyl)-N(R⁵)CO₂R⁵, —(C₁-C₁₂ alkyl)-OC(O)N(R⁵)₂, —O—(C₁-C₁₂ alkyl)-N(R⁵)CO₂R⁵, and —O—(C₁-C₁₂ alkyl)-OC(O)N(R⁵)₂.

14. The immunoconjugate of claim 13 wherein R⁵ is C₁-C₈ alkyl and R⁶ is —O—(C₁-C₁₂ alkyl).

15. The immunoconjugate of claim 13 wherein R⁵ is —CH₂CH₂CH₃ and R⁶ is selected from —CH₂CH₂CH₂NHCO₂(t-Bu), —OCH₂CH₂NHCO₂(cyclobutyl), and —CH₂CH₂CH₂NHCO₂(cyclobutyl).

16. The immunoconjugate of claim 13 wherein R⁵ and R⁶ are each independently selected from —CH₂CH₂CH₃, —OCH₂CH₃, —OCH₂CF₃, —CH₂CH₂CF₃, —OCH₂CH₂OH, and —CH₂CH₂CH₂OH.

17. The immunoconjugate of claim 16 wherein R⁵ is —CH₂CH₂CH₃ and R⁶ is —OCH₂CH₃.

18. The immunoconjugate of any one of claims 1 to 12 wherein R⁶ is selected from the group consisting of

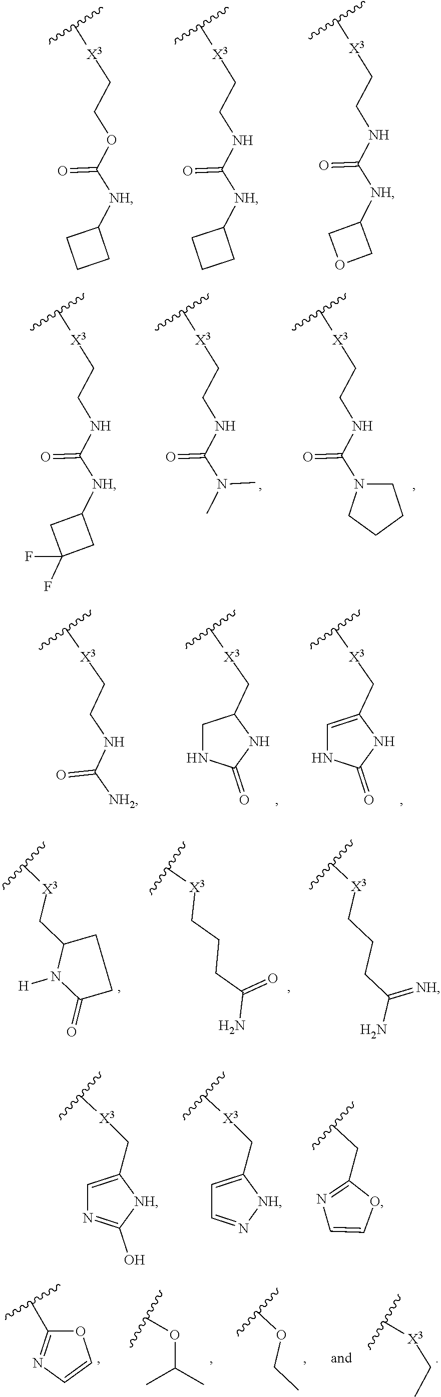

19. The immunoconjugate of any one of claims 1 to 12 where R¹ is attached to L.

20. The immunoconjugate of any one of claims 1 to 12 where R² is attached to L.

21. The immunoconjugate of any one of claims 1 to 12 where R³ is attached to L.

22. The immunoconjugate of any one of claims 1 to 12 where R⁴ is attached to L.

23. The immunoconjugate of any one of claims 1 to 12 where R⁵ or R⁶ is attached to L.

24. The immunoconjugate of any one of claims 1 to 12 wherein L is —C(═O)-PEG- or —C(═O)-PEG-C(═O)—.

25. The immunoconjugate of any one of claims 1 to 12 wherein L is attached to a cysteine thiol of the antibody.

26. The immunoconjugate of any one of claims 1 to 12 wherein for the PEG, m is 1 or 2, and n is an integer from 2 to 10.

27. The immunoconjugate of claim 26 wherein n is 10.

28. The immunoconjugate of any one of claims 1 to 12 wherein L comprises PEP and PEP is a dipeptide and has the formula:

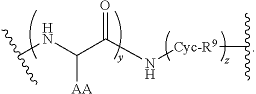

29. The immunoconjugate of claim 28 wherein AA is independently selected from H, —CH₃, —CH(CH₃)₂, —CH₂(C₆H₅), —CH₂CH₂CH₂CH₂NH₂, —CH₂CH₂CH₂NHC(NH)NH₂, —CHCH(CH₃)CH₃, —CH₂SO₃H, and —CH₂CH₂CH₂NHC(O)NH₂; or two AA form a 5-membered ring proline amino acid.

30. The immunoconjugate of claim 28 wherein PEP is a dipeptide and has the formula: 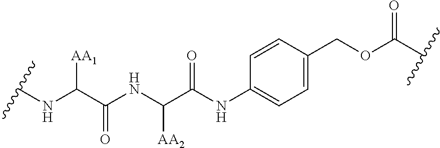
wherein AA₁ and AA₂ are independently selected from a side chain of a naturally-occurring amino acid.

31. The immunoconjugate of claim 30 wherein AA₁ is —CH(CH₃)₂, and AA₂ is —CH₂CH₂CH₂NHC(O)NH₂.

32. The immunoconjugate of any one of claims 1 to 12 wherein L is selected from the structures: 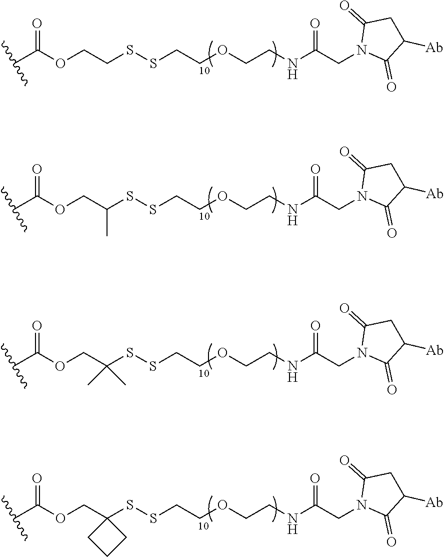
where the wavy line indicates the attachment to one of R¹, R², R³, R⁴, R⁵, and R⁶.

33. An aza-benzazepine-linker compound of Formula II: 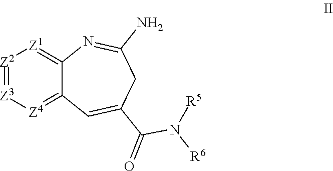
wherein
Z¹ is selected from CR¹ and N;
Z² is selected from CR² and N;
Z³ is selected from CR³ and N;
Z⁴ is selected from CR⁴ and N;
where one or two of Z¹, Z², Z³, and Z⁴ are N;
R¹, R², R³, R⁴, R⁵, and R⁶ are independently selected from the group consisting of H, C(═O), C(═O)N(R⁵), O, N(R⁵), S, S(O)₂, S(O)₂N(R⁵), C₁-C₁₂ alkyl, C₂-C₆ alkenyl, C₂-C₆ alkynyl, C₃-C₁₂ carbocyclyl, C₆-C₂₀ aryl, C₂-C₉ heterocyclyl, and C₁-C₂₀ heteroaryl, each of which are independently and optionally substituted with one or more groups selected from:
—(C₁-C₁₂ alkyldiyl)-N(R⁷)—*;
—(C₁-C₁₂ alkyldiyl)-N(R⁷)₂;
—(C₁-C₁₂ alkyldiyl)-OR⁷;
—(C₃-C₁₂ carbocyclyl);
—(C₃-C₁₂ carbocyclyl)-*;
—(C₃-C₁₂ carbocyclyl)-(C₁-C₁₂ alkyldiyl)-NR⁷—*;
—(C₃-C₁₂ carbocyclyl)-(C₁-C₁₂ alkyldiyl)-N(R⁷)₂;
—(C₃-C₁₂ carbocyclyl)-NR⁷—C(═NR⁷)NR⁷—*;
—(C₆-C₂₀ aryl);
—(C₆-C₂₀ aryldiyl)-*;
—(C₆-C₂₀ aryldiyl)-N(R⁷)—*;
—(C₆-C₂₀ aryldiyl)-(C₁-C₁₂ alkyldiyl)-N(R⁷)—*;
—(C₆-C₂₀ aryldiyl)-(C₁-C₁₂ alkyldiyl)-(C₂-C₂₀ heterocyclyldiyl)-*;
—(C₆-C₂₀ aryldiyl)-(C₁-C₁₂ alkyldiyl)-N(R⁷)₂;
—(C₆-C₂₀ aryldiyl)-(C₁-C₁₂ alkyldiyl)-NR⁷—C(═NR^{7a})N(R⁷)—*;
—(C₂-C₂₀ heterocyclyl);
—(C₂-C₂₀ heterocyclyl)-*;
—(C₂-C₉ heterocyclyl)-(C₁-C₁₂ alkyldiyl)-NR⁷—*;
—(C₂-C₉ heterocyclyl)-(C₁-C₁₂ alkyldiyl)-N(R⁷)₂;
—(C₂-C₉ heterocyclyl)-C(═O)—(C₁-C₁₂ alkyldiyl)-N(R⁷)—*;
—(C₂-C₉ heterocyclyl)-NR⁷—C(═NR^{7a})NR⁷—*;
—(C₂-C₉ heterocyclyl)-NR⁷—(C₆-C₂₀ aryldiyl)-(C₁-C₁₂ alkyldiyl)-N(R⁷)—*;
—(C₂-C₉ heterocyclyl)-(C₆-C₂₀ aryldiyl)-*;
—(C₁-C₂₀ heteroaryl);
—(C₁-C₂₀ heteroaryl)-*;
—(C₁-C₂₀ heteroaryl)-(C₁-C₁₂ alkyldiyl)-N(R⁷)—*;
—(C₁-C₂₀ heteroaryl)-(C₁-C₁₂ alkyldiyl)-N(R⁷)₂;
—(C₁-C₂₀ heteroaryl)-NR⁷—C(═NR^{7a})N(R⁷)—*;
—(C₁-C₂₀ heteroaryl)-N(R⁷)C(═O)—(C₁-C₁₂ alkyldiyl)-N(R⁷)—*;
—C(═O)—*;
—C(═O)—(C₁-C₁₂ alkyldiyl)-N(R⁷)—*;
—C(═O)—(C₂-C₂₀ heterocyclyldiyl)-*;
—C(═O)N(R⁷)₂;
—C(═O)N(R⁷)—*;
—C(═O)N(R⁷)—(C₁-C₁₂ alkyldiyl)-N(R⁷)C(═O)R⁷;
—C(═O)N(R⁷)—(C₁-C₁₂ alkyldiyl)-N(R⁷)C(═O)N(R⁷)₂;
—C(═O)NR⁷—(C₁-C₁₂ alkyldiyl)-N(R⁷)CO₂R⁷;
—C(═O)NR⁵—(C₁-C₁₂ alkyldiyl)-N(R⁵⁷)C(═NR^{57a})N(R⁵⁷)₂;
—C(═O)NR⁵—(C₁-C₁₂ alkyldiyl)-NR⁵⁷C(═NR^{7a})R⁷;
—C(═O)NR⁵—(C₁-C₈ alkyldiyl)-NR⁷(C₂-C₅ heteroaryl);
—C(═O)NR⁷—(C₁-C₂₀ heteroaryldiyl)-N(R⁷)—*;
—C(═O)NR⁷—(C₁-C₂₀ heteroaryldiyl)-*;
—C(═O)NR⁷—(C₁-C₂₀ heteroaryldiyl)-(C₁-C₁₂ alkyldiyl)-N(R⁷)₂;
—C(═O)NR⁷—(C₁-C₂₀ heteroaryldiyl)-(C₂-C₂₀ heterocyclyldiyl)-C(═O)NR⁷—(C₁-C₁₂ alkyldiyl)-NR⁷—*;
—N(R⁷)₂;
—N(R⁷)—*;
—N(R⁷)C(═O)R⁷;
—N(R⁷)C(═O)—*;
—N(R⁷)C(═O)N(R⁷)₂;
—N(R⁷)C(═O)N(R⁷)—*;
—N(R⁷)CO₂R⁷;
—NR⁷C(═NR^{7a})N(R⁷)₂;
—NR⁷C(═NR^{7a})N(R⁷)—*;
—NR⁷C(═NR^{7a})R⁷;
—N(R⁷)C(═O)—(C₁-C₁₂ alkyldiyl)-N(R⁷)—*;
—N(R⁷)—(C₂-C₅ heteroaryl);
—N(R⁷)—S(═O)₂—(C₁-C₁₂ alkyl);
—O—(C₁-C₁₂ alkyl);
—O—(C₁-C₁₂ alkyldiyl)-N(R⁷)₂;
—O—(C₁-C₁₂ alkyldiyl)-N(R⁷)—*;
—O—C(═O)N(R⁷)₂;
—O—C(═O)N(R⁷)—*;
—O—(R⁷)—*;
—OR⁷;
—S(═O)₂—(C₂-C₂₀ heterocyclyldiyl)-*;
—S(═O)₂—(C₂-C₂₀ heterocyclyldiyl)-(C₁-C₁₂ alkyldiyl)-N(R⁷)₂;
—S(═O)₂—(C₂-C₂₀ heterocyclyldiyl)-(C₁-C₁₂ alkyldiyl)-NR⁷—*; and
—S(═O)₂—(C₂-C₂₀ heterocyclyldiyl)-(C₁-C₁₂ alkyldiyl)-OH;
or R⁵ and R⁶ together form a 5- or 6-membered heterocyclyl ring;
R⁷ is independently selected from the group consisting of H, C₆-C₂₀ aryl, C₃-C₁₂ carbocyclyl, C₆-C₂₀ aryldiyl, C₁-C₁₂ alkyl, and C₁-C₁₂ alkyldiyl, or two R⁵ groups together form a 5- or 6-membered heterocyclyl ring;
R^{7a} is selected from the group consisting of C₆-C₂₀ aryl and C₁-C₂₀ heteroaryl;
where the asterisk * indicates the attachment site of L, and where one of R¹, R², R³, R⁴, R⁵ and R⁶ is attached to L; and
alkyl, alkyldiyl, alkenyl, alkenyldiyl, alkynyl, alkynyldiyl, aryl, aryldiyl, carbocyclyl, carbocyclyldiyl, heterocyclyl, heterocyclyldiyl, heteroaryl, and heteroaryldiyl are independently and optionally substituted with one or more groups independently selected from F, Cl, Br, I, —CN, —CH₃, —CH₂CH₃, —CH═CH₂, —C═CH, —C—CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —O(CH₂CH₂O)r-(CH₂)ₘCO₂H, —O(CH₂CH₂O)~H, —OCH₂F, —OCHF₂, —OCF₃, —OP(O)(OH)₂, —S(O)₂N(CH₃)₂, —SCH₃, —S(O)₂CH₃, and —S(O)₃H.

34. The aza-benzazepine-linker compound of claim 33 wherein L is selected from:
Q-C(═O)-PEG-;
Q-C(═O)-PEG-C(═O)N(R⁸)—(C₁-C₁₂ alkyldiyl)-C(═O)-Gluc-;
Q-C(═O)-PEG-O—;
Q-C(═O)-PEG-O—C(═O)—;
Q-C(═O)-PEG-C(═O)—;
Q-C(═O)-PEG-C(═O)-PEP-;
Q-C(═O)-PEG-N(R⁸)—;
Q-C(═O)-PEG-N(R⁸)—C(═O)—;
Q-C(═O)-PEG-N(R⁸)-PEG-C(═O)-PEP-;
Q-C(═O)-PEG-N⁺(R⁸)₂-PEG-C(═O)-PEP-;
Q-C(═O)-PEG-C(═O)-PEP-N(R⁸)—(C₁-C₁₂ alkyldiyl)-;
Q-C(═O)-PEG-C(═O)-PEP-N(R⁸)—(C₁-C₁₂ alkyldiyl)N(R⁸)C(═O)—(C₂-C₅ monoheterocyclyldiyl)-;
Q-C(═O)-PEG-SS-(C₁-C₁₂ alkyldiyl)-OC(═O)—;
Q-C(═O)-PEG-SS-(C₁-C₁₂ alkyldiyl)-C(═O)—;
Q-C(═O)—(C₁-C₁₂ alkyldiyl)-C(═O)-PEP-;
Q-C(═O)—(C₁-C₁₂ alkyldiyl)-C(═O)-PEP-N(R⁸)—(C₁-C₁₂ alkyldiyl)-;
Q-C(═O)—(C₁-C₁₂ alkyldiyl)-C(═O)-PEP-N(R⁸)—(C₁-C₁₂ alkyldiyl)-N(R⁸)—C(═O);
Q-C(═O)—(C₁-C₁₂ alkyldiyl)-C(═O)-PEP-N(R⁸)—(C₁-C₁₂ alkyldiyl)-N(R⁸)C(═O)—(C₂-C₅ monoheterocyclyldiyl)-;
Q-(CH₂)ₘC(═O)N(R⁸)-PEG-;
Q-(CH₂)ₘC(═O)N(R⁸)-PEG-C(═O)N(R⁸)—(C₁-C₁₂ alkyldiyl)-C(═O)-Gluc-;
Q-(CH₂)ₘC(═O)N(R⁸)-PEG-O—;
Q-(CH₂)ₘC(═O)N(R⁸)-PEG-O—C(═O)—;
Q-(CH₂)ₘC(═O)N(R⁸)-PEG-C(═O)—;
Q-(CH₂)ₘC(═O)N(R⁸)-PEG-N(R⁸)-Q-(CH₂)ₘC(═O)N(R⁸)-PEG-N(R⁸)—C(═O)—;
Q-(CH₂)ₘC(═O)N(R⁸)-PEG-C(═O)-PEP-;
Q-(CH₂)ₘC(═O)N(R⁸)-PEG-SS-(C₁-C₁₂ alkyldiyl)-OC(═O)—;
Q-(CH₂)ₘC(═O)-PEP-N(R⁸)—(C₁-C₁₂ alkyldiyl)-;
Q-(CH₂)ₘC(═O)-PEP-N(R⁸)—(C₁-C₁₂ alkyldiyl)N(R⁸)C(═O)—; and
Q-(CH₂)ₘC(═O)-PEP-N(R⁸)—(C₁-C₁₂ alkyldiyl)N(R⁸)C(═O)—(C₂-C₅ monoheterocyclyldiyl)-;
R⁸ is independently H or C₁-C₆ alkyl;
PEG has the formula: —(CH₂CH₂O)ₙ—(CH₂)ₘ—; m is an integer from 1 to 5, and n is an integer from 2 to 50;
Gluc has the formula: 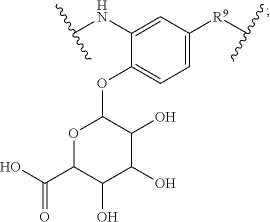
PEP has the formula: 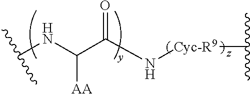
where AA is independently selected from a natural or unnatural amino acid side chain, or one or more of AA, and an adjacent nitrogen atom form a 5-membered ring proline amino acid, and the wavy line indicates a point of attachment;
Cyc is selected from C₆-C₂₀ aryldiyl and C₁-C₂₀ heteroaryldiyl, optionally substituted with one or more groups selected from F, Cl, NO₂, —OH, —OCH₃, and a glucuronic acid having the structure: 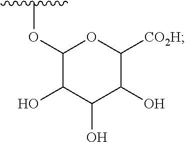 [SMILES: *OC1OC(C(=O)O)C(O)C(O)C1O]
R⁹ is selected from the group consisting of —CH(R¹⁰)O—, —CH₂—, —CH₂N(R¹⁰)—, and —CH(R¹⁰)O—C(═O)—, where R¹⁰ is selected from H, C₁-C₆ alkyl, C(═O)—C₁-C₆ alkyl, and —C(═O)N(R¹)₂, where R¹¹ is independently selected from the group consisting of H, C₁-C₁₂ alkyl, and —(CH₂CH₂O)ₙ—(CH₂)ₘ—OH, where m is an integer from 1 to 5, and n is an integer from 2 to 50, or two R¹¹ groups together form a 5- or 6-membered heterocyclyl ring;
y is an integer from 2 to 12;
z is 0 or 1; and
Q is selected from the group consisting of N-hydroxysuccinimidyl, N-hydroxysulfosuccinimidyl, maleimide, and phenoxy substituted with one or more groups independently selected from F, Cl, NO₂, and SO₃—;

35. The aza-benzazepine-linker compound of claim 34 wherein Q is selected from:

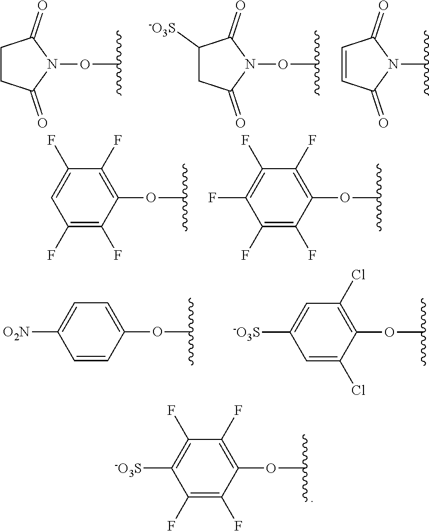

36. The aza-benzazepine-linker compound of claim 34 wherein Q is phenoxy substituted with one or more F.

37. The aza-benzazepine-linker compound of claim 34 wherein Q is 2,3,5,6-tetrafluorophenoxy.

38. The aza-benzazepine-linker compound of claim 34 wherein Q is maleimide.

39. An aza-benzazepine-linker compound selected from Table 2a or Table 2b.

40. An immunoconjugate prepared by conjugation of an antibody with an aza-benzazepine-linker compound selected from Table 2a or Table 2b.

41. A pharmaceutical composition comprising a therapeutically effective amount of an immunoconjugate according to any one of claims 1 to 32, and one or more pharmaceutically acceptable diluent, vehicle, carrier or excipient.

42. A method for treating cancer comprising administering a therapeutically effective amount of an immunoconjugate according to any one of claims 1 to 32 to a patient in need thereof, wherein the cancer is selected from cervical cancer, endometrial cancer, ovarian cancer, prostate cancer, pancreatic cancer, esophageal cancer, bladder cancer, urinary tract cancer, urothelial carcinoma, lung cancer, non-small cell lung cancer, Merkel cell carcinoma, colon cancer, colorectal cancer, gastric cancer, and breast cancer.

43. The method of claim 42, wherein the cancer is susceptible to a pro-inflammatory response induced by TLR7 and/or TLR8 agonism.

44. The method of claim 42, wherein the cancer is selected from triple-negative breast cancer, metastatic Merkel cell carcinoma, and gastroesophageal junction adenocarcinoma.

45. The method of claim 42, wherein the immunoconjugate is administered to the patient intravenously, intratumorally, or subcutaneously.

46. The method of claim 42, wherein the immunoconjugate is administered to the patient at a dose of about 0.01 to 20 mg per kg of body weight.

47. Use of an immunoconjugate according to any one of claims 1 to 32 for treating cancer, wherein the cancer is selected from cervical cancer, endometrial cancer, ovarian cancer, prostate cancer, pancreatic cancer, esophageal cancer, bladder cancer, urinary tract cancer, urothelial carcinoma, lung cancer, non-small cell lung cancer, Merkel cell carcinoma, colon cancer, colorectal cancer, gastric cancer, and breast cancer.

48. A method of preparing an immunoconjugate of Formula I of claim 1 wherein the aza-benzazepine-linker compound of claim 34 is conjugated with the antibody.
